(19) United States
(12) Patent Application Publication
(10) Pub. No.: US 2024/0173426 A1
(43) Pub. Date: May 30, 2024

(54) COMPOSITIONS AND METHODS FOR FIBROBLAST GROWTH FACTOR RECEPTOR 3-MEDIATED DELIVERY TO ASTROCYTES

(71) Applicant: Regeneron Pharmaceuticals, Inc., Tarrytown, NY (US)

(72) Inventors: Jeffery Haines, New York, NY (US); Keith Crosby, Pleasantville, NY (US); Yan Yang, Mamaroneck, NY (US); Christopher Daly, New York, NY (US); William Olson, Yorktown Heights, NY (US); Tammy T. Huang, Cross River, NY (US); Drew Dudgeon, Montvale, NJ (US); Robert Babb, River Edge, NY (US)

(73) Assignee: Regeneron Pharmaceuticals, Inc., Tarrytown, NY (US)

(21) Appl. No.: 18/509,030

(22) Filed: Nov. 14, 2023

Related U.S. Application Data
(60) Provisional application No. 63/383,673, filed on Nov. 14, 2022.
(60) Provisional application No. 63/587,585, filed on Oct. 3, 2023.

(51) Int. Cl. A61K 47/68; A61K 47/69; C12N 9/22; C12N 15/11; C12N 15/113

(52) U.S. Cl. CPC A61K 47/6849; A61K 47/6811; A61K 47/6815; A61K 47/6889; A61K 47/6929; C12N 9/22; C12N 15/11; C12N 15/113; C12N 2310/14; C12N 2310/20

(57) ABSTRACT

The present invention provides, in part, protein-drug conjugates comprising an anti-fibroblast growth factor receptor 3 (FGFR3) (e.g., human FGFR3) antigen-binding protein (e.g., scFv, Fab) conjugated to a molecular cargo (e.g., polynucleotides, polypeptides, liposomes or lipid nanoparticles) for delivery of the molecular cargo to a targeted tissue (e.g., brain). Methods for treating various diseases or disorders, such as neurological diseases, with the conjugates are provided.

### CROSS REFERENCE TO RELATED APPLICATIONS

This application claims priority to U.S. Provisional Application No. 63/383,673, filed Nov. 14, 2022, and U.S. Provisional Application No. 63/587,585, filed Oct. 3, 2023, the disclosure of each of which is hereby incorporated by reference in its entirety.

### SEQUENCE LISTING

The instant application contains a Sequence Listing which has been submitted electronically in XML file format and is hereby incorporated by reference in its entirety. Said XML copy, created on Nov. 6, 2023, is named 250298_000564_SL.xml and is 476,394 bytes in size.

### FIELD OF THE INVENTION

The present invention relates to protein-drug conjugates including an anti-fibroblast growth factor receptor 3 (FGFR3) antigen-binding protein conjugated to a molecular cargo, as well as method of treating diseases with such protein-drug conjugates.

### BACKGROUND OF THE INVENTION

A lack of understanding of the molecular signatures of disease, together with a limited toolbox of robust model systems, has contributed to a failure to develop successful disease modifying therapies targeting various neurological diseases and/or disorders. Such neurological disorders range from progressive neurodegenerative diseases (e.g., Alzheimer's disease, Parkinson's disease), to neurodevelopmental diseases (e.g., Alexander disease, multiple sulfatase deficiency), and can also include those associated with physical injury (e.g., traumatic brain injury, spinal cord injury, and stroke). Advancements are being made on a number of fronts to identify agents that can prevent, slow or halt disease progression, yet present therapies provide very little relief. Accordingly, there remains a need to develop therapies that can disrupt the onset and/or the course of neurological diseases, in particular, in order to improve the quality of the lives of those suffering from such diseases.

### SUMMARY OF THE INVENTION

The present disclosure provides, among other things, a protein-drug conjugate comprising an antigen-binding protein that binds specifically to fibroblast growth factor receptor 3 (FGFR3) and is conjugated to a molecular cargo.

In one aspect, provided herein is a protein-drug conjugate comprising an antigen-binding protein that binds specifically to fibroblast growth factor receptor 3 (FGFR3) and is conjugated to a molecular cargo.

In some embodiments, the antigen binding protein comprises an antibody or antigen-binding fragment thereof. In some embodiments, In some embodiments, the protein drug conjugate comprises a heavy chain variable region (HCVR or V H) and/or a light chain variable region (LCVR or V L). In some embodiments, the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BITE), trispecific antibody, or chemically modified derivatives thereof.

In some embodiments, the antigen-binding protein comprises a fragment antigen-binding region (Fab).

In some embodiments, the antigen-binding protein comprises a single chain fragment variable (scFv). In some embodiments, the scFv comprises domains arranged in the following orientation from N-terminus to C-terminus: HCVR-LCVR. In some embodiments, the scFv comprises domains arranged in the following orientation from N-terminus to C-terminus: LCVR-HCVR. In some embodiments, the scFv variable regions are connected by a linker. In some embodiments, the linker is a peptide linker. In some embodiments, the peptide linker is -(GGGGS)ₙ—(SEQ ID NO: 321); wherein n is 1-10.

In various embodiments, the antigen-binding protein binds specifically to FGFR3b and/or FGFR3c. In some embodiments, the antigen-binding protein binds specifically to FGFR3b. In some embodiments, the antigen-binding protein binds specifically to FGFR3c. In some embodiments, the antigen-binding protein binds specifically to FGFR3b and FGFR3c. In some embodiments, the FGFR3b is monomeric and/or dimeric FGFR3b. In some embodiments, the antigen-binding protein binds specifically to monomeric FGFR3b. In some embodiments, the antigen-binding protein binds specifically to dimeric FGFR3b. In some embodiments, the antigen-binding protein binds specifically to monomeric and dimeric FGFR3b. In some embodiments, the FGFR3c is monomeric and/or dimeric FGFR3c. In some embodiments, the antigen-binding protein binds specifically to monomeric FGFR3c. In some embodiments, the antigen-binding protein binds specifically to dimeric FGFR3c. In some embodiments, the antigen-binding protein binds specifically to monomeric and dimeric FGFR3c.

In various embodiments, the antigen-binding protein binds specifically to human FGFR3b and/or FGFR3c. In some embodiments, the antigen-binding protein binds specifically to human FGFR3b. In some embodiments, the antigen-binding protein binds specifically to human FGFR3c. In some embodiments, the antigen-binding protein binds specifically to human FGFR3b and FGFR3c.

In some embodiments, the antigen-binding protein binds to human FGFR3b with a K_{D} of about 1×10⁻⁷ M or a stronger affinity. In some embodiments, the antigen-binding protein binds to human FGFR3b with a K_{D} of about 1×10⁻⁸ M or a stronger affinity. In some embodiments, the antigen-binding protein binds to human FGFR3b with a K_{D} of about 1×10⁻⁹ M or a stronger affinity.

In some embodiments, the antigen-binding protein binds to human FGFR3c with a K_{D} of about 1×10⁻⁷ M or a stronger affinity. In some embodiments, the antigen-binding protein binds to human FGFR3c with a K_{D} of about 1×10⁻⁸ M or a stronger affinity.

In some embodiments, the antigen-binding protein comprises:
(i) a HCVR that comprises the HCDR1, HCDR2 and HCDR3 of a HCVR comprising the amino acid sequence set forth in SEQ ID NO: 2, 22, 42, 62, 82, 102, 122, 140, 159, 169, 179, 199 or 219 (or a variant thereof);
and/or
(ii) a LCVR that comprises the LCDR1, LCDR2 and LCDR3 of a LCVR comprising the amino acid sequence set forth in SEQ ID NO: 10, 30, 50, 70, 90, 110, 130, 148, 187, 207, or 227 (or a variant thereof).

In some embodiments, the antigen-binding protein comprises:
(1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 10 (or a variant thereof);
(2) a HCVR comprising the HCDR1, HCDR2 and HCDR3 of a HCVR that comprises the amino acid sequence set forth in SEQ ID NO: 22; and a LCVR comprising the LCDR1, LCDR2 and LCDR3 of a LCVR that comprises the amino acid sequence set forth in SEQ ID NO: 30 (or a variant thereof);
(3) a HCVR comprising the HCDR1, HCDR2 and HCDR3 of a HCVR that comprises the amino acid sequence set forth in SEQ ID NO: 42; and a LCVR comprising the LCDR1, LCDR2 and LCDR3 of a LCVR that comprises the amino acid sequence set forth in SEQ ID NO: 50 (or a variant thereof);
(4) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 70 (or a variant thereof);
(5) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 90 (or a variant thereof); or
(6) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 110 (or a variant thereof);
(7) a HCVR comprising the HCDR1, HCDR2 and HCDR3 of a HCVR that comprises the amino acid sequence set forth in SEQ ID NO: 122 (or a variant thereof); and LCVR comprising the LCDR1, LCDR2 and LCDR3 of a LCVR that comprises the amino acid sequence set forth in SEQ ID NO: 130 (or a variant thereof);
(8) a HCVR comprising the HCDR1, HCDR2 and HCDR3 of a HCVR that comprises the amino acid sequence set forth in SEQ ID NO: 140 (or a variant thereof); and a LCVR comprising the LCDR1, LCDR2 and LCDR3 of a LCVR that comprises the amino acid sequence set forth in SEQ ID NO: 148 (or a variant thereof);
(9) a HCVR comprising the HCDR1, HCDR2 and HCDR3 of a HCVR that comprises the amino acid sequence set forth in SEQ ID NO: 159 (or a variant thereof); and LCVR comprising the LCDR1, LCDR2 and LCDR3 of a LCVR that comprises the amino acid sequence set forth in SEQ ID NO: 148 (or a variant thereof);
(10) a HCVR comprising the HCDR1, HCDR2 and HCDR3 of a HCVR that comprises the amino acid sequence set forth in SEQ ID NO: 169 (or a variant thereof); and LCVR comprising the LCDR1, LCDR2 and LCDR3 of a LCVR that comprises the amino acid sequence set forth in SEQ ID NO: 148 (or a variant thereof);
(11) a HCVR comprising the HCDR1, HCDR2 and HCDR3 of a HCVR that comprises the amino acid sequence set forth in SEQ ID NO: 179 (or a variant thereof); and LCVR comprising the LCDR1, LCDR2 and LCDR3 of a LCVR that comprises the amino acid sequence set forth in SEQ ID NO: 187 (or a variant thereof);
(12) a HCVR comprising the HCDR1, HCDR2 and HCDR3 of a HCVR that comprises the amino acid sequence set forth in SEQ ID NO: 199 (or a variant thereof); and LCVR comprising the LCDR1, LCDR2 and LCDR3 of a LCVR that comprises the amino acid sequence set forth in SEQ ID NO: 207 (or a variant thereof); or
(13) a HCVR comprising the HCDR1, HCDR2 and HCDR3 of a HCVR that comprises the amino acid sequence set forth in SEQ ID NO: 219 (or a variant thereof); and LCVR comprising the LCDR1, LCDR2 and LCDR3 of a LCVR that comprises the amino acid sequence set forth in SEQ ID NO: 227 (or a variant thereof).

In some embodiments, the antigen-binding protein comprises:
(a) a HCVR that comprises:
an HCDR1 comprising the amino acid sequence set forth in SEQ ID NO: 4 (or a variant thereof),
an HCDR2 comprising the amino acid sequence set forth in SEQ ID NO: 6 (or a variant thereof), and
an HCDR3 comprising the amino acid sequence set forth in SEQ ID NO: 8 (or a variant thereof); and
a LCVR that comprises:
an LCDR1 comprising the amino acid sequence set forth in SEQ ID NO: 12 (or a variant thereof),
an LCDR2 comprising the amino acid sequence set forth in SEQ ID NO: 14 (or a variant thereof), and
an LCDR3 comprising the amino acid sequence set forth in SEQ ID NO: 16 (or a variant thereof);
(b) a HCVR that comprises:
an HCDR1 comprising the amino acid sequence set forth in SEQ ID NO: 24 (or a variant thereof),
an HCDR2 comprising the amino acid sequence set forth in SEQ ID NO: 26 (or a variant thereof), and
an HCDR3 comprising the amino acid sequence set forth in SEQ ID NO: 28 (or a variant thereof); and
a LCVR that comprises:
an LCDR1 comprising the amino acid sequence set forth in SEQ ID NO: 32 (or a variant thereof),
an LCDR2 comprising the amino acid sequence set forth in SEQ ID NO: 34 (or a variant thereof), and
an LCDR3 comprising the amino acid sequence set forth in SEQ ID NO: 36 (or a variant thereof);
(c) a HCVR that comprises:
an HCDR1 comprising the amino acid sequence set forth in SEQ ID NO: 44 (or a variant thereof),
an HCDR2 comprising the amino acid sequence set forth in SEQ ID NO: 46 (or a variant thereof), and
an HCDR3 comprising the amino acid sequence set forth in SEQ ID NO: 48 (or a variant thereof); and
a LCVR that comprises:
an LCDR1 comprising the amino acid sequence set forth in SEQ ID NO: 52 (or a variant thereof),
an LCDR2 comprising the amino acid sequence set forth in SEQ ID NO: 54 (or a variant thereof), and
an LCDR3 comprising the amino acid sequence set forth in SEQ ID NO: 56 (or a variant thereof);
(d) a HCVR that comprises:
an HCDR1 comprising the amino acid sequence set forth in SEQ ID NO: 64 (or a variant thereof),
an HCDR2 comprising the amino acid sequence set forth in SEQ ID NO: 66 (or a variant thereof), and
an HCDR3 comprising the amino acid sequence set forth in SEQ ID NO: 68 (or a variant thereof); and
a LCVR that comprises:
an LCDR1 comprising the amino acid sequence set forth in SEQ ID NO: 72 (or a variant thereof),
an LCDR2 comprising the amino acid sequence set forth in SEQ ID NO: 74 (or a variant thereof), and
an LCDR3 comprising the amino acid sequence set forth in SEQ ID NO: 76 (or a variant thereof);
(e) a HCVR that comprises:
an HCDR1 comprising the amino acid sequence set forth in SEQ ID NO: 84 (or a variant thereof),
an HCDR2 comprising the amino acid sequence set forth in SEQ ID NO: 86 (or a variant thereof), and
an HCDR3 comprising the amino acid sequence set forth in SEQ ID NO: 88 (or a variant thereof); and
a LCVR that comprises:
an LCDR1 comprising the amino acid sequence set forth in SEQ ID NO: 92 (or a variant thereof),
an LCDR2 comprising the amino acid sequence set forth in SEQ ID NO: 94 (or a variant thereof), and
an LCDR3 comprising the amino acid sequence set forth in SEQ ID NO: 96 (or a variant thereof);
(f) a HCVR that comprises:
an HCDR1 comprising the amino acid sequence set forth in SEQ ID NO: 104 (or a variant thereof),
an HCDR2 comprising the amino acid sequence set forth in SEQ ID NO: 106 (or a variant thereof), and
an HCDR3 comprising the amino acid sequence set forth in SEQ ID NO: 108 (or a variant thereof); and
a LCVR that comprises:
an LCDR1 comprising the amino acid sequence set forth in SEQ ID NO: 112 (or a variant thereof),
an LCDR2 comprising the amino acid sequence set forth in SEQ ID NO: 114 (or a variant thereof), and
an LCDR3 comprising the amino acid sequence set forth in SEQ ID NO: 116 (or a variant thereof); or
(g) a HCVR that comprises:
an HCDR1 comprising the amino acid sequence set forth in SEQ ID NO: 124 (or a variant thereof),
an HCDR2 comprising the amino acid sequence set forth in SEQ ID NO: 126 (or a variant thereof), and
an HCDR3 comprising the amino acid sequence set forth in SEQ ID NO: 128 (or a variant thereof), and
a LCVR that comprises:
an LCDR1 comprising the amino acid sequence set forth in SEQ ID NO: 132 (or a variant thereof),
an LCDR2 comprising the amino acid sequence set forth in SEQ ID NO: 34 (or a variant thereof), and
an LCDR3 comprising the amino acid sequence set forth in SEQ ID NO: 134; (or a variant thereof)
(h) a HCVR that comprises:
an HCDR1 comprising the amino acid sequence set forth in SEQ ID NO: 142 (or a variant thereof),
an HCDR2 comprising the amino acid sequence set forth in SEQ ID NO: 144 (or a variant thereof), and
an HCDR3 comprising the amino acid sequence set forth in SEQ ID NO: 146 (or a variant thereof), and
a LCVR that comprises:
an LCDR1 comprising the amino acid sequence set forth in SEQ ID NO: 150 (or a variant thereof),
an LCDR2 comprising the amino acid sequence set forth in SEQ ID NO: 14 (or a variant thereof), and
an LCDR3 comprising the amino acid sequence set forth in SEQ ID NO: 153 (or a variant thereof);
(i) a HCVR that comprises:
an HCDR1 comprising the amino acid sequence set forth in SEQ ID NO: 161 (or a variant thereof),
an HCDR2 comprising the amino acid sequence set forth in SEQ ID NO: 163 (or a variant thereof), and
an HCDR3 comprising the amino acid sequence set forth in SEQ ID NO: 165 (or a variant thereof), and
a LCVR that comprises:

an LCDR1 comprising the amino acid sequence set forth in SEQ ID NO: 150 (or a variant thereof),
an LCDR2 comprising the amino acid sequence set forth in SEQ ID NO: 14 (or a variant thereof), and
an LCDR3 comprising the amino acid sequence set forth in SEQ ID NO: 153 (or a variant thereof);
(j) a HCVR that comprises:
an HCDR1 comprising the amino acid sequence set forth in SEQ ID NO: 171 (or a variant thereof),
an HCDR2 comprising the amino acid sequence set forth in SEQ ID NO: 173 (or a variant thereof), and
an HCDR3 comprising the amino acid sequence set forth in SEQ ID NO: 175 (or a variant thereof), and
a LCVR that comprises:
an LCDR1 comprising the amino acid sequence set forth in SEQ ID NO: 150 (or a variant thereof),
an LCDR2 comprising the amino acid sequence set forth in SEQ ID NO: 14 (or a variant thereof), and
an LCDR3 comprising the amino acid sequence set forth in SEQ ID NO: 153 (or a variant thereof);
(k) a HCVR that comprises:
an HCDR1 comprising the amino acid sequence set forth in SEQ ID NO: 181 (or a variant thereof),
an HCDR2 comprising the amino acid sequence set forth in SEQ ID NO: 183 (or a variant thereof), and
an HCDR3 comprising the amino acid sequence set forth in SEQ ID NO: 185 (or a variant thereof), and
a LCVR that comprises:
an LCDR1 comprising the amino acid sequence set forth in SEQ ID NO: 189 (or a variant thereof),
an LCDR2 comprising the amino acid sequence set forth in SEQ ID NO: 191 (or a variant thereof), and
an LCDR3 comprising the amino acid sequence set forth in SEQ ID NO: 193 (or a variant thereof);
(I) a HCVR that comprises:
an HCDR1 comprising the amino acid sequence set forth in SEQ ID NO: 201 (or a variant thereof),
an HCDR2 comprising the amino acid sequence set forth in SEQ ID NO: 203 (or a variant thereof), and
an HCDR3 comprising the amino acid sequence set forth in SEQ ID NO: 205 (or a variant thereof), and
a LCVR that comprises:
an LCDR1 comprising the amino acid sequence set forth in SEQ ID NO: 209 (or a variant thereof),
an LCDR2 comprising the amino acid sequence set forth in SEQ ID NO: 211 (or a variant thereof), and
an LCDR3 comprising the amino acid sequence set forth in SEQ ID NO: 213 (or a variant thereof);
and/or
(m) a HCVR that comprises:
an HCDR1 comprising the amino acid sequence set forth in SEQ ID NO: 221 (or a variant thereof),
an HCDR2 comprising the amino acid sequence set forth in SEQ ID NO: 223 (or a variant thereof), and
an HCDR3 comprising the amino acid sequence set forth in SEQ ID NO: 225 (or a variant thereof), and
a LCVR that comprises:
an LCDR1 comprising the amino acid sequence set forth in SEQ ID NO: 32 (or a variant thereof),
an LCDR2 comprising the amino acid sequence set forth in SEQ ID NO: 34 (or a variant thereof), and
an LCDR3 comprising the amino acid sequence set forth in SEQ ID NO: 76 (or a variant thereof).

In some embodiments, the antigen-binding protein comprises:
(i) a HCVR that comprises the amino acid sequence set forth in SEQ ID NO: 2 (or a variant thereof); and a LCVR that comprises the amino acid sequence set forth in SEQ ID NO: 10 (or a variant thereof);
(ii) a HCVR that comprises the amino acid sequence set forth in SEQ ID NO: 22 (or a variant thereof); and a LCVR that comprises the amino acid sequence set forth in SEQ ID NO: 30 (or a variant thereof);
(iii) a HCVR that comprises the amino acid sequence set forth in SEQ ID NO: 42 (or a variant thereof); and a LCVR that comprises the amino acid sequence set forth in SEQ ID NO: 50 (or a variant thereof);
(iv) a HCVR that comprises the amino acid sequence set forth in SEQ ID NO: 62 (or a variant thereof); and a LCVR that comprises the amino acid sequence set forth in SEQ ID NO: 70 (or a variant thereof);
(v) a HCVR that comprises the amino acid sequence set forth in SEQ ID NO: 82 (or a variant thereof); and a LCVR that comprises the amino acid sequence set forth in SEQ ID NO: 90 (or a variant thereof);
(vi) a HCVR that comprises the amino acid sequence set forth in SEQ ID NO: 102 (or a variant thereof); and a LCVR that comprises the amino acid sequence set forth in SEQ ID NO: 110 (or a variant thereof);
(vii) a HCVR that comprises the amino acid sequence set forth in SEQ ID NO: 122 (or a variant thereof); and a LCVR that comprises the amino acid sequence set forth in SEQ ID NO: 130 (or a variant thereof);
(viii) a HCVR that comprises the amino acid sequence set forth in SEQ ID NO: 140 (or a variant thereof); and a LCVR that the amino acid sequence set forth in SEQ ID NO: 148 (or a variant thereof);
(ix) a HCVR that comprises the amino acid sequence set forth in SEQ ID NO: 159 (or a variant thereof); and a LCVR that comprises the amino acid sequence set forth in SEQ ID NO: 148 (or a variant thereof);
(x) a HCVR that comprises the amino acid sequence set forth in SEQ ID NO: 169 (or a variant thereof); and LCVR that comprises the amino acid sequence set forth in SEQ ID NO: 148 (or a variant thereof);
(xi) a HCVR that comprises the amino acid sequence set forth in SEQ ID NO: 179 (or a variant thereof); and a LCVR that comprises the amino acid sequence set forth in SEQ ID NO: 187 (or a variant thereof);
(xii) a HCVR that comprises the amino acid sequence set forth in SEQ ID NO: 199 (or a variant thereof); and LCVR that comprises the amino acid sequence set forth in SEQ ID NO: 207 (or a variant thereof); or
(xiii) a HCVR that comprises the amino acid sequence set forth in SEQ ID NO: 219 (or a variant thereof); and LCVR that comprises the amino acid sequence set forth in SEQ ID NO: 227 (or a variant thereof).

In some embodiments, the antigen-binding protein comprises:
(a) a heavy chain that comprises the amino acid sequence set forth in SEQ ID NO: 18, and a light chain that comprises the amino acid sequence set forth in SEQ ID NO: 20;
(b) a heavy chain that comprises the amino acid sequence set forth in SEQ ID NO: 38, and a light chain that comprises the amino acid sequence set forth in SEQ ID NO: 40;
(c) a heavy chain that comprises the amino acid sequence set forth in SEQ ID NO: 58, and a light chain that comprises the amino acid sequence set forth in SEQ ID NO: 60;
(d) a heavy chain that comprises the amino acid sequence set forth in SEQ ID NO: 78, and a light chain that comprises the amino acid sequence set forth in SEQ ID NO: 80;
(e) a heavy chain that comprises the amino acid sequence set forth in SEQ ID NO: 98, and a light chain that comprises the amino acid sequence set forth in SEQ ID NO: 100;
(f) a heavy chain that comprises the amino acid sequence set forth in SEQ ID NO: 118, and a light chain that comprises the amino acid sequence set forth in SEQ ID NO: 120;
(g) a heavy chain that comprises the amino acid sequence set forth in SEQ ID NO: 136, and a light chain that comprises the amino acid sequence set forth in SEQ ID NO: 138;
(h) a heavy chain that comprises the amino acid sequence set forth in SEQ ID NO: 155, and a light chain that comprises the amino acid sequence set forth in SEQ ID NO: 157;
(i) a heavy chain that comprises the amino acid sequence set forth in SEQ ID NO: 167, and a light chain that comprises the amino acid sequence set forth in SEQ ID NO: 157;
(j) a heavy chain that comprises the amino acid sequence set forth in SEQ ID NO: 177, and a light chain that comprises the amino acid sequence set forth in SEQ ID NO: 157;
(k) a heavy chain that comprises the amino acid sequence set forth in SEQ ID NO: 195, and a light chain that comprises the amino acid sequence set forth in SEQ ID NO: 197;
(l) a heavy chain that comprises the amino acid sequence set forth in SEQ ID NO: 215, and a light chain that comprises the amino acid sequence set forth in SEQ ID NO: 217; or
(m) a heavy chain that comprises the amino acid sequence set forth in SEQ ID NO: 229, and a light chain that comprises the amino acid sequence set forth in SEQ ID NO: 231.

In some embodiments, the antigen-binding protein binds specifically to monomeric FGFR3b. In some embodiments, the antigen-binding protein that binds specifically to monomeric FGFR3b comprises:
(a) a heavy chain that comprises the amino acid sequence set forth in SEQ ID NO: 18, and a light chain that comprises the amino acid sequence set forth in SEQ ID NO: 20;
(b) a heavy chain that comprises the amino acid sequence set forth in SEQ ID NO: 38, and a light chain that comprises the amino acid sequence set forth in SEQ ID NO: 40;
(c) a heavy chain that comprises the amino acid sequence set forth in SEQ ID NO: 58, and a light chain that comprises the amino acid sequence set forth in SEQ ID NO: 60;
(d) a heavy chain that comprises the amino acid sequence set forth in SEQ ID NO: 78, and a light chain that comprises the amino acid sequence set forth in SEQ ID NO: 80;
(e) a heavy chain that comprises the amino acid sequence set forth in SEQ ID NO: 98, and a light chain that comprises the amino acid sequence set forth in SEQ ID NO: 100;
(f) a heavy chain that comprises the amino acid sequence set forth in SEQ ID NO: 118, and a light chain that comprises the amino acid sequence set forth in SEQ ID NO: 120;
(g) a heavy chain that comprises the amino acid sequence set forth in SEQ ID NO: 136, and a light chain that comprises the amino acid sequence set forth in SEQ ID NO: 138;
(h) a heavy chain that comprises the amino acid sequence set forth in SEQ ID NO: 155, and a light chain that comprises the amino acid sequence set forth in SEQ ID NO: 157;
(i) a heavy chain that comprises the amino acid sequence set forth in SEQ ID NO: 167, and a light chain that comprises the amino acid sequence set forth in SEQ ID NO: 157;
(j) a heavy chain that comprises the amino acid sequence set forth in SEQ ID NO: 177, and a light chain that comprises the amino acid sequence set forth in SEQ ID NO: 157;
(k) a heavy chain that comprises the amino acid sequence set forth in SEQ ID NO: 195, and a light chain that comprises the amino acid sequence set forth in SEQ ID NO: 197;
(l) a heavy chain that comprises the amino acid sequence set forth in SEQ ID NO: 215, and a light chain that comprises the amino acid sequence set forth in SEQ ID NO: 217; and/or
(m) a heavy chain that comprises the amino acid sequence set forth in SEQ ID NO: 229, and a light chain that comprises the amino acid sequence set forth in SEQ ID NO: 231.

In some embodiments, the antigen-binding protein binds specifically to dimeric FGFR3b. In some embodiments, the antigen-binding protein that binds specifically to dimeric FGFR3b comprises:
(a) a heavy chain that comprises the amino acid sequence set forth in SEQ ID NO: 18, and a light chain that comprises the amino acid sequence set forth in SEQ ID NO: 20;
(b) a heavy chain that comprises the amino acid sequence set forth in SEQ ID NO: 38, and a light chain that comprises the amino acid sequence set forth in SEQ ID NO: 40;
(c) a heavy chain that comprises the amino acid sequence set forth in SEQ ID NO: 58, and a light chain that comprises the amino acid sequence set forth in SEQ ID NO: 60;
(d) a heavy chain that comprises the amino acid sequence set forth in SEQ ID NO: 78, and a light chain that comprises the amino acid sequence set forth in SEQ ID NO: 80;
(e) a heavy chain that comprises the amino acid sequence set forth in SEQ ID NO: 98, and a light chain that comprises the amino acid sequence set forth in SEQ ID NO: 100;
(f) a heavy chain that comprises the amino acid sequence set forth in SEQ ID NO: 118, and a light chain that comprises the amino acid sequence set forth in SEQ ID NO: 120;
(g) a heavy chain that comprises the amino acid sequence set forth in SEQ ID NO: 136, and a light chain that comprises the amino acid sequence set forth in SEQ ID NO: 138;
(h) a heavy chain that comprises the amino acid sequence set forth in SEQ ID NO: 167, and a light chain that comprises the amino acid sequence set forth in SEQ ID NO: 157;
(i) a heavy chain that comprises the amino acid sequence set forth in SEQ ID NO: 177, and a light chain that comprises the amino acid sequence set forth in SEQ ID NO: 157;
(j) a heavy chain that comprises the amino acid sequence set forth in SEQ ID NO: 195, and a light chain that comprises the amino acid sequence set forth in SEQ ID NO: 197;
(k) a heavy chain that comprises the amino acid sequence set forth in SEQ ID NO: 215, and a light chain that comprises the amino acid sequence set forth in SEQ ID NO: 217; and/or
(l) a heavy chain that comprises the amino acid sequence set forth in SEQ ID NO: 229, and a light chain that comprises the amino acid sequence set forth in SEQ ID NO: 231.

In some embodiments, the antigen-binding protein binds specifically to monomeric and dimeric FGFR3b.

In some embodiments, the antigen-binding protein binds specifically to monomeric FGFR3c. In some embodiments, the antigen-binding protein that binds specifically to monomeric FGFR3c comprises
(a) a heavy chain that comprises the amino acid sequence set forth in SEQ ID NO: 98, and a light chain that comprises the amino acid sequence set forth in SEQ ID NO: 100;
(b) a heavy chain that comprises the amino acid sequence set forth in SEQ ID NO: 155, and a light chain that comprises the amino acid sequence set forth in SEQ ID NO: 157;
(c) a heavy chain that comprises the amino acid sequence set forth in SEQ ID NO: 177, and a light chain that comprises the amino acid sequence set forth in SEQ ID NO: 157;
(d) a heavy chain that comprises the amino acid sequence set forth in SEQ ID NO: 195, and a light chain that comprises the amino acid sequence set forth in SEQ ID NO: 197;
(e) a heavy chain that comprises the amino acid sequence set forth in SEQ ID NO: 215, and a light chain that comprises the amino acid sequence set forth in SEQ ID NO: 217; and/or
(f) a heavy chain that comprises the amino acid sequence set forth in SEQ ID NO: 229, and a light chain that comprises the amino acid sequence set forth in SEQ ID NO: 231.

In some embodiments, the antigen-binding protein comprises a heavy chain that comprises the amino acid sequence set forth in SEQ ID NO: 18, and a light chain that comprises the amino acid sequence set forth in SEQ ID NO: 20.

In some embodiments, the antigen-binding protein binds to the same epitope on FGFR3 as an antibody comprising an HCVR/LCVR amino acid sequence pair as set forth in Table 1-1.

In some embodiments, the antigen-binding protein competes for binding to FGFR3 with an antibody comprising an HCVR/LCVR amino acid sequence pair as set forth in Table 1-1.

In one aspect, provided herein is a protein-drug conjugate comprising an antigen-binding protein that binds specifically to fibro-blast growth factor receptor 3 (FGFR3), wherein the antigen-binding protein is conjugated to a molecular cargo and comprises an antibody or antigen-binding fragment thereof, wherein the antibody or antigen-binding fragment thereof binds to one or more epitopes of FGFR3 selected from: a. an epitope comprising the sequence GPTVVVVK (SEQ ID NO: 378) and/or an epitope comprising the sequence TQR; b. an epitope comprising the sequence ADVR (SEQ ID NO: 376) and/or an epitope compris-ing the sequence IGVAEK (SEQ ID NO: 377); c. an epitope comprising the sequence HCKVY (SEQ ID NO: 379), and/or an epitope com-prising the sequence KSWISE (SEQ ID NO: 380), and/or an epitope comprising the se-quence ADVR (SEQ ID NO: 376); e. an epitope comprised within or overlapping with the sequence GPTVVVVK (SEQ ID NO: 378) and/or an epitope comprised within or overlapping with the sequence TQR; f. an epitope comprised within or overlapping with the sequence ADVR (SEQ ID NO: 376) and/or an epitope comprised within or overlapping with the sequence IGVAEK (SEQ ID NO: 377); and g. an epitope comprised within or overlapping with the sequence HCKVY (SEQ ID NO: 379), and/or an epitope comprised within or overlapping with the sequence KSWISE (SEQ ID NO: 380), and/or an epitope comprised within or overlapping with the sequence ADVR (SEQ ID NO: 376). In some embodiments, the antibody or antigen-binding fragment thereof binds to one or more epitopes of FGFR3 selected from: a. an epitope consisting of the sequence GPTVVVVK (SEQ ID NO: 378) and/or an epitope consisting of the sequence TQR; b. an epitope consisting of the sequence ADVR (SEQ ID NO: 376) and/or an epitope consist-ing of the sequence IGVAEK (SEQ ID NO: 377); and c. an epitope consisting of the sequence HCKVY (SEQ ID NO: 379), and/or an epitope con-sisting of the sequence KSWISE (SEQ ID NO: 380), and/or an epitope consisting of the se-quence ADVR (SEQ ID NO: 376).

In one aspect, provided herein A protein-drug conjugate comprising an antigen-binding protein that binds specifically to fibroblast growth factor receptor 3 (FGFR3), wherein the antigen-binding protein is conjugated to a molecular cargo and comprises an antibody or antigen-binding fragment thereof, wherein the antibody or antigen-binding fragment thereof binds to one or more epitopes of FGFR3 selected from: a. an epitope comprising the sequence SCPPPGGGPMGPTVVVVKDGTGLVPSER (SEQ ID NO: 363) and/or an epitope comprising the sequence YSCRQRLTQRVL (SEQ ID NO: 364); b. an epitope comprising the sequence LLAVPAAN (SEQ ID NO: 365), and/or an epitope comprising the sequence VLERSPHRPILQAG (SEQ ID NO: 366) and/or an epitope comprising the sequence YVTVLKSWISE (SEQ ID NO: 367) and/or or an epitope comprising the sequence ADVRLR (SEQ ID NO: 368) and/or an epitope comprising the sequence LCRATNFIGVAEKAFW (SEQ ID NO: 369); c. an epitope comprising the sequence GQQEQLVFGSGDAVE (SEQ ID NO: 370) and/or an epitope comprising the sequence VLVGPQRL (SEQ ID NO: 371); d. an epitope comprising the sequence VLERSPHRPILQAG (SEQ ID NO: 372) and/or an epitope comprising the sequence HCKVYSDAQP (SEQ ID NO: 373) and/or an epitope comprising the sequence YVTVLKSWISESVEADVRLR (SEQ ID NO: 374) and/or an epitope comprising the sequence LCRATNFIGVAEKAF (SEQ ID NO: 375); e. an epitope comprised within or overlapping with the sequence SCPPPGGGPMGPTVVVVKDGTGLVPSER (SEQ ID NO: 363) and/or an epitope comprised within or overlapping with the sequence YSCRQRLTQRVL (SEQ ID NO: 364); f. an epitope comprised within or overlapping with the sequence LLAVPAAN (SEQ ID NO: 365), and/or an epitope comprised within or overlapping with the sequence VLERSPHRPILQAG (SEQ ID NO: 366) and/or an epitope comprised within or overlapping with the sequence YVTVLKSWISE (SEQ ID NO: 367) and/or or an epitope comprised within or overlapping with the sequence ADVRLR (SEQ ID NO: 368) and/or an epitope comprised within or overlapping with the sequence LCRATNFIGVAEKAFW (SEQ ID NO: 369); g. an epitope comprised within or overlapping with the sequence GQQEQLVFGSGDAVE (SEQ ID NO: 370) and/or an epitope comprised within or overlapping with the sequence VLVGPQRL (SEQ ID NO: 371); and h. an epitope comprised within or overlapping with the sequence VLERSPHRPILQAG (SEQ ID NO: 372) and/or an epitope comprised within or overlapping with the sequence HCKVYSDAQP (SEQ ID NO: 373) and/or an epitope comprised within or overlapping with the sequence YVTVLKSWISESVEADVRLR (SEQ ID NO: 374) and/or an epitope comprised within or overlapping with the sequence LCRATNFIGVAEKAF (SEQ ID NO: 375). In some embodiments, the antibody or antigen-binding fragment thereof binds to one or more epitopes of FGFR3 selected from: a. an epitope consisting of the sequence SCPPPGGGPMGPTVWVKDGTGLVPSER (SEQ ID NO: 363) and/or an epitope consisting of the sequence YSCRQRLTQRVL (SEQ ID NO: 364); b. an epitope consisting of the sequence LLAVPAAN (SEQ ID NO: 365), and/or an epitope consisting of the sequence VLERSPHRPILQAG (SEQ ID NO: 366) and/or an epitope consisting of the sequence YVTVLKSWISE (SEQ ID NO: 367) and/or or an epitope consisting of the sequence ADVRLR (SEQ ID NO: 368) and/or an epitope consisting of the sequence LCRATNFIGVAEKAFW (SEQ ID NO: 369); c. an epitope consisting of the sequence GQQEQLVFGSGDAVE (SEQ ID NO: 370) and/or an epitope consisting of the sequence VLVGPQRL (SEQ ID NO: 371); and d. an epitope consisting of the sequence VLERSPHRPILQAG (SEQ ID NO: 372) and/or an epitope consisting of the sequence HCKVYSDAQP (SEQ ID NO: 373) and/or an epitope consisting of the sequence YVTVLKSWISESVEADVRLR (SEQ ID NO: 374) and/or an epitope consisting of the sequence LCRATNFIGVAEKAF (SEQ ID NO: 375).

In various embodiments of the protein-drug conjugate described herein, the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BITE), trispecific antibody, or chemically modified derivatives thereof.

In various embodiments of the protein-drug conjugate described herein, the molecular cargo is conjugated to: (i) a HCVR of the antigen-binding protein, (ii) a LCVR of the antigen-binding protein, (iii) a heavy chain of the antigen-binding protein, and/or (iv) a light chain of the antigen-binding protein.

In some embodiments, the molecular cargo is conjugated to the antigen-binding protein via a glutamine residue and/or a lysine residue.

In some embodiments, the glutamine residue is: (i) introduced to the N-terminus and/or C-terminus of a heavy chain of the antigen-binding protein, (ii) introduced to the N-terminus and/or C-terminus of a light chain of the antigen-binding protein, (iii) naturally present in a CH2 or CH3 domain of the antigen-binding protein, (iv) introduced to the antigen-binding protein by modifying one or more amino acids, and/or (v) Q295 or mutated from N297 to Q297 (N297Q).

In some embodiments, the antigen-binding protein comprises a glutamine-containing tag, and the molecular cargo is conjugated to the antigen-binding protein via a glutamine residue of the glutamine-containing tag.

In some embodiments, the glutamine-containing tag comprises an amino acid sequence selected from the group consisting of LLQGG (SEQ ID NO: 290), LLQG (SEQ ID NO: 291), LSLSQG (SEQ ID NO: 292), GGGLLQGG (SEQ ID NO: 293), GLLQG (SEQ ID NO: 294), LLQ, GSPLAQSHGG (SEQ ID NO: 295), GLLQGGG (SEQ ID NO: 296), GLLQGG (SEQ ID NO: 297), GLLQ (SEQ ID NO: 298), LLQLLQGA (SEQ ID NO: 299), LLQGA (SEQ ID NO: 300), LLQYQGA (SEQ ID NO: 301), LLQGSG (SEQ ID NO: 302), LLQYQG (SEQ ID NO: 303), LLQLLQG (SEQ ID NO: 304), SLLQG (SEQ ID NO: 305), LLQLQ (SEQ ID NO: 306), LLQLLQ (SEQ ID NO: 307), and LLQGR (SEQ ID NO: 308).

In some embodiments, the antigen-binding protein and the molecular cargo are conjugated via a linker.

In some embodiments, the molecular cargo comprises a polynucleotide molecule, a polypeptide molecule, a carrier, a viral particle, a viral capsid protein, or a small molecule.

In some embodiments, the molecular cargo comprises a polynucleotide molecule. In some embodiments, the polynucleotide molecule is an interfering nucleic acid molecule, a guide RNA, a ribozyme, an aptamer, a mixmer, a multimer, or an mRNA. In some embodiments, the interfering nucleic acid molecule is an siRNA, an shRNA, a miRNA, an antisense oligonucleotide, or a gapmer.

In some embodiments, the interfering nucleic acid molecule is an siRNA. In some embodiments, the siRNA comprises a sense strand of 21 nucleotides in length. In some embodiments, the siRNA comprises an antisense strand of 23 nucleotides in length. In some embodiments, the siRNA comprises two phosphorothioate linkages at the first and second internucleoside linkages at the 5′ end of the sense strand. In some embodiments, the siRNA comprises two phosphorothioate linkages at the first and second internucleoside linkages at the 3′ and/or 5′ ends of the antisense strand.

In some embodiments, the interfering nucleic acid molecule is an antisense oligonucleotide.

In some embodiments, the polynucleotide molecule is a guide RNA.

In various embodiments, the polynucleotide molecule targets a gene or gene product associated with a neurological disease and/or disorder. In some embodiments, the gene is APOE4, GFAP, MECP2, AQP4, or STAT3.

In various embodiments, the polynucleotide molecule comprises one or more modified nucleotides.

In some embodiments, the molecular cargo comprises a polypeptide molecule. In some embodiments, the polypeptide molecule is an enzyme, a neuroprotective molecule, or an antigen-binding protein that binds to a target other than FGFR3. In some embodiments, the polypeptide molecule is associated with a neurological disease and/or disorder. In some embodiments, the polypeptide molecule is a protective ApoE isoform or variant, ATPase 13A2 (encoded by ATP13A2), sulfatase modifying factor 1 (encoded by SUMF1), fragile X messenger ribonucleoprotein (FMRP) (encoded by FMR1), or glutamate transporter-1 (encoded by GLT1). In some embodiments, the protective ApoE isoform or variant is ApoE2, ApoE Christchurch, or ApoE Jacksonville. In some embodiments, the polypeptide molecule is a neurotrophic factor, an antibody or antibody fragment, an antibody receptor fusion protein, or a suppressor of cytokine signaling. In some embodiments, the neurotrophic factor is ciliary neurotrophic factor (CNTF), brain derived neurotrophic factor (BDNF), nerve growth factor (NGF), glial cell line-derived neurotrophic factor (GDNF), or insulin-like growth factor 1 (IGF). In some embodiments, the antibody receptor fusion protein is anti-amyloid beta Gas6 fusion protein. In some embodiments, the suppressor of cytokine signaling is suppressor of cytokine signaling 3 (Socs3).

In some embodiments, the molecular cargo is conjugated to the antigen-binding protein at a drug-to-antibody ratio (DAR) of a least 1 to at least 10.

In some embodiments, the molecular cargo is conjugated to the antigen-binding protein ata DAR of 1, 2, 3, or 4.

In some embodiments, the molecular cargo is conjugated to the antigen-binding protein ata DAR of 2.

In some embodiments, the molecular cargo is conjugated to the antigen-binding protein at a DAR of 4.

In some embodiments, provided herein is a protein-drug conjugate for use in treating or preventing a neurological disease or disorder.

In various embodiments, the neurological disease or disorder is a neurodegenerative disease, a neurodevelopmental disease, a physical injury, a neuropsychiatric disease, or a brain cancer. In some embodiments, the neurodegenerative disease is Alzheimer's disease, Parkinson's disease, Huntington's disease, amyotrophic lateral sclerosis (ALS), or prion disease (transmissible spongiform encephalopathy). In some embodiments, the neurodevelopmental disease is Alexander disease, multiple sulfatase deficiency, autism, epilepsy, Rett syndrome, or Fragile X. In some embodiments, the physical injury is traumatic brain injury, spinal cord injury, stroke, or brain edema. In some embodiments, the neuropsychiatric disease or disorder is major depressive disorder, anxiety disorder, or bipolar disorder. In some embodiments, the brain cancer is glioma. In some embodiments, the glioma is an astrocytoma.

In some embodiments, the molecular cargo comprises a carrier. In some embodiments, the carrier is a lipid-based carrier. In some embodiments, the lipid-based carrier is a lipid nanoparticle (LNP), a liposome, a lipidoid, or a lipoplex.

In some embodiments, the lipid-based carrier is a lipid nanoparticle (LNP). In some embodiments, the LNP further comprises a polynucleotide molecule and/or a polypeptide molecule. In some embodiments, the LNP comprises one or more components of a gene editing system. In some embodiments, the LNP comprises (a) a Cas nuclease, or a nucleic acid encoding the Cas nuclease, and/or (b) a guide RNA, or one or more DNAs encoding the guide RNA. In some embodiments, the Cas nuclease is a Cas9 protein. In some embodiments, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some embodiments, the nucleic acid encoding the Cas nuclease is codon-optimized for expression in a mammalian cell. In some embodiments, the nucleic acid encoding the Cas nuclease is codon-optimized for expression in a human cell. In some embodiments, the nucleic acid encoding the Cas nuclease is an mRNA. In various embodiments, the guide RNA is a single guide RNA (sgRNA). In some embodiments, the LNP comprises a zinc finger nuclease (ZFN), or a transcription activator-like effector nuclease (TALEN).

In some embodiments, the LNP comprises a cationic lipid, a neutral lipid, a helper lipid, a stealth lipid, or any combinations thereof. In some embodiments, the neutral lipid is distearoylphosphatidylcholine (DSPC). In some embodiments, the helper lipid is cholesterol. In some embodiments, the stealth lipid is PEG2k-DMG.

In another aspect, provided herein is a pharmaceutical composition comprising the protein-drug conjugate described herein and a pharmaceutically acceptable carrier.

In another aspect, provided herein is a composition or kit comprising the protein-drug conjugate described herein or pharmaceutical composition described herein and a further therapeutic agent.

In another aspect, provided herein is a complex comprising the protein-drug conjugate described herein bound to fibroblast growth factor receptor 3 (FGFR3).

In another aspect, provided herein is a method for making a protein-drug conjugate described herein comprising
(a) contacting the antigen-binding protein, with the molecular cargo under the conditions favorable for conjugation of the antigen-binding protein to the molecular cargo; and
(b) optionally, isolating the protein-drug conjugate produced in step (a).

In another aspect, provided herein is a method for making a protein-drug conjugate described herein, wherein the molecular cargo comprises a polypeptide molecule, comprising
(a) culturing a host cell comprising a polynucleotide encoding the protein-drug conjugate under conditions that allow expression of the protein-drug conjugate; and
(b) optionally, isolating the protein-drug conjugate produced in step (a).

In another aspect, provided herein is a protein-drug conjugate which is produced by or obtainable by the method described above.

In another aspect, provided herein is a vessel or injection device comprising the protein-drug conjugate described herein.

In another aspect, provided herein is a method for administering the protein-drug conjugate described herein to a subject comprising introducing the protein-drug conjugate into the body of the subject.

In another aspect, provided herein is a method for delivering a molecular cargo to a tissue or cell type expressing FGFR3 in the body of a subject comprising administering to the subject the protein-drug conjugate described herein or the pharmaceutical composition described herein. In some embodiments, the tissue is brain, spinal cord, or eye. In some embodiments, the cell type is astrocyte or astrocyte-derived tumor cell.

In another aspect, provided herein is a method for treating or preventing a neurological disease or disorder in a subject in need thereof comprising administering to the subject an effective amount of the protein-drug conjugate of any one described herein. In some embodiments, the neurological disease or disorder is associated with astrocytes. In some embodiments, the neurological disease or disorder is a neurodegenerative disease, neurodevelopmental disease, physical injury, neuropsychiatric disease, or a brain cancer. In some embodiments, the neurodegenerative disease is Alzheimer's disease, Parkinson's disease, Huntington's disease, amyotrophic lateral sclerosis, or prion disease (transmissible spongiform encephalopathy). In some embodiments, the neurodevelopmental disease is an Alexander disease, multiple sulfatase deficiency, autism, epilepsy, Rett syndrome, or Fragile X. In some embodiments, the physical injury is traumatic brain injury, spinal cord injury, stroke, or brain edema. In some embodiments, the neuropsychiatric disease or disorder is major depressive disorder, anxiety disorder, or bipolar disorder. In some embodiments, the brain cancer is glioma. In some embodiments, the glioma is an astrocytoma. In some embodiments, the method further comprises administering an additional treatment to the subject.

In various embodiments of the methods described herein, the protein-drug conjugate is administered into the body of the subject via intrathecal, intracisternal, intracerebroventricular, or intraparenchymal administration into the central nervous system.

In various embodiments of the methods described herein, the protein-drug conjugate is administered into the body of the subject via intravitreal or intraocular administration into the eye.

In various embodiments of the methods described herein, the protein-drug conjugate is administered into the body of the subject via systemic administration. In one embodiment, the protein-drug conjugate is administered into the body of the subject via intranasal administration.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1B depict schematic representations of the general architecture of fibroblast growth factor receptor 3 (FGFR3), and isoforms FGFR3b and FGFR3c. Schematic showing the orientation of an FGFR dimer within the plasma membrane (FIG. 1A, top). The FGF3 receptor domain structure is comprised of an extracellular domain comprising a ligand binding site, a transmembrane domain, and an intracellular domain comprising spit tyrosine kinases. The receptor is depicted stabilized by a heparin/heparan sulfate (HS) chain of HS proteoglycan, HSPG. Schematic of the FGF monomer exemplifying the three IgG-like loops (Ig-I, Ig-II, and Ig-III) of the extracellular ligand-binding domain and the split kinase domain (FIG. 1A, bottom). Schematic showing a structural comparison of alternatively spliced FGFR3b and FGFR3c isoforms (FIG. 1B). Ig-I, Ig-II, and Ig-II loops of the extracellular domain ligand-binding domain are encoded by exons 7-9. FGFR3b includes exon 8, and excludes exon 9, whereas FGFR3c includes exon 9 and excludes exon 8. Thus, inclusion/exclusion of exon 8 and 9 directs expression of IIIb versus IIIc splice forms.

FIGS. 2A-2B illustrate FGFR3 is highly expressed on mouse and human astrocytes. Graphs of RNA sequencing (RNASeq) data depicting expression of FGFR1-FGFR4 transcript in mouse (FIG. 2A) and human (FIG. 2B) nervous and supporting cells.

FIG. 3 shows expression of total FGFR3, and FGFR3b and FGFR3c isoforms, in mouse brain, primary mouse astrocytes, and primary human astrocytes. Data are plotted as relative to glyceraldehyde 3-phosphate dehydrogenase (GAPDH) Ct values, and normalized to the relative expression of each isoform from the mouse brain (1/2ΔΔCt, mean±standard deviation [SD]).

FIGS. 4A-4C illustrate FGFR3 antibodies internalize into live astrocytes as evidenced by perinuclear speckles (puncta). A schematic depicting an exemplary experimental timeline (FIG. 4A). Photomicrograph showing immunofluorescence detection of actin cytoskeleton and perinumclear FGFR3 puncta in human astrocytes using DEAD staining approaches described herein (FIG. 4B). Photomicrographs showing immunofluorescence detection of vimentin (VIM) cytoskeleton and perinuclear FGFR3 puncta in human and mouse astrocytes using LIVE staining approaches described herein (FIG. 4C). Non-degrader (H4H30063P) FGFR3 antibodies showed robust internalization as evidenced by prevalent puncta surrounding the nucleus.

FIGS. 5A-5C illustrate validation of FGFR3 expression and internalization in the U87-FGFR3b-FLuc-GFP line. A schematic depicting an exemplary experimental timeline (FIG. 5A). Photomicrographs showing immunofluorescent detection of FGFR3b and FelD1 (negative control) in GFP-positive U87 cells stained with DAPI (FIG. 5B). FGFR3 are visualized as prevalent fluorescent puncta surrounding the nucleus (DAPI staining), confirming FGFR3 expression and internalization in the U87-FGFR3b-FLuc-GFP line. A graph showing percent normalized TaqMan FGFR3 expression (mean±standard deviation [SD]) in U87 parental cells, and in U87 cell lines expressing human FGFR3b (U87-hFGFR3b), and human FGFR3c (U87-hFGFR3c) isoforms using hFGFR3b and hFGFR3c probes (FIG. 5C).

FIGS. 6A-6C shows GFP and luciferase siRNA knockdown of their respective targets in U87-FGFR3b/c-GFP-luciferase cells. Schematic depicting treatment of the U87-FGFR3b or U87-FGFR3c cell lines, including description of the GFP-P2A-FLuc mRNA (FIG. 6A). The schematic illustrates, in particular, that since luciferase and GFP are present within one contiguous mRNA, a GFP siRNA may knockdown luciferase expression and/or a luciferase siRNA may knockdown GFP expression. Graphs showing mean GFP fluorescence intensity (FIG. 6B) and luciferase activity (FIG. 6C) within the two different siRNA (GFP siRNA and luciferase siRNA) versus non-targeting siRNA, and as compared to untreated controls. Cells were treated with parental siRNA (i.e., unmodified siRNA), revealing that the GFP siRNA was more potent than luciferase siRNA (arrows), a finding which served as an impetus to advance the GFP siRNA via introduction of truncation modifications to this molecule described herein.

FIG. 7 shows exemplary GFP and luciferase siRNA sequences.

FIGS. 8A-8B illustrate modified GFP-targeting siRNA knockdown in U87-FGFR3b-GFP-luciferase cells. Schematic depicting treatment of the U87-FGFR3b cell line, including description of the GFP-P2A-FLuc mRNA (FIG. 8A). A graph showing luciferase activity within 3′-modified (mod) GFP siRNA (3′ truncated siRNA) and 5′-modified (mod) GFP siRNA (5′ truncated siRNA) cells versus GFP siRNA and untreated controls (FIG. 8B).

FIGS. 9A-9B show modified GFP-targeting siRNA molecules. Chemical structure of N6 linker (Horizon Discovery) (FIG. 9A, top), and corresponding N6 modified GFP siRNA sequence, based on reference sequence as described in Caplen et al., 2001 (FIG. 9A, bottom). Exemplary 5′ modified GFP siRNA sequence (truncated) (FIG. 9B, top). Exemplary 3′ modified GFP siRNA sequence (truncated) (FIG. 9B, bottom).

FIGS. 10A-10C illustrate internalized FGFR3b antibody H4H30105P2 co-localizes with early and late endosomes, but not Rab4-positive recycling endosomes, and internalized FGFR3b antibody H4H30063P co-localizes with early and late endosomes, lysosomes, as well as Rab-4 positive recycling endosomes. Schematic depicting treatment of the U87-FGFR3b cell line (FIG. 10A). Photomicrographs depicting immunofluorescent detection of FGFR3b and early endosome marker EEA1 (leftmost), recycling endosome marker Rab4 (left middle), late endosome marker Rab7 (right middle), and lysosome marker Lamp1 (rightmost) in GFP-positive U87 cells stained with DAPI (FIG. 10B). Endosomal trafficking of FGFR3b antibody H4H30105P2 (non-conjugated) illustrates achievement of robust internalization as evidenced by perinuclear speckles (puncta). FGFR3b antibody H4H30105P2 co-localized with the early endosomal marker EEA1 and the late endosomal marker Rab7, but not with the recycling endosomal marker Rab4. Photomicrographs depicting immunofluorescent detection of FGFR3b and early endosome marker EEA1 (leftmost), recycling endosome marker Rab4 (left middle), late endosome marker Rab7 (right middle), and lysosome marker Lamp1 (rightmost) in GFP-positive U87 cells stained with DAPI (FIG. 10C). Endosomal trafficking of FGFR3b antibody H4H30063P (non-conjugated) illustrates achievement of robust internalization as evidenced by perinuclear speckles (puncta). FGFR3b antibody H4H30063P co-localized with the early endosomal marker EEA1, the late endosomal marker Rab7, and the lysosomal marker Lamp1, as well as with the recycling endosomal marker Rab4.

FIG. 11 shows a schematic depicting potential fate(s) of FGFR3 receptors following internalization with FGFR3 antibody conjugated to a molecular cargo. It is not yet known which fate pathway (e.g., degradation or recycling) is most beneficial for delivery for antibody-conjugated molecular cargoes.

FIGS. 12A-12B show screening of FGFR3 antibodies in the U87-FGFR3b-Fluc-GFP cell line. Photomicrographs depicting immunofluorescent detection of FGFR3b in GFP-positive U87 cells stained with DAPI illustrate robust internalization of FGFR3 antibodies in the U87-hFGFR3b cell line (FIG. 12A). Photomicrographs depicting immunofluorescent detection of FGFR3 in GFP-positive U87 cells stained with DAPI illustrate predominately surface staining with small diffuse puncta in the U87-hFGFR3c cell line for FGFR3 antibodies exhibiting best binder (i.e., strong surface binding), weak binders and non-binders properties (FIG. 12B).

FIG. 13 shows HDX epitope mapping results for anti-FGFR3b H4H30117P2, H4H30063P.

FIG. 14 shows HDX epitope mapping results for anti-FGFR3b H4H30045P and H4H30108P2.

FIG. 15 shows HDX protection by FGFR3 antibodies. Regions with above 20% and 25% HDX protection are shown.

FIGS. 16 and 17 show HDX epitope mapping of FGFR3 antibodies.

FIG. 18 shows HDX epitope mapping results for H4H30063P. Figure discloses SEQ ID NO: 238.

FIG. 19 shows HDX epitope mapping results for H4H30108P2. Figure discloses SEQ ID NO: 238.

FIG. 20 shows HDX epitope mapping results for H4H30117P2. Figure discloses SEQ ID NO: 238.

FIG. 21 shows HDX epitope mapping results for H4H30045P. Figure discloses SEQ ID NO: 238.

FIG. 22 shows exemplary Hydrogen-Deuterium Exchange Mass Spectrometry experimental process.

### DETAILED DESCRIPTION OF THE INVENTION

Provided herein are antigen-binding proteins that specifically binds to fibroblast growth factor receptor 3 (FGFR3) or an antigenic fragment thereof that are conjugated to a molecular cargo. Such conjugates are useful, for example, for delivery of the molecular cargo to various tissues (e.g., central nervous system (CNS) tissues, or eye) and/or cells (e.g., astrocytes) in the body. The conjugates described herein have an ability to efficiently deliver molecular cargoes to the nervous system including the brain and the spinal cord and, in particular, astrocytes residing therein and, thus, can be used for treatment of diseases and disorders such as neurodegenerative and neurodevelopmental diseases and disorders.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A polynucleotide includes DNA and RNA. The present disclosure includes any polynucleotide described herein which is operably linked to a promoter or other expression control sequence.

The term “FGFR3” refers to fibroblast growth factor receptor 3. The fibroblast growth factor receptor 3 (FGFR3) belongs to a family of structurally related tyrosine kinase receptors including four different genes (FGFR1-4). These receptors have three glycosylated extracellular immunoglobulin-like domains (Ig-like), a transmembrane domain and a split intracellular tyrosine-kinase domain. Ligand binding induces FGFR dimerization, resulting in autophosphorylation of the kinase domain and interaction with and phosphorylation of effector signaling proteins. Alternative mRNA splicing mechanisms generate many different receptor isoforms, which differ in ligand specificity. The isoforms FGFR3b and FGFR3c result from a mutually exclusive splicing event, in which the second half of the third Ig-like domain is encoded by either the 151 nucleotides of exon 8 or the 145 nucleotides of exon 9. In some embodiments, the FGFR3 referred herein is human FGFR3.

In an embodiment, the human FGFR3c isoform comprises the amino acid sequence:

(SEQ ID NO: 232)
ESLGTEQRVVGRAAEVPGPEPGQQEQLVFGSGDAVELSCPPPGGGPMGP
TVWVKDGTGLVPSERVLVGPQRLQVLNASHEDSGAYSCRQRLTQRVLCH
FSVRVTDAPSSGDDEDGEDEAEDTGVDTGAPYWTRPERMDKKLLAVPAA
NTVRFRCPAAGNPTPSISWLKNGREFRGEHRIGGIKLRHQQWSLVMESV
VPSDRGNYTCVVENKFGSIRQTYTLDVLERSPHRPILQAGLPANQTAVL
GSDVEFHCKVYSDAQPHIQWLKHVEVNGSKVGPDGTPYVTVLKTAGANT
TDKELEVLSLHNVTFEDAGEYTCLAGNSIGFSHHSAWLVVLPAEEELVE
ADEAGSVYAG

In an embodiment, the human FGFR3b isoform comprises the amino acid sequence:

(SEQ ID NO: 233)
ESLGTEQRVVGRAAEVPGPEPGQQEQLVFGSGDAVELSCPPPGGGPMGP
TVWVKDGTGLVPSERVLVGPQRLQVLNASHEDSGAYSCRQRLTQRVLCH
FSVRVTDAPSSGDDEDGEDEAEDTGVDTGAPYWTRPERMDKKLLAVPAA
NTVRFRCPAAGNPTPSISWLKNGREFRGEHRIGGIKLRHQQWSLVMESV
VPSDRGNYTCVVENKFGSIRQTYTLDVLERSPHRPILQAGLPANQTAVL
GSDVEFHCKVYSDAQPHIQWLKHVEVNGSKVGPDGTPYVTVLKSWISES
VEADVRLRLANVSERDGGEYLCRATNFIGVAEKAFWLSVHGPRAAEEEL
VEADEAGSVYAG

In an embodiment, an FGFR3 referred to herein comprises one or more of the following mutations: S249C, R248C, G372C, Y375C, K650E, or FGFR3-TACC3. See e.g., Singh et al., Transforming fusions of FGFR and TACO genes in human glioblastoma. Science (New York, NY) 2012; 337:1231-1235.

### FGFR3 Binding Protein Conjugates

The present disclosure provides FGFR3 binding protein-drug conjugates. A FGFR3 binding protein-drug conjugate comprises an optional signal peptide, connected to an antigen-binding protein (e.g., an antibody or an antigen-binding fragment of an antibody such as a fragment antigen-binding region (Fab) or single chain fragment variable (scFv)) that binds specifically to FGFR3, preferably, human FGFR3, which is conjugated (optionally by a linker) to molecular cargo. The FGFR3 binding proteins described herein can deliver the conjugated molecular cargo to a desired tissue (e.g., nervous tissue) and/or desired cell type (e.g., astrocytes) in the body.

An antigen-binding protein that specifically binds to FGFR3 may bind at about 25° C., to FGFR3 or a fusion protein thereof, for example, a tag such as His6 (SEQ ID NO: 235) and/or myc fused to e.g., human FGFR3b or monkey FGFR3b, e.g., in a surface plasmon resonance assay, with a K_{D} of about 1×10⁻⁷ M or a higher affinity. Such an antigen-binding protein may be referred to as “anti-FGFR3”.

In an embodiment, an FGFR3 binding protein-drug conjugate includes an anti-FGFR3 scFv comprising the arrangement of variable regions as follows LCVR-HCVR or HCVR-LCVR, wherein the HCVR and LCVR are optionally connected by a linker and the scFv is connected, optionally by a linker, to a molecular cargo (e.g., LCVR-(Gly₄ Ser)₃-HCVR-molecular cargo (“GGGGSGGGGSGGGGS” disclosed as SEQ ID NO: 246); or LCVR-(Gly₄ Ser)₃-HCVR-molecular cargo (“GGGGSGGGGSGGGGS” disclosed as SEQ ID NO: 246).

The term “conjugate” means a body in which two substances are linked covalently, or non-covalently. The term “covalently linked” refers to a characteristic of at least two molecules being linked together by way of one or more covalent bond(s). In various embodiments, two molecules can be covalently linked together by a single bond, e.g., a disulfide bridge or a disulfide bond, that operates as a linker between the molecules. In some embodiments, two or more molecules may be covalently linked together by way of a molecule that operates as a linker that joins the at least two molecules together via multiple covalent bonds. In certain embodiments, a linker can be a cleavable linker or a non-cleavable linker. In the conjugate, the two substances may be linked directly or may be linked via a linker. In the present disclosure, one of the two substances is an antigen-binding protein, e.g., an antibody or antigen-binding fragment thereof, and the other is a drug (e.g., a polynucleotide, a polypeptide, a liposome or LNP, or a viral particle or viral capsid protein disclosed herein). In the present disclosure, the linker may be a cleavable linker or may be a non-cleavable linker. In some embodiments, two polypeptide molecules that are covalently linked, either directly or indirectly (e.g., by a linker), may be expressed from one single polynucleotide molecule.

As used herein, the term “antibody-drug conjugate” or “ADC” means a conjugate of an antibody or antigen-binding fragment thereof with a drug (e.g., a polynucleotide, a polypeptide, a liposome or LNP, or a viral particle or viral capsid protein disclosed herein). The affinity to an antigen is imparted to a drug by linking an antibody or antigen-binding fragment thereof with the drug (e.g., a polynucleotide, a polypeptide, a liposome or LNP, or a viral particle or viral capsid protein disclosed herein), thereby increasing the efficiency of delivering the drug to a target site in vivo. “Antibody-drug conjugates” or “ADCs” as used herein also encompass fusion proteins wherein the antibody or antigen-binding fragment thereof is fused with another polypeptide molecule.

In an embodiment, the assignment of amino acids to each framework or CDR domain in an immunoglobulin is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, also included herein are antibodies and antigen-binding fragments including the CDRs of a V_{H} and the CDRs of a V_{L}, which V_{H} and V_{L} comprise amino acid sequences as set forth herein (see e.g., sequences of Table 1-1, or a variant thereof), wherein the CDRs are as defined according to Kabat and/or Chothia.

Protein-drug conjugates described herein include antibodies that bind specifically to the human FGFR3.

The term “antibody”, as used herein, refers to immunoglobulin molecules comprising four polypeptide chains, two heavy chains (HCs) and two light chains (LCs), inter-connected by disulfide bonds (e.g., IgG). In an embodiment, each antibody heavy chain (HC) comprises a heavy chain variable region (“HCVR” or “V H”) (e.g., SEQ ID NO: 2, 22, 42, 62, 82, 102 or a variant thereof) and a heavy chain constant region; and each antibody light chain (LC) comprises a light chain variable region (“LCVR or “V_{L}”) (e.g., SEQ ID NO: 10, 30, 50, 70, 90, 110 or a variant thereof) and a light chain constant region (CL). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Anti-FGFR3 antibodies described herein can also be conjugated to a molecular cargo.

In an embodiment, the assignment of amino acids to each framework or CDR domain in an immunoglobulin is in accordance with the definitions of Sequences of Proteins of Immunological Interest, Kabat et al.; National Institutes of Health, Bethesda, Md.; 5th ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, the present disclosure includes antibodies and antigen-binding fragments including the CDRs of a VH and the CDRs of a VL, which VH and VL comprise amino acid sequences as set forth herein (see e.g., sequences of Table 1-1, or a variant thereof), wherein the CDRs are as defined according to Kabat and/or Chothia.

An FGFR3 binding protein described herein may be an antigen-binding fragment of an antibody which may be conjugated to a molecular cargo. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)2 fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, one-armed antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

In some embodiments, an anti-FGFR3 protein-drug conjugate described herein may comprise an scFv which is conjugated to a molecular cargo. An scFv (single chain fragment variable) has variable regions of heavy (V H) and light (V L) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 Å) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991; 203:46-88). In an embodiment, the linker comprises an amino acid sequence of such length to separate the variable domains by about 3.5 nm. In an embodiment of the invention, an anti-FGFR3 scFv-drug conjugate includes an scFv comprising the arrangement of variable regions as follows LCVR-HCVR or HCVR-LCVR, wherein the HCVR and LCVR are optionally connected by a linker and the scFv is connected, optionally by a linker, to a molecular cargo (e.g., LCVR-(Gly₄ Ser)₃ (SEQ ID NO: 246)-HCVR-molecular cargo; or LCVR-(Gly₄ Ser)₃ (SEQ ID NO: 246)-HCVR-molecular cargo).

In some embodiments, an anti-FGFR3 protein-drug conjugate described herein may comprise a Fab which is conjugated to a molecular cargo.

In some embodiments, an anti-FGFR3 protein-drug conjugate described herein comprise a bivalent antibody which is conjugated to a molecular cargo.

In some embodiments, an anti-FGFR3 protein-drug conjugate described herein comprises a monovalent or “one-armed” antibody which is conjugated to a molecular cargo. The monovalent or “one-armed” antibodies as used herein refer to immunoglobulin proteins comprising a single variable domain. For example, the one-armed antibody may comprise a single variable domain within a Fab wherein the Fab is linked to at least one Fc fragment. In certain embodiments, the one-armed antibody comprises: (i) a heavy chain comprising a heavy chain constant region and a heavy chain variable region, (ii) a light chain comprising a light chain constant region and a light chain variable region, and (iii) a polypeptide comprising a Fc fragment or a truncated heavy chain. In certain embodiments, the Fc fragment or a truncated heavy chain comprised in the separate polypeptide is a “dummy Fc,” which refers to an Fc fragment that is not linked to an antigen binding domain. The one-armed antibodies described herein may comprise any of the HCVR/LCVR pairs or CDR amino acid sequences as set forth in Table 1-1 herein. One-armed antibodies comprising a full-length heavy chain, a full-length light chain and an additional Fc domain polypeptide can be constructed using standard methodologies (see e.g., WO2010151792, which is incorporated herein by reference in its entirety), wherein the heavy chain constant region differs from the Fc domain polypeptide by at least two amino acids (e.g., H95R and Y96F according to the IMGT exon numbering system; or H435R and Y436F according to the EU numbering system). Such modifications are useful in purification of the monovalent antibodies (see WO2010151792).

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a VH domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-CH1, (ii) V_{H}-CH2; (iii) V_{H}-CH3; (iv) V_{H}—CH1-CH2; (v) V_{H}-CH1-CH2-CH3; (vi) V_{H}-CH2-CH3; (vii) V_{H}-CL; (viii) V_{L}-CH1, (ix) V_{L}-CH2; (x) V_{L}-CH3; (xi) V_{L}-CH1-CH2; (xii) V_{L}-CH1-CH2-CH3, (xiii) V_{L}-CH2-CH3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present disclosure includes an antigen-binding fragment of an antigen-binding protein such as an antibody set forth herein, for example, H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2.

Antigen-binding proteins (e.g., antibodies and antigen-binding fragments) may be monospecific or multi-specific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2).

The term “specifically binds” or “binds specifically” refers to those antigen-binding proteins (e.g., antibodies or antigen-binding fragments thereof) having a binding affinity to an antigen, such as human FGFR3 protein (e.g., FGFR3b and/or FGFR3c isoform), mouse FGFR3 protein (e.g., FGFR3b and/or FGFR3c isoform) or cynomolgus monkey FGFR3 protein (e.g., FGFR3b and/or FGFR3c isoform), expressed as K_{D}, of at least about 10⁻⁹ M (e.g., 0.01, 0.1, 0.2, 0.3, 0.4, 0.5, 0.6, 0.7, 0.8, 0.9 or 1.0 nM), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present disclosure includes antigen-binding proteins that specifically bind to FGFR3 protein (e.g., FGFR3b and/or FGFR3c isoform). “Anti-FGFR3” refers to an antigen-binding protein (or other molecule), for example an antibody or antigen-binding fragment thereof, that binds specifically to FGFR3 (e.g., FGFR3b and/or FGFR3c isoform).

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

The present disclosure includes antigen-binding proteins, e.g., antibodies or antigen-binding fragments, that bind to the same epitope as an antigen-binding protein described herein (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2).

An antigen is a molecule, such as a peptide (e.g., FGFR3 or a fragment thereof (an antigenic fragment)), to which, for example, an antibody or antigen-binding fragment thereof binds. The specific region on an antigen that an antibody recognizes and binds to is called the epitope. Antigen-binding proteins (e.g., antibodies) described herein that specifically bind to such antigens are part of the present disclosure.

The term “epitope” refers to an antigenic determinant (e.g., on FGFR3b and/or FGFR3c) that interacts with a specific antigen-binding site of an antigen-binding protein, e.g., a variable region of an antibody, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which antigen-binding proteins described herein bind may be included in fragments of FGFR3, e.g., human FGFR3b and/or FGFR3c, for example the extracellular domain thereof. Antigen-binding proteins (e.g., antibodies) described herein that bind to such epitopes are also contemplated.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present disclosure includes antigen-binding proteins that compete for binding to FGFR3, e.g., an FGFR3b and/or FGFR3c epitope as discussed herein, with an antigen-binding protein described herein, e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2. The term “competes” as used herein, refers to an antigen-binding protein (e.g., antibody or antigen-binding fragment thereof) that binds to an antigen (e.g., FGFR3) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between anti-FGFR3 antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment described herein which is modified in some way retains the ability to specifically bind to FGFR3 (e.g., FGFR3b and/or FGFR3c), e.g., retains at least 10% of its FGFR3 binding activity (when compared to the parental antibody) when that activity is expressed on a molar basis. Preferably, an antibody or antigen-binding fragment described herein retains at least 20%, 50%, 70%, 80%, 90%, 95% or 100% or more of the FGFR3 binding affinity as the parental antibody. It is also intended that an antibody or antigen-binding fragment described herein may include conservative or non-conservative amino acid substitutions (referred to as “conservative variants” or “function conserved variants” of the antibody) that do not substantially alter its biologic activity.

An FGFR3 binding protein described herein may be a monoclonal antibody or an antigen-binding fragment of a monoclonal antibody which may be conjugated to a molecular cargo. The present disclosure includes monoclonal FGFR3 binding proteins, e.g., antibodies and antigen-binding fragments thereof (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2), as well as monoclonal compositions comprising a plurality of isolated monoclonal antigen-binding proteins. The term “monoclonal antibody” or “mAb”, as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, an FGFR3 binding protein, e.g., antibody or antigen-binding fragment (which may be conjugated to a molecular cargo) comprises a heavy chain constant domain, e.g., of the type IgA (e.g., IgA1 or IgA2), IgD, IgE, IgG (e.g., IgG1, IgG2, IgG3 and IgG4) or IgM. In an embodiment, an antigen-binding protein, e.g., antibody or antigen-binding fragment, comprises a light chain constant domain, e.g., of the type kappa or lambda. In an embodiment, a V_{H} as set forth herein is linked to a human heavy chain constant domain (e.g., IgG) and a V_{L} as set forth herein is linked to a human light chain constant domain (e.g., kappa). The present disclosure includes antigen-binding proteins comprising the variable domains set forth herein (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2), which are linked to a heavy and/or light chain constant domain, e.g., as set forth herein.

The present disclosure includes human FGFR3 binding proteins which may be conjugated to a molecular cargo. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See e.g., U.S. Pat. Nos. 8,502,018; 6,596,541 or 5,789,215. The anti-FGFR3 human mAbs described herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject. The present disclosure includes human antigen-binding proteins (e.g., antibodies or antigen-binding fragments thereof such as H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2).

The present disclosure includes anti-FGFR3 chimeric antigen-binding proteins, e.g., antibodies and antigen-binding fragments thereof (which may be conjugated to a molecular cargo), and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567, and Morrison et al., (1984) Proc. Natl. Acad. Sci. USA 81: 6851-6855). The present disclosure includes chimeric antibodies comprising the variable domains which are set forth herein (e.g., from H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2) and a non-human constant domain.

The term “recombinant” FGFR3 binding proteins, such as antibodies or antigen-binding fragments thereof (which may be conjugated to a molecular cargo), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library. The present disclosure includes recombinant antigen-binding proteins, such as antibodies and antigen-binding fragments as set forth herein (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2).

An antigen-binding fragment of an antibody will, in an embodiment, comprise less than a full antibody but still binds specifically to antigen, e.g., FGFR3, e.g., including at least one variable domain. The variable domain may be of any size or amino acid composition and will generally comprise at least one (e.g., 3) CDR(s), which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

A “variant” of a polypeptide, such as an immunoglobulin chain (e.g., an H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2 V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2, 10, 18, 20, 22, 30, 38, 40, 42, 50, 58, 60, 62, 70, 78, 80, 82, 90, 98, 100, 102, 110, 118 or 120); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment), and/or comprising the amino acid sequence but having one or more (e.g., 1, 2, 3, 4, 5, 6, 7, 8, 9 or 10) mutations (e.g., point mutation, insertion, truncation, and/or deletion).

Moreover, a variant of a polypeptide may include a polypeptide such as an immunoglobulin chain (e.g., an H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2 V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. See Table 1-1. For example, the present disclosure includes FGFR3 binding proteins which include an immunoglobulin light chain (or V_{L}) variant comprising the amino acid sequence set forth in SEQ ID NO: 10 but having one or more of such mutations and/or an immunoglobulin heavy chain (or V_{H}) variant comprising the amino acid sequence set forth in SEQ ID NO: 2 but having one or more of such mutations. In an embodiment, an FGFR3 binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to significantly disrupt biological activity. The present disclosure includes FGFR3 binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

“H4H30063 P” “H4H30066P”; “H4H30071P”, “H4H30089P2”; “H4H30093P2”; H4H30076P″; “H4H30105P2”; “H4H30108P2”; “H4H30117P2”, “H4H30045P”; “H4H30061P”, “H4H30095P2” and “H4H30102P2” unless otherwise stated, refer to FGFR3 binding proteins, e.g., antibodies and antigen-binding fragments thereof (including multispecific antigen-binding proteins), comprising an immunoglobulin heavy chain variable region (V_{H}) and an immunoglobulin light chain variable region (V_{L}) comprising the amino acid sequence pair specifically set forth in SEQ ID NOs: 2 & 10; 22 & 30; 42 & 50; 62 & 70; 82 & 90; and 102 & 110 (or a variant of any of said sequences), respectively; or comprising an immunoglobulin heavy chain (HC) and an immunoglobulin light chain (LC) comprising the amino acid sequence pair specifically set forth in SEQ ID NOs: 18 & 20; 38 & 40; 58 & 60; 78 & 80; 98 & 100; 118 & 120; 136 & 138; 155 & 157; 167 & 157; 177 & 157; 195 & 197; 215 & 217; or 229 & 231 (or a variant of any of said sequences), respectively; or that comprise a heavy chain or V_{H} that comprises the CDRs thereof (CDR-H1 (or a variant thereof), CDR-H2 (or a variant thereof) and CDR-H3 (or a variant thereof)) and/or a light chain or V_{L} that comprises the CDRs thereof (CDR-L1 (or a variant thereof), CDR-L2 (or a variant thereof) and CDR-L3 (or a variant thereof)). In an embodiment, the V_{H} is linked to an IgG constant heavy chain domain, for example, human IgG constant heavy chain domain (e.g., IgG1 or IgG4 (e.g., comprising the S228P and/or S108P mutation)) and/or the V_{L} is linked to a light chain constant domain, for example a human light chain constant domain (e.g., lambda or kappa constant light chain domain). Polynucleotides encoding one or more of any such immunoglobulin chains (e.g., V_{H}, V_{L}, HC and/or LC) forms part of the present disclosure.

Antibodies and antigen-binding fragments described herein (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2) comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to FGFR3 comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

In an embodiment, an FGFR3 protein-drug conjugate (e.g., in scFv, Fab, or other antibody or antigen-binding fragment thereof format) can exhibit one or more of the following characteristics:
Binds (e.g., in a surface plasmon resonance assay) to monomeric human FGFR3b (e.g., tagged, for example, at the C-terminus, with myc-myc-His₆) at 25° C. with an affinity (K_{D}) of about 16 nM or greater affinity (e.g., about 16 nM, 12 nM, 10 nM, 7 nM, 5 nM, 4 nM, 3 nM, 2 nM, 1 nM, 0.5 nM, 0.22 nM, 0.2 nM, 0.19 nM, 0.14 nM, 0.1 nM);
Binds (e.g., in a surface plasmon resonance assay) to monomeric Cynomolgous monkey FGFR3b (e.g., tagged, for example, at the C-terminus, with myc-myc-His₆) at 25° C. with an affinity (K_{D}) of about 20 nM or greater affinity (e.g., about 20 nM, 16 nM, 15 nM, 10 nM, 8 nM, 5 nM, 4 nM, 3 nM, 2 nM, 1 nM, 0.9 nM, 0.65 nM, 0.3 nM, 0.28 nM, 0.2 nM, 0.15 nM, 0.1 nM);
Binds (e.g., in a surface plasmon resonance assay) monomeric murine FGFR3b (e.g., tagged, for example, at the C-terminus, with myc-myc-His₆) at 25° C. with an affinity (K_{D}) of about 70 nM or greater affinity (e.g., about 70 nM, 20 nM, 17 nM, 12 nM, 10 nM, 9 nM, 8 nM, 0.1 nM); Does not bind significantly to monomeric human FGFR3c (e.g., tagged, for example, at the C-terminus, with myc-myc-His₆) at 25° C. (e.g., in a surface plasmon resonance assay);
Binds (e.g., in a surface plasmon resonance assay) dimeric human FGFR3b (e.g., tagged, for example, at the C-terminus, with a mouse Fc (mFc)) at 25° C. with an affinity of about 0.6 nM of greater affinity (e.g., about 0.58 nM, 0.17 nM, 0.11 nM, 0.04 nM, 0.03 nM, 0.02 nM, 0.01 nM, 0.023 nM, 0.061 nM, 0.031 nM, 0.016 nM, 0.034 nM, 0.027 nM);
Blocks about 68% or more binding of FGF1 acidic to human FGFR3b-mFc at 200 nM antibody (e.g., 90% or 05%);
Binds to monomeric Cynomolgous and monomeric mouse FGFR3b with a K_{D} that is within about 0.1 nM of the K_{D} for binding to monomeric or dimeric human FGFR3b;
Blocks binding of 4 nM human FGFR3b-mFc to human FGF1 acidic protein with an IC 50 of about 15 nM or a lower concentration (e.g., IC 50 of about 1, 2 or 3 nM); or
Competes for binding to hFGFR3b.mmH with another anti-FGFR3 antibody as set forth in Table 5-1 herein.

The amino acid sequences of domains in FGFR3 binding proteins of conjugates of the present disclosure are summarized below in Table 1-1. For example, anti-FGFR3 antibodies and antigen-binding fragments thereof (e.g., scFvs and Fabs) comprising the HCVR and LCVR of the molecules in Table 1-1; or comprising the CDRs thereof, conjugated to a molecular cargo, form part of the present disclosure.

TABLE 1-1
SEQ ID NOs of Amino Acid Sequences of Domains in Antibodies or Antigen-binding Fragments
(e.g., Fabs or scFv Molecules) in Protein-Drug Conjugates of the Present Disclosure.

| | anti-FGFR3 | | | | | | | | | | |
|---|---|---|---|---|---|---|---|---|---|---|---|
| # | Molecule | HCVR | HCDR1 | HCDR2 | HCDR3 | LCVR | LCDR1 | LCDR2 | LCDR3 | HC* | LC |
| 1 | H4H30063P | 2 | 4 | 6 | 8 | 10 | 12 | 14 | 16 | 18 | 20 |
| 2 | H4H30066P | 22 | 24 | 26 | 28 | 30 | 32 | 34 | 36 | 38 | 40 |
| 3 | H4H30071P | 42 | 44 | 46 | 48 | 50 | 52 | 54 | 56 | 58 | 60 |
| 4 | H4H30089P2 | 62 | 64 | 66 | 68 | 70 | 72 | 74 | 76 | 78 | 80 |
| 5 | H4H30093P2 | 82 | 84 | 86 | 88 | 90 | 92 | 94 | 96 | 98 | 100 |
| 6 | H4H30102P2 | 102 | 104 | 106 | 108 | 110 | 112 | 114 | 116 | 118 | 120 |
| 7 | H4H30076P | 122 | 124 | 126 | 128 | 130 | 132 | 34 | 134 | 136 | 138 |
| 8 | H4H30105P2 | 140 | 142 | 144 | 146 | 148 | 150 | 14 | 153 | 155 | 157 |
| 9 | H4H30108P2 | 159 | 161 | 163 | 165 | 148 | 150 | 14 | 153 | 167 | 157 |
| 10 | H4H30117P2 | 169 | 171 | 173 | 175 | 148 | 150 | 14 | 153 | 177 | 157 |
| 11 | H4H30045P | 179 | 181 | 183 | 185 | 187 | 189 | 191 | 193 | 195 | 197 |
| 12 | H4H30061P | 199 | 201 | 203 | 205 | 207 | 209 | 211 | 213 | 215 | 217 |
| 13 | H4H30095P2 | 219 | 221 | 223 | 225 | 227 | 32 | 34 | 76 | 229 | 231 |

*In an embodiment, the heavy chain lacks the C-terminal Lysine.

Sequences of domains or chains in antibodies or antigen-binding fragments (e.g., Fabs or scFv molecules) in protein-drug conjugates described herein are set forth below. The present disclosure includes any antibody or antigen-binding fragment thereof that includes an HCVR and LCVR having amino acid sequences as set forth below or an HCVR and LCVR having the HCDRs and LCDRs thereof, respectively.

H4H30063P
HCVR Nucleotide Sequence
CAGGTGCAGCTGCAGGAGTCGGGCCCAGGACTGGTGAAGCCTTCGGAGACCCTGTCCCTCACCTGCACTGTCTCT
GGTGACTCCATTAATAGTTACTTCTGGAGCTGGATCCGGCAGTTGCCAGGGAAGGAACTGGAGTGGATTGGCCAT
ATCTATTCTAGTGGGAGTACCAGATACAACCCCTCCCTCCAGAGTCGAGTCACCATATCAATAGACACGTCCAAG
AACCAGTTCTCCCTGAAGCTGAGTTCTGTGACCGCTGCGGACACGGCCGTATATTACTGTGCGAGGGGCGCCAGC
GCAGTTGACTACTGGGGCCAGGGAACCCTGGTCACCGTCTCCTCA
(SEQ ID NO: 1)
HCVR Amino Acid Sequence
QVQLQESGPGLVKPSETLSLTCTVSGDSINSYFWSWIRQLPGKELEWIGHIYSSGSTRYNPSLQSRVTISIDTSK
NQFSLKLSSVTAADTAVYYCARGASAVDYWGQGTLVTVSS
(SEQ ID NO: 2)
HCDR1 Nucleotide Sequence
GGT GAC TCC ATT AAT AGT TAC TTC
(SEQ ID NO: 3)
HCDR1 Amino Acid Sequence
G D S I N S Y F
(SEQ ID NO: 4)
HCDR2 Nucleotide Sequence
ATC TAT TCT AGT GGG AGT ACC
(SEQ ID NO: 5)
HCDR2 Amino Acid Sequence
I Y S S G S T
(SEQ ID NO: 6)
HCDR3 Nucleotide Sequence
GCG AGG GGC GCC AGC GCA GTT GAC TAC
(SEQ ID NO: 7)
HCDR3 Amino Acid Sequence
A R G A S A V D Y
(SEQ ID NO: 8)
LCVR Nucleotide Sequence
GAAATTGTGTTGACGCAGTCTCCAGGCACCCTGTCTTTGTCTCCAGGGGAAAGAGCCACCCTCTCCTGCAGGACC
AGTCAGAGTATTAGCAGCGGCTATTTAGCCTGGTACCAGCAGAAACCTGGCCAGGCTCCCAGGCTCCTCATCTAT
GGTGCATCCAGAAGGGCCACTGGCATCCCAGACAGGTTCAGTGGCAGTGGGTCTGGGACAGACTTCACTCTCACC
ATCAGCAGACTGGAGCCTGAAGACTTTGTAGTGTATTACTGTCAACAATATGGTAGCTCACCATACACTTTTGGC
CAGGGGACCAAGCTGGAGATCAAA
(SEQ ID NO: 9)
LCVR Amino Acid Sequence
EIVLTQSPGTLSLSPGERATLSCRTSQSISSGYLAWYQQKPGQAPRLLIYGASRRATGIPDRFSGSGSGTDFTLT
ISRLEPEDFVVYYCQQYGSSPYTFGQGTKLEIK
(SEQ ID NO: 10)
LCDR1 Nucleotide Sequence
CAG AGT ATT AGC AGC GGC TAT
(SEQ ID NO: 11)
LCDR1 Amino Acid Sequence
Q S I S S G G Y
(SEQ ID NO: 12)
LCDR2 Nucleotide Sequence
GGT GCA TCC
(SEQ ID NO: 13)
LCDR2 Amino Acid Sequence
G A S
(SEQ ID NO: 14)
LCDR3 Nucleotide Sequence
CAA CAA TAT GGT AGC TCA CCA TAC ACT
(SEQ ID NO: 15)
LCDR3 Amino Acid Sequence
Q Q Y G S S P Y T
(SEQ ID NO: 16)
Heavy Chain Nucleotide Sequence
CAGGTGCAGCTGCAGGAGTCGGGCCCAGGACTGGTGAAGCCTTCGGAGACCCTGTCCCTCACCTGCACTGTCTCT
GGTGACTCCATTAATAGTTACTTCTGGAGCTGGATCCGGCAGTTGCCAGGGAAGGAACTGGAGTGGATTGGCCAT
ATCTATTCTAGTGGGAGTACCAGATACAACCCCTCCCTCCAGAGTCGAGTCACCATATCAATAGACACGTCCAAG
AACCAGTTCTCCCTGAAGCTGAGTTCTGTGACCGCTGCGGACACGGCCGTATATTACTGTGCGAGGGGCGCCAGC
GCAGTTGACTACTGGGGCCAGGGAACCCTGGTCACCGTCTCCTCAGCCTCCACCAAGGGCCCATCGGTCTTCCCC
CTGGCGCCCTGCTCCAGGAGCACCTCCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACACCTTCCCGGCTGTCCTACAGTCCTCA
GGACTCTACTCCCTCAGCAGCGTGGTGACCGTGCCCTCCAGCAGCTTGGGCACGAAGACCTACACCTGCAACGTA
GATCACAAGCCCAGCAACACCAAGGTGGACAAGAGAGTTGAGTCCAAATATGGTCCCCCATGCCCACCCTGCCCA
GCACCTGAGTTCCTGGGGGGACCATCAGTCTTCCTGTTCCCCCCAAAACCCAAGGACACTCTCATGATCTCCCGG
ACCCCTGAGGTCACGTGCGTGGTGGTGGACGTGAGCCAGGAAGACCCCGAGGTCCAGTTCAACTGGTACGTGGAT
GGCGTGGAGGTGCATAATGCCAAGACAAAGCCGCGGGAGGAGCAGTTCAACAGCACGTACCGTGTGGTCAGCGTC
CTCACCGTCCTGCACCAGGACTGGCTGAACGGCAAGGAGTACAAGTGCAAGGTCTCCAACAAAGGCCTCCCGTCC
TCCATCGAGAAAACCATCTCCAAAGCCAAAGGGCAGCCCCGAGAGCCACAGGTGTACACCCTGCCCCCATCCCAG
GAGGAGATGACCAAGAACCAGGTCAGCCTGACCTGCCTGGTCAAAGGCTTCTACCCCAGCGACATCGCCGTGGAG
TGGGAGAGCAATGGGCAGCCGGAGAACAACTACAAGACCACGCCTCCCGTGCTGGACTCCGACGGCTCCTTCTTC
CTCTACAGCAGGCTCACCGTGGACAAGAGCAGGTGGCAGGAGGGGAATGTCTTCTCATGCTCCGTGATGCATGAG
GCTCTGCACAACCACTACACACAGAAGTCCCTCTCCCTGTCTCTGGGTAAATGA
(SEQ ID NO: 17)
Heavy Chain Amino Acid Sequence
QVQLQESGPGLVKPSETLSLTCTVSGDSINSYFWSWIRQLPGKELEWIGHIYSSGSTRYNPSLQSRVTISIDTSK
NQFSLKLSSVTAADTAVYYCARGASAVDYWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPCPPCP
APEFLGGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALHNHYTQKSLSLSLGK
(SEQ ID NO: 18)
Light Chain Nucleotide Sequence
GAAATTGTGTTGACGCAGTCTCCAGGCACCCTGTCTTTGTCTCCAGGGGAAAGAGCCACCCTCTCCTGCAGGACC
AGTCAGAGTATTAGCAGCGGCTATTTAGCCTGGTACCAGCAGAAACCTGGCCAGGCTCCCAGGCTCCTCATCTAT
GGTGCATCCAGAAGGGCCACTGGCATCCCAGACAGGTTCAGTGGCAGTGGGTCTGGGACAGACTTCACTCTCACC
ATCAGCAGACTGGAGCCTGAAGACTTTGTAGTGTATTACTGTCAACAATATGGTAGCTCACCATACACTTTTGGC
CAGGGGACCAAGCTGGAGATCAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 19)
Light Chain Amino Acid Sequence
EIVLTQSPGTLSLSPGERATLSCRTSQSISSGYLAWYQQKPGQAPRLLIYGASRRATGIPDRESGSGSGTDFTLT
ISRLEPEDFVVYYCQQYGSSPYTFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
(SEQ ID NO: 20)
H4H30066P
HCVR Nucleotide Sequence
CAGGTGCAGCTGGTACAGTCTGGGGCTGAGGTGAAGAAGCCTGGGGCCTCAGTGAAGGTCTCCTGCAAGGTTTCC
GGATACACCCTCACTGAATTATCCATGCACTGGGTGCGACAAGCTCCTGGAAAAGGGCTTGAGTGGATGGGAGGT
TTTGATCCTGAAGATGGTGAAATAATCTACGCACAGAAGTTCCAGGGCAGAGTCACCATGACCGAGGACACATCT
ACAGACACAGCCTACATGGACCTGAGCAGTCTGACATCTGAAGACACGGCCGTGTATTACTGTGCAACGGAGAAG
CAGCAACTGGTACGAAAATACTACTTCTACTACGGTTTGGCCGTCTGGGGCCAAGGGACCACGGTCACCGTCTCC
TCA
(SEQ ID NO: 21)
HCVR Amino Acid Sequence
QVQLVQSGAEVKKPGASVKVSCKVSGYTLTELSMHWVRQAPGKGLEWMGGFDPEDGEIIYAQKFQGRVTMTEDTS
TDTAYMDLSSLTSEDTAVYYCATEKQQLVRKYYFYYGLAVWGQGTTVTVSS
(SEQ ID NO: 22)
HCDR1 Nucleotide Sequence
GGA TAC ACC CTC ACT GAA TTA TCC
(SEQ ID NO: 23)
HCDR1 Amino Acid Sequence
G Y T L T E L S
(SEQ ID NO: 24)
HCDR2 Nucleotide Sequence
TTT GAT CCT GAA GAT GGT GAA ATA
(SEQ ID NO: 25)
HCDR2 Amino Acid Sequence
F D P E D G E I
(SEQ ID NO: 26)
HCDR3 Nucleotide Sequence
GCA ACG GAG AAG CAG CAA CTG GTA CGA AAA TAC TAC TTC TAC TAC GGT TTG GCC GTC
(SEQ ID NO: 27)
HCDR3 Amino Acid Sequence
A R T E K Q Q L V R K Y Y F Y Y G L A V
(SEQ ID NO: 28)
LCVR Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGTTATTTAAATTGGTATCAGCAGAAACCAGGGAAAGTCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTCCCCCATTCACTTTCGGCCCT
GGGACCAAAGTGGATATCAAA
(SEQ ID NO: 29)
LCVR Amino Acid Sequence
DIQMTQSPSSLSASVGDRVTITCRASQSISSYLNWYQQKPGKVPKLLIYAASSLQSGVPSRFSGSGSGTDFTLTI
SSLQPEDFATYYCQQSYSPPFTFGPGTKVDIK(SEQ ID NO: 30)
LCDR1 Nucleotide Sequence
CAG AGC ATT AGC AGT TAT
(SEQ ID NO: 31)
LCDR1 Amino Acid Sequence
Q S I S S Y
(SEQ ID NO: 32)
LCDR2 Nucleotide Sequence
GCT GCA TCC
(SEQ ID NO: 33)
LCDR2 Amino Acid Sequence
A A S
(SEQ ID NO: 34)
LCDR3 Nucleotide Sequence
CAA CAG AGT TAC AGT CCC CCA TTC ACT
(SEQ ID NO: 35)
LCDR3 Amino Acid Sequence
Q Q S Y S P P F T
(SEQ ID NO: 36)
Heavy Chain Nucleotide Sequence
CAGGTGCAGCTGGTACAGTCTGGGGCTGAGGTGAAGAAGCCTGGGGCCTCAGTGAAGGTCTCCTGCAAGGTTTCC
GGATACACCCTCACTGAATTATCCATGCACTGGGTGCGACAAGCTCCTGGAAAAGGGCTTGAGTGGATGGGAGGT
TTTGATCCTGAAGATGGTGAAATAATCTACGCACAGAAGTTCCAGGGCAGAGTCACCATGACCGAGGACACATCT
ACAGACACAGCCTACATGGACCTGAGCAGTCTGACATCTGAAGACACGGCCGTGTATTACTGTGCAACGGAGAAG
CAGCAACTGGTACGAAAATACTACTTCTACTACGGTTTGGCCGTCTGGGGCCAAGGGACCACGGTCACCGTCTCC
TCAGCCTCCACCAAGGGCCCATCGGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAGGTGGACAAGAGAGTTGAG
TCCAAATATGGTCCCCCATGCCCACCCTGCCCAGCACCTGAGTTCCTGGGGGGACCATCAGTCTTCCTGTTCCCC
CCAAAACCCAAGGACACTCTCATGATCTCCCGGACCCCTGAGGTCACGTGCGTGGTGGTGGACGTGAGCCAGGAA
GACCCCGAGGTCCAGTTCAACTGGTACGTGGATGGCGTGGAGGTGCATAATGCCAAGACAAAGCCGCGGGAGGAG
CAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGA
GAGCCACAGGTGTACACCCTGCCCCCATCCCAGGAGGAGATGACCAAGAACCAGGTCAGCCTGACCTGCCTGGTC
AAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAG
GGGAATGTCTTCTCATGCTCCGTGATGCATGAGGCTCTGCACAACCACTACACACAGAAGTCCCTCTCCCTGTCT
CTGGGTAAATGA
(SEQ ID NO: 37)
Heavy Chain Amino Acid
QVQLVQSGAEVKKPGASVKVSCKVSGYTLTELSMHWVRQAPGKGLEWMGGFDPEDGEIIYAQKFQGRVTMTEDTS
TDTAYMDLSSLTSEDTAVYYCATEKQQLVRKYYFYYGLAVWGQGTTVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDKRVE
SKYGPPCPPCPAPEFLGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHEALHNHYTQKSLSLS
LGK
(SEQ ID NO: 38)
Light Chain Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGTTATTTAAATTGGTATCAGCAGAAACCAGGGAAAGTCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTCCCCCATTCACTTTCGGCCCT
GGGACCAAAGTGGATATCAAACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAG
(SEQ ID NO: 39)
Light Chain Amino Acid
DIQMTQSPSSLSASVGDRVTITCRASQSISSYLNWYQQKPGKVPKLLIYAASSLQSGVPSRFSGSGSGTDFTLTI
SSLQPEDFATYYCQQSYSPPFTFGPGTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
(SEQ ID NO: 40)
H4H30071P
HCVR Nucleotide Sequence
CAGGTACAGCTGCAGCAGTCAGGTCCAGGACTGGTGAAGCCCTCGCAGACCCTCTCACTCACCTGTGCCATCTCC
GGGGACAGTGTCTCTAGGAACAGTGCTGCTTGGAACTGGATCAGGCAGTCCCCATCGAGTGGCCTTGAGTGGCTG
GGAAGGACATACTACAGGTCCAAGTGGTTTTATGATTATGCAATATCTGTGAAAAGTCGAATAACCGTCAACCCA
GACACATCCAAGAACCAATTCTCCCTTCACCTGAACTCTGTGACTCCCGAAGACACGGCTGTCTATTACTGTGCG
AGAGGCTACGGTGGCTACGAGGACTACTTTGACTACTGGGGCCAGGGAACCCTGGTCACCGTCTCCTCA
(SEQ ID NO: 41)
HCVR Amino Acid Sequence
QVQLQQSGPGLVKPSQTLSLTCAISGDSVSRNSAAWNWIRQSPSSGLEWLGRTYYRSKWFYDYAISVKSRITVNP
DTSKNQFSLHLNSVTPEDTAVYYCARGYGGYEDYFDYWGQGTLVTVSS
(SEQ ID NO: 42)
HCDR1 Nucleotide Sequence
GGG GAC AGT GTC TCT AGG AAC AGT GCT GCT
(SEQ ID NO: 43)
HCDR1 Amino Acid Sequence
G D S V S R N S A A
(SEQ ID NO: 44)
HCDR2 Nucleotide Sequence
ACA TAC TAC AGG TCC AAG TGG TTT TAT
(SEQ ID NO: 45)
HCDR2 Amino Acid Sequence
T Y Y R S K W F Y
(SEQ ID NO: 46)
HCDR3 Nucleotide Sequence
GCG AGA GGC TAC GGT GGC TAC GAG GAC TAC TTT GAC TAC
(SEQ ID NO: 47)
HCDR3 Amino Acid Sequence
A R G Y G G Y E D Y F D Y
(SEQ ID NO: 48)
LCVR Nucleotide Sequence
GACATCCAGATGACCCAGTCTCCATCTTCCGTGTCTGCATCTGTAGGAGACAGAGTCACCATCACTTGTCGGGCG
AGTCAGGGTATTAGCAGCTGGTTAGCCTGGTATCAGCAGAAACCAGGGAAAGCCCCTAAGCTCCTGATCTATGGT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTTTTGTCAACAGGGTAGCAGTTTCCCGTACACTTTTGGCCAG
GGGACCAAGCTGGAGATCAAA
(SEQ ID NO: 49)
LCVR Amino Acid Sequence
DIQMTQSPSSVSASVGDRVTITCRASQGISSWLAWYQQKPGKAPKLLIYGASSLQSGVPSRFSGSGSGTDFTLTI
SSLQPEDFATYFCQQGSSFPYTFGQGTKLEIK(SEQ ID NO: 50)
LCDR1 Nucleotide Sequence
CAG GGT ATT AGC AGC TGG
(SEQ ID NO: 51)
LCDR1 Amino Acid Sequence
Q G I S S W
(SEQ ID NO: 52)
LCDR2 Nucleotide Sequence
GGT GCA TCC
(SEQ ID NO: 53)
LCDR2 Amino Acid Sequence
G A S
(SEQ ID NO: 54)
LCDR3 Nucleotide Sequence
CAA CAG GGT AGC AGT TTC CCG TAC ACT
(SEQ ID NO: 55)
LCDR3 Amino Acid Sequence
Q Q G S S F P Y T
(SEQ ID NO: 56)
Heavy Chain Nucleotide Sequence
CAGGTACAGCTGCAGCAGTCAGGTCCAGGACTGGTGAAGCCCTCGCAGACCCTCTCACTCACCTGTGCCATCTCC
GGGGACAGTGTCTCTAGGAACAGTGCTGCTTGGAACTGGATCAGGCAGTCCCCATCGAGTGGCCTTGAGTGGCTG
GGAAGGACATACTACAGGTCCAAGTGGTTTTATGATTATGCAATATCTGTGAAAAGTCGAATAACCGTCAACCCA
GACACATCCAAGAACCAATTCTCCCTTCACCTGAACTCTGTGACTCCCGAAGACACGGCTGTCTATTACTGTGCG
AGAGGCTACGGTGGCTACGAGGACTACTTTGACTACTGGGGCCAGGGAACCCTGGTCACCGTCTCCTCAGCCTCC
ACCAAGGGCCCATCGGTCTTCCCCCTGGCGCCCTGCTCCAGGAGCACCTCCGAGAGCACAGCCGCCCTGGGCTGC
CTGGTCAAGGACTACTTCCCCGAACCGGTGACGGTGTCGTGGAACTCAGGCGCCCTGACCAGCGGCGTGCACACC
TTCCCGGCTGTCCTACAGTCCTCAGGACTCTACTCCCTCAGCAGCGTGGTGACCGTGCCCTCCAGCAGCTTGGGC
ACGAAGACCTACACCTGCAACGTAGATCACAAGCCCAGCAACACCAAGGTGGACAAGAGAGTTGAGTCCAAATAT
GGTCCCCCATGCCCACCCTGCCCAGCACCTGAGTTCCTGGGGGGACCATCAGTCTTCCTGTTCCCCCCAAAACCC
AAGGACACTCTCATGATCTCCCGGACCCCTGAGGTCACGTGCGTGGTGGTGGACGTGAGCCAGGAAGACCCCGAG
GTCCAGTTCAACTGGTACGTGGATGGCGTGGAGGTGCATAATGCCAAGACAAAGCCGCGGGAGGAGCAGTTCAAC
AGCACGTACCGTGTGGTCAGCGTCCTCACCGTCCTGCACCAGGACTGGCTGAACGGCAAGGAGTACAAGTGCAAG
GTCTCCAACAAAGGCCTCCCGTCCTCCATCGAGAAAACCATCTCCAAAGCCAAAGGGCAGCCCCGAGAGCCACAG
GTGTACACCCTGCCCCCATCCCAGGAGGAGATGACCAAGAACCAGGTCAGCCTGACCTGCCTGGTCAAAGGCTTC
TACCCCAGCGACATCGCCGTGGAGTGGGAGAGCAATGGGCAGCCGGAGAACAACTACAAGACCACGCCTCCCGTG
CTGGACTCCGACGGCTCCTTCTTCCTCTACAGCAGGCTCACCGTGGACAAGAGCAGGTGGCAGGAGGGGAATGTC
TTCTCATGCTCCGTGATGCATGAGGCTCTGCACAACCACTACACACAGAAGTCCCTCTCCCTGTCTCTGGGTAAA
TGA
(SEQ ID NO: 57)
Heavy Chain Amino Acid
QVQLQQSGPGLVKPSQTLSLTCAISGDSVSRNSAAWNWIRQSPSSGLEWLGRTYYRSKWFYDYAISVKSRITVNP
DTSKNQFSLHLNSVTPEDTAVYYCARGYGGYEDYFDYWGQGTLVTVSSASTKGPSVFPLAPCSRSTSESTAALGC
LVKDYFPEPVTVSWNSGALTSGVHTFPAVLQSSGLYSLSSVVTVPSSSLGTKTYTCNVDHKPSNTKVDKRVESKY
GPPCPPCPAPEFLGGPSVFLFPPKPKDTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREPQVYTLPPSQEEMTKNQVSLTCLVKGF
YPSDIAVEWESNGQPENNYKTTPPVLDSDGSFFLYSRLTVDKSRWQEGNVFSCSVMHEALHNHYTQKSLSLSLGK
(SEQ ID NO: 58)
Light Chain Nucleotide Sequence
GACATCCAGATGACCCAGTCTCCATCTTCCGTGTCTGCATCTGTAGGAGACAGAGTCACCATCACTTGTCGGGCG
AGTCAGGGTATTAGCAGCTGGTTAGCCTGGTATCAGCAGAAACCAGGGAAAGCCCCTAAGCTCCTGATCTATGGT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTTTTGTCAACAGGGTAGCAGTTTCCCGTACACTTTTGGCCAG
GGGACCAAGCTGGAGATCAAACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAG
(SEQ ID NO: 59)
Light Chain Amino Acid
DIQMTQSPSSVSASVGDRVTITCRASQGISSWLAWYQQKPGKAPKLLIYGASSLQSGVPSRFSGSGSGTDFTLTI
SSLQPEDFATYFCQQGSSFPYTFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC
(SEQ ID NO: 60)
H4H30089P2
HCVR Nucleotide Sequence
GAGGTGCAGCTGGTGGAGTCTGGGGGAGGCTTGGTACAGCCTGGAGGGTCCCTGAGACTCTCCTGTATAGTCTCT
GGATTCATCTTCAGTAGTTATGAAATGAGCTGGCTCCGCCAGGCTCCAGGGAAGGGCCTGGAGTGGATTTCATAT
ATTAGTAGTAGTGGTCGTGTCATATACTATGCAGACTCTGTGAAGGGCCGATTCACCATCTCCAGAGACAGCGCC
AAGAATTCACTGTATCTGGAAATGAATAGTCTGAGAGCCGAAGACACGGCTATATATTATTGTACGAGAAAGTGG
GATAGTAGTGGCCCATTTGACTTCTGGGGCCAGGGAACCCTGGTCACCGTCTCCTCA
(SEQ ID NO: 61)
HCVR Amino Acid Sequence
EVQLVESGGGLVQPGGSLRLSCIVSGFIFSSYEMSWLRQAPGKGLEWISYISSSGRVIYYADSVKGRFTISRDSA
KNSLYLEMNSLRAEDTAIYYCTRKWDSSGPFDFWGQGTLVTVSS
(SEQ ID NO: 62)
HCDR1 Nucleotide Sequence
GGA TTC ATC TTC AGT AGT TAT GAA
(SEQ ID NO: 63)
HCDR1 Amino Acid Sequence
G F I F S S Y E
(SEQ ID NO: 64)
HCDR2 Nucleotide Sequence
ATT AGT AGT AGT GGT CGT GTC ATA
(SEQ ID NO: 65)
HCDR2 Amino Acid Sequence
I S S S G R V I
(SEQ ID NO: 66)
HCDR3 Nucleotide Sequence
ACG AGA AAG TGG GAT AGT AGT GGC CCA TTT GAC TTC
(SEQ ID NO: 67)
HCDR3 Amino Acid Sequence
T R K W D S S G P F D F
(SEQ ID NO: 68)
LCVR Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
(SEQ ID NO: 69)
LCVR Amino Acid Sequence
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
(SEQ ID NO: 70)
LCDR1 Nucleotide Sequence
CAG AGC ATT AGC AGC TAT
(SEQ ID NO: 71)
LCDR1 Amino Acid Sequence
Q S I S S Y
(SEQ ID NO: 72)
LCDR2 Nucleotide Sequence
GCT GCA TCC
(SEQ ID NO: 73)
LCDR2 Amino Acid Sequence
A A S
(SEQ ID NO: 74)
LCDR3 Nucleotide Sequence
CAA CAG AGT TAC AGT ACC CCT CCG ATC ACC
(SEQ ID NO: 75)
LCDR3 Amino Acid Sequence
Q Q S Y S T P P I T
(SEQ ID NO: 76)
Heavy Chain Nucleotide Sequence
GAGGTGCAGCTGGTGGAGTCTGGGGGAGGCTTGGTACAGCCTGGAGGGTCCCTGAGACTCTCCTGTATAGTCTCT
GGATTCATCTTCAGTAGTTATGAAATGAGCTGGCTCCGCCAGGCTCCAGGGAAGGGCCTGGAGTGGATTTCATAT
ATTAGTAGTAGTGGTCGTGTCATATACTATGCAGACTCTGTGAAGGGCCGATTCACCATCTCCAGAGACAGCGCC
AAGAATTCACTGTATCTGGAAATGAATAGTCTGAGAGCCGAAGACACGGCTATATATTATTGTACGAGAAAGTGG
GATAGTAGTGGCCCATTTGACTTCTGGGGCCAGGGAACCCTGGTCACCGTCTCCTCAGCCTCCACCAAGGGCCCA
TCGGTCTTCCCCCTGGCGCCCTGCTCCAGGAGCACCTCCGAGAGCACAGCCGCCCTGGGCTGCCTGGTCAAGGAC
TACTTCCCCGAACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAATATGGTCCCCCATGC
CCACCCTGCCCAGCACCTGAGTTCCTGGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAGTTCAACAGCACGTACCGT
GTGGTCAGCGTCCTCACCGTCCTGCACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCCCCGAGAGCCACAGGTGTACACCCTG
CCCCCATCCCAGGAGGAGATGACCAAGAACCAGGTCAGCCTGACCTGCCTGGTCAAAGGCTTCTACCCCAGCGAC
ATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCC
GTGATGCATGAGGCTCTGCACAACCACTACACACAGAAGTCCCTCTCCCTGTCTCTGGGTAAATGA
(SEQ ID NO: 77)
Heavy Chain Amino Acid
EVQLVESGGGLVQPGGSLRLSCIVSGFIFSSYEMSWLRQAPGKGLEWISYISSSGRVIYYADSVKGRFTISRDSA
KNSLYLEMNSLRAEDTAIYYCTRKWDSSGPFDFWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK
(SEQ ID NO: 78)
Light Chain Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 79)
Light Chain Amino Acid
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
(SEQ ID NO: 80)
H4H30093P2
HCVR Nucleotide Sequence
CAGGTGCAGCTGGTGGAGTCTGGGGGAGGCTTGGTCAAGCCTGGAGGGTCCCTAAGACTCTCCTGTGCAGCCTCT
GGATTCACCTTCAATGACTACCCAATGAGCTGGATCCGCCAGGCTCCAGGGAAGGGACTGGAGTGGGTTTCATAC
ATTACTAGCAGTAGTGGTAGTACCATATACTACGCAGACTCTGTGAAGGGCCGATTCACCATCTCCAGGGACAAC
GCCAAGAACTCACTGTATCTGCAAATGAACAGCCTGAGAGCCGAGGACACGGCCGTGTATTACTGTGCGAGAGAG
GTTGTAGTGGCTACGATTGGGGGCTACTACGGTATGGACGTCTGGGGCCAAGGGACCACGGTCACCGTCTCCTCA
(SEQ ID NO: 81)
HCVR Amino Acid Sequence
QVQLVESGGGLVKPGGSLRLSCAASGFTFNDYPMSWIRQAPGKGLEWVSYITSSSGSTIYYADSVKGRFTISRDN
AKNSLYLQMNSLRAEDTAVYYCAREVVVATIGGYYGMDVWGQGTTVTVSS
(SEQ ID NO: 82)
HCDR1 Nucleotide Sequence
GGA TTC ACC TTC AAT GAC TAC CCA
(SEQ ID NO: 83)
HCDR1 Amino Acid Sequence
G F T F N D Y P
(SEQ ID NO: 84)
HCDR2 Nucleotide Sequence
ATT ACT AGC AGT AGT GGT AGT ACC ATA
(SEQ ID NO: 85)
HCDR2 Amino Acid Sequence
I T S S S G S T I
(SEQ ID NO: 86)
HCDR3 Nucleotide Sequence
GCG AGA GAG GTT GTA GTG GCT ACG ATT GGG GGC TAC TAC GGT ATG GAC GTC
(SEQ ID NO: 87)
HCDR3 Amino Acid Sequence A R E V V V A T I G G Y Y G M D V
(SEQ ID NO: 88)
LCVR Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
(SEQ ID NO: 89)
LCVR Amino Acid Sequence
DIQMTQSPSSLSASVGDRVTITCRASQSISSYLNWYQQKPGKAPKLLIYAASSLQSGVPSRFSGSGSGTDETLTI
SSLQPEDFATYYCQQSYSTPPITFGQGTRLEIK
(SEQ ID NO: 90)
LCDR1 Nucleotide Sequence
CAG AGC ATT AGC AGC TAT
(SEQ ID NO: 91)
LCDR1 Amino Acid Sequence
Q S I S S Y
(SEQ ID NO: 92)
LCDR2 Nucleotide Sequence
GCT GCA TCC
(SEQ ID NO: 93)
LCDR2 Amino Acid Sequence
A A S
(SEQ ID NO: 94)
LCDR3 Nucleotide Sequence
CAA CAG AGT TAC AGT ACC CCT CCG ATC ACC
(SEQ ID NO: 95)
LCDR3 Amino Acid Sequence
Q Q S Y D Y P P I T
(SEQ ID NO: 96)
Heavy Chain Nucleotide Sequence
CAGGTGCAGCTGGTGGAGTCTGGGGGAGGCTTGGTCAAGCCTGGAGGGTCCCTAAGACTCTCCTGTGCAGCCTCT
GGATTCACCTTCAATGACTACCCAATGAGCTGGATCCGCCAGGCTCCAGGGAAGGGACTGGAGTGGGTTTCATAC
ATTACTAGCAGTAGTGGTAGTACCATATACTACGCAGACTCTGTGAAGGGCCGATTCACCATCTCCAGGGACAAC
GCCAAGAACTCACTGTATCTGCAAATGAACAGCCTGAGAGCCGAGGACACGGCCGTGTATTACTGTGCGAGAGAG
GTTGTAGTGGCTACGATTGGGGGCTACTACGGTATGGACGTCTGGGGCCAAGGGACCACGGTCACCGTCTCCTCA
GCCTCCACCAAGGGCCCATCGGTCTTCCCCCTGGCGCCCTGCTCCAGGAGCACCTCCGAGAGCACAGCCGCCCTG
GGCTGCCTGGTCAAGGACTACTTCCCCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAGCGTGGTGACCGTGCCCTCCAGCAGC
TTGGGCACGAAGACCTACACCTGCAACGTAGATCACAAGCCCAGCAACACCAAGGTGGACAAGAGAGTTGAGTCC
AAATATGGTCCCCCATGCCCACCCTGCCCAGCACCTGAGTTCCTGGGGGGACCATCAGTCTTCCTGTTCCCCCCA
AAACCCAAGGACACTCTCATGATCTCCCGGACCCCTGAGGTCACGTGCGTGGTGGTGGACGTGAGCCAGGAAGAC
CCCGAGGTCCAGTTCAACTGGTACGTGGATGGCGTGGAGGTGCATAATGCCAAGACAAAGCCGCGGGAGGAGCAG
TTCAACAGCACGTACCGTGTGGTCAGCGTCCTCACCGTCCTGCACCAGGACTGGCTGAACGGCAAGGAGTACAAG
TGCAAGGTCTCCAACAAAGGCCTCCCGTCCTCCATCGAGAAAACCATCTCCAAAGCCAAAGGGCAGCCCCGAGAG
CCACAGGTGTACACCCTGCCCCCATCCCAGGAGGAGATGACCAAGAACCAGGTCAGCCTGACCTGCCTGGTCAAA
GGCTTCTACCCCAGCGACATCGCCGTGGAGTGGGAGAGCAATGGGCAGCCGGAGAACAACTACAAGACCACGCCT
CCCGTGCTGGACTCCGACGGCTCCTTCTTCCTCTACAGCAGGCTCACCGTGGACAAGAGCAGGTGGCAGGAGGGG
AATGTCTTCTCATGCTCCGTGATGCATGAGGCTCTGCACAACCACTACACACAGAAGTCCCTCTCCCTGTCTCTG
GGTAAATGA
(SEQ ID NO: 97)
Heavy Chain Amino Acid
QVQLVESGGGLVKPGGSLRLSCAASGFTENDYPMSWIRQAPGKGLEWVSYITSSSGSTIYYADSVKGRFTISRDN
AKNSLYLQMNSLRAEDTAVYYCAREVVVATIGGYYGMDVWGQGTTVTVSSASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEGNVFSCSVMHEALHNHYTQKSLSLSL
GK
(SEQ ID NO: 98)
Light Chain Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 99)
Light Chain Amino Acid
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
(SEQ ID NO: 100)
H4H30102P2
HCVR Nucleotide Sequence
GAGGTGCAGCTGGTGGAGTCTGGGGGAGACTTGGTACAGCCTGGAGGGTCCCTGAGACTCTCCTGTGCAGCCTCT
GGATTCACCTTCAGTAGTTATGAAATGAACTGGGTCCGCCAGGCTCCAGGGAAGGGGCTGGAGTGGGTTTCATAC
ATTAGTAATAGTGGTTCTACCATATACTACGCAGACTCTGTGAAGGGCCGATTCACCATCTCCAGAGACAACGCC
GAGACCTCACTGTATCTGCAAATGAACAGCCTGAGAGCCGAGGACACGGCTGTTTATTACTGTGCGAGAGAGGGA
TGGGGCGCATATTGTGCTGGTGATTGCTATTCTGGTTTTGATATTTGGGGCCAAGGGACAATGGTCACCGTCTCT
TCA
(SEQ ID NO: 101)
HCVR Amino Acid Sequence
EVQLVESGGDLVQPGGSLRLSCAASGFTFSSYEMNWVRQAPGKGLEWVSYISNSGSTIYYADSVKGRFTISRDNA
ETSLYLQMNSLRAEDTAVYYCAREGWGAYCAGDCYSGFDIWGQGTMVTVSS
(SEQ ID NO: 102)
HCDR1 Nucleotide Sequence
GGA TTC ACC TTC AGT AGT TAT GAA
(SEQ ID NO: 103)
HCDR1 Amino Acid Sequence
G F T F S S Y E
(SEQ ID NO: 104)
HCDR2 Nucleotide Sequence
ATT AGT AAT AGT GGT TCT ACC ATA
(SEQ ID NO: 105)
HCDR2 Amino Acid Sequence
I S N S G S T I
(SEQ ID NO: 106)
HCDR3 Nucleotide Sequence
GCG AGA GAG GGA TGG GGC GCA TAT TGT GCT GGT GAT TGC TAT TCT GGT TTT GAT ATT
(SEQ ID NO: 107)
HCDR3 Amino Acid Sequence
A R E G W G A Y C A G D C Y S G F D I
(SEQ ID NO: 108)
LCVR Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
(SEQ ID NO: 109)
LCVR Amino Acid Sequence
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
(SEQ ID NO: 110)
LCDR1 Nucleotide Sequence
CAG AGC ATT AGC AGC TAT
(SEQ ID NO: 111)
LCDR1 Amino Acid Sequence
Q S I S S Y
(SEQ ID NO: 112)
LCDR2 Nucleotide Sequence
GCT GCA TCC
(SEQ ID NO: 113)
LCDR2 Amino Acid Sequence
A A S
(SEQ ID NO: 114)
LCDR3 Nucleotide Sequence
CAA CAG AGT TAC AGT ACC CCT CCG ATC ACC
(SEQ ID NO: 115)
LCDR3 Amino Acid Sequence
Q Q S S S T P P I T
(SEQ ID NO: 116)
Heavy Chain Nucleotide Sequence
GAGGTGCAGCTGGTGGAGTCTGGGGGAGACTTGGTACAGCCTGGAGGGTCCCTGAGACTCTCCTGTGCAGCCTCT
GGATTCACCTTCAGTAGTTATGAAATGAACTGGGTCCGCCAGGCTCCAGGGAAGGGGCTGGAGTGGGTTTCATAC
ATTAGTAATAGTGGTTCTACCATATACTACGCAGACTCTGTGAAGGGCCGATTCACCATCTCCAGAGACAACGCC
GAGACCTCACTGTATCTGCAAATGAACAGCCTGAGAGCCGAGGACACGGCTGTTTATTACTGTGCGAGAGAGGGA
TGGGGCGCATATTGTGCTGGTGATTGCTATTCTGGTTTTGATATTTGGGGCCAAGGGACAATGGTCACCGTCTCT
TCAGCCTCCACCAAGGGCCCATCGGTCTTCCCCCTGGCGCCCTGCTCCAGGAGCACCTCCGAGAGCACAGCCGCC
CTGGGCTGCCTGGTCAAGGACTACTTCCCCGAACCGGTGACGGTGTCGTGGAACTCAGGCGCCCTGACCAGCGGC
GTGCACACCTTCCCGGCTGTCCTACAGTCCTCAGGACTCTACTCCCTCAGCAGCGTGGTGACCGTGCCCTCCAGC
AGCTTGGGCACGAAGACCTACACCTGCAACGTAGATCACAAGCCCAGCAACACCAAGGTGGACAAGAGAGTTGAG
TCCAAATATGGTCCCCCATGCCCACCCTGCCCAGCACCTGAGTTCCTGGGGGGACCATCAGTCTTCCTGTTCCCC
CCAAAACCCAAGGACACTCTCATGATCTCCCGGACCCCTGAGGTCACGTGCGTGGTGGTGGACGTGAGCCAGGAA
GACCCCGAGGTCCAGTTCAACTGGTACGTGGATGGCGTGGAGGTGCATAATGCCAAGACAAAGCCGCGGGAGGAG
CAGTTCAACAGCACGTACCGTGTGGTCAGCGTCCTCACCGTCCTGCACCAGGACTGGCTGAACGGCAAGGAGTAC
AAGTGCAAGGTCTCCAACAAAGGCCTCCCGTCCTCCATCGAGAAAACCATCTCCAAAGCCAAAGGGCAGCCCCGA
GAGCCACAGGTGTACACCCTGCCCCCATCCCAGGAGGAGATGACCAAGAACCAGGTCAGCCTGACCTGCCTGGTC
AAAGGCTTCTACCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAGGCTCACCGTGGACAAGAGCAGGTGGCAGGAG
GGGAATGTCTTCTCATGCTCCGTGATGCATGAGGCTCTGCACAACCACTACACACAGAAGTCCCTCTCCCTGTCT
CTGGGTAAATGA
(SEQ ID NO: 117)
Heavy Chain Amino Acid
EVQLVESGGDLVQPGGSLRLSCAASGFTFSSYEMNWVRQAPGKGLEWVSYISNSGSTIYYADSVKGRFTISRDNA
ETSLYLQMNSLRAEDTAVYYCAREGWGAYCAGDCYSGEDIWGQGTMVTVSSASTKGPSVFPLAPCSRSTSESTAA
LGCLVKDYFPEPVTVSWNSGALTSGVHTFPAVLQSSGLYSLSSVVTVPSSSLGTKTYTCNVDHKPSNTKVDKRVE
SKYGPPCPPCPAPEFLGGPSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHEALHNHYTQKSLSLS
LGK
(SEQ ID NO: 118)
Light Chain Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 119)
Light Chain Amino Acid
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
(SEQ ID NO: 120)
H4H30076P
HCVR Nucleotide Sequence
GAGGTGCAGCTGGTGGAGTCTGGAGGAGGCTTGGTCCAGCCTGGGGGGTCCCTGAGACTCTCCTGTGCAGCCTCT
GGGTTCACCGTCAGTAGCAACTACATGAGCTGGGTCCGCCAGGCTCCAGGGAAGGGGCTGGAGTGGGTCTCAATT
ATTTATAGCGGTGGTCACACATACTACTCAGACTCCGTGAAGGGCCGATTCACCATCTCCAGACACAATTCCAAG
AACACTCTGTATCTTCAAATGAACAGCCTGAGAGGTGGGGACACGGCCGTGTATTACTGTGCGAGAGGGTATACC
AGTGGCTGGTACGGATTTGACTTCTGGGGCCAGGGAACCCTGGTCACCGTCTCCTCA
(SEQ ID NO: 121)
HCVR Amino Acid Sequence
EVQLVESGGGLVQPGGSLRLSCAASGFTVSSNYMSWVRQAPGKGLEWVSIIYSGGHTYYSDSVKGRFTISRHNSK
NTLYLQMNSLRGGDTAVYYCARGYTSGWYGFDFWGQGTLVTVSS
(SEQ ID NO: 122)
HCDR1 Nucleotide Sequence
GGG TTC ACC GTC AGT AGC AAC TAC
(SEQ ID NO: 123)
HCDR1 Amino Acid Sequence
G F T V S S N Y
(SEQ ID NO: 124)
HCDR2 Nucleotide Sequence
ATT TAT AGC GGT GGT CAC ACA
(SEQ ID NO: 125)
HCDR2 Amino Acid Sequence
I Y S G G H T
(SEQ ID NO: 126)
HCDR3 Nucleotide Sequence
GCG AGA GGG TAT ACC AGT GGC TGG TAC GGA TTT GAC TTC
(SEQ ID NO: 127)
HCDR3 Amino Acid Sequence
A R G Y T S G W Y G F D F
(SEQ ID NO: 128)
LCVR Nucleotide Sequence
GACATCCAGATGACCCAGTCTCCATCTTCCGTGTCTGCATCTGTAGGAGACAGAGTCACCATCACTTGTCGGGCG
AGTCAGGGTATTAGCACCTGGTTAGCCTGGTATCAGCAGAAACCAGGGAAAGCCCCTAAGCTCCTGATCTATGCT
GCATCCAGTTTGCAAAGTGGGGTCCCGTCAAGATTCAGCGGCACTGGATCTGGGACAGATTTCACTCTCACCATC
AGCAGCCTGCAGCCTGAAGATTTTGCAACTTATTATTGTCAGCAGACTAACAGTTTCCCGTGGACGTTCGGCCAA
GGGACCAAGGTGGAAATCAAA
(SEQ ID NO: 129)
LCVR Amino Acid Sequence
DIQMTQSPSSVSASVGDRVTITCRASQGISTWLAWYQQKPGKAPKLLIYAASSLQSGVPSRFSGTGSGTDFTLTI
SSLQPEDFATYYCQQTNSFPWTFGQGTKVEIK
(SEQ ID NO: 130)
LCDR1 Nucleotide Sequence
CAG GGT ATT AGC ACC TGG
(SEQ ID NO: 131)
LCDR1 Amino Acid Sequence
Q G I S T W
(SEQ ID NO: 132)
LCDR2 Nucleotide Sequence
GCT GCA TCC
(SEQ ID NO: 33)
LCDR2 Amino Acid Sequence
A A S
(SEQ ID NO: 34)
LCDR3 Nucleotide Sequence
CAG CAG ACT AAC AGT TTC CCG TGG ACG
(SEQ ID NO: 133)
LCDR3 Amino Acid Sequence
A A S
(SEQ ID NO: 134)
Heavy Chain Nucleotide Sequence
GAGGTGCAGCTGGTGGAGTCTGGAGGAGGCTTGGTCCAGCCTGGGGGGTCCCTGAGACTCTCCTGTGCAGCCTCT
GGGTTCACCGTCAGTAGCAACTACATGAGCTGGGTCCGCCAGGCTCCAGGGAAGGGGCTGGAGTGGGTCTCAATT
ATTTATAGCGGTGGTCACACATACTACTCAGACTCCGTGAAGGGCCGATTCACCATCTCCAGACACAATTCCAAG
AACACTCTGTATCTTCAAATGAACAGCCTGAGAGGTGGGGACACGGCCGTGTATTACTGTGCGAGAGGGTATACC
AGTGGCTGGTACGGATTTGACTTCTGGGGCCAGGGAACCCTGGTCACCGTCTCCTCAGCCTCCACCAAGGGCCCA
TCGGTCTTCCCCCTGGCGCCCTGCTCCAGGAGCACCTCCGAGAGCACAGCCGCCCTGGGCTGCCTGGTCAAGGAC
TACTTCCCCGAACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAATATGGTCCCCCATGC
CCACCCTGCCCAGCACCTGAGTTCCTGGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAGTTCAACAGCACGTACCGT
GTGGTCAGCGTCCTCACCGTCCTGCACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCCCCGAGAGCCACAGGTGTACACCCTG
CCCCCATCCCAGGAGGAGATGACCAAGAACCAGGTCAGCCTGACCTGCCTGGTCAAAGGCTTCTACCCCAGCGAC
ATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCC
GTGATGCATGAGGCTCTGCACAACCACTACACACAGAAGTCCCTCTCCCTGTCTCTGGGTAAATGA
(SEQ ID NO: 135)
Heavy Chain Amino Acid Sequence
EVQLVESGGGLVQPGGSLRLSCAASGFTVSSNYMSWVRQAPGKGLEWVSIIYSGGHTYYSDSVKGRFTISRHNSK
NTLYLQMNSLRGGDTAVYYCARGYTSGWYGFDFWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK*
(SEQ ID NO: 136)
Light Chain Nucleotide Sequence
GACATCCAGATGACCCAGTCTCCATCTTCCGTGTCTGCATCTGTAGGAGACAGAGTCACCATCACTTGTCGGGCG
AGTCAGGGTATTAGCACCTGGTTAGCCTGGTATCAGCAGAAACCAGGGAAAGCCCCTAAGCTCCTGATCTATGCT
GCATCCAGTTTGCAAAGTGGGGTCCCGTCAAGATTCAGCGGCACTGGATCTGGGACAGATTTCACTCTCACCATC
AGCAGCCTGCAGCCTGAAGATTTTGCAACTTATTATTGTCAGCAGACTAACAGTTTCCCGTGGACGTTCGGCCAA
GGGACCAAGGTGGAAATCAAACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAG
(SEQ ID NO: 137)
Light Chain Amino Acid Sequence
DIQMTQSPSSVSASVGDRVTITCRASQGISTWLAWYQQKPGKAPKLLIYAASSLQSGVPSRFSGTGSGTDFTLTI
SSLQPEDFATYYCQQTNSFPWTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC*
(SEQ ID NO: 138)
H4H30105P2
HCVR Nucleotide Sequence
GAGGTGCAGCTGGTGCAGTCTGGAGCAGAGGTGAAAAAGCCCGGGGAGTCTCTGAAGATCTCCTGTAAGGGTTCT
GGATACAGCTTTACCAGCTACTGGATCGCCTGGGTGCGCCAGATGCCCGGGAAAGGCCTGGAGTGGATGGGGATC
ATCTATCCTGGTGACTCTGATACCAGATACAGCCCGTCCTTCCAAGGCCAGGTCACCATGTCAGCCGACAAGTCC
ATCAGGATCGCCTACCTGCAGTGGAGCAGCCTGAAGGCCTCGGACACCGCCATGTATTATTGTGCGAGACTTGAT
TATAGCGGCAGCTGGTTTGACTACTGGGGCCAGGGAACCCTGGTCACCGTCTCCTCA
(SEQ ID NO: 139)
HCVR Amino Acid Sequence
EVQLVQSGAEVKKPGESLKISCKGSGYSFTSYWIAWVRQMPGKGLEWMGIIYPGDSDTRYSPSFQGQVTMSADKS
IRIAYLQWSSLKASDTAMYYCARLDYSGSWFDYWGQGTLVTVSS
(SEQ ID NO: 140)
HCDR1 Nucleotide Sequence
GGA TAC AGC TTT ACC AGC TAC TGG
(SEQ ID NO: 141)
HCDR1 Amino Acid Sequence
G Y S F T S Y W
(SEQ ID NO: 142)
HCDR2 Nucleotide Sequence
ATC TAT CCT GGT GAC TCT GAT ACC
(SEQ ID NO: 143)
HCDR2 Amino Acid Sequence
I Y P G D S D T
(SEQ ID NO: 144)
HCDR3 Nucleotide Sequence
GCG AGA CTT GAT TAT AGC GGC AGC TGG TTT GAC TAC
(SEQ ID NO: 145)
HCDR3 Amino Acid Sequence
A R L D Y S G S W F D Y
(SEQ ID NO: 146)
LCVR Nucleotide Sequence
GAAATAGTTTTGACACAGAGTCCCGGCACACTGTCACTCTCTCCCGGGGAAAGAGCCACCTTGTCATGTAGAGCA
AGTCAGTCAGTCTCTAGCTCTTATCTCGCCTGGTACCAGCAGAAGCCGGGACAGGCCCCTAGACTGCTGATCTAC
GGGGCAAGTTCCAGGGCCACCGGAATCCCCGACCGGTTCAGTGGAAGCGGAAGCGGAACCGATTTTACTTTGACG
ATTTCTAGACTGGAGCCAGAGGATTTCGCCGTTTACTATTGTCAACAGTACGGAAGCAGCCCGTGGACGTTTGGC
CAGGGCACGAAGGTAGAAATCAAG
(SEQ ID NO: 147)
LCVR Amino Acid Sequence
EIVLTQSPGTLSLSPGERATLSCRASQSVSSSYLAWYQQKPGQAPRLLIYGASSRATGIPDRESGSGSGTDFTLT
ISRLEPEDFAVYYCQQYGSSPWTFGQGTKVEIK
(SEQ ID NO: 148)
LCDR1 Nucleotide Sequence
CAG TCA GTC TCT AGC TCT TAT
(SEQ ID NO: 149)
LCDR1 Amino Acid Sequence
Q S V S S S Y
(SEQ ID NO: 150)
LCDR2 Nucleotide Sequence
GGG GCA AGT
(SEQ ID NO: 151)
LCDR2 Amino Acid Sequence
G A S
(SEQ ID NO: 14)
LCDR3 Nucleotide Sequence
CAA CAG TAC GGA AGC AGC CCG TGG ACG
(SEQ ID NO: 152)
LCDR3 Amino Acid Sequence
Q Q Y G S S P W T
(SEQ ID NO: 153)
Heavy Chain Nucleotide Sequence
GAGGTGCAGCTGGTGCAGTCTGGAGCAGAGGTGAAAAAGCCCGGGGAGTCTCTGAAGATCTCCTGTAAGGGTTCT
GGATACAGCTTTACCAGCTACTGGATCGCCTGGGTGCGCCAGATGCCCGGGAAAGGCCTGGAGTGGATGGGGATC
ATCTATCCTGGTGACTCTGATACCAGATACAGCCCGTCCTTCCAAGGCCAGGTCACCATGTCAGCCGACAAGTCC
ATCAGGATCGCCTACCTGCAGTGGAGCAGCCTGAAGGCCTCGGACACCGCCATGTATTATTGTGCGAGACTTGAT
TATAGCGGCAGCTGGTTTGACTACTGGGGCCAGGGAACCCTGGTCACCGTCTCCTCAGCCTCCACCAAGGGCCCA
TCGGTCTTCCCCCTGGCGCCCTGCTCCAGGAGCACCTCCGAGAGCACAGCCGCCCTGGGCTGCCTGGTCAAGGAC
TACTTCCCCGAACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAATATGGTCCCCCATGC
CCACCCTGCCCAGCACCTGAGTTCCTGGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAGTTCAACAGCACGTACCGT
GTGGTCAGCGTCCTCACCGTCCTGCACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCCCCGAGAGCCACAGGTGTACACCCTG
CCCCCATCCCAGGAGGAGATGACCAAGAACCAGGTCAGCCTGACCTGCCTGGTCAAAGGCTTCTACCCCAGCGAC
ATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCC
GTGATGCATGAGGCTCTGCACAACCACTACACACAGAAGTCCCTCTCCCTGTCTCTGGGTAAATGA
(SEQ ID NO: 154)
Heavy Chain Amino Acid Sequence
EVQLVQSGAEVKKPGESLKISCKGSGYSFTSYWIAWVRQMPGKGLEWMGIIYPGDSDTRYSPSFQGQVTMSADKS
IRIAYLQWSSLKASDTAMYYCARLDYSGSWFDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQFNWYVDGVEVHNAKTKPREEQENSTYR
VVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK*
(SEQ ID NO: 155)
Light Chain Nucleotide Sequence
GAAATAGTTTTGACACAGAGTCCCGGCACACTGTCACTCTCTCCCGGGGAAAGAGCCACCTTGTCATGTAGAGCA
AGTCAGTCAGTCTCTAGCTCTTATCTCGCCTGGTACCAGCAGAAGCCGGGACAGGCCCCTAGACTGCTGATCTAC
GGGGCAAGTTCCAGGGCCACCGGAATCCCCGACCGGTTCAGTGGAAGCGGAAGCGGAACCGATTTTACTTTGACG
ATTTCTAGACTGGAGCCAGAGGATTTCGCCGTTTACTATTGTCAACAGTACGGAAGCAGCCCGTGGACGTTTGGC
CAGGGCACGAAGGTAGAAATCAAGCGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 156)
Light Chain Amino Acid Sequence
EIVLTQSPGTLSLSPGERATLSCRASQSVSSSYLAWYQQKPGQAPRLLIYGASSRATGIPDRESGSGSGTDFTLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC*
(SEQ ID NO: 157)
H4H30108P2
HCVR Nucleotide Sequence
CAGGTCCAGCTGGTGCAGTCTGGGGCTGAGGTGAAGAAGCCTGGGGCCTCAGTGAAGGTCTCCTGCAAGGCTTCT
GGATACACCTTCAACAGTTATGATGTCAACTGGGTGCGACAGGCCACTGGACAAGGGCTTGAGTGGATGGGATGG
ATGAACCCTCACAGTGGTAACACAGGCTACGCACAGAAGTTCCAGGGCAGAGTCACTATGACCAGGGACACCTCC
ACAAGCACAAGCTATATGGAGTTGAGCAGCCTGACATCTGAGGACACGGCCGTATATTACTGTGCGAGAGGCCCT
TTTTCTCTACTTTCAGCTGAATTCTTCCAGCACTGGGGCCAGGGCACCCTGGTCACCGTCTCCTCA
(SEQ ID NO: 158)
HCVR Amino Acid Sequence
QVQLVQSGAEVKKPGASVKVSCKASGYTFNSYDVNWVRQATGQGLEWMGWMNPHSGNTGYAQKFQGRVTMTRDTS
TSTSYMELSSLTSEDTAVYYCARGPFSLLSAEFFQHWGQGTLVTVSS
(SEQ ID NO: 159)
HCDR1 Nucleotide Sequence
GGA TAC ACC TTC AAC AGT TAT GAT
(SEQ ID NO: 160)
HCDR1 Amino Acid Sequence
G Y T F N S Y D
(SEQ ID NO: 161)
HCDR2 Nucleotide Sequence
ATG AAC CCT CAC AGT GGT AAC ACA
(SEQ ID NO: 162)
HCDR2 Amino Acid Sequence
M N P H S G N T
(SEQ ID NO: 163)
HCDR3 Nucleotide Sequence
GCG AGA GGC CCT TTT TCT CTA CTT TCA GCT GAA TTC TTC CAG CAC
(SEQ ID NO: 164)
HCDR3 Amino Acid Sequence
A R G P F S L L S A E F F Q H
(SEQ ID NO: 165)
LCVR Nucleotide Sequence
GAAATAGTTTTGACACAGAGTCCCGGCACACTGTCACTCTCTCCCGGGGAAAGAGCCACCTTGTCATGTAGAGCA
AGTCAGTCAGTCTCTAGCTCTTATCTCGCCTGGTACCAGCAGAAGCCGGGACAGGCCCCTAGACTGCTGATCTAC
GGGGCAAGTTCCAGGGCCACCGGAATCCCCGACCGGTTCAGTGGAAGCGGAAGCGGAACCGATTTTACTTTGACG
ATTTCTAGACTGGAGCCAGAGGATTTCGCCGTTTACTATTGTCAACAGTACGGAAGCAGCCCGTGGACGTTTGGC
CAGGGCACGAAGGTAGAAATCAAG
(SEQ ID NO: 147)
LCVR Amino Acid Sequence
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIK
(SEQ ID NO: 148)
LCDR1 Nucleotide Sequence
CAG TCA GTC TCT AGC TCT TAT
(SEQ ID NO: 149)
LCDR1 Amino Acid Sequence
Q S V S S S Y
(SEQ ID NO: 150)
LCDR2 Nucleotide Sequence
GGG GCA AGT
(SEQ ID NO: 151)
LCDR2 Amino Acid Sequence
G A S
(SEQ ID NO: 14)
LCDR3 Nucleotide Sequence
CAA CAG TAC GGA AGC AGC CCG TGG ACG
(SEQ ID NO: 152)
LCDR3 Amino Acid Sequence
Q Q Y G S S P W T
(SEQ ID NO: 153)
Heavy Chain Nucleotide Sequence
CAGGTCCAGCTGGTGCAGTCTGGGGCTGAGGTGAAGAAGCCTGGGGCCTCAGTGAAGGTCTCCTGCAAGGCTTCT
GGATACACCTTCAACAGTTATGATGTCAACTGGGTGCGACAGGCCACTGGACAAGGGCTTGAGTGGATGGGATGG
ATGAACCCTCACAGTGGTAACACAGGCTACGCACAGAAGTTCCAGGGCAGAGTCACTATGACCAGGGACACCTCC
ACAAGCACAAGCTATATGGAGTTGAGCAGCCTGACATCTGAGGACACGGCCGTATATTACTGTGCGAGAGGCCCT
TTTTCTCTACTTTCAGCTGAATTCTTCCAGCACTGGGGCCAGGGCACCCTGGTCACCGTCTCCTCAGCCTCCACC
AAGGGCCCATCGGTCTTCCCCCTGGCGCCCTGCTCCAGGAGCACCTCCGAGAGCACAGCCGCCCTGGGCTGCCTG
GTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACG
AAGACCTACACCTGCAACGTAGATCACAAGCCCAGCAACACCAAGGTGGACAAGAGAGTTGAGTCCAAATATGGT
CCCCCATGCCCACCCTGCCCAGCACCTGAGTTCCTGGGGGGACCATCAGTCTTCCTGTTCCCCCCAAAACCCAAG
GACACTCTCATGATCTCCCGGACCCCTGAGGTCACGTGCGTGGTGGTGGACGTGAGCCAGGAAGACCCCGAGGTC
CAGTTCAACTGGTACGTGGATGGCGTGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTC
TCATGCTCCGTGATGCATGAGGCTCTGCACAACCACTACACACAGAAGTCCCTCTCCCTGTCTCTGGGTAAATGA
(SEQ ID NO: 166)
Heavy Chain Amino Acid Sequence
QVQLVQSGAEVKKPGASVKVSCKASGYTFNSYDVNWVRQATGQGLEWMGWMNPHSGNTGYAQKFQGRVTMTRDTS
TSTSYMELSSLTSEDTAVYYCARGPFSLLSAEFFQHWGQGTLVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVQFNWYVDGVEVHNAKTKPREEQENS
TYRVVSVLTVLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQKSLSLSLGK*
(SEQ ID NO: 167)
Light Chain Nucleotide Sequence
GAAATAGTTTTGACACAGAGTCCCGGCACACTGTCACTCTCTCCCGGGGAAAGAGCCACCTTGTCATGTAGAGCA
AGTCAGTCAGTCTCTAGCTCTTATCTCGCCTGGTACCAGCAGAAGCCGGGACAGGCCCCTAGACTGCTGATCTAC
GGGGCAAGTTCCAGGGCCACCGGAATCCCCGACCGGTTCAGTGGAAGCGGAAGCGGAACCGATTTTACTTTGACG
ATTTCTAGACTGGAGCCAGAGGATTTCGCCGTTTACTATTGTCAACAGTACGGAAGCAGCCCGTGGACGTTTGGC
CAGGGCACGAAGGTAGAAATCAAGCGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 156)
Light Chain Amino Acid Sequence
EIVLTQSPGTLSLSPGERATLSCRASQSVSSSYLAWYQQKPGQAPRLLIYGASSRATGIPDRFSGSGSGTDFTLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC*
(SEQ ID NO: 157)
H4H30117P2
HCVR Nucleotide Sequence
CAGGTCCAGCTGGTACAGTCTGGGGCTGAGGTGAAGAAGCCTGGGTCCTCGGTGAAGGTCTCCTGCAAGGCTTCT
GGAGACACCTTCAGTAACTATGTTATCGGCTGGGTGCGACAGGCCCCTGGACAAGGGCTTGAGTGGATGGGAGGG
ATCATCCCTATCTTTGGTACAACAAACTACGCACAGCAGTTCCAGGGCAGAGTCACGATTACCACGGACGAATCC
ACGAGCACGGCCTACATGGAGCTGAGCAGCCTGAGATCTGAGGACACGGCCGTGTATTACTGTGCGAGAGATGGG
AACTACGGTGACTACTTTGACTACTGGGGCCAGGGAACCCTGGTCACCGTCTCCTCA
(SEQ ID NO: 168)
HCVR Amino Acid Sequence
QVQLVQSGAEVKKPGSSVKVSCKASGDTFSNYVIGWVRQAPGQGLEWMGGIIPIFGTTNYAQQFQGRVTITTDES
TSTAYMELSSLRSEDTAVYYCARDGNYGDYFDYWGQGTLVTVSS
(SEQ ID NO: 169)
HCDR1 Nucleotide Sequence
GGA GAC ACC TTC AGT AAC TAT GTT
(SEQ ID NO: 170)
HCDR1 Amino Acid Sequence
G D T F S N Y V
(SEQ ID NO: 171)
HCDR2 Nucleotide Sequence
ATC ATC CCT ATC TTT GGT ACA ACA
(SEQ ID NO: 172)
HCDR2 Amino Acid Sequence
I I P I F G T T
(SEQ ID NO: 173)
HCDR3 Nucleotide Sequence
GCG AGA GAT GGG AAC TAC GGT GAC TAC TTT GAC TAC
(SEQ ID NO: 174)
HCDR3 Amino Acid Sequence
A R D G N Y G D Y F D Y
(SEQ ID NO: 175)
LCVR Nucleotide Sequence
GAAATAGTTTTGACACAGAGTCCCGGCACACTGTCACTCTCTCCCGGGGAAAGAGCCACCTTGTCATGTAGAGCA
AGTCAGTCAGTCTCTAGCTCTTATCTCGCCTGGTACCAGCAGAAGCCGGGACAGGCCCCTAGACTGCTGATCTAC
GGGGCAAGTTCCAGGGCCACCGGAATCCCCGACCGGTTCAGTGGAAGCGGAAGCGGAACCGATTTTACTTTGACG
ATTTCTAGACTGGAGCCAGAGGATTTCGCCGTTTACTATTGTCAACAGTACGGAAGCAGCCCGTGGACGTTTGGC
CAGGGCACGAAGGTAGAAATCAAG
(SEQ ID NO: 147)
LCVR Amino Acid Sequence
EIVLTQSPGTLSLSPGERATLSCRASQSVSSSYLAWYQQKPGQAPRLLIYGASSRATGIPDRESGSGSGTDFTLT
ISRLEPEDFAVYYCQQYGSSPWTFGQGTKVEIK
(SEQ ID NO: 148)
LCDR1 Nucleotide Sequence
CAG TCA GTC TCT AGC TCT TAT
(SEQ ID NO: 149)
LCDR1 Amino Acid Sequence
Q S V S S S Y
(SEQ ID NO: 150)
LCDR2 Nucleotide Sequence
GGG GCA AGT
(SEQ ID NO: 151)
LCDR2 Amino Acid Sequence
G A S
(SEQ ID NO: 14)
LCDR3 Nucleotide Sequence
CAA CAG TAC GGA AGC AGC CCG TGG ACG
(SEQ ID NO: 152)
LCDR3 Amino Acid Sequence
Q Q Y G S S P W T
(SEQ ID NO: 153)
Heavy Chain Nucleotide Sequence
CAGGTCCAGCTGGTACAGTCTGGGGCTGAGGTGAAGAAGCCTGGGTCCTCGGTGAAGGTCTCCTGCAAGGCTTCT
GGAGACACCTTCAGTAACTATGTTATCGGCTGGGTGCGACAGGCCCCTGGACAAGGGCTTGAGTGGATGGGAGGG
ATCATCCCTATCTTTGGTACAACAAACTACGCACAGCAGTTCCAGGGCAGAGTCACGATTACCACGGACGAATCC
ACGAGCACGGCCTACATGGAGCTGAGCAGCCTGAGATCTGAGGACACGGCCGTGTATTACTGTGCGAGAGATGGG
AACTACGGTGACTACTTTGACTACTGGGGCCAGGGAACCCTGGTCACCGTCTCCTCAGCCTCCACCAAGGGCCCA
TCGGTCTTCCCCCTGGCGCCCTGCTCCAGGAGCACCTCCGAGAGCACAGCCGCCCTGGGCTGCCTGGTCAAGGAC
TACTTCCCCGAACCGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGGCACGAAGACCTAC
ACCTGCAACGTAGATCACAAGCCCAGCAACACCAAGGTGGACAAGAGAGTTGAGTCCAAATATGGTCCCCCATGC
CCACCCTGCCCAGCACCTGAGTTCCTGGGGGGACCATCAGTCTTCCTGTTCCCCCCAAAACCCAAGGACACTCTC
ATGATCTCCCGGACCCCTGAGGTCACGTGCGTGGTGGTGGACGTGAGCCAGGAAGACCCCGAGGTCCAGTTCAAC
TGGTACGTGGATGGCGTGGAGGTGCATAATGCCAAGACAAAGCCGCGGGAGGAGCAGTTCAACAGCACGTACCGT
GTGGTCAGCGTCCTCACCGTCCTGCACCAGGACTGGCTGAACGGCAAGGAGTACAAGTGCAAGGTCTCCAACAAA
GGCCTCCCGTCCTCCATCGAGAAAACCATCTCCAAAGCCAAAGGGCAGCCCCGAGAGCCACAGGTGTACACCCTG
CCCCCATCCCAGGAGGAGATGACCAAGAACCAGGTCAGCCTGACCTGCCTGGTCAAAGGCTTCTACCCCAGCGAC
ATCGCCGTGGAGTGGGAGAGCAATGGGCAGCCGGAGAACAACTACAAGACCACGCCTCCCGTGCTGGACTCCGAC
GGCTCCTTCTTCCTCTACAGCAGGCTCACCGTGGACAAGAGCAGGTGGCAGGAGGGGAATGTCTTCTCATGCTCC
GTGATGCATGAGGCTCTGCACAACCACTACACACAGAAGTCCCTCTCCCTGTCTCTGGGTAAATGA
(SEQ ID NO: 176)
Heavy Chain Amino Acid Sequence
QVQLVQSGAEVKKPGSSVKVSCKASGDTFSNYVIGWVRQAPGQGLEWMGGIIPIFGTTNYAQQFQGRVTITTDES
TSTAYMELSSLRSEDTAVYYCARDGNYGDYFDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKVDKRVESKYGPPC
PPCPAPEFLGGPSVFLFPPKPKDTLMISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK*
(SEQ ID NO: 177)
Light Chain Nucleotide Sequence
GAAATAGTTTTGACACAGAGTCCCGGCACACTGTCACTCTCTCCCGGGGAAAGAGCCACCTTGTCATGTAGAGCA
AGTCAGTCAGTCTCTAGCTCTTATCTCGCCTGGTACCAGCAGAAGCCGGGACAGGCCCCTAGACTGCTGATCTAC
GGGGCAAGTTCCAGGGCCACCGGAATCCCCGACCGGTTCAGTGGAAGCGGAAGCGGAACCGATTTTACTTTGACG
ATTTCTAGACTGGAGCCAGAGGATTTCGCCGTTTACTATTGTCAACAGTACGGAAGCAGCCCGTGGACGTTTGGC
CAGGGCACGAAGGTAGAAATCAAGCGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 156)
Light Chain Amino Acid Sequence
EIVLTQSPGTLSLSPGERATLSCRASQSVSSSYLAWYQQKPGQAPRLLIYGASSRATGIPDRFSGSGSGTDETLT
ISRLEPEDFAVYYCQQYGSSPWTFGQGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC*
(SEQ ID NO: 157)
H4H30045P
HCVR Nucleotide Sequence
CAGGTTCAGCTGGTGCAGTCTGGAGCTGAGGTGAAGAAGCCTGGGGCCTCAGTGAAGGTCTCCTGCAAGACTTCT
GGTTACAGATTCGTCAACTATGGTTTCAGCTGGGTGCGCCAGGCCCCTGGACAAGGCCTTGAATGGATGGGATGG
ATCAGCCCTTATAATGGTAACACAAACTATATACAGAATCTCCAGGACAGAATCACCATGACCACAGACACCTCT
ACGAACACAGCCTACATGGAACTGACGAACCTGAGATCTGACGACACGGCCGTATATTACTGTGCGACCTTAACT
GGGGTTCACTTTGACTACTGGGGCCAGGGAACCCTGGTCACCGTCTCCTCA
(SEQ ID NO: 178)
HCVR Amino Acid Sequence
QVQLVQSGAEVKKPGASVKVSCKTSGYRFVNYGFSWVRQAPGQGLEWMGWISPYNGNTNYIQNLQDRITMTTDTS
TNTAYMELTNLRSDDTAVYYCATLTGVHFDYWGQGTLVTVSS
(SEQ ID NO: 179)
HCDR1 Nucleotide Sequence
GGT TAC AGA TTC GTC AAC TAT GGT
(SEQ ID NO: 180)
HCDR1 Amino Acid Sequence
G Y R F V N Y G
(SEQ ID NO: 181)
HCDR2 Nucleotide Sequence
ATC AGC CCT TAT AAT GGT AAC ACA
(SEQ ID NO: 182)
HCDR2 Amino Acid Sequence
I S P Y N G N T
(SEQ ID NO: 183)
HCDR3 Nucleotide Sequence
GCG ACC TTA ACT GGG GTT CAC TTT GAC TAC
(SEQ ID NO: 184)
HCDR3 Amino Acid Sequence
A T L T G V H F D Y
(SEQ ID NO: 185)
LCVR Nucleotide Sequence
GCCATCCAGATGACCCAGTCTCCATCCTCCCTGTCTGCATCTGTAGGAGACAGAGTCACCATCACTTGCCGGGCA
AGTCAGGGCATTAGAAATGATTTAGGCTGGTATCAGCAGAAACCAGGGAAAGCCCCTAATCTCCTGATCTTTGAA
ACATCTCGTTTACAAAGTGGGGTCCCTTCGAGGTTCAGCGGCAGTGGTTCTGGCACAGATTTCACTCTCACCATC
AACAGCCTTCAGCCTGAAGATTTTGCAACTTATTACTGTCTACAAGATTACAATTACCCGTGGACGTTCGGCCAA
GGGACCAAGGTGGAAATCAAA
(SEQ ID NO: 186)
LCVR Amino Acid Sequence
AIQMTQSPSSLSASVGDRVTITCRASQGIRNDLGWYQQKPGKAPNLLIFETSRLQSGVPSRFSGSGSGTDFTLTI
NSLQPEDFATYYCLQDYNYPWTFGQGTKVEIK
(SEQ ID NO: 187)
LCDR1 Nucleotide Sequence
CAG GGC ATT AGA AAT GAT
(SEQ ID NO: 188)
LCDR1 Amino Acid Sequence
Q G I R N D
(SEQ ID NO: 189)
LCDR2 Nucleotide Sequence
GAA ACA TCT
(SEQ ID NO: 190)
LCDR2 Amino Acid Sequence
E T S
(SEQ ID NO: 191)
LCDR3 Nucleotide Sequence
CTA CAA GAT TAC AAT TAC CCG TGG ACG
(SEQ ID NO: 192)
LCDR3 Amino Acid Sequence
L Q D Y N Y P W T
(SEQ ID NO: 193)
Heavy Chain Nucleotide Sequence
CAGGTTCAGCTGGTGCAGTCTGGAGCTGAGGTGAAGAAGCCTGGGGCCTCAGTGAAGGTCTCCTGCAAGACTTCT
GGTTACAGATTCGTCAACTATGGTTTCAGCTGGGTGCGCCAGGCCCCTGGACAAGGCCTTGAATGGATGGGATGG
ATCAGCCCTTATAATGGTAACACAAACTATATACAGAATCTCCAGGACAGAATCACCATGACCACAGACACCTCT
ACGAACACAGCCTACATGGAACTGACGAACCTGAGATCTGACGACACGGCCGTATATTACTGTGCGACCTTAACT
GGGGTTCACTTTGACTACTGGGGCCAGGGAACCCTGGTCACCGTCTCCTCAGCCTCCACCAAGGGCCCATCGGTC
TTCCCCCTGGCGCCCTGCTCCAGGAGCACCTCCGAGAGCACAGCCGCCCTGGGCTGCCTGGTCAAGGACTACTTC
CCCGAACCGGTGACGGTGTCGTGGAACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAGCTTGGGCACGAAGACCTACACCTGC
AACGTAGATCACAAGCCCAGCAACACCAAGGTGGACAAGAGAGTTGAGTCCAAATATGGTCCCCCATGCCCACCC
TGCCCAGCACCTGAGTTCCTGGGGGGACCATCAGTCTTCCTGTTCCCCCCAAAACCCAAGGACACTCTCATGATC
TCCCGGACCCCTGAGGTCACGTGCGTGGTGGTGGACGTGAGCCAGGAAGACCCCGAGGTCCAGTTCAACTGGTAC
GTGGATGGCGTGGAGGTGCATAATGCCAAGACAAAGCCGCGGGAGGAGCAGTTCAACAGCACGTACCGTGTGGTC
AGCGTCCTCACCGTCCTGCACCAGGACTGGCTGAACGGCAAGGAGTACAAGTGCAAGGTCTCCAACAAAGGCCTC
CCGTCCTCCATCGAGAAAACCATCTCCAAAGCCAAAGGGCAGCCCCGAGAGCCACAGGTGTACACCCTGCCCCCA
TCCCAGGAGGAGATGACCAAGAACCAGGTCAGCCTGACCTGCCTGGTCAAAGGCTTCTACCCCAGCGACATCGCC
GTGGAGTGGGAGAGCAATGGGCAGCCGGAGAACAACTACAAGACCACGCCTCCCGTGCTGGACTCCGACGGCTCC
TTCTTCCTCTACAGCAGGCTCACCGTGGACAAGAGCAGGTGGCAGGAGGGGAATGTCTTCTCATGCTCCGTGATG
CATGAGGCTCTGCACAACCACTACACACAGAAGTCCCTCTCCCTGTCTCTGGGTAAATGA
(SEQ ID NO: 194)
Heavy Chain Amino Acid Sequence
QVQLVQSGAEVKKPGASVKVSCKTSGYRFVNYGFSWVRQAPGQGLEWMGWISPYNGNTNYIQNLQDRITMTTDTS
TNTAYMELTNLRSDDTAVYYCATLTGVHFDYWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK*
(SEQ ID NO: 195)
Light Chain Nucleotide Sequence
GCCATCCAGATGACCCAGTCTCCATCCTCCCTGTCTGCATCTGTAGGAGACAGAGTCACCATCACTTGCCGGGCA
AGTCAGGGCATTAGAAATGATTTAGGCTGGTATCAGCAGAAACCAGGGAAAGCCCCTAATCTCCTGATCTTTGAA
ACATCTCGTTTACAAAGTGGGGTCCCTTCGAGGTTCAGCGGCAGTGGTTCTGGCACAGATTTCACTCTCACCATC
AACAGCCTTCAGCCTGAAGATTTTGCAACTTATTACTGTCTACAAGATTACAATTACCCGTGGACGTTCGGCCAA
GGGACCAAGGTGGAAATCAAACGAACTGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGACAGCAAGGACAGCACCTACAGCCTC
AGCAGCACCCTGACGCTGAGCAAAGCAGACTACGAGAAACACAAAGTCTACGCCTGCGAAGTCACCCATCAGGGC
CTGAGCTCGCCCGTCACAAAGAGCTTCAACAGGGGAGAGTGTTAG
(SEQ ID NO: 196)
Light Chain Amino Acid Sequence
AIQMTQSPSSLSASVGDRVTITCRASQGIRNDLGWYQQKPGKAPNLLIFETSRLQSGVPSRFSGSGSGTDFTLTI
NSLQPEDFATYYCLQDYNYPWTFGQGTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSENRGEC*
(SEQ ID NO: 197)
H4H30061P
HCVR Nucleotide Sequence
GAGGTGCAGCTGGTGGAGTCTGGGGGAGGCTTGGCCCAGCCTGGGGGGTCCCTGAAACTCTCTTGTGAAGCCTCT
GGATTCACGTTCAGTGATTCTGCAATGCACTGGGTCCGCCAGGCTTCCGGAAAAGGGCTGGAGTGGGTTGGTCGT
ATTAGAAGCAAAGCTAATAGTTACGCGACAGGATATGCTGCGTCGGTGAAAGGCAGGTTCACCATCTCCAGAGAT
GATTCAAAGAACATGGCGTTTCTGGAAATGAACAGCCTGAAAACCGAAGACACGGCCGTATATTACTGTCTCCGA
CAAACTTACGGTGACCCCTGGGGCCAGGGAACCCTGGTCACCGTCTCCTCA
(SEQ ID NO: 198)
HCVR Amino Acid Sequence
EVQLVESGGGLAQPGGSLKLSCEASGFTFSDSAMHWVRQASGKGLEWVGRIRSKANSYATGYAASVKGRFTISRD
DSKNMAFLEMNSLKTEDTAVYYCLRQTYGDPWGQGTLVTVSS
(SEQ ID NO: 199)
HCDR1 Nucleotide Sequence
GGA TTC ACG TTC AGT GAT TCT GCA
(SEQ ID NO: 200)
HCDR1 Amino Acid Sequence
G F T F S D S A
(SEQ ID NO: 201)
HCDR2 Nucleotide Sequence
ATT AGA AGC AAA GCT AAT AGT TAC GCG ACA
(SEQ ID NO: 202)
HCDR2 Amino Acid Sequence
I R S K A N S Y A T
(SEQ ID NO: 203)
HCDR3 Nucleotide Sequence
CTC CGA CAA ACT TAC GGT GAC CCC
(SEQ ID NO: 204)
HCDR3 Amino Acid Sequence
L R Q T Y G D P
(SEQ ID NO: 205)
LCVR Nucleotide Sequence
GATGTTGTGATGACTCAGTCTCCACTCTCCCTGTCCGTCACCCTTGGACAGCCGGCCTCCATCTCCTGCAGGTCT
AGTCTAAGCCTCGTATACAGTGATGGAAACAACTACTTGAATTGGTTTCAGCAGAGGCCAGGCCAATCTCCAAGG
CGCCTACTTTATAAAGTTTTTAACCGGGACTCTGGGGTCCCAGACAGATTCAGCGGCAGTGGGTCAGGCACTGAT
TTCACACTGAAAATCAGCAGGGTGGAGGCTGAGGATGTTGGGGTTTATTACTGCATGCAAGGAACACACTGGCCG
TGGACGTTCGGCCAAGGGACCAAGGTGGAAATCAAA
(SEQ ID NO: 206)
LCVR Amino Acid Sequence
DVVMTQSPLSLSVTLGQPASISCRSSLSLVYSDGNNYLNWFQQRPGQSPRRLLYKVFNRDSGVPDRFSGSGSGTD
FTLKISRVEAEDVGVYYCMQGTHWPWTFGQGTKVEIK
(SEQ ID NO: 207)
LCDR1 Nucleotide Sequence
CTA AGC CTC GTA TAC AGT GAT GGA AAC AAC TAC
(SEQ ID NO: 208)
LCDR1 Amino Acid Sequence
L S L V Y S D G N N Y
(SEQ ID NO: 209)
LCDR2 Nucleotide Sequence
AAA GTT TTT
(SEQ ID NO: 210)
LCDR2 Amino Acid Sequence
K V F
(SEQ ID NO: 211)
LCDR3 Nucleotide Sequence
ATG CAA GGA ACA CAC TGG CCG TGG ACG
(SEQ ID NO: 212)
LCDR3 Amino Acid Sequence
M Q G T H W P W T
(SEQ ID NO: 213)
Heavy Chain Nucleotide Sequence
GAGGTGCAGCTGGTGGAGTCTGGGGGAGGCTTGGCCCAGCCTGGGGGGTCCCTGAAACTCTCTTGTGAAGCCTCT
GGATTCACGTTCAGTGATTCTGCAATGCACTGGGTCCGCCAGGCTTCCGGAAAAGGGCTGGAGTGGGTTGGTCGT
ATTAGAAGCAAAGCTAATAGTTACGCGACAGGATATGCTGCGTCGGTGAAAGGCAGGTTCACCATCTCCAGAGAT
GATTCAAAGAACATGGCGTTTCTGGAAATGAACAGCCTGAAAACCGAAGACACGGCCGTATATTACTGTCTCCGA
CAAACTTACGGTGACCCCTGGGGCCAGGGAACCCTGGTCACCGTCTCCTCAGCCTCCACCAAGGGCCCATCGGTC
TTCCCCCTGGCGCCCTGCTCCAGGAGCACCTCCGAGAGCACAGCCGCCCTGGGCTGCCTGGTCAAGGACTACTTC
CCCGAACCGGTGACGGTGTCGTGGAACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAGCTTGGGCACGAAGACCTACACCTGC
AACGTAGATCACAAGCCCAGCAACACCAAGGTGGACAAGAGAGTTGAGTCCAAATATGGTCCCCCATGCCCACCC
TGCCCAGCACCTGAGTTCCTGGGGGGACCATCAGTCTTCCTGTTCCCCCCAAAACCCAAGGACACTCTCATGATC
TCCCGGACCCCTGAGGTCACGTGCGTGGTGGTGGACGTGAGCCAGGAAGACCCCGAGGTCCAGTTCAACTGGTAC
GTGGATGGCGTGGAGGTGCATAATGCCAAGACAAAGCCGCGGGAGGAGCAGTTCAACAGCACGTACCGTGTGGTC
AGCGTCCTCACCGTCCTGCACCAGGACTGGCTGAACGGCAAGGAGTACAAGTGCAAGGTCTCCAACAAAGGCCTC
CCGTCCTCCATCGAGAAAACCATCTCCAAAGCCAAAGGGCAGCCCCGAGAGCCACAGGTGTACACCCTGCCCCCA
TCCCAGGAGGAGATGACCAAGAACCAGGTCAGCCTGACCTGCCTGGTCAAAGGCTTCTACCCCAGCGACATCGCC
GTGGAGTGGGAGAGCAATGGGCAGCCGGAGAACAACTACAAGACCACGCCTCCCGTGCTGGACTCCGACGGCTCC
TTCTTCCTCTACAGCAGGCTCACCGTGGACAAGAGCAGGTGGCAGGAGGGGAATGTCTTCTCATGCTCCGTGATG
CATGAGGCTCTGCACAACCACTACACACAGAAGTCCCTCTCCCTGTCTCTGGGTAAATGA
(SEQ ID NO: 214)
Heavy Chain Amino Acid Sequence
EVQLVESGGGLAQPGGSLKLSCEASGFTFSDSAMHWVRQASGKGLEWVGRIRSKANSYATGYAASVKGRFTISRD
DSKNMAFLEMNSLKTEDTAVYYCLRQTYGDPWGQGTLVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPCPP
CPAPEFLGGPSVFLFPPKPKDTLMISRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPPSQEEMTKNQVSLTCLVKGFYPSDIA
VEWESNGQPENNYKTTPPVLDSDGSFFLYSRLTVDKSRWQEGNVFSCSVMHEALHNHYTQKSLSLSLGK*
(SEQ ID NO: 215)
Light Chain Nucleotide Sequence
GATGTTGTGATGACTCAGTCTCCACTCTCCCTGTCCGTCACCCTTGGACAGCCGGCCTCCATCTCCTGCAGGTCT
AGTCTAAGCCTCGTATACAGTGATGGAAACAACTACTTGAATTGGTTTCAGCAGAGGCCAGGCCAATCTCCAAGG
CGCCTACTTTATAAAGTTTTTAACCGGGACTCTGGGGTCCCAGACAGATTCAGCGGCAGTGGGTCAGGCACTGAT
TTCACACTGAAAATCAGCAGGGTGGAGGCTGAGGATGTTGGGGTTTATTACTGCATGCAAGGAACACACTGGCCG
TGGACGTTCGGCCAAGGGACCAAGGTGGAAATCAAACGAACTGTGGCTGCACCATCTGTCTTCATCTTCCCGCCA
TCTGATGAGCAGTTGAAATCTGGAACTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAGAAACACAAAGTCTACGCCTGCGAA
GTCACCCATCAGGGCCTGAGCTCGCCCGTCACAAAGAGCTTCAACAGGGGAGAGTGTTAG
(SEQ ID NO: 216)
Light Chain Amino Acid Sequence
DVVMTQSPLSLSVTLGQPASISCRSSLSLVYSDGNNYLNWFQQRPGQSPRRLLYKVENRDSGVPDRESGSGSGTD
FTLKISRVEAEDVGVYYCMQGTHWPWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGLSSPVTKSENRGEC*
(SEQ ID NO: 217)
H4H30095P2
HCVR Nucleotide Sequence
CAGGTTCAGCTGGTGCAGTCTGGAGTTGAGGTGAAGAAGCCTGGGGCCTCAGTGAAGGTCTCCTGCAAGGCTTCT
GGTTACACCTTTAAGTTTTATGGTATCAGTTGGGTGCGACAGGCCCCTGGACAAGGGCTTGAGTGGATGGGCTGG
ATCAGTGTTTACAATGGTAAAACAAAGTATGCACAGAAGCTCCAGGGCAGAGTCACCATGACAACAGACACATCC
ACGAGCACAGCCTACATGGAGCTGAGGAGCCTGAGATCTGACGACACGGCCGTGTATTTTTGTGCGAGAGATGGG
GACTATGAGAGTAGTGGTTATCCGTTTGACTACTGGGGCCAGGGAACCCTGGTCACCGTCTCCTCA
(SEQ ID NO: 218)
HCVR Amino Acid Sequence
QVQLVQSGVEVKKPGASVKVSCKASGYTFKFYGISWVRQAPGQGLEWMGWISVYNGKTKYAQKLQGRVTMTTDTS
TSTAYMELRSLRSDDTAVYFCARDGDYESSGYPFDYWGQGTLVTVSS
(SEQ ID NO: 219)
HCDR1 Nucleotide Sequence
GGT TAC ACC TTT AAG TTT TAT GGT
(SEQ ID NO: 220)
HCDR1 Amino Acid Sequence
G Y T F K F Y G
(SEQ ID NO: 221)
HCDR2 Nucleotide Sequence
ATC AGT GTT TAC AAT GGT AAA ACA
(SEQ ID NO: 222)
HCDR2 Amino Acid Sequence
I S V Y N G K T
(SEQ ID NO: 223)
HCDR3 Nucleotide Sequence
GCG AGA GAT GGG GAC TAT GAG AGT AGT GGT TAT CCG TTT GAC TAC
(SEQ ID NO: 224)
HCDR3 Amino Acid Sequence
RD G
(SEQ ID NO: 225)
LCVR Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAA
(SEQ ID NO: 226)
LCVR Amino Acid Sequence
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIK
(SEQ ID NO: 227)
LCDR1 Nucleotide Sequence
CAG AGC ATT AGC AGT TAT
(SEQ ID NO: 31)
LCDR1 Amino Acid Sequence
Q S I S S Y
(SEQ ID NO: 32)
LCDR2 Nucleotide Sequence
GCT GCA TCC
(SEQ ID NO: 33)
LCDR2 Amino Acid Sequence
A A S
(SEQ ID NO: 34)
LCDR3 Nucleotide Sequence
CAA CAG AGT TAC AGT ACC CCT CCG ATC ACC
(SEQ ID NO: 75)
LCDR3 Amino Acid Sequence
Q Q S Y S T P P I T
(SEQ ID NO: 76)
Heavy Chain Nucleotide Sequence
CAGGTTCAGCTGGTGCAGTCTGGAGTTGAGGTGAAGAAGCCTGGGGCCTCAGTGAAGGTCTCCTGCAAGGCTTCT
GGTTACACCTTTAAGTTTTATGGTATCAGTTGGGTGCGACAGGCCCCTGGACAAGGGCTTGAGTGGATGGGCTGG
ATCAGTGTTTACAATGGTAAAACAAAGTATGCACAGAAGCTCCAGGGCAGAGTCACCATGACAACAGACACATCC
ACGAGCACAGCCTACATGGAGCTGAGGAGCCTGAGATCTGACGACACGGCCGTGTATTTTTGTGCGAGAGATGGG
GACTATGAGAGTAGTGGTTATCCGTTTGACTACTGGGGCCAGGGAACCCTGGTCACCGTCTCCTCAGCCTCCACC
AAGGGCCCATCGGTCTTCCCCCTGGCGCCCTGCTCCAGGAGCACCTCCGAGAGCACAGCCGCCCTGGGCTGCCTG
GTCAAGGACTACTTCCCCGAACCGGTGACGGTGTCGTGGAACTCAGGCGCCCTGACCAGCGGCGTGCACACCTTC
CCGGCTGTCCTACAGTCCTCAGGACTCTACTCCCTCAGCAGCGTGGTGACCGTGCCCTCCAGCAGCTTGGGCACG
AAGACCTACACCTGCAACGTAGATCACAAGCCCAGCAACACCAAGGTGGACAAGAGAGTTGAGTCCAAATATGGT
CCCCCATGCCCACCCTGCCCAGCACCTGAGTTCCTGGGGGGACCATCAGTCTTCCTGTTCCCCCCAAAACCCAAG
GACACTCTCATGATCTCCCGGACCCCTGAGGTCACGTGCGTGGTGGTGGACGTGAGCCAGGAAGACCCCGAGGTC
CAGTTCAACTGGTACGTGGATGGCGTGGAGGTGCATAATGCCAAGACAAAGCCGCGGGAGGAGCAGTTCAACAGC
ACGTACCGTGTGGTCAGCGTCCTCACCGTCCTGCACCAGGACTGGCTGAACGGCAAGGAGTACAAGTGCAAGGTC
TCCAACAAAGGCCTCCCGTCCTCCATCGAGAAAACCATCTCCAAAGCCAAAGGGCAGCCCCGAGAGCCACAGGTG
TACACCCTGCCCCCATCCCAGGAGGAGATGACCAAGAACCAGGTCAGCCTGACCTGCCTGGTCAAAGGCTTCTAC
CCCAGCGACATCGCCGTGGAGTGGGAGAGCAATGGGCAGCCGGAGAACAACTACAAGACCACGCCTCCCGTGCTG
GACTCCGACGGCTCCTTCTTCCTCTACAGCAGGCTCACCGTGGACAAGAGCAGGTGGCAGGAGGGGAATGTCTTC
TCATGCTCCGTGATGCATGAGGCTCTGCACAACCACTACACACAGAAGTCCCTCTCCCTGTCTCTGGGTAAATGA
(SEQ ID NO: 228)
Heavy Chain Amino Acid Sequence
QVQLVQSGVEVKKPGASVKVSCKASGYTFKFYGISWVRQAPGQGLEWMGWISVYNGKTKYAQKLQGRVTMTTDTS
TSTAYMELRSLRSDDTAVYFCARDGDYESSGYPFDYWGQGTLVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYG
PPCPPCPAPEFLGGPSVFLFPPKPKDTLMISRTPEVTCVVVDVSQEDPEVQFNWYVDGVEVHNAKTKPREEQENS
TYRVVSVLTVLHQDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHYTQKSLSLSLGK*
(SEQ ID NO: 229)
Light Chain Nucleotide Sequence
GACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCGTCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCCGATCACCTTCGGC
CAAGGGACACGACTGGAGATTAAACGAACTGTGGCTGCACCATCTGTCTTCATCTTCCCGCCATCTGATGAGCAG
TTGAAATCTGGAACTGCCTCTGTTGTGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCAGGACAGCAAGGACAGCACCTACAGC
CTCAGCAGCACCCTGACGCTGAGCAAAGCAGACTACGAGAAACACAAAGTCTACGCCTGCGAAGTCACCCATCAG
GGCCTGAGCTCGCCCGTCACAAAGAGCTTCAACAGGGGAGAGTGTTAG
(SEQ ID NO: 230)
Light Chain Amino Acid Sequence
DIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPITFGQGTRLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC*
(SEQ ID NO: 231)

The present disclosure provides anti-FGFR3 protein-drug conjugates comprising an antibody or antigen-binding fragment thereof that specifically binds to FGFR3 (e.g., monomeric or dimeric human FGFR3b and/or FGFR3c) or an antigenic fragment thereof comprising: a heavy chain variable region (HCVR) that comprises the HCDR1, HCDR2 and HCDR3 of a HCVR that comprises the amino acid sequence set forth in SEQ ID NO: 2, 22, 42, 62, 82, 102, 122, 140, 159, 169, 179, 199 or 219 (e.g., fused to an IgG4 Fc having a S108P mutation), and a light chain variable region (LCVR) that comprises the LCDR1, LCDR2 and LCDR3 of a LCVR that comprises the amino acid sequence set forth in SEQ ID NO: 10, 30, 50, 70, 90, 110, 130, 148, 187, 207, or 227.

The present disclosure also provides an anti-FGFR3 protein-drug conjugate comprising an isolated antibody or antigen-binding fragment thereof that specifically binds to FGFR3 (e.g., monomeric or dimeric human FGFR3b and/or FGFR3c) or an antigenic fragment thereof comprising: (a) a heavy chain variable region (HCVR) that comprises the HCDR1, HCDR2 and HCDR3 of a HCVR that comprises the amino acid sequence set forth in SEQ ID NO: 2, and a light chain variable region (LCVR) that comprises the LCDR1, LCDR2 and LCDR3 of a LCVR that comprises the amino acid sequence set forth in SEQ ID NO: 10, (b) a heavy chain variable region (HCVR) that comprises the HCDR1, HCDR2 and HCDR3 of a HCVR that comprises the amino acid sequence set forth in SEQ ID NO: 22, and a light chain variable region (LCVR) that comprises the LCDR1, LCDR2 and LCDR3 of a LCVR that comprises the amino acid sequence set forth in SEQ ID NO: 30, (c) a heavy chain variable region (HCVR) that comprises the HCDR1, HCDR2 and HCDR3 of a HCVR that comprises the amino acid sequence set forth in SEQ ID NO: 42, and a light chain variable region (LCVR) that comprises the LCDR1, LCDR2 and LCDR3 of a LCVR that comprises the amino acid sequence set forth in SEQ ID NO: 50, (d) a heavy chain variable region (HCVR) that comprises the HCDR1, HCDR2 and HCDR3 of a HCVR that comprises the amino acid sequence set forth in SEQ ID NO: 62, and a light chain variable region (LCVR) that comprises the LCDR1, LCDR2 and LCDR3 of a LCVR that comprises the amino acid sequence set forth in SEQ ID NO: 70, (e) a heavy chain variable region (HCVR) that comprises the HCDR1, HCDR2 and HCDR3 of a HCVR that comprises the amino acid sequence set forth in SEQ ID NO: 82, and a light chain variable region (LCVR) that comprises the LCDR1, LCDR2 and LCDR3 of a LCVR that comprises the amino acid sequence set forth in SEQ ID NO: 90, (f) a heavy chain variable region (HCVR) that comprises the HCDR1, HCDR2 and HCDR3 of a HCVR that comprises the amino acid sequence set forth in SEQ ID NO: 102, and a light chain variable region (LCVR) that comprises the LCDR1, LCDR2 and LCDR3 of a LCVR that comprises the amino acid sequence set forth in SEQ ID NO: 110, and or (g) a heavy chain variable region (HCVR) that comprises the HCDR1, HCDR2 and HCDR3 of a HCVR that comprises the amino acid sequence set forth in SEQ ID NO: 122, and a light chain variable region (LCVR) that comprises the LCDR1, LCDR2 and LCDR3 of a LCVR that comprises the amino acid sequence set forth in SEQ ID NO: 130, (h) a heavy chain variable region (HCVR) that comprises the HCDR1, HCDR2 and HCDR3 of a HCVR that comprises the amino acid sequence set forth in SEQ ID NO: 140, and a light chain variable region (LCVR) that comprises the LCDR1, LCDR2 and LCDR3 of a LCVR that comprises the amino acid sequence set forth in SEQ ID NO: 148, (i) a heavy chain variable region (HCVR) that comprises the HCDR1, HCDR2 and HCDR3 of a HCVR that comprises the amino acid sequence set forth in SEQ ID NO: 159, and a light chain variable region (LCVR) that comprises the LCDR1, LCDR2 and LCDR3 of a LCVR that comprises the amino acid sequence set forth in SEQ ID NO: 148, (j) a heavy chain variable region (HCVR) that comprises the HCDR1, HCDR2 and HCDR3 of a HCVR that comprises the amino acid sequence set forth in SEQ ID NO: 169, and a light chain variable region (LCVR) that comprises the LCDR1, LCDR2 and LCDR3 of a LCVR that comprises the amino acid sequence set forth in SEQ ID NO: 148, (k) a heavy chain variable region (HCVR) that comprises the HCDR1, HCDR2 and HCDR3 of a HCVR that comprises the amino acid sequence set forth in SEQ ID NO: 179, and a light chain variable region (LCVR) that comprises the LCDR1, LCDR2 and LCDR3 of a LCVR that comprises the amino acid sequence set forth in SEQ ID NO: 187, (I) a heavy chain variable region (HCVR) that comprises the HCDR1, HCDR2 and HCDR3 of a HCVR that comprises the amino acid sequence set forth in SEQ ID NO: 199, and a light chain variable region (LCVR) that comprises the LCDR1, LCDR2 and LCDR3 of a LCVR that comprises the amino acid sequence set forth in SEQ ID NO: 207, and/or (m) a heavy chain variable region (HCVR) that comprises the HCDR1, HCDR2 and HCDR3 of a HCVR that comprises the amino acid sequence set forth in SEQ ID NO: 219, and a light chain variable region (LCVR) that comprises the LCDR1, LCDR2 and LCDR3 of a LCVR that comprises the amino acid sequence set forth in SEQ ID NO: 227.

The present disclosure also provides anti-FGFR3 protein-drug conjugates comprising an isolated antibody or antigen-binding fragment thereof that specifically binds to FGFR3 (e.g., monomeric or dimeric human FGFR3b and/or FGFR3c) or an antigenic fragment thereof comprising: (a) a heavy chain variable region that comprises an HCDR1 that comprises the amino acid sequence set forth in SEQ ID NO: 4, an HCDR2 that comprises the amino acid sequence set forth in SEQ ID NO: 6, and an HCDR3 that comprises the amino acid sequence set forth in SEQ ID NO: 8, and a light chain variable region that comprises an LCDR1 that comprises the amino acid sequence set forth in SEQ ID NO: 12, an LCDR2 that comprises the amino acid sequence set forth in SEQ ID NO: 14, and an LCDR3 that comprises the amino acid sequence set forth in SEQ ID NO: 16; (b) a heavy chain variable region that comprises an HCDR1 that comprises the amino acid sequence set forth in SEQ ID NO: 24, an HCDR2 that comprises the amino acid sequence set forth in SEQ ID NO: 26, and an HCDR3 that comprises the amino acid sequence set forth in SEQ ID NO: 28, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 36; (c) a heavy chain variable region that comprises an HCDR1 that comprises the amino acid sequence set forth in SEQ ID NO: 44, an HCDR2 that comprises the amino acid sequence set forth in SEQ ID NO: 46, and an HCDR3 that comprises the amino acid sequence set forth in SEQ ID NO: 48, and a light chain variable region that comprises an LCDR1 that comprises the amino acid sequence set forth in SEQ ID NO: 52, an LCDR2 that comprises the amino acid sequence set forth in SEQ ID NO: 54, and an LCDR3 that comprises the amino acid sequence set forth in SEQ ID NO: 56; (d) a heavy chain variable region that comprises an HCDR1 that comprises the amino acid sequence set forth in SEQ ID NO: 64, an HCDR2 that comprises the amino acid sequence set forth in SEQ ID NO: 66, and an HCDR3 that comprises the amino acid sequence set forth in SEQ ID NO: 68, and a light chain variable region that comprises an LCDR1 that comprises the amino acid sequence set forth in SEQ ID NO: 72, an LCDR2 that comprises the amino acid sequence set forth in SEQ ID NO: 74, and an LCDR3 that comprises the amino acid sequence set forth in SEQ ID NO: 76; (e) a heavy chain variable region that comprises an HCDR1 that comprises the amino acid sequence set forth in SEQ ID NO: 84, an HCDR2 that comprises the amino acid sequence set forth in SEQ ID NO: 86, and an HCDR3 that comprises the amino acid sequence set forth in SEQ ID NO: 88, and a light chain variable region that comprises an LCDR1 that comprises the amino acid sequence set forth in SEQ ID NO: 92, an LCDR2 that comprises the amino acid sequence set forth in SEQ ID NO: 94, and an LCDR3 that comprises the amino acid sequence set forth in SEQ ID NO: 96; (f) a heavy chain variable region that comprises an HCDR1 that comprises the amino acid sequence set forth in SEQ ID NO: 104, an HCDR2 that comprises the amino acid sequence set forth in SEQ ID NO: 106, and an HCDR3 that comprises the amino acid sequence set forth in SEQ ID NO: 108, and a light chain variable region that comprises an LCDR1 that comprises the amino acid sequence set forth in SEQ ID NO: 112, an LCDR2 that comprises the amino acid sequence set forth in SEQ ID NO: 114, and an LCDR3 that comprises the amino acid sequence set forth in SEQ ID NO: 116; and/or (g) a heavy chain variable region that comprises an HCDR1 that comprises the amino acid sequence set forth in SEQ ID NO: 124, an HCDR2 that comprises the amino acid sequence set forth in SEQ ID NO: 126, and an HCDR3 that comprises the amino acid sequence set forth in SEQ ID NO: 128, and a light chain variable region that comprises an LCDR1 that comprises the amino acid sequence set forth in SEQ ID NO: 132, an LCDR2 that comprises the amino acid sequence set forth in SEQ ID NO: 34, and an LCDR3 that comprises the amino acid sequence set forth in SEQ ID NO: 134; (h) a heavy chain variable region that comprises an HCDR1 that comprises the amino acid sequence set forth in SEQ ID NO: 142, an HCDR2 that comprises the amino acid sequence set forth in SEQ ID NO: 144, and an HCDR3 that comprises the amino acid sequence set forth in SEQ ID NO: 146, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (i) a heavy chain variable region that comprises an HCDR1 that comprises the amino acid sequence set forth in SEQ ID NO: 161, an HCDR2 that comprises the amino acid sequence set forth in SEQ ID NO: 163, and an HCDR3 that comprises the amino acid sequence set forth in SEQ ID NO: 165, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (j) a heavy chain variable region that comprises an HCDR1 that comprises the amino acid sequence set forth in SEQ ID NO: 171, an HCDR2 that comprises the amino acid sequence set forth in SEQ ID NO: 173, and an HCDR3 that comprises the amino acid sequence set forth in SEQ ID NO: 175, and a light chain variable region that comprises an LCDR1 that comprises the amino acid sequence set forth in SEQ ID NO: 150, an LCDR2 that comprises the amino acid sequence set forth in SEQ ID NO: 14, and an LCDR3 that comprises the amino acid sequence set forth in SEQ ID NO: 153; (k) a heavy chain variable region that comprises an HCDR1 that comprises the amino acid sequence set forth in SEQ ID NO: 181, an HCDR2 that comprises the amino acid sequence set forth in SEQ ID NO: 183, and an HCDR3 that comprises the amino acid sequence set forth in SEQ ID NO: 185, and a light chain variable region that comprises an LCDR1 that comprises the amino acid sequence set forth in SEQ ID NO: 189, an LCDR2 that comprises the amino acid sequence set forth in SEQ ID NO: 191, and an LCDR3 that comprises the amino acid sequence set forth in SEQ ID NO: 193; (I) a heavy chain variable region that comprises an HCDR1 that comprises the amino acid sequence set forth in SEQ ID NO: 201, an HCDR2 that comprises the amino acid sequence set forth in SEQ ID NO: 203, and an HCDR3 that comprises the amino acid sequence set forth in SEQ ID NO: 205, and a light chain variable region that comprises an LCDR1 that comprises the amino acid sequence set forth in SEQ ID NO: 209, an LCDR2 that comprises the amino acid sequence set forth in SEQ ID NO: 211, and an LCDR3 that comprises the amino acid sequence set forth in SEQ ID NO: 213; and/or (m) a heavy chain variable region that comprises an HCDR1 that comprises the amino acid sequence set forth in SEQ ID NO: 221, an HCDR2 that comprises the amino acid sequence set forth in SEQ ID NO: 223, and an HCDR3 that comprises the amino acid sequence set forth in SEQ ID NO: 225, and a light chain variable region that comprises an LCDR1 that comprises the amino acid sequence set forth in SEQ ID NO: 32, an LCDR2 that comprises the amino acid sequence set forth in SEQ ID NO: 34, and an LCDR3 that comprises the amino acid sequence set forth in SEQ ID NO: 76.

The present disclosure further provides anti-FGFR3 protein-drug conjugates comprising an isolated antibody or antigen-binding fragment thereof that specifically binds to FGFR3 or an antigenic fragment thereof comprising a heavy chain variable region that comprises the amino acid sequence set forth in SEQ ID NO: 2, 22, 42, 62, 82, 102, 122, 140, 159, 169, 179, 199 or 219, and a light chain variable region that comprises the amino acid sequence set forth in SEQ ID NO: 10, 30, 50, 70, 90, 110, 130, 148, 187, 207, or 227.

In addition, the present disclosure provides an isolated antibody or antigen-binding fragment thereof that specifically binds to FGFR3 (e.g., monomeric or dimeric human FGFR3b and/or FGFR3c) or an antigenic fragment thereof comprising: (a) a heavy chain variable region that comprises the amino acid sequence set forth in SEQ ID NO: 2, and a light chain variable region that comprises the amino acid sequence set forth in SEQ ID NO: 10; (b) a heavy chain variable region that comprises the amino acid sequence set forth in SEQ ID NO: 22, and a light chain variable region that comprises the amino acid sequence set forth in SEQ ID NO: 30; (c) a heavy chain variable region that comprises the amino acid sequence set forth in SEQ ID NO: 42, and a light chain variable region that comprises the amino acid sequence set forth in SEQ ID NO: 50; (d) a heavy chain variable region that comprises the amino acid sequence set forth in SEQ ID NO: 62, and a light chain variable region that comprises the amino acid sequence set forth in SEQ ID NO: 70; (e) a heavy chain variable region that comprises the amino acid sequence set forth in SEQ ID NO: 82, and a light chain variable region that comprises the amino acid sequence set forth in SEQ ID NO: 90; (f) a heavy chain variable region that comprises the amino acid sequence set forth in SEQ ID NO: 102, and a light chain variable region that comprises the amino acid sequence set forth in SEQ ID NO: 110; e.g., wherein the heavy chain variable region is fused to an IgG4 Fc having a S108P mutation; and/or (g) a heavy chain variable region that comprises the amino acid sequence set forth in SEQ ID NO: 122, and a light chain variable region that comprises the amino acid sequence set forth in SEQ ID NO: 130; (h) a heavy chain variable region that comprises the amino acid sequence set forth in SEQ ID NO: 140, and a light chain variable region that comprises the amino acid sequence set forth in SEQ ID NO: 148; (i) a heavy chain variable region that comprises the amino acid sequence set forth in SEQ ID NO: 159, and a light chain variable region that comprises the amino acid sequence set forth in SEQ ID NO: 148; (j) a heavy chain variable region that comprises the amino acid sequence set forth in SEQ ID NO: 169, and a light chain variable region that comprises the amino acid sequence set forth in SEQ ID NO: 148; (k) a heavy chain variable region that comprises the amino acid sequence set forth in SEQ ID NO: 179, and a light chain variable region that comprises the amino acid sequence set forth in SEQ ID NO: 187; (I) a heavy chain variable region that comprises the amino acid sequence set forth in SEQ ID NO: 199, and a light chain variable region that comprises the amino acid sequence set forth in SEQ ID NO: 207; and/or (m) a heavy chain variable region that comprises the amino acid sequence set forth in SEQ ID NO: 219, and a light chain variable region that comprises the amino acid sequence set forth in SEQ ID NO: 227.

The present disclosure provides anti-FGFR3 protein-drug conjugates comprising an isolated antibody or antigen-binding fragment thereof that specifically binds to FGFR3 (e.g., monomeric or dimeric human FGFR3b and/or FGFR3c) or an antigenic fragment thereof comprising (a) a heavy chain that comprises the amino acid sequence set forth in SEQ ID NO: 18, 38, 58, 78, 98, 118, 136, 155, 167, 177, 195, 215 or 229, and a light chain that comprises the amino acid sequence set forth in SEQ ID NO: 20, 40, 60, 80, 100 120, 138, 157, 197, 217 or 231.

The present disclosure also provides anti-FGFR3 protein-drug conjugates comprising an isolated antibody or antigen-binding fragment thereof that specifically binds to FGFR3 or an antigenic fragment thereof comprising: (a) a heavy chain that comprises the amino acid sequence set forth in SEQ ID NO: 18, and a light chain that comprises the amino acid sequence set forth in SEQ ID NO: 20; (b) a heavy chain variable region that comprises the amino acid sequence set forth in SEQ ID NO: 38, and a light chain variable region that comprises the amino acid sequence set forth in SEQ ID NO: 40; (c) a heavy chain variable region that comprises the amino acid sequence set forth in SEQ ID NO: 58, and a light chain variable region that comprises the amino acid sequence set forth in SEQ ID NO: 60; (d) a heavy chain variable region that comprises the amino acid sequence set forth in SEQ ID NO: 78, and a light chain variable region that comprises the amino acid sequence set forth in SEQ ID NO: 80; (e) a heavy chain variable region that comprises the amino acid sequence set forth in SEQ ID NO: 98, and a light chain variable region that comprises the amino acid sequence set forth in SEQ ID NO: 100; (f) a heavy chain variable region that comprises the amino acid sequence set forth in SEQ ID NO: 118, and a light chain variable region that comprises the amino acid sequence set forth in SEQ ID NO: 120; and/or (g) a heavy chain that comprises the amino acid sequence set forth in SEQ ID NO: 136, and a light chain that comprises the amino acid sequence set forth in SEQ ID NO: 138; (h) a heavy chain that comprises the amino acid sequence set forth in SEQ ID NO: 155, and a light chain that comprises the amino acid sequence set forth in SEQ ID NO: 157; (i) a heavy chain that comprises the amino acid sequence set forth in SEQ ID NO: 167, and a light chain that comprises the amino acid sequence set forth in SEQ ID NO: 157; (j) a heavy chain that comprises the amino acid sequence set forth in SEQ ID NO: 177, and a light chain that comprises the amino acid sequence set forth in SEQ ID NO: 157; (k) a heavy chain that comprises the amino acid sequence set forth in SEQ ID NO: 195, and a light chain that comprises the amino acid sequence set forth in SEQ ID NO: 197; (I) a heavy chain that comprises the amino acid sequence set forth in SEQ ID NO: 215, and a light chain that comprises the amino acid sequence set forth in SEQ ID NO: 217; and/or (m) a heavy chain that comprises the amino acid sequence set forth in SEQ ID NO: 229, and a light chain that comprises the amino acid sequence set forth in SEQ ID NO: 231.

The present disclosure provides anti-FGFR3 protein-drug conjugates comprising an isolated antibody or antigen-binding fragment thereof that specifically binds to FGFR3 or antigenic fragment thereof comprising a heavy chain that comprises the amino acid sequence set forth in SEQ ID NO: 18, 38, 58, 78, 98, 118, 136, 155, 167, 177, 195, 215, or 229 and a light chain that comprises the amino acid sequence set forth in SEQ ID NO: 20, 40, 60, 80, 100, 120, 138, 157, 197, 217, or 231.

The present disclosure also provides anti-FGFR3 protein-drug conjugates comprising an isolated antibody or antigen-binding fragment thereof that specifically binds to FGFR3 or an antigenic fragment thereof comprising: (a) a heavy chain that comprises the amino acid sequence set forth in SEQ ID NO: 18, and a light chain that comprises the amino acid sequence set forth in SEQ ID NO: 20; (b) a heavy chain that comprises the amino acid sequence set forth in SEQ ID NO: 38, and a light chain that comprises the amino acid sequence set forth in SEQ ID NO: 40; (c) a heavy chain that comprises the amino acid sequence set forth in SEQ ID NO: 58, and a light chain that comprises the amino acid sequence set forth in SEQ ID NO: 60; (d) a heavy chain that comprises the amino acid sequence set forth in SEQ ID NO: 78, and a light chain that comprises the amino acid sequence set forth in SEQ ID NO: 80; (e) a heavy chain that comprises the amino acid sequence set forth in SEQ ID NO: 98, and a light chain that comprises the amino acid sequence set forth in SEQ ID NO: 100; (f) a heavy chain that comprises the amino acid sequence set forth in SEQ ID NO: 118, and a light chain that comprises the amino acid sequence set forth in SEQ ID NO: 120; (g) a heavy chain that comprises the amino acid sequence set forth in SEQ ID NO: 136, and a light chain that comprises the amino acid sequence set forth in SEQ ID NO: 138; (h) a heavy chain that comprises the amino acid sequence set forth in SEQ ID NO: 155, and a light chain that comprises the amino acid sequence set forth in SEQ ID NO: 157; (i) a heavy chain that comprises the amino acid sequence set forth in SEQ ID NO: 167, and a light chain that comprises the amino acid sequence set forth in SEQ ID NO: 157; (j) a heavy chain that comprises the amino acid sequence set forth in SEQ ID NO: 177, and a light chain that comprises the amino acid sequence set forth in SEQ ID NO: 157; (k) a heavy chain that comprises the amino acid sequence set forth in SEQ ID NO: 195, and a light chain that comprises the amino acid sequence set forth in SEQ ID NO: 197; (I) a heavy chain that comprises the amino acid sequence set forth in SEQ ID NO: 215, and a light chain that comprises the amino acid sequence set forth in SEQ ID NO: 217; or (m) a heavy chain that comprises the amino acid sequence set forth in SEQ ID NO: 229, and a light chain that comprises the amino acid sequence set forth in SEQ ID NO: 231.

In some embodiments, the antigen-binding protein binds to the same epitope on FGFR3 (e.g., FGFR3b or FGFR3c) as an antibody comprising an HCVR/LCVR amino acid sequence pair as set forth in Table 1-1.

In some embodiments, the antigen-binding protein competes for binding to FGFR3 (e.g., FGFR3b or FGFR3c) with an antibody comprising an HCVR/LCVR amino acid sequence pair as set forth in Table 1-1.

As discussed, an anti-FGFR3 protein-drug conjugate may comprise an anti-FGFR3 scFv comprising an optional signal peptide (e.g., mROR signal sequence), connected to an scFv (e.g., including a V_{L} and a V_{H} optionally connected by a linker), connected to an option linker, connected to a molecular cargo. In various embodiments, the optional signal peptide is, for example, the signal peptide from Mus musculus Ror1 (e.g., comprising or consisting of the amino acids MHRPRRRGTRPPPLALLAALLLAARGADA (SEQ ID NO: 245)).

In some embodiments, an anti-FGFR3 scFv described herein, in VL-(Gly4Ser)3 (SEQ ID NO: 246)-VH format, comprises an amino acid sequence as set forth in Table 1-1. In other embodiments, the present disclosure includes scFvs that are in the format VH-(Gly4Ser)3 (SEQ ID NO: 246)-VL. Optionally, an anti-FGFR3 scFv of the present disclosure further includes a tag sequence LLQGSG (SEQ ID NO: 247) and/or HHHHHH (SEQ ID NO: 235). The tag sequence LLQGSG (SEQ ID NO: 247) or HHHHHH (SEQ ID NO: 235) may be included at the N-terminus and/or C-terminus of the anti-FGFR3 scFv. In one embodiment, an anti-FGFR3 scFv of the present invention further includes an N-terminal LLQGSG (SEQ ID NO: 247) and/or a C-terminal HHHHHH (SEQ ID NO: 235).

In some embodiments, the FGFR3 binding protein described herein comprises a humanized antibody or antigen binding fragment thereof, human antibody or antigen binding fragment thereof, murine antibody or antigen binding fragment thereof, chimeric antibody or antigen binding fragment thereof, monoclonal antibody or antigen binding fragment thereof (e.g., monovalent Fab′, divalent Fab2, F(ab)′3 fragments, single-chain variable fragment (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, camelid antibody or antigen binding fragment thereof, single heavy chain antibody, bispecific antibody or biding fragment thereof, (e.g., bisscFv, ora bi-specific T-cell engager (BITE)), trispecific antibody (e.g., F(ab)′3 fragments or a triabody), or a chemically modified derivative thereof.

The term “humanized antibody”, as used herein, includes antibodies in which CDR sequences derived from the germline of another mammalian species, such as a mouse, have been grafted onto human framework sequences, or otherwise modified to increase their similarity to antibody variants produced naturally in humans.

In some cases, the FGFR3 binding protein is an antibody which comprises one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The anti-FGFR3 antibodies and antigen-binding fragments described herein may be modified after translation, e.g., glycosylated.

For example, antibodies and antigen-binding fragments described herein may be glycosylated (e.g., N-glycosylated and/or O-glycosylated) or aglycosylated. Typically, antibodies and antigen-binding fragments are glycosylated at the conserved residue N297 of the IgG Fc domain. Some antibodies and fragments include one or more additional glycosylation sites in a variable region. In an embodiment, the glycosylation site is in the following context: FN297S or YN297S.

In an embodiment, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In some cases, the anti-FGFR3 antigen-binding protein is an aglycosylated antibody, i.e., an antibody that does not comprise a glycosylation sequence that might interfere with a transglutamination reaction, for instance an antibody that does not have a saccharide group at N297 on one or more heavy chains according to the EU numbering system. In particular embodiments, an antibody heavy chain has an N297 mutation. In particular embodiments, an antibody heavy chain has an N297Q or an N297D mutation. The N-linked glycan found at position 297 can be found as a core structure, common to all IgG found in human beings and rodents. Antibodies comprising such above-described mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

In some cases, the antigen-binding protein is a deglycosylated antibody, i.e., an antibody in which a saccharide group at is removed to facilitate transglutaminase-mediated conjugation. Saccharides include, but are not limited to, N-linked oligosaccharides. In some embodiments, deglycosylation is performed at residue N297 chains according to the EU numbering system. In some embodiments, removal of saccharide groups is accomplished enzymatically, included but not limited to via PNGase.

In an embodiment, an antibody or fragment described herein is afucosylated.

The antibodies and antigen-binding fragments described herein may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

In some embodiments, an antibody disclosed herein comprises Q295 which can be native to the antibody heavy chain sequence. In some embodiments, an antibody heavy chain disclosed herein may comprise Q295. In some embodiments, an antibody heavy chain disclosed herein may comprise Q295 and an amino acid substitution N297D.

According to certain embodiments of the present disclosure, anti-FGFR3 (e.g., monomeric or dimeric FGFR3b and/or FGFR3c) antibodies and antigen-binding fragments (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2) are provided comprising an Fc domain comprising one or more mutations which enhance or diminish antibody binding to the FcRn receptor, e.g., at acidic pH as compared to neutral pH (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2). For example, the present disclosure includes anti-FGFR3 antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., LN/F/W or T),
254 (e.g., S or T), and/or
256 (e.g., S/R/Q/E/D or T);
   and/or a modification at position:
428 and/or 433 (e.g., H/L/R/S/P/Q or K), and/or
434 (e.g., A, W, H, F or Y);
   and/or a modification at position:
250 and/or 428;
   and/or a modification at position:
307 or 308 (e.g., 308F, V308F), and/or 434.

In an embodiment, the modification comprises:
a 428L (e.g., M428L) and 434S (e.g., N434S) modification;
a 428L, 2591 (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes anti-FGFR3 antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
2571 and 3111 (e.g., P2571 and Q3111);
2571 and 434H (e.g., P2571 and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In yet another embodiment, the modification comprises a 265A (e.g., D265A) and/or a 297A (e.g., N297A) modification.

In an embodiment, the heavy chain constant domain is gamma4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

All possible combinations of the foregoing Fc domain mutations, and other mutations within the antibody variable domains disclosed herein, are contemplated within the scope of the present disclosure.

The anti-FGFR3 antibodies described herein may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments, the modified Fc domain is a variant IgG1 Fc or a variant IgG4 Fc comprising a substitution in the hinge region. For example, a modified Fc for use in the context of the present disclosure may comprise a variant IgG1 Fc wherein at least one amino acid of the IgG1 Fc hinge region is replaced with the corresponding amino acid from the IgG2 Fc hinge region. Alternatively, a modified Fc for use in the context of the present disclosure may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present disclosure are set forth in US Patent Application Publication No. 2014/0243504, the disclosure of which is hereby incorporated by reference in its entirety, as well as any functionally equivalent variants of the modified Fc regions set forth therein.

The present disclosure also includes antigen-binding proteins, antibodies or antigen-binding fragments, comprising a HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies of the disclosure comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., WO2014/022540).

Other modified Fc domains and Fc modifications that can be used in the context of the present disclosure include any of the modifications as set forth in US2014/0171623, U.S. Pat. No. 8,697,396; US2014/0134162, WO2014/043361, the disclosures of which are hereby incorporated by reference in their entireties. Methods of constructing antibodies or other antigen-binding fusion proteins comprising a modified Fc domain as described herein are known in the art.

In some cases, the anti-FGFR3 antibodies may comprise one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcyR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The present disclosure provides a vessel (e.g., a plastic or glass vial, e.g., with a cap or a chromatography column, hollow bore needle or a syringe cylinder) comprising an anti-FGFR3 protein-drug conjugates, e.g., FGFR3 binding protein-drug conjugates or anti-FGFR3 Fab-drug conjugates described herein.

The present disclosure also provides an injection device comprising an anti-FGFR3 protein-drug conjugate, e.g., anti-FGFR3 scFv-drug conjugates or anti-FGFR3 Fab-drug conjugates described herein, or a pharmaceutical composition thereof. The injection device may be packaged into a kit. An injection device is a device that introduces a substance into the body of a subject via a parenteral route, e.g., intrathecal, intracisternal (e.g., cisterna magna), intracerebroventricular, intraparenchymal, intraocular, intravitreal, intramuscular, subcutaneous or intravenous. For example, an injection device may be a syringe or an auto-injector (e.g., pre-filled with the pharmaceutical formulation) which, for example, includes a cylinder or barrel for holding fluid to be injected (e.g., comprising the antibody or fragment or a pharmaceutical formulation thereof), a needle for piecing skin, blood vessels or other tissue for injection of the fluid; and a plunger for pushing the fluid out of the cylinder and through the needle bore and into the body of the subject.

The present disclosure provides methods for administering an anti-FGFR3 (e.g., monomeric or dimeric FGFR3b and/or FGFR3c) antigen-binding protein, e.g., antibody or antigen-binding fragment thereof (e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2) to a subject, comprising introducing the protein or a pharmaceutical formulation thereof into the body of the subject. For example, in an embodiment, the method comprises piercing the body of the subject, e.g., with a needle of a syringe, and injecting the antigen-binding protein or a pharmaceutical formulation thereof into the body of the subject, e.g., into the eye, vein, artery, muscular tissue or subcutis of the subject.

The present disclosure further provides methods for delivering a molecular cargo, wherein the molecular cargo is conjugated to, e.g., an antigen-binding protein described herein, e.g., an anti-FGFR3 scFv or an anti-FGFR3 Fab described herein, to a target tissue (e.g., nervous tissue in the central nervous system, eye) or a target cell (e.g., astrocyte) in a subject, comprising introducing the protein-drug conjugate into the body of the subject (e.g., a human), for example, parenterally (e.g., via intrathecal, intracerebroventricular, intracisternal (e.g., cisterna magna), or intraparenchymal injection). For example, the method comprises piercing the body of the subject with a needle of a syringe and injecting the protein-drug conjugate into the body of the subject, e.g., into the brain or spinal cord of the subject. For example, the protein-drug conjugate may be introduced into the subject via intrathecal, intracisternal (e.g., cisterna magna), intracerebroventricular, or intraparenchymal injection into the central nervous system.

The present disclosure further provides a cell line useful for screening the FGFR3 binding proteins or anti-FGFR3 protein-drug conjugates described herein. The cell lines described herein express FGFR3b and/or FGFR3c on the cell surface, and optionally further comprise an exogenous nucleic acid to express one or more reporter proteins. In some embodiments, the cell line is modified from a brain cell line, such as a glioblastoma cell line. In one embodiment, the cell line is modified from the U87 glioblastoma cell line.

In some embodiments, the cell line comprise an exogenous nucleic acid (e.g., mRNA) to express two reporter proteins. Non-limiting examples of reporter proteins that can be used in the present application include fluorescent proteins, such as green fluorescent protein (GFP), red fluorescent protein (RFP), yellow fluorescent protein (YFP), blue fluorescent protein (BFP), or luminescent proteins, such as firefly luciferase, Renilla luciferase, or Nanoluc luciferase. In some embodiments, the cell lines comprise an exogenous nucleic acid to express both GFP and firefly luciferase. The genes encoding the two reporter proteins are optionally separated by a sequence encoding a self-cleaving peptide or an internal ribosomal entry site (IRES). The self-cleaving peptide can be a 2A peptide such as T2A, P2A, E2A, or F2A peptide.

In some embodiments, the cell lines can be used to assess binding and/or internalization properties of the FGFR3 binding proteins or anti-FGFR3 protein-drug conjugates described herein. The cell lines allow for high throughput screening of FGFR3 binding proteins or anti-FGFR3 protein-drug conjugates to identify the best candidate for therapeutic delivery, and/or allow for testing of the most efficient siRNA modifications, linker chemistries, as well as LNP chemistries.

In some embodiments, the cell lines can be used to screen for interfering nucleic acids (e.g., siRNAs) or gRNAs to identify genes or factors that could further promote endosomal escape (or alleviate the burden of lack of endosomal escape) of FGFR3 protein-drug conjugates to allow the cargo to be better delivered to the target cells.

### Molecular Cargoes

In some aspects, the present disclosure includes methods and compositions for delivering a conjugated molecular cargo to a cell or tissue. In certain aspects the antigen-binding protein that binds specifically to fibroblast growth factor receptor 3 (FGFR3) disclosed herein, e.g., an antibody or an antigen-binding fragment thereof (e.g., an scFv), may be conjugated (e.g., covalently conjugated) to the molecular cargo.

As used herein, the term “molecular cargo” refers to a molecule that operates to effect a biological outcome. As a non-limiting example, the molecular cargo may operate to modulate the transcription of a DNA sequence, to modulate the expression of a protein, or to modulate the activity of a protein, to delete or disrupt an endogenous gene (or fragment thereof), to achieve an enzymatic activity, to supplement or replace a deficient endogenous protein, to insert an exogenous gene (or fragment thereof), or to replace an endogenous gene (or fragment thereof) with an exogenous gene (or fragment thereof). In various embodiments, the molecular cargo may comprise a polynucleotide. In various embodiments, the molecular cargo may comprise a polypeptide. In various embodiments, the molecular cargo comprises a lipid nanoparticle, liposome, or non-lipid nanoparticle described herein, which optionally comprises one or more polynucleotide and/or a protein molecules. In various embodiments, the molecular cargo may comprise a small molecule. In various embodiments, the molecular cargo may comprise a viral particle (e.g., AAV) or a viral capsid protein.

In some embodiments, the anti-FGFR3 antibody or an antigen-binding fragment thereof disclosed herein may be used, for example, to deliver the conjugated molecular cargo to a cell or a tissue that expresses FGFR3 (e.g., the brain or the spinal cord) for diagnosing and or treating a disease (e.g., a neurological disease). In some embodiments, the molecular cargoes conjugated to the anti-FGFR3 antibody or antigen-binding fragment thereof may be taken up by, e.g., astrocytes, via binding to the FGFR3, which may be endocytosed, e.g., via clathrin-mediated endocytosis, or clatherin- and dynamin-independent pathways (Haugsten et al., PLoS One. 2011, 6(7): e21708). In some embodiments, the anti-FGFR3 antibody or an antigen-binding fragment thereof described herein can exhibit superior activity, e.g., in delivering a molecular cargo into a target tissue (e.g., brain or spinal cord) or a target cell (e.g., an astrocyte).

In some embodiments, the molecular cargo comprises a polynucleotide molecule. The terms “polynucleotide” and “nucleic acid” are used interchangeably herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with optional substitutions, e.g., methoxy or 2′ halide substitutions. In some embodiments, polynucleotides up to about 30 nucleotides in length can be referred to herein as an “oligonucleotide”. Oligonucleotides may be of a variety of different lengths, e.g., depending on the form. In some embodiments, an oligonucleotide is 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the oligonucleotide is 8 to 30 nucleotides in length, 10 to 15 nucleotides in length, 10 to 20 nucleotides in length, 15 to 25 nucleotides in length, 21 to 23 nucleotides in lengths.

In some embodiments, the molecular cargo comprises a polypeptide molecule. The terms “polypeptide” and “protein” used interchangeably herein encompass native or artificial proteins, protein fragments and polypeptide analogs of a protein sequence. A polypeptide or protein may be monomeric or polymeric.

In some embodiments, the molecular cargo described herein may comprise a carrier, such as a liposome or lipid nanoparticle (LNP). A lipid particle, e.g., a liposome or lipid nanoparticle disclosed herein, may include a lipid formulation that can be used to deliver a therapeutic nucleic acid (e.g., gRNA) to a target site of interest (e.g., cell, tissue, organ, and the like). Without wishing to be bound by theory, carriers may be used, e.g., as a means for delivery of a polynucleotide disclosed herein and/or a protein disclosed herein. In some embodiments, a carrier (e.g., liposome or LNP) may be useful for the delivery of a nucleic acid (e.g., DNA or RNA), protein (e.g., RNA-guided DNA binding agent), or a combination thereof. By way of a non-limiting example, a carrier (e.g., liposome or LNP) may be used to deliver various components of a gene editing system, for example, a CRISPR/Cas system or additional gene editing systems described herein.

In some embodiments, the molecular cargo comprises a small molecule. A small molecule (SM) can enter cells easily because it has a low molecular weight (typically, up to about 1 kDa). Once inside the cells, it can affect other molecules, such as proteins including, e.g., apolipoprotein (apo) E risk alleles (e.g., ApoE4), glial fibrillary acidic protein (Gfap), methyl CpG binding protein 2 (MeCp2), Aquaporin-4 (Aqp4), and signal transducer and activator of transcription 3 (Stat3). This is different from many large molecular weight molecules such as antibodies. As an example, a small molecule may be conjugated to an FGFR3 binding protein to form an anti-FGFR3:SM conjugate. A SM for delivery by way of anti-FGFR3-mediated delivery may be suitable for targeting pathological consequences of, e.g., a neurodegenerative disease, a neurodevelopmental disease, physical injury, or a disease or disorder of neuropsychiatric origin (e.g., cell death).

Exemplary molecular cargoes are described in further detail herein, however, it should be appreciated that the exemplary molecular cargoes provided herein are not intended to be limiting.

### Polynucleotide Molecules

Non-limiting examples of polynucleotide molecules that are useful as molecular cargoes in the protein-drug conjugates of the present disclosure include, but are not limited to, interfering nucleic acids (e.g., shRNAs, siRNAs, microRNAs, antisense oligonucleotides), gapmers, mixmers, ribozymes, phosphorodiamidite morpholinos, peptide nucleic acids, aptamers, and guide nucleic acids (e.g., Cas9 guide RNAs), mRNAs, etc. In various embodiments, a polynucleotide may comprise one or more modified nucleotides. In various embodiments, a polynucleotide may comprise one or more modified inter-nucleotide linkage. Polynucleotides may be single-stranded or double-stranded.

In some embodiments, the molecular cargo comprises at least one polynucleotide molecule. In some embodiments, the molecular cargo comprises at least 2, at least 3, at least 4, at least 5, or at least 10 polynucleotide molecules.

In some embodiments, the polynucleotide molecule is DNA. In some embodiments, the polynucleotide molecule is RNA.

In various embodiments, a polynucleotide described herein (e.g., interfering nucleic acid or guide RNA) may comprise a region of complementarity to a target nucleic acid which can be in the range of 8 to 15, 8 to 30, 8 to 40, or 10 to 50, or 5 to 50, or 5 to 40 nucleotides in length. In certain embodiments, a region of complementarity of a polynucleotide to a target nucleic acid may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity may be complementary with at least 10 consecutive nucleotides of a target nucleic acid. In some embodiments, a polynucleotide may contain 1, 2, 3, 4 or 5 base mismatches compared to the portion of the consecutive nucleotides of target nucleic acid. In some embodiments the polynucleotide may have up to 3 mismatches over 15 bases, or up to 4 mismatches over 10 bases. In some embodiments, the polynucleotide is complementary (e.g., at least 80%, at least 85% at least 90%, at least 95%, or 100%) to a target sequence of any one of the polynucleotides described herein. In various embodiments, such target sequence may be 100% complementary to the polynucleotide described herein. In some embodiments, any one or more of the thymine bases (T's) in any one of the polynucleotides described herein may be uracil bases (U's), and/or any one or more of the U's may be T's. A target sequence described herein may comprise a sequence of nucleic acid in a target gene that has complementarity to the guide sequence of the gRNA. The interaction of the target sequence and the guide sequence directs an RNA-guided DNA-binding agent (e.g., Cas protein) to bind, and potentially nick or cleave (depending on the activity of the agent), within the target sequence.

The polynucleotides described herein may be modified, e.g., comprise a modified nucleotide, a modified internucleoside linkage, and/or a modified sugar moiety, or combinations thereof. In addition, polynucleotides can possess one or more of the following properties: have improved cell uptake compared to unmodified polynucleotides; are not toxic to cells or mammals are not immune stimulatory; avoid pattern recognition receptors do not mediate alternative splicing; are nuclease resistant; have improved endosomal exit internally in a cell; or minimizes TLR stimulation. Any of the various modified chemistries or formats of polynucleotides disclosed herein may be combined with together. As a non-limiting example, one, two, three, four, five, six, seven, eight or more different types of modifications may be included within the same polynucleotide.

In various embodiments, particular nucleotide modification(s) may be used that render a polynucleotide into which the modification(s) are incorporated more resistant to nuclease digestion than the native oligoribonucleotide or oligodeoxynucleotide molecules; such modified polynucleotides survive intact for a longer time than unmodified polynucleotides. Exemplary modified polynucleotides include those comprising modified backbones, for example, modified internucleoside linkages such as, methyl phosphonates, phosphotriesters, phosphorothioates short chain alkyl or cycloalkyl intersugar linkages heterocyclic intersugar linkages or short chain heteroatomic or. As such, polynucleotides described herein may be stabilized against nucleolytic degradation, e.g., via incorporation of a modification, e.g., a nucleotide modification.

In various embodiments, a polynucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, or 2 to 45, nucleotides of the polynucleotide may be modified nucleotides. The polynucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the polynucleotide can be modified nucleotides. In some embodiments, the polynucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the polynucleotide are modified nucleotides. In some embodiments, the polynucleotides can have every nucleotide except 1, 2, 3, 4, 5, 6, 7, 8, 9, 10, 11, 12, 13, 14, or 15 nucleotides modified.

In various embodiments, the polynucleotide disclosed herein may comprise at least one nucleoside, e.g., modified at the 2′ position of the sugar. In some embodiments, all of the nucleosides in the polynucleotide are 2′-modified nucleosides. In some embodiments, a polynucleotide comprises at least one 2′-modified nucleoside.

In various embodiments, the polynucleotide disclosed herein may one or more non-bicyclic 2′-modified nucleosides, e.g., 2′-O-dimethylaminoethyloxyethyl (2′-O-DMAEOE)2′-O-methyl (2′-O-Me), 2′-O-dimethylaminoethyl (2′-O-DMAOE), 2′-O-methoxyethyl (2′-MOE), 2′-deoxy, 2′-O—N-methylacetamido (2′-O-NMA) modified nucleoside, 2′-fluoro (2′-F), 2′-O-aminopropyl (2′-O-AP), or 2′-O-dimethylaminopropyl (2′-O-DMAP).

In some embodiments, the polynucleotide described herein may comprise one or more 2′-4′ bicyclic nucleosides in which the ribose ring may comprise a bridge moiety, e.g., connecting two atoms in the ring (e.g., connecting the 2′-O atom to the 4′-C atom via an ethylene (ENA) bridge, a methylene (LNA) bridge, or a (S)-constrained ethyl (cEt) bridge). Non-limiting examples of ENAs are disclosed in PCT Publication No. WO 2005/042777; Morita et al., Nucleic Acid Res., Suppl 1:241-242, 2001; Koizumi, Curr. Opin. Mol. Ther., 8:144-149, 2006, Surono et al., Hum. Gene Ther., 15:749-757, 2004; and Horie et al., Nucleic Acids Symp. Ser (Oxf), 49:171-172, 2005; the disclosures of which are incorporated herein by reference in their entireties. Non-limiting examples of LNAs are disclosed in PCT Patent Application Publication No. WO2008/043753, the contents of which are incorporated herein by reference in its entirety. Non-limiting examples of cEt are disclosed in in U.S. Pat. Nos. 7,569,686, 7,101,993, and 7,399,845 each of which is herein incorporated by reference in its entirety.

In various embodiments, the polynucleotide described herein may comprise a modified nucleoside disclosed in, for example, U.S. Pat. Nos. 8,022,193; 7,569,686; 7,399,845; 7,741,457; 7,335,765; 7,816,333; 8,957,201; 7,314,923, the entire contents of each of which are incorporated herein by reference for all purposes.

In various embodiments, the polynucleotide comprises at least one modified nucleoside that results in an increase in Tm of the polynucleotide in a range of 1° C. to 10° C. compared with a polynucleotide that does not have the at least one modified nucleoside. The polynucleotide may have a plurality of modified nucleosides that result in a total increase in Tm of the polynucleotide in a range of 2° C., 3° C., 4° C., 5° C., 6° C., 7° C., 8° C., 9° C., 10° C., 15° C., 20° C., 25° C., 30° C., 35° C., 40° C., 45° C., 50° C., 55° C., 60° C. or more as compared to a polynucleotide which does not have the modified nucleoside.

In some embodiments, the polynucleotide may comprise a mix of nucleosides of different kinds. A polynucleotide may comprise a mix of deoxyribonucleosides or ribonucleosides and 2′-O-Me modified nucleosides. A polynucleotide may comprise a mix of 2′-4′ bicyclic nucleosides and 2′-MOE, 2′-fluoro, or 2′-O-Me modified nucleosides. A polynucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt). A polynucleotide may comprise a mix of 2′-deoxyribonucleosides or ribonucleosides and 2′-fluoro modified nucleosides. A polynucleotide may comprise a mix of 2′-fluoro modified nucleosides and 2′-O-Me modified nucleosides.

In various embodiments, the oligonucleotide may comprise alternating nucleosides of different types. In certain embodiments, the oligonucleotide may comprise alternating deoxyribonucleosides or ribonucleosides and 2′-O-Me modified nucleosides. In certain embodiments, a polynucleotide may comprise alternating 2′-deoxyribonucleosides or ribonucleosides and 2′-fluoro modified nucleosides. In certain embodiments, the oligonucleotide may comprise alternating 2′-fluoro modified nucleosides and 2′-O-Me modified nucleosides. In certain embodiments, the oligonucleotide may comprise alternating 2′-4′ bicyclic nucleosides and 2′-MOE, 2′-fluoro, or 2′-O-Me modified nucleosides. In certain embodiments, the oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In various embodiments, a polynucleotide described herein may comprise one or more abasic residues, a 5-vinylphosphonate modification, and/or one or more inverted abasic residues.

In various embodiments, the oligonucleotide may comprise a phosphorothioate or other modified internucleoside linkage. In various embodiments, the oligonucleotide may comprise phosphorothioate internucleoside linkages. In various embodiments, the oligonucleotide comprises phosphorothioate internucleoside linkages between at least two nucleotides. In various embodiments, the oligonucleotide comprises phosphorothioate internucleoside linkages between all nucleotides. By way of a non-limiting example, in certain embodiments, oligonucleotides comprise modified internucleoside linkages at the first, second, and/or (e.g., and) third internucleoside linkage at the 5′ or 3′ end of the nucleotide sequence.

Non-limiting examples of phosphorus-containing linkages include aminoalkylphosphotriesters phosphorothioates, chiral phosphorothioates, phosphotriesters, phosphorodithioates, methyl and other alkyl phosphonates comprising 3′alkylene phosphonates and chiral phosphonates, phosphinates, phosphoramidates comprising 3′-amino phosphoramidate and aminoalkylphosphoramidates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 5,625,050; 4,469,863; 4,476,301; 5,023,243; 5,550,111; 5,177,196; 5,587,361; 5,188,897; 5,264,423; 5,276,019; 5,519,126; 5,278,302; 5,286,717; 5,321,131; 5,399,676; 5,405,939; 5,453,496; 5,455, 233; 5,466,677; 5,476,925; 5,536,821; 5,541,306; 5,563, 253; 5,571,799; and 3,687,808.

In various embodiments, a polynucleotide described herein may have heteroatom backbones, e.g., or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497), morpholino backbones (see Summerton and Weller, U.S. Pat. No. 5,034,506); amide backbones (see De Mesmaeker et al. Ace. Chem. Res. 1995, 28:366-374); or MMI or methylene(methylimino) backbones.

Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 6-O-methylguanine, 4-thio-pyrimidines, 4-amino-pyrimidines, 4-dimethylhydrazine-pyrimidines, and 4-O-alkyl-pyrimidines, U.S. Pat. No. 5,378,825 and PCT Publication No. WO 93/13121). For general discussion see Adams et al, The Biochemistry of the Nucleic Acids 5-36, 11th ed., 1992. Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleotides and one or more nucleotide analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42): 13233-41). RNA and DNA have different sugar moieties and can differ by the presence of uracil or analogs thereof in RNA and thymine or analogs thereof in DNA.

Interfering Nucleic Acids

In some embodiments, a conjugated molecular cargo may comprise a polynucleotide molecule(s) which is capable of modifying expression of one or more genes (e.g., inhibiting gene expression and/or translation, modulating RNA splicing or inducing exon skipping) in a target cell. In some embodiments, the polynucleotide molecule may be an interfering nucleic acid molecule, e.g., an siRNA, an shRNA, a miRNA, or an antisense oligonucleotide (ASO), that targets, e.g., an RNA (e.g., an mRNA).

In some embodiments, the interfering nucleic acid molecule may modify expression of one more genes associated with a neurological disease and/or disorder listed in Table 1-4. In some embodiments, the interfering nucleic acid molecule may inhibit the expression of one or more genes encoding an apolipoprotein (apo) E risk allele (e.g., ApoE4), glial fibrillary acidic protein (Gfap), methyl CpG binding protein 2 (MeCp2), Aquaporin-4 (Aqp4), or signal transducer and activator of transcription 3 (Stat3).

In certain embodiments, interfering nucleic acid molecules that selectively target and inhibit the activity or expression of a product (e.g., an mRNA product) of a targeted gene are used in compositions and methods described herein. An interfering nucleic acid molecule may inhibit the expression or activity of a product (e.g., an mRNA product) of at least one targeted gene by at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, or 100%. An agent disclosed herein may comprise a nucleobase sequence that is at least 80%, at least 85%, at least 90%, at least 95%, or 100% complementarity to a product (e.g., an mRNA product) of at least targeted gene. Without wishing to be bound by theory, “complementarity” of nucleic acids can mean that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

Interfering nucleic acids can include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence.

Typically, at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is single-stranded RNA. In some embodiments, the interfering nucleic acid molecule is double-stranded RNA. The double-stranded RNA molecule may have a 1-3 nucleotide 3′ and/or 5′ overhang in either a sense strand and/or an antisense strand. In some embodiments, the double-stranded RNA molecule has a 2 nucleotide 3′ overhang. In some embodiments, the two RNA strands are connected via a hairpin structure, forming a shRNA molecule. shRNA molecules can contain hairpins derived from microRNA molecules.

Interfering nucleic acid molecules described herein can contain RNA bases, non-RNA bases or a mixture of RNA bases and non-RNA bases. For example, interfering nucleic acid molecules described herein can be primarily composed of RNA bases or modified RNA bases, but also contain DNA bases, modified DNA bases, and/or non-naturally occurring nucleotides. The term “ribonucleotide” or “nucleotide” can, in the case of a modified RNA or nucleotide surrogate, also refer to a modified nucleotide, or surrogate replacement moiety at one or more positions.

In some embodiments, the interfering nucleic acid molecule is a small interfering RNAs (siRNA), also known as short interfering RNA or silencing RNA. siRNAs are a class of double-stranded RNA molecules, typically about 20-25 base pairs in length that target nucleic acids (e.g., mRNAs) for degradation via the RNA interference (RNAi) pathway in cells. Such siRNA molecules typically include a region of sufficient homology to the target region, and are of sufficient length in terms of nucleotides, such that the siRNA molecules down-regulate target nucleic acid. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

Specificity of siRNA molecules may be measured via the binding of the antisense strand of the molecule to its target RNA. Effective siRNA molecules are often fewer than 30 to 35 base pairs in length, e.g., to prevent stimulation of non-specific RNA interference pathways in the cell by way of the interferon response, however longer siRNA may also be effective. In various embodiments, the siRNA molecules are 5, 6, 7, 8, 9, 10, 11, 12, 13, 14, 15, 16, 17, 18, 19, 20, 21, 22, 23, 24, 25, 26, 27, 28, 29, or 30 base pairs in length. In various embodiments, the siRNA molecules are about 35 to about 70 more base pairs in length. In some embodiments, the siRNA molecules are more than 70 base pairs in length. In some embodiments, the siRNA molecules are 8 to 40 base pairs in length, 10 to 20 base pairs in length, 10 to 30 base pairs in length, 15 to 20 base pairs in length, 19 to 23 base pairs in length, 21 to 24 base pairs in length. In some embodiments, the sense and antisense strands of the siRNA molecules are each independently about 19 to about 24 nucleotides in length. In some embodiments, the sense strand of an siRNA molecule is 23 nucleotides in length and the antisense strand is 21 nucleotides in length. In some embodiments, both the sense strand and the antisense strand of an siRNA molecule are 21 nucleotides in length.

After selection of a suitable target RNA sequence, siRNA molecules that comprise a nucleotide sequence complementary to all or a portion of the target sequence, i.e., an antisense sequence, may be designed and prepared using suitable methods (see, e.g., U.S. Patent Publication Nos. 2004/0077574 and 2008/0081791 and PCT Publication No. WO 2004/016735). In some embodiments, the siRNA molecule may be single-stranded (i.e. a ssRNA molecule comprising just an antisense strand) or double stranded (i.e. a dsRNA molecule comprising an antisense strand and a complementary sense strand that hybridizes to form the dsRNA). In various embodiments, the siRNA molecules may comprise a duplex, asymmetric duplex, hairpin or asymmetric hairpin secondary structure, comprising self-complementary sense and/or antisense strands.

In various embodiments, the antisense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiment, the antisense strand of the siRNA molecule is about 35 to about 70 nucleotides in length. In various embodiment, the antisense strand of the siRNA molecule is more than 70 nucleotides in length. In some embodiments, the antisense strand is 8 to 40 nucleotides in length, 10 to 20 nucleotides in length, 10 to 30 nucleotides in length, 15 to 20 nucleotides in length, 19 to 23 nucleotides in length, or 21 to 24 nucleotides in length.

In some embodiments, the sense strand of the siRNA molecule is 5, 6, 7, 8, 9, 10, 11, 12, 13, 14, 15, 16, 17, 18, 19, 20, 21, 22, 23, 24, 25, 26, 27, 28, 29, or 30 nucleotides in length. In various embodiments, the sense strand of the siRNA molecule is about 30 to about 70 nucleotides in length. In various embodiments, the sense strand of the siRNA molecule more than 70 nucleotides in length. In some embodiments, the sense strand is 8 to 40 nucleotides in length, 10 to 20 nucleotides in length, 10 to 30 nucleotides in length, 15 to 20 nucleotides in length, 19 to 23 nucleotides in length, 21 to 24 nucleotides in length.

In various embodiments, siRNA molecules can comprise an antisense strand comprising a region of complementarity to a target region in a target mRNA. In some embodiments, the region of complementarity is at least 70%, at least 75%, at least 80%, at least 85%, at least 90%, at least 91%, at least 92%, at least 93%, at least 94%, at least 95%, at least 96%, at least 97%, at least 98%, at least 99% or 100% complementary to a target region in a target mRNA. In some embodiments, the target region may comprise a region of consecutive nucleotides in the target mRNA. In some embodiments, it may not be requisite for a region of complementarity to be 100% complementary to that of its target to be specifically hybridizable or specific for a target RNA sequence.

In some embodiments, siRNA molecules disclosed herein may comprise an antisense strand that comprises a region of complementarity to a target RNA sequence and the region of complementarity is in the range of 8 to 20, 8 to 35, 8 to 45, or 10 to 50, or 5 to 55, or 5 to 40 nucleotides in length. In some embodiments, a region of complementarity is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of a target RNA sequence. In some embodiments, siRNA molecules comprise an antisense strand having a nucleotide sequence that contains no more than 1, 2, 3, 4, 5, 6, 7, 8, 9, or 10 base mismatches compared to the portion of the consecutive nucleotides of target RNA sequence. In some embodiments, siRNA molecules comprise a nucleotide sequence that has up to 3 mismatches over 15 bases, or up to 4 mismatches over 10 bases with a target sequence. In some embodiments, siRNA molecules comprises an antisense strand having a nucleotide sequence that has up 0, 1, 2, or 3 mismatches over 15-22 bases with a target sequence. In some embodiments, siRNA molecules comprises an antisense strand having a nucleotide sequence that has 0, 1, or 2 mismatches over 15-22 bases with a target sequence. In some embodiments, siRNA molecules comprises an antisense strand having a nucleotide sequence that has 0 or 1 mismatch over 15-22 bases with a target sequence. In some embodiments, siRNA molecules comprises an antisense strand having a nucleotide sequence that has 0 mismatches over 15-22 bases with a target sequence.

In various embodiments, siRNA molecules may comprise an antisense strand comprising a nucleotide sequence that is at least 70%, at least 75%, at least 85%, at least 90%, at least 95%, or 100% complementary to the target RNA sequence of the antisense oligonucleotides disclosed herein. In some embodiments, siRNA molecules comprise an antisense strand comprising a nucleotide sequence that is at least 70%, at least 75%, at least 85%, at least 90%, at least 95%, or 100% identical to any of the antisense oligonucleotides provided herein. In some embodiments, siRNA molecules comprise an antisense strand comprising at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, or more consecutive nucleotides of any of the antisense oligonucleotides provided herein.

In some embodiments, double-stranded siRNA can comprise sense and anti-sense RNA strands that are different lengths or the same length. In some embodiments, double-stranded siRNA molecules may also be generated from a single oligonucleotide in a stem-loop structure. The self-complementary sense and antisense regions of the siRNA molecule having a stem-loop structure may be linked by means of a nucleic acid based or a non-nucleic acid-based linker. In some embodiments, an siRNA having a stem-loop structure comprises a circular single-stranded RNA having two or more loop structures and a stem comprising self-complementary sense and antisense strands. In some embodiments, the circular RNA may be processed in vivo or in vitro to produce an active siRNA molecule which may be capable of mediating RNAi. Small hairpin RNA (shRNA) molecules are therefore also contemplated herein. Such molecules may comprise a specific antisense sequence together with the reverse complement (sense) sequence, which may be separated by a spacer or loop sequence in some instances. A reverse complement described herein may comprise a sequence that is a complement sequence of a reference sequence, wherein the complement sequence is written in the reverse orientation. Due to codon usage redundancy, a reverse complement can diverge from a reference sequence that encodes the same polypeptide. As used herein, “reverse complement” also includes sequences that are, e.g., at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% identical to the reverse complement sequence of a reference sequence. Cleavage of the spacer or loop can provide a single-stranded RNA molecule and its reverse complement, such that they may anneal to form a dsRNA molecule. In various embodiments, additional optional processing steps may result in removal or addition of 1, 2, 3, 4, 5 or more nucleotides from the 3′ end and/or the 5′ end of one or both strands. A spacer may be of a suitable length to allow the antisense and sense sequences to anneal and form a double-stranded structure or stem prior to cleavage of the spacer. In certain embodiments subsequent optional processing steps may result in removal or addition of 1, 2, 3, 4, 5 or more nucleotides from the 3′ end and/or the 5′ end of one or both strands. In some embodiments, a spacer sequence can be an unrelated nucleotide sequence that may be, e.g., situated between two complementary nucleotide sequence regions that, when annealed into a double-stranded nucleic acid, can comprise a shRNA.

The length of the siRNA molecules can vary from about 10 to about 120 nucleotides depending on the type of siRNA molecule being designed. Generally, between about 10 and about 55 of these nucleotides may be complementary to the RNA target sequence. For instance, when the siRNA is a double-stranded siRNA or single-stranded siRNA, the length can vary from about 10 to about 55 nucleotides, whereas when the siRNA is a shRNA or circular molecule, the length can vary from about 30 nucleotides to about 110 nucleotides.

In various embodiments, an siRNA molecule can comprise a 3′ overhang at one end of the molecule. In some embodiments, the other end can be blunt-ended or may also comprise an overhang (e.g., 5′ and/or 3′). When the siRNA molecule comprises an overhang at both ends of the molecule, the length of the overhangs may be different or the same. In some embodiments, an siRNA molecule described herein may comprises 3′ overhangs of about 1 to about 3 nucleotides on both ends of the molecule. In some embodiments, the siRNA molecule comprises 3′ overhangs of about 1 to about 3 nucleotides on both the sense strand and the antisense strand. In some embodiments, the siRNA molecule comprises 3′ overhangs of about 1 to about 3 nucleotides on the antisense strand. In some embodiments, the siRNA molecule may comprise 3′ overhangs of about 1 to about 3 nucleotides on the sense strand.

In various embodiments, the siRNA molecule comprises one or more modified nucleotides (e.g., 1, 2, 3, 4, 5, 6, 7, 8, 9, 10, 11, 12, 13, 14, 15 or more). In some embodiments, all of the nucleotides of the sense strand and/or the antisense strand of the siRNA molecule are modified. In certain embodiments, the siRNA molecule can comprise one or more modified nucleotides and/or one or more modified internucleotide linkages. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand.

In some embodiments, the modified nucleotide may comprise a modified sugar moiety (e.g., a 2′ modified nucleotide). In some embodiments, the siRNA molecule can comprise one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the siRNA molecule can a modified nucleotide (e.g., a 2′-modified nucleotide). In some embodiments, the siRNA molecule may comprise one or more phosphorodiamidate morpholinos. In some embodiments, each nucleotide of the siRNA molecule consists of a phosphorodiamidate morpholino.

In various embodiments, the siRNA molecule may comprise a phosphorothioate or other modified internucleotide linkage. In various embodiments, the siRNA molecule may comprise, e.g., a phosphorothioate internucleoside linkage(s). In some embodiments, the siRNA molecule may comprise a phosphorothioate internucleoside linkage(s) between two or more nucleotides. In some embodiments, the siRNA molecule may comprise a phosphorothioate internucleoside linkage(s) between all nucleotides. In some embodiments, the siRNA molecule may comprise modified internucleotide linkages at the first, second, and/or third internucleoside linkage at the 5′ or 3′ end of the siRNA molecule. In some embodiments, the siRNA molecule may comprise modified internucleotide linkages at the first and second internucleoside linkages at the 5′ and/or 3′ end of the siRNA molecule. In some embodiments, the siRNA molecule may comprise modified internucleotide linkages at the first and second internucleoside linkages at the 5′ end of the siRNA molecule sense strand. In some embodiments, the siRNA molecule may comprise modified internucleotide linkages at the first and second internucleoside linkages at the 5′ and 3′ ends of the siRNA molecule antisense strand. In some embodiments, the siRNA molecule may comprise modified internucleotide linkages at the first and second internucleoside linkages at the 5′ end of the siRNA molecule sense strand and at the first and second internucleoside linkages at the 5′ and 3′ ends of the siRNA molecule antisense strand. In some embodiments, the siRNA molecule may comprise modified internucleotide linkages at the first internucleoside linkage at the 5′ and 3′ ends of the siRNA molecule sense strand, at the first, second, and third internucleoside linkages at the 5′ end of the siRNA molecule antisense strand, and at the first internucleoside linkage at the 3′ end of the siRNA molecule antisense strand.

In various embodiments, the modified internucleotide linkages may comprise phosphorus-containing linkages. In some embodiments, phosphorus-containing linkages which may be used in the methods or compositions described herein include, without limitation, chiral phosphorothioates, phosphorothioates, phosphorodithioates, aminoalkylphosphotriesters, phosphotriesters, methyl and other alkyl phosphonates comprising 3′-alkylene phosphonates and chiral phosphonates, phosphoramidates comprising 3′-amino phosphoramidate and aminoalkylphosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′, see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the various modified formats or chemistries of siRNA molecules disclosed herein may be combined together. For example, without limitation, 1, 2, 3, 4, 5, 6, 7, 8, 9, or 10 or more different types of modifications may be included within the same siRNA molecule.

In various embodiments, the antisense strand may comprise one or more modified nucleotides (e.g., 1, 2, 3, 4, 5, 6, 7, 8, 9, 10, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, antisense strand contains a phosphorothioate or other modified internucleotide linkage. In some embodiments, the antisense strand may comprise phosphorothioate internucleoside linkage(s). In some embodiments, the antisense strand may comprise phosphorothioate internucleoside linkage(s) between two or more nucleotides. In some embodiments, the antisense strand may comprise phosphorothioate internucleoside linkage(s) between all nucleotides. In some embodiments, the antisense strand may comprise modified internucleotide linkages at the first, second, and/or third nucleotide at the 5′ or 3′ end of the antisense strand. In some embodiments, the antisense strand may comprise modified internucleotide linkages at the first and second nucleotide positions (e.g., between the first and second and between the second and third nucleotides) at the 5′ and 3′ ends of the antisense strand.

In various embodiments, the modified internucleotide linkages may comprise phosphorus-containing linkages of the antisense strand. In some embodiments, phosphorus-containing linkages which may be used in methods and compositions described herein include, without limitation, chiral phosphorothioates, phosphorothioates, phosphorodithioates, aminoalkylphosphotriesters, phosphotriesters, methyl and other alkyl phosphonates comprising 3′-alkylene phosphonates and chiral phosphonates, phosphoramidates comprising 3′-amino phosphoramidate and aminoalkyl phosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the modified formats or chemistries of the antisense strand disclosed herein may be combined together. For example, without limitation, 1, 2, 3, 4, 5, 6, 7, 8, 9, or 10 or more different types of modifications may be included within the same antisense strand.

In some embodiments, the sense strand comprises one or more modified nucleotides (e.g., 1, 2, 3, 4, 5, 6, 7, 8, 9, 11, 12, 13, 14, 15 or more). In some embodiments, the antisense strand may comprise one or more modified nucleotides and/or one or more modified internucleotide linkage(s). In some embodiments, the modified nucleotide may comprise a modified sugar moiety (e.g., a 2′ modified nucleotide). In some embodiments, the antisense strand comprises one or more 2′ modified nucleotides, e.g., a 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-0-dimethylaminoethyloxyethyl (2′-O-DMAEOE), or 2′-O—N-methylacetamido (2′-O-NMA). In various embodiments, each nucleotide of the antisense strand can be a modified nucleotide (e.g., a 2′-modified nucleotide). In some embodiments, the antisense strand may comprise one or more phosphorodiamidate morpholinos. In some embodiments, the antisense strand consists of a phosphorodiamidate morpholino oligomer (PMO).

In some embodiments, the sense strand contains a phosphorothioate or other modified internucleotide linkage. In some embodiments, the sense strand may comprise phosphorothioate internucleoside linkage(s). In some embodiments, the sense strand may comprise phosphorothioate internucleoside linkage(s) between two or more nucleotides. In some embodiments, the sense strand may comprise phosphorothioate internucleoside linkages between all nucleotides. For example, in some embodiments, the sense strand comprises modified internucleotide linkages at the first, second, and/or third nucleotide at the 5′ or 3′ end of the sense strand. In some embodiments, the sense strand may comprise modified internucleotide linkages at the first and second nucleotide positions (e.g., between the first and second and between the second and third nucleotides) at the 5′ end of the sense strand.

In various embodiments, the modified internucleotide linkages may comprise phosphorus-containing linkages of the sense strand. In some embodiments, phosphorus-containing linkages which may be used in the methods and compositions described herein include, without limitation, chiral phosphorothioates, phosphorothioates, phosphorodithioates, aminoalkylphosphotriesters, phosphotriesters, methyl and other alkyl phosphonates comprising 3′-alkylene phosphonates and chiral phosphonates, phosphoramidates comprising 3′-amino phosphoramidate and aminoalkyl phosphoramidates, phosphinates, thionoalkylphosphonates, thionophosphoramidates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 5,625,050; 3,687,808; 4,469,863; 4,476,301; 5,177,196; 5,455, 233; 5,264,423; 5,276,019; 5,278,302; 5,286,717; 5,321,131; 5,399,676; 5,405,939; 5,519,126; 5,453,496; 5,466,677; 5,476,925; 5,536,821; 5,023,243; 5,541,306; 5,550,111; 5,563, 253; 5,571,799; 5,587,361; and 5,188,897.

Any of the modified chemistries or formats of the sense strand described herein can be combined together. For example, without limitation, 1, 2, 3, 4, 5, 6, 7, 8, 9, or 10 or more different types of modifications may be included within the same sense strand.

In various embodiments, the antisense and/or sense strand of the siRNA molecule may comprise one or more modifications capable of enhancing or reducing, e.g., RNA-induced silencing complex (RISC) loading. In some embodiments, the antisense strand of the siRNA molecule may comprise one or more modifications capable of enhancing RISC loading. In various embodiments, the sense strand of the siRNA molecule may comprise one or more modifications capable of reducing RISC loading and/or reducing off-target effects. In various embodiments, the antisense strand of the siRNA molecule may comprise a 2′-O-methoxyethyl (2′-MOE) modification. In some embodiments, the addition of the 2′-O-methoxyethyl (2′-MOE) group, e.g., at the cleavage site may improve the silencing activity and/or specificity of siRNAs, e.g., by facilitating the oriented RNA-induced silencing complex (RISC) loading of the modified strand, e.g., as disclosed in Song et al., (2017) Mol Ther Nucleic Acids 9:242-250, incorporated herein by reference in its entirety. In various embodiments, the antisense strand of the siRNA molecule may comprise a 2′-O-Me-phosphorodithioate modification. In some embodiment, the 2′-O-Me-phosphorodithioate modification may increase RISC loading, e.g., as disclosed in Wu et al., (2014) Nat Commun 5:3459, incorporated herein by reference in its entirety.

In various embodiments, the sense strand of the siRNA molecule may comprise a 5′-nitroindole modification. In some embodiments, the 5′-nitroindole modification may decrease the RNAi potency of the sense strand and/or reduces off-target effects, e.g., as disclosed in Zhang et al., (2012) Chembiochem 13(13): 1940-1945, incorporated herein by reference in its entirety. In various embodiments, the sense strand may comprise a 2′-O-methyl (2′-O-Me) modification. In some embodiments, the 2′-0-Me modification may reduce RISC loading and/or the off-target effects of the sense strand, e.g., as disclosed in Zheng et al., FASEB (2013) 27(10): 4017-4026, incorporated herein by reference in its entirety. In various embodiments, the sense strand of the siRNA molecule may be fully substituted with morpholino, 2′-MOE and/or 2′-O-Me residues, and may not be recognized by RISC, e.g., as disclosed in Kole et al., (2012) Nature reviews. Drug Discovery 11(2): 125-140, incorporated herein by reference in its entirety.

In various embodiments, the sense strand of the siRNA molecule may comprise a 5′-morpholino modification. In various embodiments, the 5′-morpholino modification may reduce RISC loading of the sense strand and/or improves RNAi activity and/or antisense strand selection, e.g., as disclosed in Kumar et al., (2019) Chem Commun (Camb) 55(35):5139-5142, incorporated herein by reference in its entirety. In various embodiments, the sense strand of the siRNA molecule may be modified, for example, with a synthetic RNA-like high affinity nucleotide analogue called Locked Nucleic Acid (LNA) that may reduce RISC loading of the sense strand and promote antisense strand incorporation into RISC, e.g., as disclosed in Elman et al., (2005) Nucleic Acids Res. 33(1): 439-447, incorporated herein by reference in its entirety. In various embodiments, the sense strand of the siRNA molecule may comprise a 5′ unlocked nucleic acid (UNA) modification. In various embodiments, the 5′ unlocked nucleic acid (UNA) modification may reduce RISC loading of the sense strand and/or improve silencing capability of the antisense strand, e.g., as disclosed in Snead et al., (2013) Mol Ther Nucleic Acids 2(7): e103, incorporated herein by reference in its entirety.

In some embodiments, the antisense strand of the siRNA molecule may comprise a 2′-MOE modification and/or the sense strand may comprise an 2′-O-Me modification (see e.g., Song et al., (2017) Mol Ther Nucleic Acids 9:242-250). In some embodiments at least one (e.g., at least 2, at least 3, at least 4, at least 5, at least 6, at least 5, at least 8, at least 9, at least 10 or more) siRNA molecule may be conjugated, for example, covalently to an FGFR3 binding protein described herein. In some embodiments, the FGFR3 binding protein may be conjugated to the 5′ end of the sense strand of the siRNA molecule. In some embodiments, the FGFR3 binding protein may be conjugated to the 3′ end of the sense strand of the siRNA molecule. In some embodiments, the FGFR3 binding protein may be conjugated internally to the sense strand of the siRNA molecule. In some embodiments, the FGFR3 binding protein may be conjugated to the 5′ end of the antisense strand of the siRNA molecule. In some embodiments, the FGFR3 binding protein may be conjugated to the 3′ end of the antisense strand of the siRNA molecule. In some embodiments, the FGFR3 binding protein be conjugated internally to the antisense strand of the siRNA molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-termini of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (e.g., C3-C12 (e.g., C3, C6, C9, C12), abasic, tri ethylene glycol, hexaethylene glycol), biotin or fluorescein reagents that come as phosphoramidites and that have another DMT-protected hydroxyl group, allowing multiple couplings during RNA synthesis.

In some embodiments, the sense strand is 23 nucleotides in length and the antisense strand is 21 nucleotides in length. In some embodiments, the sense strand is 23 nucleotides in length and the antisense strand is 21 nucleotides in length, wherein the 3’ and 5′ terminal nucleotide positions of the sense strand are inverted abasic residues. The sense strand 3′ and 5′ terminal inverted abasic residues may be overhangs. The inverted abasic residues may be linked via a 3′-3′ phosphodiester linkage. In some embodiments, the antisense strand of the siRNA molecule contains 1-2 phosphorothioate linkages at the 3′ and/or 5′ ends. In some embodiments, the antisense strand contain two or three phosphorothioate internucleotide linkages at the 5′-terminus and 1 phosphorothioate internucleotide linkage at the 3′-terminus. The siRNA molecule may be linked to a targeting moiety at the 5′ or 3′ end of the sense strand.

In some embodiments, the sense strand is 21 nucleotides in length and the antisense strand is 23 nucleotides in length, wherein the antisense strand contains a 2 nucleobase 3′ overhang. In some embodiments, the antisense strand of the siRNA molecule contains 1-3 phosphorothioate linkages at the 3′ and 5′ ends and the sense strand of the siRNA molecule contains 1-2 phosphorothioate linkages at the 5′ end. In some embodiments, the antisense strand of the siRNA molecule contains 2-3 phosphorothioate linkages at the 5′ end and 2 phosphorothioate linkages at the 3′, and the sense strand of the siRNA molecule contains 2 phosphorothioate linkages at the 5′ end. The siRNA molecule may be linked to a targeting moiety at the 5′ or 3′ end of the sense strand.

In some embodiments, the siRNA molecules described herein may be conjugated to a moiety that directs delivery to the CNS, e.g., a lipophilic ligand, optionally a C16 ligand, as described in WO2021119226A1, which is incorporated herein by reference in its entirety. In one embodiment, the lipophilic moiety is a lipid, cholesterol, retinoic acid, cholic acid, adamantane acetic acid, 1-pyrene butyric acid, dihydrotestosterone, I,3-bis-O(hexadecyl)glycerol, geranyloxyhexyanol, hexadecylglycerol, bomeol, menthol, 1,3-propanediol, heptadecyl group, palmitic acid, myristic acid, 03-(oleoyl)lithocholic acid, 03-(oleoyl)cholenic acid, dimethoxytrityl, or phenoxazine.

In one embodiment, the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne. In one embodiment, the lipophilic moiety contains a saturated or unsaturated C6-C18 hydrocarbon chain. In one embodiment, the lipophilic moiety contains a saturated or unsaturated C16 hydrocarbon chain. In one embodiment, the saturated or unsaturated C16 hydrocarbon chain is conjugated to position 6, from the 5′-end of the strand.

In one embodiment, the lipophilic moiety is conjugated via a carrier that replaces one or more nucleotide(s) in the internal position(s) or the double stranded region. In one embodiment, the carrier is a cyclic group selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone. In one embodiment, the lipophilic moiety is conjugated to the double-stranded siRNA agent via a linker containing an ether, thioether, urea, carbonate, amine, amide, maleimide-thioether, disulfide, phosphodiester, sulfonamide linkage, a product of a click reaction, or carbamate. In one embodiment, the lipophilic moiety is conjugated to a nucleobase, sugar moiety, or intemucleosidic linkage.

In one embodiment, the lipophilic moiety or targeting ligand is conjugated via a bio-cleavable linker selected from the group consisting of DNA, RNA, disulfide, amide, functionalized monosaccharides or oligosaccharides of galactosamine, glucosamine, glucose, galactose, mannose, and combinations thereof.

In one embodiment, the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In some embodiments, the interfering nucleic acid molecule is a short hairpin RNA (shRNA). A “small hairpin RNA” or “short hairpin RNA” or “shRNA” described herein may include a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure may be cleaved by the cellular machinery into siRNA, which is then bound to the RNA-induced silencing complex (RISC).

Non-limiting examples of shRNAs include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. Patent Publication No. 2011/0071208, the disclosure of which is herein incorporated by reference in its entirety for all purposes.

In some embodiments, the interfering nucleic acid molecule is a microRNA (miRNA). miRNAs represent a large group of small RNAs produced naturally in organisms, some of which regulate the expression of target genes. miRNAs are short hairpin RNAs about 18 to about 25 nucleotides in length that function in RNA silencing and post-translational regulation of gene expression. Typically, miRNAs are generated from large RNA precursors (termed pri-miRNAs) that are processed in the nucleus into approximately 70 nucleotide pre-miRNAs, which fold into imperfect stem-loop structures. These pre-miRNAs typically undergo an additional processing step within the cytoplasm where mature miRNAs of 18-25 nucleotides in length are excised from one side of the pre-miRNA hairpin by an RNase III enzyme, Dicer. miRNAs are not translated into proteins, but instead bind to specific messenger RNAs, thereby blocking translation. In some embodiments, miRNAs base-pair imprecisely with their targets to inhibit translation.

miRNAs as described herein can include pri-miRNA, pre-miRNA, mature miRNA or fragments of variants thereof that retain the biological activity of mature miRNA. In some embodiments, the size range of the miRNA can be from 21 nucleotides to 170 nucleotides. In one embodiment, the size range of the miRNA is from 70 to 170 nucleotides in length. In another embodiment, mature miRNAs of from 21 to 25 nucleotides in length can be used.

In certain embodiments, the interfering nucleic acid molecule is an antisense oligonucleotide (ASO). An ASO can down regulate a target by inducing RNase H endonuclease cleavage of a target RNA, by steric hindrance of ribosomal activity, by inhibiting 5′ cap formation, or by altering splicing. An ASO can be, but is not limited to, a gapmer or a morpholino. An antisense oligonucleotide typically comprises a short nucleotide sequence which is substantially complementary to a target nucleotide sequence in a pre-mRNA molecule, heterogeneous nuclear RNA (hnRNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. Antisense oligonucleotides are often synthetic and chemically modified.

Antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

In some embodiments, an interfering nucleic acid molecule described herein is a gapmer. A “Gapmer” is oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” A gapmer can have 5′ and 3′ wings each having 2-6 nucleotides and a gap having 7-12 nucleotides. In some embodiments, a gapmer can have a 3-10-3 configuration or a 5-10-5 configuration.

A gapmer commonly has the formula 5′-X-Y-Z-3′, with X and Z as flanking regions around a gap region Y. In some embodiments, flanking region X of formula 5′-X-Y-Z-3′ is also called X region, flanking sequence X, 5′ wing region X, or 5′ wing segment. In some embodiments, flanking region Z of formula 5′-X-Y-Z-3′ is also called Z region, flanking sequence Z, 3′ wing region Z, or 3′ wing segment. In some embodiments, gap region Y of formula 5′-X-Y-Z-3′ is also called Y region, Y segment, gap-segment Y, gap segment, or gap region. In some embodiments, each nucleoside in the gap region Y is a 2′-deoxyribonucleoside, and neither the 5′ wing region X or the 3′ wing region Z comprises any 2′-deoxyribonucleosides.

In some embodiments, the gap region of the gapmer polynucleotide may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, or at least five or more nucleotides. In some embodiments, each internucleotide linkage in the gap segment comprises a phosphorothioate linkage. In some embodiments, the gap region and two flanking regions each independently comprise modified internucleoside linkages (e.g., phosphorothioate internucleoside linkages or other linkages) between at least two, at least three, at least four, or at least five or more nucleotides. In some embodiments, each internucleotide linkage in the 5′ or 3′ wing region comprises a phosphorothioate linkage. In some embodiments, each internucleotide linkage in the gapmer comprises a phosphorothioate linkage.

In some embodiments, the Y region may comprise a contiguous stretch of nucleotides, e.g., a region of 5 or more DNA nucleotides, which can be capable of recruiting an RNase including but not limited to Rnase H. In some embodiments, the gapmer may bind to a target nucleic acid such that an Rnase is recruited to cleave the target nucleic acid. In some embodiments, the Y region may be flanked both 5′ and 3′ by regions X and Z comprising high-affinity modified nucleosides, e.g., 1-10 high-affinity modified nucleosides. Exemplary high affinity modified nucleosides include, without limitation, 2′-4′ bicyclic nucleosides (e.g., LNA, cEt, ENA) and 2′-modified nucleosides (e.g., 2′-MOE, 2′O-Me, 2′-F). In some embodiments, the flanking sequences X and Z may be of 1-30 nucleotides, 1-20 nucleotides, 1-10 nucleotides, or 1-5 nucleotides in length. The flanking sequences X and Z may be of similar length or of dissimilar lengths. In some embodiments, the flanking sequences X and Z are each 5 nucleotides in length. In some embodiments, the flanking sequences X and Z are each 3 nucleotides in length. In some embodiments, the gap-segment Y may be a nucleotide sequence of 5-30 nucleotides, 5-20 nucleotides, or 5-10 nucleotides in length. In some embodiments, the gap segment is 10 nucleotides in length.

A gapmer may be produced using suitable methods. Preparation of gapmers is described in, for example, U.S. Pat. Nos. 10,260,069; 10,017,764; 9,695,418; 9,428,534; 9,428,534; 9,045,754; 8,580,756; 8,580,756; 7,750,131; 7,683,036; 7,569,686; 7,432,250; 7,399,845; 7,101,993; 7,015,315; 5,898,031; 5,700,922; 5,652,356; 5,652,355; 5,623,065; 5,565,350; 5,491,133; 5,403,711; 5,366,878; 5,256,775; 5,220,007; 5,149,797; and 5,013,830; U.S. Patent Publication Nos. US2010/0197762, US2005/0074801, US2009/0221685, US2009/0286969, and US2011/0112170, PCT Publication Nos. WO2005/023825, WO2004/069991, WO2008/049085 and WO2009/090182, each of which is herein incorporated by reference in its entirety.

In some embodiments, a gapmer is 10-50 nucleosides in length. For example, a gapmer may be 10-50, 10-45, 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, a gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length. In some embodiments, a gapmer is about 16 to about 20 nucleosides in length. In some embodiments, a gapmer is 16 nucleotides in length. In some embodiments, a gapmer is 20 nucleotides in length.

In some embodiments, the 5′ wing region and the 3′ wing region of a gapmer are independently 1-20 nucleosides (e.g., 1, 2, 3, 4, 5, 6, 7, 8, 9, 10, 11, 12, 13, 14, 15, 16, 17, 18, 19, or 20 nucleosides) long. For example, the 5′ wing region and the 3′ wing region of the gapmer may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′ wing region and the 3′ wing region of the gapmer are of the same length. In some embodiments, the 5′ wing region and the 3′ wing region of a gapmer are of different lengths. In some embodiments, the 5′ wing region is longer than the 3′ wing region of a gapmer. In some embodiments, the 5′ wing region is shorter than the 3′ wing region of the gapmer.

In some embodiments, the gap region in a gapmer is 5-20 nucleosides in length. For example, the gap region Y may be 5-20, 5-15, 5-10, 10-20, 10-15, or 15-20 nucleosides in length. In some embodiments, the gap region is 5, 6, 7, 8, 9, 10, 11, 12, 13, 14, 15, 16, 17, 18, 19, or 20 nucleosides in length. In some embodiments, one or more nucleosides in the gap region Y is a 2′-deoxyribonucleoside. In some embodiments, every nucleotide in the gap region is a deoxyribonucleoside. In some embodiments, one or more of the nucleosides in the gap region is a modified nucleoside (e.g., a 2′ modified nucleoside such as those described herein). In some embodiments, one or more cytosines in the gap region Y are 5-methyl-cytosines. In some embodiments, every cytosine in the gap region Y is a 5-methyl-cytosine. In some embodiments, every cytosine in a gapmer is a 5-methyl-cytosine.

In some embodiments, one or more nucleosides in the 5′ wing region or the 3′ wing region of a gapmer are modified nucleotides. In some embodiments, the modified nucleotide may be a 2′-modified nucleoside, e.g., 2′-4′ bicyclic nucleoside ora non-bicyclic 2′-modified nucleoside. In some embodiments, the nucleoside may be a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-dimethylaminoethyl (2′-O-DMAOE), 2′-O-dimethylaminopropyl (2′-O-DMAP), 2′-O-methoxyethyl (2′-MOE), 2′-O-aminopropyl (2′-O-AP), 2′-O-dimethylaminoethyloxyethyl (2′-O-DMAEOE), or 2′-O—N-methylacetamido (2′-O-NMA)). In some embodiments, every nucleotide in a wing region is a modified nucleotide. In some embodiments, every nucleotide in a wing region is a 2′-MOE, LNA or cET nucleotide.

In some embodiments, a gapmer described herein may comprises one or more modified nucleoside linkages in each of the X, Y, and Z regions. In some embodiments, each internucleoside linkage may comprise phosphorothioate linkage. In some embodiments, each of the X, Y, and Z regions independently comprises a combination of phosphodiester linkages and phosphorothioate linkages. In some embodiments, each internucleoside linkage in the gap region Y may be a phosphorothioate linkage, the 5′ wing region X comprises a combination of phosphorothioate linkages and phosphodiester linkages, and the 3′ wing region Z comprises a combination of phosphorothioate linkages and phosphodiester linkages.

In some embodiments, each nucleotide in the gap region of a gapmer is a deoxyribonucleotide and each nucleotide in a wing region is a 2′-MOE nucleotide. In some embodiments, each nucleotide in the gap region of a gapmer is a deoxyribonucleotide, each nucleotide in a wing region is a 2′-MOE nucleotide, and every cytosine in the gapmer is a 5-methyl-cytosine. In some embodiments, each nucleotide in the gap region of a gapmer is a deoxyribonucleotide, each nucleotide in a wing region is a 2′-MOE nucleotide, every cytosine in the gapmer is a 5-methyl-cytosine and every internucleotide linkage is a phosphorothioate linkage.

In some embodiments, each nucleotide in the gap region of a gapmer is a deoxyribonucleotide and each nucleotide in a wing region is a LNA nucleotide. In some embodiments, each nucleotide in the gap region of a gapmer is a deoxyribonucleotide, each nucleotide in a wing region is a LNA nucleotide, and every cytosine in the gapmer is a 5-methyl-cytosine. In some embodiments, each nucleotide in the gap region of a gapmer is a deoxyribonucleotide, each nucleotide in a wing region is a LNA nucleotide, every cytosine in the gapmer is a 5-methyl-cytosine and every internucleotide linkage is a phosphorothioate linkage. In some embodiments, each nucleotide in the gap region of a gapmer is a deoxyribonucleotide and each nucleotide in a wing region is a cET nucleotide. In some embodiments, each nucleotide in the gap region of a gapmer is a deoxyribonucleotide, each nucleotide in a wing region is a cET nucleotide, and every cytosine in the gapmer is a 5-methyl-cytosine. In some embodiments, each nucleotide in the gap region of a gapmer is a deoxyribonucleotide, each nucleotide in a wing region is a cET nucleotide, every cytosine in the gapmer is a 5-methyl-cytosine and every internucleotide linkage is a phosphorothioate linkage.

The interfering nucleic acids can employ a variety of oligonucleotide chemistries. Examples of oligonucleotide chemistries include, without limitation, peptide nucleic acid (PNA), locked nucleic acid (LNA), phosphorothioate, 2′-O-Me-modified oligonucleotides, and morpholino chemistries, including combinations of any of the foregoing. In general, PNA and LNA chemistries can utilize shorter targeting sequences because of their relatively high target binding strength relative to 2′-O-Me oligonucleotides. Phosphorothioate and 2′-O-Me-modified chemistries are often combined to generate 2′-0-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications. The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™ has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 5,539,082; 5,714,331; and 5,719,262, 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids described herein may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties. Alternatively, non-phosphorous containing linkers may be employed. In some embodiments, an antisense oligonucleotide comprises an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases SI and PI, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′ 0-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′-O-Me oligonucleotides (phosphodiester or phosphothioate) can be synthesized according to routine techniques in the art (see, e.g., Yoo et al., Nucleic Acids Res. 32:2008-16, 2004).

Interfering nucleic acid molecules can be prepared, for example, by chemical synthesis, in vitro transcription, or digestion of long dsRNA by RNase III or Dicer. These can be introduced into cells by transfection, electroporation, or other methods known in the art. See Hannon, G J, 2002, Nature 418: 244-251; Bernstein E et al., 2002, RNA 7: 1509-1521; Hutvagner G et al., Curr. Opin. Genetics & Development 12: 225-232; Brummelkamp, 2002, Science 296: 550-553; Lee N S, et al. 2002. Nature Biotechnol. 20:500-505; Miyagishi M, and Taira K. 2002. Nature Biotechnol. 20:497-500; Paddison P J, et al., 2002. Genes & Dev. 16:948-958; Paul C P, et al., 2002. Nature Biotechnol. 20:505-508; Sui G et al., 2002. Proc. Natl. Acad. Sci. USA 99(6):5515-5520; Yu J-Y et al., 2002. Proc. Natl. Acad. Sci. USA 99(9):6047-6052. Each of the foregoing is incorporated by reference in its entirety.

Guide RNAs

In some embodiments, a conjugated molecular cargo comprises a guide RNA or a DNA encoding a guide RNA. A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and Cash, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 248) or GUUUUAGAGCUAUGCUGUUUUG (SEQ ID NO: 249). Any of the DNA-targeting segments disclosed herein can be joined to the 5′ end of SEQ ID NO: 248 or 249 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

(SEQ ID NO: 250)
AGCAUAGCAAGUUAAAAUAAGGCUAGUCCGUUAUCAACUUGAAAAAGUGG
CACCGAGUCGGUGCUUU,
(SEQ ID NO: 251)
AAACAGCAUAGCAAGUUAAAAUAAGGCUAGUCCGUUAUCAACUUGAAAAA
GUGGCACCGAGUCGGUGCUUUU,
or
(SEQ ID NO: 252)
GUUGGAACCAUUCAAAACAGCAUAGCAAGUUAAAAUAAGGCUAGUCCGUU
AUCAACUUGAAAAAGUGGCACCGAGUCGGUGC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of: GUUUUAGAGCUAGAAAUAGCAAGUUAAAAUAAGGCUAGUCCGUUAUCAACUUGAA AAAGUGGCACCGAGUCGGUGCU (version 1; SEQ ID NO: 253); GUUGGAACCAUUCAAAACAGCAUAGCAAGUUAAAAUAAGGCUAGUCCGUUAUCAA CUUGAAAAAGUGGCACCGAGUCGGUGC (version 2; SEQ ID NO: 254); GUUUUAGAGCUAGAAAUAGCAAGUUAAAAUAAGGCUAGUCCGUUAUCAACUUGAA AAAGUGGCACCGAGUCGGUGC (version 3; SEQ ID NO: 255); and GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 256); GUUUUAGAGCUAGAAAUAGCAAGUUAAAAUAAGGCUAGUCCGUUAUCAACUUGAA AAAGUGGCACCGAGUCGGUGCUUUUUUU (version 5; SEQ ID NO: 257); GUUUUAGAGCUAGAAAUAGCAAGUUAAAAUAAGGCUAGUCCGUUAUCAACUUGAA AAAGUGGCACCGAGUCGGUGCUUUU (version 6; SEQ ID NO: 258); GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 259); or GU UU UAGAGCUAGAAAUAGCAAGU UAAAAUAAGGCUAG UCCGUUAUCAACU UGG CACCGAGUCGGUGC (version 8; SEQ ID NO: 260). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

In some cases, a guide RNA for use in a transcriptional activation system comprising a dCas9-VP64 fusion protein paired with MS2-p65-HSF1 can be used. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂ CH₂O)ₙCH₂ CH₂ OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C1-6 alkylene or C1-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, O(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′—C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA), halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)nCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUm AmGmCAAGUUAAAAUAAGGCUAGUCCGUUAUCAmAmCmUmUmGmAmAmAmAmA mGmUmGmGmCmAmCmCmGmAmGmUmCmGmGmUmGmCmU*mU*mU*mU (SEQ ID NO: 261), where “N” may be any natural or non-natural nucleotide. The totality of N residues can comprise a DNA-targeting segment as described herein. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be conjugated to the FGFR3 binding protein disclosed herein, such as an scFv or an antibody or an antigen-binding fragment thereof, in the form of RNA, either as two molecules (separate crRNA and tracrRNA) or as one molecule (sgRNA), and optionally in the form of a complex with a Cas protein.

The gRNA can be conjugated to the FGFR3 binding protein disclosed herein, such as an scFv or an antibody or an antigen-binding fragment thereof, in the form of DNA encoding the gRNA. The DNA encoding the gRNA can encode a single RNA molecule (sgRNA) or separate RNA molecules (e.g, separate crRNA and tracrRNA). In the latter case, the DNA encoding the gRNA can be provided as one DNA molecule or as separate DNA molecules encoding the crRNA and tracrRNA, respectively.

Multiple gRNAs can be conjugated to the FGFR3 binding protein disclosed herein, such as an scFv or an antibody or an antigen-binding fragment thereof. The gRNAs can be the same or different gRNAs, or can target the same gene or different genes. In some embodiments, 1, 2, 3, 4, 5 or more guide RNAs are conjugated to the FGFR3 binding protein disclosed herein, such as an scFv or an antibody or an antigen-binding fragment thereof.

Alternatively, the gRNA, either in the form of RNA or DNA, may be incorporated into a carrier (e.g., liposomes or LNPs) which is conjugated to the FGFR3 binding protein disclosed herein, such as an scFv or an antibody or an antigen-binding fragment thereof. The carrier can further comprise a Cas protein, such as a Cas9 protein, or a nucleic acid (e.g., mRNA) encoding a Cas protein. Carriers such as liposomes or lipid nanoparticles are described in further detail below.

Multiple gRNAs can be incorporated into a carrier (e.g., liposome or LNP) which is conjugated to the FGFR3 binding protein disclosed herein, such as an scFv or an antibody or an antigen-binding fragment thereof. The gRNAs can be the same or different gRNAs, or can target the same gene or different genes. In some embodiments, 1, 2, 3, 4, 5 or more guide RNAs are incorporated into a carrier (e.g., liposome or LNP) which is conjugated to the FGFR3 binding protein disclosed herein, such as an scFv or an antibody or an antigen-binding fragment thereof.

When a gRNA is provided in the form of DNA, the gRNA after being delivered to the target cell can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand”.

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

The target sequence (e.g., guide RNA target sequence) for the DNA-binding protein can be anywhere within a targeted gene that is suitable for altering expression of the targeted gene. As one example, the target sequence can be within a regulatory element, such as an enhancer or promoter, or can be in proximity to a regulatory element. For example, the target sequence can be within about 10, 20, 30, 40, 50, 100, 200, 300, 400, 500, or 1,000 nucleotides of the start codon.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g, 3 base pairs) upstream or downstream of the PAM sequence (e.g., within the guide RNA target sequence). In the case of SpCas9, the PAM sequence (i.e., on the non-complementary strand) can be 5′-NiGG-3′, where Ni is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN2-3′, where N2 is any DNA nucleotide and is immediately 5′ of the sequence to which the DNA-targeting segment of the guide RNA hybridizes on the complementary strand of the target DNA. In some such cases, Ni and N2 can be complementary and the Ni—N2 base pair can be any base pair (e.g, Ni=C and N2=G; Ni=G and N2=C; Ni=A and N2=T; or Ni=T, and N2=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3. In the case of DpbCasX, the PAM can have the sequence 5′-TTCN-3′. In the case of Cash, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. The guanine at the 5′ end can facilitate transcription by RNA polymerase in cells. Other examples of guide RNA target sequences plus PAMs can include two guanine nucleotides at the 5′ end to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length, including the 5′ G or GG and the 3′ GG or NGG. Yet other guide RNA target sequences plus PAMs can have between 14 and 20 nucleotides in length.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

Other Types of Polynucleotide Molecules

In some embodiments, a molecular cargo, e.g., a polynucleotide molecule described herein may comprise a ribozyme (ribonucleic acid enzyme). Without wishing to be bound by theory, a ribozyme is a molecule, commonly an RNA molecule, that is capable of performing specific biochemical reactions, akin to the action of protein enzymes. Ribozymes comprise molecules possessing catalytic activities such as, but not limited to, the capacity to cleave at specific phosphodiester linkages in RNA molecules to which they have hybridized, e.g., RNA-containing substrates, lncRNAs, mRNAs, and ribozymes.

Ribozymes may take on one of several physical structures, one such structure is termed “hammerhead”. A hammerhead ribozyme can comprise, e.g., a catalytic core comprising nine conserved bases, two regions complementary to the target RNA flanking regions the catalytic core, and a double-stranded stem and loop structure (stem-loop II). The flanking regions may permit the binding of the ribozyme to the target RNA, in particular, by forming double-stranded stems I and III. Cleavage may occur in trans (cleavage of an RNA substrate other than that containing the ribozyme) or in cis (cleavage of the same RNA molecule that contains the hammerhead motif) adjacent to a specific ribonucleotide triplet by a transesterification reaction from a 3′, 5′-phosphate diester to a 2′, 3′-cyclic phosphate diester. In certain embodiments, this catalytic activity may require the presence of specific, highly conserved sequences in the catalytic region of the ribozyme.

Modifications in ribozyme structure can include the replacement or substitution of non-core portions of the molecule with non-nucleotidic molecules. As a non-limiting example, Ma et al. (Biochem. (1993) 32:1751-1758; Nucleic Acids Res. (1993) 21:2585-2589) replaced the six-nucleotide loop of the TAR ribozyme hairpin with non-nucleotidic, ethylene glycol-related linkers. Thomson et al. (Nucleic Acids Res. (1993) 21:5600-5603) replaced loop II with linear, non-nucleotidic linkers of 13, 17, and 19 atoms in length. Benseler et al. (J. Am. Chem. Soc. (1993) 115:8483-8484) describes hammerhead-like molecules where two of the base pairs of stem II, and all four of the nucleotides of loop II may be replaced with non-nucleoside linkers based on bis(propanediol) phosphate, hexaethylene glycol, bis(triethylene glycol) phosphate, propanediol, or tris(propanediol)bisphosphate.

Ribozyme polynucleotides may be generated using any of various suitable methods known in the art (see, e.g., U.S. Pat. Nos. 5,436,143 and 5,650,502; and PCT Publications Nos. WO94/13688; WO91/18624, WO92/01806; and WO 92/07065) or can be obtained from commercial sources (e.g., US Biochemicals), the contents of each of which are incorporated herein by reference in their entirety. In some embodiments, the ribozyme polynucleotide described herein can incorporate nucleotide analogs, e.g., to increase the resistance of the oligonucleotide to degradation by nucleases in a cell. The ribozyme may be synthesized in any known manner, e.g., by use of a commercially available synthesizer produced, e.g., by Applied Biosystems, Inc. or Milligen. The ribozyme RNA sequences may be synthesized conventionally, for example, by using RNA polymerases such as T7 or SP6. The ribozyme may also be produced in recombinant vectors by suitable means.

In some embodiments, internucleotidic phosphorus atoms of the polynucleotide molecules disclosed herein may be chiral, and the properties of the polynucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43, the contents of which are incorporated herein by reference in their entirety). In some embodiments, phosphorothioate-containing oligonucleotides may comprise nucleoside units that can be joined together by either substantially all Rp or substantially all Sp phosphorothioate inter-sugar linkages. In some embodiments, such phosphorothioate oligonucleotides comprising substantially chirally pure inter-sugar linkages may be produced via chemical synthesis or enzymatic approaches, as disclosed, e.g., in U.S. Pat. No. 5,587,261, the contents of which are incorporated herein by reference in their entirety. In some embodiments, chirally controlled polynucleotide molecules described may provide selective cleavage patterns of a target nucleic acid. As a non-limiting example, a chirally controlled polynucleotide molecule may provide single site cleavage within a complementary sequence of a nucleic acid, as disclosed, for example, in US Patent Publication No. 2017/0037399, the contents of which are incorporated herein by reference in their entirety.

In some embodiments, the polynucleotide molecule described herein may be a morpholino-based compound. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties). Morpholino-based oligomeric compounds are also described in, e.g., U.S. Pat. No. 5,034,506, and Genesis, volume 30, issue 3, 2001; Heasman, J., Dev. Biol., 2002, 243, 209-214; Dwaine A. Braasch and David R. Corey, Biochemistry, 2002, 41(14), 4503-4510); Nasevicius et al., Nat. Genet., 2000, 26, 216-220; Lacerra et al., Proc. Natl. Acad. Sci., 2000, 97, 9591-9596, the disclosures of which are incorporated herein by reference in their entireties.

In some embodiments, a polynucleotide molecule described herein may comprise an aptamer. An aptamer may comprise any nucleic acid which specifically binds specifically to a target, e.g., proteinor nucleic acid in a cell. In some embodiments, the aptamer is a DNA aptamer or an RNA aptamer. In some embodiments, a nucleic acid aptamer may comprise a single-stranded RNA (ssDNA or ssRNA) or DNA. In certain embodiments, a single-stranded nucleic acid aptamer may form loop(s) and/or helice(s) structures. The nucleic acid that forms the nucleic acid aptamer may comprise naturally occurring nucleotides, modified nucleotides with hydrocarbon or PEG linkers inserted between one or more nucleotides, modified nucleotides, naturally occurring nucleotides with hydrocarbon linkers (e.g., an alkylene) or a polyether linker (e.g., a PEG linker) inserted between one or more nucleotides, or a combination of thereof. Aptamers and method of producing aptamers are described in, e.g., U.S. Pat. Nos. 8,318,438, 5,650,275; 5,683,867; 5,670,637; 5,696,249; 5,789,157; 5,843,653; 5,270,163; 5,567,588, 5,864,026; 5,989,823; 6,569,630; and PCT Publication No. WO 99/31275, Lorsch and Szostak, 1996; Jayasena, 1999; each incorporated herein by reference.

In some embodiments, a polynucleotide molecule described herein may be a mixmer or comprise a mixmer sequence pattern. In some embodiments, mixmers can be polynucleotides that comprise both naturally and non-naturally occurring nucleosides or comprise two different types of non-naturally occurring nucleosides commonly in an alternating pattern. Mixmers may have higher binding affinity than unmodified polynucleotides and may be used, in particular, to specifically bind a target molecule, e.g., to block a binding site on the target molecule. In some embodiments, mixmers may not recruit an RNase to a target molecule and hence do not promote cleavage of the target molecule. Such polynucleotides that may be incapable of recruiting, e.g., RNase H have been described, e.g., see WO2007/112753 or WO2007/112754.

In some embodiments, a mixmer disclosed herein may comprise a repeating pattern of naturally occurring nucleosides and nucleoside analogues, or, e.g., one type of nucleoside analogue and a second type of nucleoside analogue. Yet, a mixmer need not comprise a repeating pattern and may instead comprise any arrangement of modified naturally occurring nucleosides and nucleosides or any arrangement of one type of modified nucleoside and a second type of modified nucleoside. Such repeating pattern, may, for example comprise every second or every third nucleoside as a modified nucleoside, e.g., LNA. In certain embodiments, the remaining nucleosides may be naturally occurring nucleosides, e.g., DNA, or may be a 2′ substituted nucleoside analogue, e.g., 2′ fluoro analogues or 2′-MOE, or any other some modified nucleoside(s) disclosed herein. It is understood that the repeating pattern of modified nucleoside, such as LNA units, may be combined with modified nucleoside at fixed positions (e.g., at the 5′ and/or 3′ termini).

In some embodiments, a mixmer may not comprise a region of more than 6. more than 5, more than 4, more than 3, or more than 2 consecutive naturally occurring nucleosides (e.g., DNA nucleosides). In some embodiments, the mixmer may comprise at least a region comprising at least two consecutive modified nucleosides, for example, at least two consecutive LNAs. In some embodiments, the mixmer may comprise at least a region consisting of at least three consecutive modified nucleoside units, e.g., at least three consecutive LNAs.

In some embodiments, the mixmer may not comprise a region of more than 8, more than 7, more than 6, more than 5, more than 4, more than 3, or more than 2 consecutive nucleoside analogues, e.g., LNAs. In some embodiments, LNA units may be replaced with other nucleoside analogues including, but not limited to, those referred to herein.

In some embodiments, mixmers may be designed to comprise a mixture of affinity enhancing modified nucleosides, such as, without limitation, in LNA nucleosides and 2′-O-Me nucleosides. In some embodiments, a mixmer may comprise modified internucleoside linkages (e.g., phosphorothioate internucleoside linkages or other linkages) between at least two, at least three, at least four, at least five, at least six or more nucleosides.

In some embodiments, a mixmer may comprise one or more morpholino nucleosides. In some embodiments, a mixmer may comprise morpholino nucleosides mixed (e.g., in an alternating manner) with one or more other nucleosides (e.g., DNA, RNA nucleosides) or modified nucleosides (e.g., 2′-O-Me nucleosides, LNA).

In some embodiments, mixmers may be useful for splice correcting or exon skipping, for example, as described in Chen S. et al., Molecules 2016, 21, 1582, Touznik A., et al., Scientific Reports, volume 7, Article number: 3672 (2017), the contents of each which are incorporated herein by reference.

A mixmer may be produced using any suitable method. Preparation of mixmers is described in, for example, U.S. Pat. No. 7,687,617, and U.S. Patent Application Publication Nos. US2012/0322851, US2009/0209748, US2009/0298916, US2006/0128646, and US2011/0077288. Additional examples of multimers are described, for example, in U.S. Pat. No. 5,693,773, US Patent Application Publication Nos. 2015/0247141; 2015/0315588; US 2011/0158937; the contents of each of which are incorporated herein by reference in their entireties.

In some embodiments, polynucleotide molecules comprising molecular cargos disclosed herein may comprise multimers (e.g., concatemers) of two or more polynucleotide molecules connected, e.g., by a linker. Polynucleotides in a multimer may be the same or different (e.g., targeting different sites on the same gene different genes or products thereof).

In some embodiments, multimers may comprise two or more polynucleotide molecules linked together by a cleavable linker. In some embodiments, multimers may comprise two or more polynucleotide molecules linked together, e.g., by a non-cleavable linker. In some embodiments, a multimer may comprise 2, 3, 4, 5, 6, 7, 8, 9, 10, 11, 12, 13, 14, 15, 16, 17, 18, 19, 20 or more polynucleotide molecules linked together. In some embodiments, a multimer may comprises 2 to 5, 2 to 10, 4 to 20 or 5 to 30 polynucleotide molecules linked together.

In some embodiments, a multimer may comprises two or more polynucleotide molecules linked in a linear arrangement, e.g., end-to-end. In some embodiments, a multimer may comprises two or more polynucleotide molecules linked end-to-end via a polynucleotide-based linker (e.g., an abasic linker, a poly-dT linker). In some embodiments, a multimer comprises a 3′ end of one polynucleotide linked to a 3′ end of another polynucleotide. In some embodiments, a multimer may comprise a 5′ end of one polynucleotide linked to a 3′ end of another polynucleotide. In some embodiments, a multimer comprises a 5′ end of one polynucleotide linked to a 5′ end of another polynucleotide. In some embodiments, multimers may comprise a branched structure comprising multiple polynucleotides linked together by a branching linker.

In some embodiments, a polynucleotide molecule described herein can target splicing. In some embodiments, the polynucleotide can targets splicing by inducing exon skipping and restoring the reading frame within a gene. For example, without limitation, the oligonucleotide may induce skipping of an exon encoding a frameshift mutation and/or an exon that encodes a premature stop codon. In some embodiments, a polynucleotide may induce exon skipping by, e.g., blocking spliceosome recognition of a splice site. In some embodiments, a polynucleotide molecule disclosed herein may induce inclusion of an exon by targeting a splice site inhibitory sequence. In some embodiments, the oligonucleotide promotes inclusion of a particular exon. In some embodiments, exon skipping results in a truncated but functional protein compared to the reference protein.

In some embodiments, the polynucleotide molecule described herein may be a messenger RNA (mRNA). mRNAs comprise an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. Bases of an mRNA can be modified bases such as pseudouridine, N-1-methyl-pseudouridine, or other naturally occurring or non-naturally occurring bases.

### Polypeptide Molecules

In some embodiments, the molecular cargo described herein comprises a polypeptide molecule. When an FGFR3 binding protein (e.g., antibody or antigen-binding fragment) described herein is covalently conjugated to a polypeptide molecule, such conjugates may also be referred to as “fusion proteins”. The term “fused” with regard to fused polypeptides refers to polypeptides joined directly or indirectly (e.g., via a linker or other polypeptide). In one embodiment, the fusion protein is encoded by a single nucleic acid that encodes the FGFR3 binding protein with the polypeptide molecule.

The anti-FGFR3 fusion proteins may be useful, for example, for delivery of the fused polypeptide molecule to various tissues (e.g., nervous tissue) and/or cells in the body, including brain and spinal cord cells such as astrocytes.

Non-limiting examples of polypeptide molecules that can be fused with an FGFR3 binding protein described herein can include, e.g., enzymes, neuroprotective proteins and molecules, or other antigen-binding proteins (e.g., antibodies and antigen-binding fragments thereof). Enzymes can include, without limitation, a hydrolase, including esterases, glycosylases, hydrolases that act on ether bonds, peptidases, linear amidases, diphosphatases, ketone hydrolases, halogenases, phosphoamidases, sulfohydrolases, sulfinases, desulfinases, and the like. In some embodiments, the enzyme is a glycosylase, including glycosidases and N-glycosylases. In some embodiments, the enzyme is a glycosidase, including alpha-amylase, beta-amylase, glucan 1,4-alpha-glucosidase, cellulose, endo-1,3(4)-beta-glucanase, inulinase, endo-1,4-beta-xylanase, endo-1,4-b-xylanase, dextranase, chitinase, polygalacturonidase, lysozyme, exo-alpha-sialidase, alpha-glucosidase, beta-glucosidase, alpha-galactosidase, beta-galactosidase, alpha-mannosidase, beta-mannosidase, beta-fructofuranosidase, alpha,alpha-trehalose, beta-glucuronidase, xylan endo-1,3-beta-xylosidase, amylo-alpha-1,6-glucosidase, hyaluronoglucosaminidase, hyaluronoglucuronidase, and the like.

In some embodiments, the present disclosure includes anti-FGFR3 fusion proteins, e.g., wherein the antigen-binding protein of the fusion is an antibody or antigen-binding fragment thereof set forth herein, and wherein the molecular cargo is a therapeutic agent useful for treating or preventing a neurological and/or neuropsychiatric disease or disorder. Exemplary neurological disease and disorders for treatment with an anti-FGFR3 fusion protein are set forth below in Table 1-4. Also provided are methods for treating or preventing a neurological disease or disorder that is listed below in Table 1-4, in a patient in need thereof, by administering an effective amount of an anti-FGFR3 fusion protein to the patient wherein the molecular cargo is, e.g., a neuroprotective protein encoded by a gene associated with a particular neurological disease or disorder or other neuroprotective molecules, e.g., as described in Table 1-4. Examples of neuroprotective proteins include protective ApoE isoforms or variants (i.e., ApoE2, ApoE Christchurch, ApoE Jacksonville), ATPase 13A2 (encoded by ATP13A2), sulfatase modifying factor 1 (encoded by SUMF1), fragile X messenger ribonucleoprotein (FMRP) (encoded by FMR1), and glutamate transporter-1 (encoded by GLT1). Non-limiting examples of other neuroprotective molecules include a neurotrophic factor including such as but not limited to ciliary neurotrophic factor (CNTF), brain derived neurotrophic factor (BDNF), nerve growth factor (NGF), glial cell line-derived neurotrophic factor (GDNF), and insulin-like growth factor 1 (IGF). Another non-limiting example of a neuroprotective molecule includes a suppressor of cytokine signaling, e.g., suppressor of cytokine signaling 3 (Socs3). Socs3 can inhibit the Jak-Stat3 pathway which is a signaling pathway that can, among other things, lead to astrocyte activation. The anti-FGFR3 fusion protein for administering to a patient in need thereof, e.g., suffering from a neurological disease or disorder, may also comprise an antibody receptor fusion protein. An example of an antibody receptor fusion protein is an anti-amyloid beta antibody-Gas6 fusion protein useful for treating or preventing Alzheimer's disease.

Example methods for preparing a fusion protein comprising an antigen-binding protein are described in, e.g., U.S. Pat. No. 11,208,458, US Patent Publication No. US 2019/0112588, and Baik et al., Mol Ther. 2021 Dec. 1; 29(12):3512-3524, the contents of all of which are incorporated herein by reference in their entireties.

The FGFR3 binding proteins may also be fused to other polypeptide molecules such as, but are not limited to, an epitope (e.g., FLAG) or a tag sequence (e.g., Hiss (SEQ ID NO: 235), and the like) to allow for the detection and/or isolation of the anti-FGFR3 antigen binding protein; a ligand or a portion thereof which binds to a transmembrane receptor protein; an enzyme or portion thereof which is catalytically active; a polypeptide or peptide which promotes oligomerization, such as a leucine zipper domain; a polypeptide or peptide which increases stability, such as an immunoglobulin constant region (e.g., an Fc domain); a half-life extending polypeptide (e.g., albumin or albumin-binding peptides/proteins); a functional or non-functional antibody, or a heavy or light chain thereof; and a polypeptide which has an activity, such as a therapeutic activity, different from the anti-FGFR3 antigen binding protein of the present disclosure.

In some embodiments, the polypeptide molecule can be a gene editing nuclease, such as Cas protein, ZFN, TALEN. Gene editing nucleases are described in further details below.

In some embodiments, anti-FGFR3 fusion proteins can be made by fusing the heterologous polypeptide molecule at either the N-terminus or at the C-terminus of the anti-FGFR3 antigen binding protein (e.g., the heavy chain and/or light chain). Heterologous sequences can be fused either directly to the anti-FGFR3 antigen binding protein, either chemically or by recombinant expression from a single polynucleotide or they may be joined via a linker or adapter molecule. A peptidyl linker or adapter molecule can be one or more amino acid residues (or -mers), e.g., 1, 2, 3, 4, 5, 6, 7, 8, or 9 residues (or -mers), preferably from 10 to 50 amino acid residues (or -mers), e.g., 10, 11, 12, 13, 14, 15, 16, 17, 18, 19, 20, 25, 30, 35, 40, 45, or 50 residues (or -mers), and more preferably from 15 to 35 amino acid residues (or -mers). A linker or adapter molecule can also be designed with a cleavage site for a protease to allow for the separation of the fused moieties.

When forming the fusion proteins of the present disclosure, a linker can be employed. The linker can be made up of amino acids linked together by peptide bonds, i.e., a peptidyl linker. In some embodiments, the linker is made up of from 1 to 20 or more amino acids linked by peptide bonds, wherein the amino acids are selected from the 20 naturally occurring amino acids. In some embodiments, the amino acids are selected from the amino acids glycine, serine, and glutamate. In some embodiments, suitable linkers include, for example, GSGEGEGSEGSG (SEQ ID NO: 266); GGSEGEGSEGGS (SEQ ID NO: 267); GGGGS (SEQ ID NO: 268); and GGGS (SEQ ID NO: 269). The present disclosure contemplates linkers of any length or composition.

### Liposomes, Lipid Nanoparticles and Other Carriers

In some embodiments, a conjugated molecular cargo described herein comprises a carrier, for example, a lipid-based carrier, such as a lipid nanoparticle (LNP), a liposome, a lipidoid, or a lipoplex, a polymeric nanoparticle, an inorganic nanoparticle, a peptide carrier, a nanoparticle mimic, or a nanotube.

In some embodiments, a conjugated molecular cargo described herein comprises a liposome or LNP. Liposomes and LNPs are vesicles including one or more lipid bilayers. In some embodiments, a liposome or LNP includes two or more concentric bilayers separated by aqueous compartments. Lipid bilayers can be functionalized and/or crosslinked to one another. Lipid bilayers can include one or more proteins, polysaccharides or other molecules.

Lipid formulations can protect biological molecules from degradation while improving their cellular uptake. Liposomes or LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such liposomes or LNPs can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine (DSPC). In another example, the other components can comprise a helper lipid such as cholesterol, an optional neutral lipid such as DSPC, and a stealth lipid such as S010, S024, S027, S031, or S033.

Liposomes are amphiphilic lipids which can form bilayers in an aqueous environment to encapsulate an aqueous core. The polypeptide (e.g., Cas protein) or polynucleotide (e.g., guide RNA) may be incorporated into the aqueous core. These lipids can have an anionic, cationic or zwitterionic hydrophilic head group. Liposomes can be formed from a single lipid or from a mixture of lipids. A mixture may comprise (1) a mixture of anionic lipids; (2) a mixture of cationic lipids; (3) a mixture of zwitterionic lipids; (4) a mixture of anionic lipids and cationic lipids; (5) a mixture of anionic lipids and zwitterionic lipids; (6) a mixture of zwitterionic lipids and cationic lipids; or (7) a mixture of anionic lipids, cationic lipids and zwitterionic lipids. Similarly, a mixture may comprise both saturated and unsaturated lipids. Exemplary phospholipids include, but are not limited to, phosphatidylethanolamines, phosphatidylcholines, phosphatidylserines, and phosphatidylglycerols. Cationic lipids include, but are not limited to, 1,2-distearyloxy-N,N-dimethyl-3-aminopropane (DSDMA), dioleoyl trimethylammonium propane (DOTAP), 1,2-dioleyloxy-N, Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids include dodecylphosphocholine, DPPC, and DOPC.

The liposomes or LNPs may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes.

In some examples, the liposomes or LNPs comprise cationic lipids. In some examples, the liposomes or LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Additional suitable cationic lipids include, but are not limited to 1,2-DiLinoleyloxy-N, N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C18 alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA. Additional cationic lipids are described in U.S. Pat. Nos. 7,745,651; 5,208,036; 5,264,618; 5,279,833; 5,283,185; 5,753,613; and 5,785,992, incorporated herein by reference.

In some embodiments, the cationic lipids may comprise a protonatable tertiary amine head group. Such lipids are referred to herein as ionizable lipids. Ionizable lipids refer to lipid species comprising an ionizable amine head group and typically comprising a pKa of less than about 7. In environments with an acidic pH, the ionizable amine head group is protonated such that the ionizable lipid preferentially interacts with negatively charged molecules (e.g., nucleic acids such as the recombinant polynucleotides described herein) thus facilitating liposome or LNP assembly and encapsulation. Therefore, in some embodiments, ionizable lipids can increase the loading of nucleic acids into liposomes or LNPs. In environments where the pH is greater than about 7 (e.g., physiologic pH of 7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle.

In some embodiments, the liposomes or LNPs may comprise one or more non-cationic helper lipids. Exemplary helper lipids include (1,2-dilauroyl-sn-glycero-3-phosphoethanolamine) (DLPE), 1,2-diphytanoyl-sn-glycero-3-phosphoethanolamine (D iPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DM PE), (1,2-dioleoyl-sn-glycero-3-phospho-(1′-rac-glycerol) (DOPG), 1,2-distearoyl-sn-glycero-3-phosphoethanolamine (DSPE), ceramides, sphingomyelins, and cholesterol.

Some such lipids suitable for use in the liposomes or LNPs described herein are biodegradable in vivo. Examples of biodegradable lipids include, but are not limited to, (9Z,12Z)-3-((4,4-bis(octyloxy)butanoyl)oxy)-2-((((3-20 (diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate) or another ionizable lipid. See, e.g., PCT Publication Nos. WO2017/173054, WO2015/095340, and WO2014/136086. In some embodiments, the term cationic and ionizable in the context of liposome or LNP lipids is interchangeable, e.g., wherein ionizable lipids are cationic depending on the pH.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the liposomes or LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DM PC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DM PC), 1-myristoyl-2-palmitoyl phosphatidylcholine (M PPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the liposomes or LNPs. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain liposome or LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DM B (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the liposomes or LNPs may further comprise one or more of PEG-modified lipids that comprise a poly(ethylene)glycol chain of up to 5 kDa in length covalently attached to a lipid comprising one or more C6-C20 alkyls. In some embodiments, the liposomes or LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)] (DSPE-PEG-amine). In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, or about 1.0%, of the total lipid content in the liposome or lipid nanoparticle.

In some embodiments, a liposome or LNP described herein may comprise a conjugated lipid that inhibits aggregation of lipid particles. Such lipid conjugates include, but are not limited to, PEG-lipid conjugates such as, e.g, PEG coupled to dialkyloxypropyls (e.g, PEG-DAA conjugates), PEG coupled to diacylglycerols (e.g, PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates), polyamide oligomers (e.g, ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain embodiments, non-ester containing linker moieties, such as amides or carbamates, are used.

The liposomes or LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The liposomes or LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some embodiments, the liposome or LNP can comprise a nuclease agent (e.g., CRISPR/Cas system, ZFN, or TALEN), can comprise a polynucleotide molecule (e.g., guide RNA), can comprise a nucleic acid construct encoding a polypeptide of interest (e.g., multidomain therapeutic protein), or can comprise both a nuclease agent (e.g., a CRISPR/Cas system) and a nucleic acid construct encoding a polypeptide of interest (e.g., a donor template for use in gene editing). Regarding CRISPR/Cas systems, the liposomes or LNPs can comprise the Cas protein in any form (e.g., protein, DNA, or mRNA) and/or can comprise the guide RNA(s) in any form (e.g., DNA or RNA). In one example, the liposomes or LNPs comprise the Cas protein in the form of mRNA (e.g., a modified RNA as described herein) and the guide RNA(s) in the form of RNA (e.g., a modified guide RNA as disclosed herein). As another example, the liposomes or LNPs can comprise the Cas protein in the form of protein and the guide RNA(s) in the form of RNA). In one example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity.

In certain liposomes or LNPs, the cargo can include a guide RNA or a nucleic acid encoding a guide RNA. In certain liposomes or LNPs, the cargo can include an mRNA encoding a Cas nuclease, such as Cas9, and a guide RNA or a nucleic acid encoding a guide RNA. In certain liposomes or LNPs, the cargo can include a nucleic acid construct encoding a polypeptide of interest (e.g., multidomain therapeutic protein) as described elsewhere herein. In certain liposomes or LNPs, the cargo can include an mRNA encoding a Cas nuclease, such as Cas9, a guide RNA or a nucleic acid encoding a guide RNA, and a nucleic acid construct encoding a polypeptide of interest (e.g., multidomain therapeutic protein). In some liposomes or LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some liposomes or LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the liposome or LNP formulation can include a ratio of Cas mRNA to gRNA nucleic acid of from about 2:1 to about 1:2. In specific examples, the ratio of Cas mRNA to gRNA can be about 2:1.

In some liposomes or LNPs, the cargo can comprise a nucleic acid construct encoding a polypeptide of interest (e.g., multidomain therapeutic protein) and gRNA. The nucleic acid construct encoding a polypeptide of interest (e.g., multidomain therapeutic protein) and gRNAs can be in different ratios. For example, the liposome or LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

Dynamic Light Scattering (“DLS”) can be used to characterize the polydispersity index (“PDI”) and size of the liposomes and LNPs. In some embodiments, the PDI may range from about 0.005 to about 0.75. In some embodiments, the PDI may range from about 0.01 to about 0.5. In some embodiments, the PDI may range from about 0.02 to about 0.4. In some embodiments, the PDI may range from about 0.03 to about 0.35. In some embodiments, the PDI may range from about 0.1 to about 0.35.

The LNPs disclosed herein may have a size of about 1 to about 250 nm. In some embodiments, the LNPs may have a size of about 10 to about 200 nm. In some embodiments, the LNPs may have a size of about 20 to about 150 nm. In some embodiments, the LNPs may have a size of about 50 to about 150 nm. In some embodiments, the LNPs may have a size of about 50 to about 100 nm. In some embodiments, the LNPs may have a size of about 50 to about 120 nm. In some embodiments, the LNPs may have a size of about 75 to about 150 nm. In some embodiments, the LNPs may have a size of about 30 to about 200 nm. In some embodiments, the average sizes (diameters) of the fully formed nanoparticles are measured by dynamic light scattering on a Malvern Zetasizer (e.g., the nanoparticle sample may be diluted in phosphate buffered saline (PBS) so that the count rate is approximately 200-400 kcts, and the data may be presented as a weighted-average of the intensity measure).

In some embodiments, the liposomes or LNPs may be formed with an average encapsulation efficiency ranging from about 50% to about 100%. In some embodiments, the liposomes or LNPs may be formed with an average encapsulation efficiency ranging from about 50% to about 70%. In some embodiments, the liposomes or LNPs may be formed with an average encapsulation efficiency ranging from about 70% to about 90%. In some embodiments, the liposomes or LNPs may be formed with an average encapsulation efficiency ranging from about 90% to about 100%. In some embodiments, the liposomes or LNPs may be formed with an average encapsulation efficiency ranging from about 75% to about 95%.

In addition to liposomes and LNPs, an FGFR3 binding protein disclosed herein, such as an scFv or an antibody or an antigen-binding fragment thereof, may be conjugated to other carriers for delivery of nucleic acid and/protein molecules. Examples of other suitable carriers include, but are not limited to, lipoids and lipoplexes, particulate or polymeric nanoparticles, inorganic nanoparticles, peptide carriers, nanoparticle mimics, nanotubes, conjugates, immune stimulating complexes (ISCOM), virus-like particles (VLPs), self-assembling proteins, or emulsion delivery systems such as cationic submicron oil-in-water emulsions.

Polymeric microparticles or nanoparticles can also be used to encapsulate or adsorb a polypeptide (e.g., Cas protein) or polynucleotide (e.g., guide RNA). The particles may be substantially non-toxic and biodegradable. The particles useful for delivering a polynucleotide (e.g., guide RNA) may have an optimal size and zeta potential. For example, the microparticles may have a diameter in the range of 0.02 μm to 8 μm. In the instances when the composition has a population of micro- or nanoparticles with different diameters, at least 80%, 85%, 90%, or 95% of those particles ideally have diameters in the range of 0.03-7 μm. The particles may also have a zeta potential of between 40-100 mV, in order to provide maximal adsorption of the polynucleotide (e.g., guide RNA) to the particles.

Non-toxic and biodegradable polymers include, but are not limited to, poly(ahydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, one or more natural polymers such as a polysaccharide, for example pullulan, alginate, inulin, and chitosan, and combinations thereof. In some embodiments, the particles are formed from poly(ahydroxy acids), such as a poly(lactides) (PLA), poly(g-glutamic acid) (g-PGA), poly(ethylene glycol) (PEG), polystyrene, copolymers of lactide and glycolide such as a poly(D,L-lactide-co-glycolide) (PLG), and copolymers of D,L-lactide and caprolactone. Useful PLG polymers can include those having a lactide/glycolide molar ratio ranging, for example, from 20:80 to 80:20 e.g., 25:75, 40:60, 45:55, 55:45, 60:40, 75:25. Useful PLG polymers include those having a molecular weight between, for example, 5,000-200,000 Da e.g., between 10,000-100,000, 20,000-70,000, 40,000-50,000 Da.

The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles.

For example, the inorganic nanoparticles may be calcium phosphate nanoparticles, silicon nanoparticles or gold nanoparticles. Inorganic nanoparticles typically have a rigid structure and comprise a shell in which a polypeptide or polynucleotide is encapsulated or a core to which the polypeptide or polynucleotide may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptides or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, polypeptides or polynucleotides of the present disclosure can be conjugated to nanoparticles. Nanoparticles that may be used for conjugation with antigens and/or antibodies of the present disclosure include but not are limited to chitosan-shelled nanoparticles, carbon nanotubes, PEGylated liposomes, poly(d,l-lactide-co-glycolide)/montmorillonite (PLGA/MMT) nanoparticles, poly(lactide-co-glycolide) (PLGA) nanoparticles, poly-(malic acid)-based nanoparticles, and other inorganic nanoparticles (e.g., nanoparticles made of magnesium-aluminium layered double hydroxides with disuccinimidyl carbonate (DSC), and TiO2 nanoparticles). Nanoparticles can be developed and conjugated to an antigens and/or antibodies contained in a composition for targeting virus-infected cells.

Oil-in-water emulsions may also be used for delivering a polypeptide or polynucleotide (e.g., mRNA) to a subject. Examples of oils useful for making the emulsions include animal (e.g., fish) oil or vegetable oil (e.g., nuts, grains and seeds). The oil may be biodegradable and biocompatible. Exemplary oils include, but are not limited to, tocopherols and squalene, a shark liver oil which is a branched, unsaturated terpenoid and combinations thereof. Terpenoids are branched chain oils that are synthesized biochemically in 5-carbon isoprene units.

The aqueous component of the emulsion can be water or can be water in which additional components have been added. For example, it may include salts to form a buffer e.g., citrate or phosphate salts, such as sodium salts. Exemplary buffers include a borate buffer, a citrate buffer, a histidine buffer a phosphate buffer, a Tris buffer, or a succinate buffer.

In some embodiments, the oil-in water emulsions include one or more cationic molecules. For example, a cationic lipid can be included in the emulsion to provide a positively charged droplet surface to which negatively-charged polynucleotide (e.g., mRNA) can attach. Exemplary cationic lipids include, but are not limited to: 1,2-dioleoyloxy-3-(trimethylammonio)propane (DOTAP), 1,2-Dimyristoyl-3-Trimethyl-AmmoniumPropane (DMTAP), N′-Dimethylaminoethane)-carbamoyl]Cholesterol (DC Cholesterol), dimethyldioctadecyl-ammonium (DDA e.g., the bromide), dipalmitoyl(C16:0)trimethyl ammonium propane (DPTAP), distearoyltrimethylammonium propane (DSTAP). Other useful cationic lipids include benzalkonium chloride (BAK), benzethonium chloride, cholesterol hemisuccinate choline ester, lipopolyamines (e.g., dioctadecylamidoglycylspermine (DOGS), dipalmitoyl phosphatidylethanol-amidospermine (DPPES)), cetramide, cetylpyridinium chloride (CPC), cetyl trimethylammonium chloride (CTAC), cationic derivatives of cholesterol (e.g., cholesteryl-3.beta.-oxysuccinamidoethylenetrimethylammonium salt, cholesteryl-3. beta.-oxysuccinamidoethylene-dimethylamine, cholesteryl-3. beta.-carboxyamidoethylenetrimethylammonium salt, and cholesteryl-3. beta.-carboxyamidoethylenedimethylami ne), N, N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-namini um chloride (DEBDA), cholesteryl (4′-trimethylammonio) butanoate), N-alkyl pyridinium salts (e.g., cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-alpha.dioleoylphosphatidylethanolamine, lipopoly-L (or D)-lysine (LPLL, LPDL), poly(L (or D)-lysine conjugated to N-glutarylphosphatidylethanolamine, dialkyldimethylammonium salts, [1-(2,3-dioleyloxy)-propyl]-N,N,N,trimethylammonium chloride, 1,2-diacyl-3-(trimethylammonio) propane (acyl group can be dimyristoyl, dipalmitoyl, distearoyl, or dioleoyl), 1,2-diacyl-3 (dimethylammonio)propane (acyl group can be dimyristoyl, dipalmitoyl, distearoyl, or dioleoyl), 1,2-dioleoyl-3-(4′-trimethyl-ammonio)butanoyl-sn-glycerol, 1,2-dioleoyl 3-succinyl-sn-glycerol choline ester, didodecyl glutamate ester with pendant amino group (C GluPhCnN), and ditetradecyl glutamate ester with pendant amino group (C14GluCnN+).

In some embodiments, in addition to the oil and cationic lipid, an emulsion can also include a non-ionic surfactant and/or a zwitterionic surfactant. Examples of useful surfactants include, but are not limited to: the polyoxyethylene sorbitan esters surfactants, e.g., polysorbate 20 and polysorbate 80; copolymers of ethylene oxide, propylene oxide, and/or butylene oxide, linear block copolymers; phospholipids, e.g., phosphatidylcholine; polyoxyethylene fatty ethers derived from lauryl, cetyl, stearyl and oleyl alcohols; polyoxyethylene-9-lauryl ether; octoxynols; (octylphenoxy)polyethoxyethanol, and sorbitan esters.

In some embodiments, a polynucleotide described herein may be incorporated into polynucleotide complexes, such as, but not limited to, nanoparticles (e.g., polynucleotide self-assembled nanoparticles, polymer-based self-assembled nanoparticles, inorganic nanoparticles, lipid nanoparticles, semiconductive/metallic nanoparticles), gels and hydrogels, polynucleotide complexes with cations and anions, microparticles, and any combination thereof. The polynucleotide complexes may be conjugated to an FGFR3 binding protein described herein, e.g., via linkage to the polynucleotide or nanoparticle/hydrogel/microparticle.

In some embodiments, the polynucleotides disclosed herein may be formulated as self-assembled nanoparticles. As a non-limiting example, polynucleotides may be used to make nanoparticles which may be used in a delivery system for the polynucleotides (See e.g., PCT Publication No. WO2012/125987). In some embodiments, the polynucleotide self-assembled nanoparticles may comprise a core of the polynucleotides disclosed herein and a polymer shell. The polymer shell may be any of the polymers described herein and are known in the art. In an additional embodiment, the polymer shell may be used to protect the polynucleotides in the core.

In some embodiments, self-assembled nanoparticles may be microsponges formed of long polymers of polynucleotide hairpins which form into crystalline “pleated” sheets before self-assembling into microsponges. These microsponges are densely-packed sponge like microparticles which may function as an efficient carrier and may be able to deliver cargo to a cell. The microsponges may be from 1 μm to 300 nm in diameter. The microsponges may be complexed with other agents known in the art to form larger microsponges. As a non-limiting example, the microsponge may be complexed with an agent to form an outer layer to promote cellular uptake such as polycation polyethyleneime (PEI). This complex can form a 250-nm diameter particle that can remain stable at high temperatures (150° C.) (Grabow and Jaegar, Nature Materials 2012, 11:269-269). Additionally, these microsponges may be able to exhibit an extraordinary degree of protection from degradation by ribonucleases. In an embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, polynucleotides.

In some embodiments, a polynucleotide disclosed herein may be formulated in inorganic nanoparticles (see U.S. Pat. No. 8,257,745). The inorganic nanoparticles may include, but are not limited to, clay substances that are water swellable. As a non-limiting example, the inorganic nanoparticle may include synthetic smectite clays which are made from simple silicates (See U.S. Pat. Nos. 5,585,108 and 8,257,745).

In some embodiments, a polynucleotide disclosed herein may be formulated in water-dispersible nanoparticle comprising a semiconductive or metallic material (U.S. Patent Application Publication No. 2012/0228565; herein incorporated by reference in its entirety) or formed in a magnetic nanoparticle (U.S. Patent Application Publication No. 2012/0265001 and 2012/0283503). The water-dispersible nanoparticles may be hydrophobic nanoparticles or hydrophilic nanoparticles.

In some embodiments, the polynucleotides disclosed herein may be encapsulated into any hydrogel known in the art which may form a gel when injected into a subject. Hydrogels are a network of polymer chains that are hydrophilic, and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may be used to encapsulate lipid nanoparticles which are biocompatible, biodegradable and/or porous.

As a non-limiting example, the hydrogel may be an aptamer-functionalized hydrogel. The aptamer-functionalized hydrogel may be programmed to release one or more polynucleotides using polynucleotide hybridization. (Battig et al., J. Am. Chem. Society. 2012 134:12410-12413). In some embodiments, the polynucleotide may be encapsulated in a lipid nanoparticle and then the lipid nanoparticle may be encapsulated into a hydrogel.

In some embodiments, the polynucleotides disclosed herein may be encapsulated into a fibrin gel, fibrin hydrogel or fibrin glue. In another embodiment, the polynucleotides may be formulated in a lipid nanoparticle or a rapidly eliminated lipid nanoparticle prior to being encapsulated into a fibrin gel, fibrin hydrogel or a fibrin glue. In yet another embodiment, the polynucleotides may be formulated as a lipoplex prior to being encapsulated into a fibrin gel, hydrogel or a fibrin glue. Fibrin gels, hydrogels and glues comprise two components, a fibrinogen solution and a thrombin solution which is rich in calcium (See e.g., Spicer and Mikos, Journal of Controlled Release 2010. 148: 49-55; Kidd et al. Journal of Controlled Release 2012. 157:80-85). The concentration of the components of the fibrin gel, hydrogel and/or glue can be altered to change the characteristics, the network mesh size, and/or the degradation characteristics of the gel, hydrogel and/or glue such as, but not limited to changing the release characteristics of the fibrin gel, hydrogel and/or glue. (See e.g., Spicer and Mikos, Journal of Controlled Release 2010. 148: 49-55; Kidd et al. Journal of Controlled Release 2012. 157:80-85; Catelas et al. Tissue Engineering 2008. 14:119-128). This feature may be advantageous when used to deliver the polynucleotide disclosed herein. (See e.g., Kidd et al. Journal of Controlled Release 2012. 157:80-85; Catelas et al. Tissue Engineering 2008. 14:119-128).

In some embodiments, a polynucleotide disclosed herein may include cations or anions. In one embodiment, the formulations include metal cations such as, but not limited to, Zn²⁺, Ca²⁺, Cu²⁺, Mg²⁺ and combinations thereof. As a non-limiting example, formulations may include polymers and a polynucleotide complexed with a metal cation (See U.S. Pat. Nos. 6,265,389 and 6,555,525).

In some embodiments, a polynucleotide may be formulated in nanoparticles and/or microparticles. These nanoparticles and/or microparticles may be molded into any size shape and chemistry. As an example, the nanoparticles and/or microparticles may be made using the PRINT® technology by LIQUIDA TECHNOLOGIES (Morrisville, N.C.) (See e.g., International Pub. Publication No. WO2007/024323).

In some embodiments, the polynucleotides disclosed herein may be formulated in NanoJackets and NanoLiposomes by Keystone Nano (State College, Pa.). NanoJackets are made of compounds that are naturally found in the body including calcium, phosphate and may also include a small amount of silicates. Nanojackets may range in size from 5 to 50 nm and may be used to deliver hydrophilic and hydrophobic compounds such as, but not limited to, polynucleotides, primary constructs and/or polynucleotide. NanoLiposomes are made of lipids such as, but not limited to, lipids which naturally occur in the body. NanoLiposomes may range in size from 60-80 nm and may be used to deliver hydrophilic and hydrophobic compounds such as, but not limited to, polynucleotides, primary constructs and/or polynucleotide. In one aspect, the polynucleotides disclosed herein are formulated in a NanoLiposome such as, but not limited to, Ceramide NanoLiposomes.

### Gene Editing System

In various embodiments, a molecular cargo of the present disclosure can include a gene editing system or components of such systems. Various known gene editing systems can be used in the methods and compositions described herein, including, e.g., a Clustered Regularly Interspersed Short Palindromic Repeats (CRISPR)/Cas system; zinc finger nuclease (ZFN) system; transcription activator-like effector nuclease (TALEN) system, or systems using meganucleases, restriction endonucleases, or recombinases. Generally, these gene editing systems are used to modify a genome within a cell by inducing a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA (gRNA) to guide specific cleavage or nicking of a target DNA sequence. Further, targeted nucleases have been developed, and additional nucleases are being developed, for example based on the Argonaute system (e.g., from T. thermophilus, known as ‘TtAgo’, see Swarts et al (2014) Nature 507(7491): 258-261), which also may have the potential for uses in genome editing and gene therapy.

Deletion of DNA may be performed using a gene editing system to knock-out or disrupt a target gene. A knock-out can be a gene knock-down or the gene can be knocked out by a mutation such as, a point mutation, an insertion, a deletion, a frameshift, or a missense mutation by techniques known in the art. Alternatively, a knock-in of an exogenous gene or replacement of a defective gene with a corrective gene can also be achieved with a gene editing system. In such instances, a donor template carrying an heterologous gene to be inserted into a genomic locus is provided along with a gene editing system. The donor template would typically include homology arms corresponding to the genomic locus which is targeted by a gene editing system.

There are various ways to incorporate a gene editing system or component(s) thereof (e.g., Cas protein, guide RNA) to an anti-FGFR3 protein-drug conjugate described herein. In some embodiments, a gene editing system or component(s) thereof (e.g., Cas protein or nucleic acid (e.g., mRNA or DNA) encoding the Cas protein, guide RNA or a DNA encoding the guide RNA) are loaded to a carrier described, such as a liposome or LNP, which is conjugated to an anti-FGFR3 antigen-binding protein described herein. In some embodiments, a guide RNA or a DNA encoding the guide RNA is conjugated to an anti-FGFR3 antigen-binding protein described herein. In some embodiments, a gene editing nuclease (e.g., Cas protein, ZFN, TALEN) or one or more nucleic acids (e.g., mRNA or DNA) encoding the gene editing nuclease is conjugated to anti-FGFR3 antigen-binding protein described herein. In some embodiments, both a guide RNA (or DNA encoding the guide DNA) and a Cas protein (or nucleic acid (e.g., mRNA or DNA) encoding the Cas protein) may be conjugated to an anti-FGFR3 antigen-binding protein described herein. In some embodiments, a guide RNA (or DNA encoding the guide RNA) is conjugated to an anti-FGFR3 antigen-binding protein described herein, and a Cas protein (or nucleic acid (e.g., mRNA or DNA) encoding the Cas protein) is loaded to a carrier described, such as a liposome or LNP, which is conjugated to an anti-FGFR3 antigen-binding protein described herein. In some embodiments, a Cas protein (or nucleic acid (e.g., mRNA or DNA) encoding the Cas protein) is conjugated to an anti-FGFR3 antigen-binding protein described herein, and a guide RNA (or DNA encoding the guide RNA) is loaded to a carrier described, such as a liposome or LNP, which is conjugated to an anti-FGFR3 antigen-binding protein described herein.

In some embodiments, the molecular cargo disclosed herein can comprise a CRISPR/Cas system or components of such systems. CRISPR/Cas systems include transcripts and other elements involved in the expression of, or directing the activity of, Cas genes. A CRISPR/Cas system can be, for example, a type I, a type II, or a type III system. Alternatively, a CRISPR/Cas system can be a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed cleavage of nucleic acids.

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csyl, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8: 14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011 GWA2_33_10, Parcubacteria bacterium GW2011 GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (P1mCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more or all of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasX proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas nuclease can be fused to a cleavage domain, an epigenetic modification domain, a transcriptional activation domain, or a transcriptional repressor domain. See WO 2014/089290, herein incorporated by reference in its entirety for all purposes. Examples of transcriptional activation domains include a herpes simplex virus VP 16 activation domain, VP64 (which is a tetrameric derivative of VP 16), a NFKB p65 activation domain, p53 activation domains 1 and 2, a CREB (cAMP response element binding protein) activation domain, an E2A activation domain, and an NFAT (nuclear factor of activated T-cells) activation domain. Other examples include activation domains from Octl, Oct-2A, SP1, AP-2, CTF1, P300, CBP, PCAF, SRC1, PvALF, ERF-2, OsGAI, HALF-1, CI, API, ARF-5, ARF-6, ARF-7, ARF-8, CPRF1, CPRF4, MYC-RP/GP, TRABIPC4, and HSF1. See, e.g., US 2016/0237456, EP3045537, and WO 2011/146121, each of which is incorporated by reference in its entirety for all purposes.

In some cases, a transcriptional activation system can be used comprising a dCas9-VP64 fusion protein paired with MS2-p65-HSFI. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Examples of transcriptional repressor domains include inducible cAMP early repressor (ICER) domains, Kruppel-associated box A (KRAB-A) repressor domains, YY 1 glycine rich repressor domains, Spl-like repressors, E(spl) repressors, IKB repressor, and MeCP2. Other examples include transcriptional repressor domains from A/B, KOX, TGF-beta-inducible early gene (TIEG), v-erbA, SID, SID4X, MBD2, MBD3, DNMT1, DNMG3A, DNMT3B, Rb, ROM2, See, e.g., EP3045537 and WO 2011/146121, each of which is incorporated by reference in its entirety for all purposes. Cas nucleases can also be fused to a heterologous polypeptide providing increased or decreased stability. The fused domain or heterologous polypeptide can be located at the N-terminus, the C-terminus, or internally within the Cas nuclease.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 270) or PKKKRRV (SEQ ID NO: 271). The NLS may be a bipartite sequence, such as the NLS of nucleoplasmin, KRPAATKKAGQAKKKK (SEQ ID NO: 272). In a specific example, a single PKKKRKV (SEQ ID NO: 270) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, ora polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, 51, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge). Examples of codon-optimized Cas9 coding sequences, Cas9 mRNAs, and Cas9 protein sequences include those described in WO2013/176772, WO2014/065596, WO2016/106121, and WO2019/067910 are hereby incorporated by reference. In particular, the Cas9 coding sequences and Cas9 amino acid sequences of the table at paragraph [0449] WO2019/067910, and the Cas9 mRNAs and coding sequences of paragraphs [0214]-[0234] of WO2019/067910 are hereby incorporated by reference. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA). Different promoters can be used to drive expression of the gRNA, such as a U6 promoter or the small tRNA Gln. Likewise, different promoters can be used to drive Cas9 expression.

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl pseudouridine can be used. As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). Likewise, Cas mRNAs can be modified by depletion of uridine using synonymous codons. For example, capped and polyadenylated Cas mRNA fully substituted with pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11): E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with el F4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides (SEQ ID NO: 391).

In some embodiments, a CRISPR/Cas system can be used to create a site of insertion at a desired locus within a host genome, at which site a construct disclosed herein can be inserted to express one or more polypeptides of interest. Methods of designing suitable guide RNAs that target any desired locus of a host genome for insertion are well known in the art. A construct comprising a transgene may be heterologous with respect to its insertion site, for example, insertion of a heterologous transgene into a “safe harbor” locus. A construct comprising a transgene may be non-heterologous with respect to its insertion site, for example, insertion of a wild-type transgene into its endogenous locus.

Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1 (PPP1 R12C), Rosa (e.g., Rosa26), AngptiS, ApoC3, ASGR2, FIX (F9), G6PC, Gys2, HGD, Lp(a), Pcsk9, SERPINAI, TF, and TTR. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67, 328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In some embodiments, the heterologous gene may be inserted into a safe harbor locus and use the safe harbor locus's endogenous signal sequence. In some embodiments, the heterologous gene may comprise its own signal sequence, may be inserted into the safe harbor locus, and may further use the safe harbor locus's endogenous signal sequence. In some embodiments, the gene may comprise its own signal sequence and an internal ribosomal entry site (IRES), may be inserted into the safe harbor locus, and may further use the safe harbor locus's endogenous signal sequence. In some embodiments, the gene may comprise its own signal sequence and IRES, may be inserted into the safe harbor locus, and does not use the safe harbor locus's endogenous signal sequence. In some embodiments, the gene may be inserted into the safe harbor locus and may comprise an IRES and does not use any signal sequence.

In some methods, two or more nuclease agents can be used. For example, two or more nuclease agents can be used, each targeting a nuclease target sequence including or proximate to the start codon. As another example, two nuclease agents can be used, one targeting a nuclease target sequence including or proximate to the start codon, and one targeting a nuclease target sequence including or proximate to the stop codon, wherein cleavage by the nuclease agents can result in deletion of the coding region between the two nuclease target sequences. As yet another example, three or more nuclease agents can be used, with one or more (e.g., two) targeting nuclease target sequences including or proximate to the start codon, and one or more (e.g., two) targeting nuclease target sequences including or proximate to the stop codon, wherein cleavage by the nuclease agents can result in deletion of the coding region between the nuclease target sequences including or proximate to the start codon and the nuclease target sequence including or proximate to the stop codon.

In some embodiments, CRISPR/Cas systems used in the compositions and methods disclosed herein can be non-naturally occurring.

In some embodiments, the Cas protein (e.g., Cas9) may be complexed with a gRNA to form a ribonucleoprotein complex (RNP). In some embodiments, a molecular cargo (e.g., liposome or LNP) described herein comprises a ribonucleoprotein complex (RNP) comprising a Cas protein (e.g., Cas9) and a gRNA.

In some embodiments, a molecular cargo (e.g., liposomes and LNPs) described herein may comprise one or more components from gene editing systems other than a CRISPR/Cas system. In some embodiments, the molecular cargo is a nuclease, such as Zinc-finger nuclease (ZFN) or a TALEN, which is effective to bind and modify at a target gene.

Any nuclease molecular cargo that induces a nick or double-strand break into a desired target sequence or any DNA-binding protein that binds to a desired target sequence can be used in the methods and compositions disclosed herein. A naturally occurring or native nuclease molecular cargo can be employed so long as the nuclease molecular cargo induces a nick or double-strand break in a desired target sequence. Likewise, a naturally occurring or native DNA-binding protein can be employed so long as the DNA-binding protein binds to the desired target sequence. Alternatively, a modified or engineered nuclease molecular cargo or DNA-binding protein can be employed. An “engineered nuclease molecular cargo or DNA-binding protein” includes a nuclease molecular cargo or DNA-binding protein that is engineered (modified or derived) from its native form to specifically recognize a desired target sequence. Thus, an engineered nuclease molecular cargo or DNA-binding protein can be derived from a native, naturally occurring nuclease molecular cargo or DNA-binding protein or it can be artificially created or synthesized. The engineered nuclease molecular cargo or DNA-binding protein can recognize a target sequence, for example, wherein the target sequence is not a sequence that would have been recognized by a native (non-engineered or non-modified) nuclease molecular cargo or DNA-binding protein. The modification of the nuclease molecular cargo or DNA-binding protein can be as little as one amino acid in a protein cleavage molecular cargo or one nucleotide in a nucleic acid cleavage molecular cargo. Producing a nick or double-strand break in a target sequence or other DNA can be referred to herein as “cutting” or “cleaving” the target sequence or other DNA.

Active variants and fragments of nuclease molecular cargoes or DNA-binding proteins (i.e., an engineered nuclease molecular cargo or DNA-binding protein) are also provided. Such active variants can comprise at least 65%, 70%, 75%, 80%, 85%, 90%, 91%, 92%, 93%, 94%, 95%, 96%, 97%, 98%, 99% or more sequence identity to the native nuclease molecular cargo or DNA-binding protein, wherein the active variants retain the ability to cut at a desired target sequence and hence retain nick or double-strand-break-inducing activity or retain the ability to bind a desired target sequence. For example, any of the nuclease molecular cargoes described herein can be modified from a native endonuclease sequence and designed to recognize and induce a nick or double-strand break at a target sequence that was not recognized by the native nuclease molecular cargo. Thus, some engineered nucleases have a specificity to induce a nick or double-strand break at a target sequence that is different from the corresponding native nuclease molecular cargo target sequence. Assays for nick or double-strand-break-inducing activity are known and generally measure the overall activity and specificity of the endonuclease on DNA substrates containing the target sequence. The target sequence can be endogenous (or native) to the cell or the target sequence can be exogenous to the cell. A target sequence that is exogenous to the cell is not naturally occurring in the genome of the cell. The target sequence can also exogenous to the polynucleotides of interest that one desires to be positioned at the target locus. In some cases, the target sequence is present only once in the genome of the host cell.

Active variants and fragments of the exemplified target sequences are also provided. Such active variants can comprise at least 65%, 70%, 75%, 80%, 85%, 90%, 91%, 92%, 93%, 94%, 95%, 96%, 97%, 98%, 99% or more sequence identity to the given target sequence, wherein the active variants retain biological activity and hence are capable of being recognized and cleaved by a nuclease molecular cargo in a sequence-specific manner. Assays to measure the double-strand break of a target sequence by a nuclease molecular cargo are known (e.g., TAQMAN® qPCR assay, Frendewey et al. (2010) Methods in Enzymology 476:295-307, herein incorporated by reference in its entirety for all purposes).

The length of the target sequence can vary, and includes, for example, target sequences that are about 30-36 bp for a zinc finger nuclease (ZFN) pair (about 15-18 bp for each ZFN), about 36 bp for a Transcription Activator-Like Effector (TALE) protein or Transcription Activator-Like Effector Nuclease (TALEN), or about 20 bp for a CRISPR/Cas9 guide RNA.

The target sequence of the DNA-binding protein or nuclease molecular cargo can be positioned anywhere in or near the target genomic locus. The target sequence can be located within a coding region of a gene, or within regulatory regions that influence the expression of the gene. A target sequence of the DNA-binding protein or nuclease molecular cargo can be located in an intron, an exon, a promoter, an enhancer, a regulatory region, or any non-protein coding region.

One type of DNA-binding protein that can be employed in the various methods and compositions disclosed herein is a Transcription Activator-Like Effector (TALE). A TALE can be fused or linked to, for example, an epigenetic modification domain, a transcriptional activation domain, or a transcriptional repressor domain. Examples of such domains are described with respect to Cas proteins, below, and can also be found, for example, in WO 2011/145121, herein incorporated by reference in its entirety for all purposes. Correspondingly, one type of nuclease molecular cargo that can be employed in the various methods and compositions disclosed herein is a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease such as Fokl. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See WO 2010/079430; Morbitzer et al. (2010) Proc. Natl. Acad. Sci. U.S.A. 107 (50:21617-21622; Scholze & Boch (2010) Virulence 1:428-432; Christian et al. (2010) Genetics 186:757-761; Li et al. (2011) Nucleic Acids Res. 39W: 359-372; and Miller et al. (2011) Nature Biotechnology 29: 143-148, each of which is herein incorporated by reference in its entirety for all purposes.

The non-specific DNA cleavage domain from the end of the Fokl endonuclease can be used to construct hybrid nucleases that are active in a yeast assay. These molecular cargoes are also active in plant cells and in animal cells. The Fokl domain functions as a dimer, requiring two constructs with unique DNA binding domains for sites in the target genome with proper orientation and spacing. Both the number of amino acid residues between the TALEN DNA binding domain and the Fokl cleavage domain and the number of bases between the two individual TALEN binding sites are parameters for achieving high levels of activity. The number of amino acid residues between the TALEN DNA binding domain and the Fokl cleavage domain may be modified by introduction of a spacer (distinct from the spacer sequence) between the plurality of TAL effector repeat sequences and the Fokl endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two-step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled, they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms.

Examples of suitable TAL nucleases, and methods for preparing suitable TAL nucleases, are disclosed, e.g., in US 2011/0239315 A1, US 2011/0269234 A1, US 2011/0145940 A1, US 2003/0232410 A1, US 2005/0208489 A1, US 2005/0026157 A1, US 2005/0064474 A1, US 2006/0188987 A1, and US 2006/0063231 A1, each of which is herein incorporated by reference in its entirety for all purposes. In some embodiments, TAL effector nucleases are engineered that cut in or near a target nucleic acid sequence in, for example, a genomic locus of interest, wherein the target nucleic acid sequence is at or near a sequence to be modified.

In some TALENs, each monomer of the TALEN comprises 33-35 TAL repeats that recognize a single base pair via two hypervariable residues. In some TALENs, the nuclease molecular cargo is a chimeric protein comprising a TAL-repeat-based DNA binding domain operably linked to an independent nuclease such as a Fokl endonuclease. For example, the nuclease molecular cargo can comprise a first TAL-repeat-based DNA binding domain and a second TAL-repeat-based DNA binding domain, wherein each of the first and the second TAL-repeat-based DNA binding domains is operably linked to a Fokl nuclease, wherein the first and the second TAL-repeat-based DNA binding domain recognize two contiguous target DNA sequences in each strand of the target DNA sequence separated by a spacer sequence of varying length (12-20 bp), and wherein the Fokl nuclease subunits dimerize to create an active nuclease that makes a double strand break at a target sequence.

Transcription Activator-Like Effector Nucleases (TALENs) are artificial restriction enzymes generated by fusing the TAL effector DNA binding domain to a DNA cleavage domain. These molecular cargoes enable efficient, programmable, and specific DNA cleavage and represent powerful tools for genome editing in situ. Transcription activator-like effectors (TALEs) can be quickly engineered to bind practically any DNA sequence. The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Pat. Nos. 8,586,363; 8,450,471; 8,440,431; 8,440,432; and 8,697,853, all of which are incorporated by reference herein in their entirety.

Another example of a DNA-binding protein is a zinc finger protein. Such zinc finger proteins can be linked or fused to, for example, an epigenetic modification domain, a transcriptional activation domain, or a transcriptional repressor domain. Examples of such domains are described with respect to Cas proteins, below, and can also be found, for example, in WO 2011/145121, herein incorporated by reference in its entirety for all purposes. Correspondingly, another example of a nuclease molecular cargo that can be employed in the various methods and compositions disclosed herein is a zinc-finger nuclease (ZFN). In some ZFNs, each monomer of the ZFN comprises three or more zinc finger-based DNA binding domains, wherein each zinc finger-based DNA binding domain binds to a 3 bp subsite. In other ZFNs, the ZFN is a chimeric protein comprising a zinc finger-based DNA binding domain operably linked to an independent nuclease such as a Fokl endonuclease. For example, the nuclease molecular cargo can comprise a first ZFN and a second ZFN, wherein each of the first ZFN and the second ZFN is operably linked to a Fokl nuclease subunit, wherein the first and the second ZFN recognize two contiguous target DNA sequences in each strand of the target DNA sequence separated by about 5-7 bp spacer, and wherein the Fokl nuclease subunits dimerize to create an active nuclease that makes a double strand break. See, e.g., US 2006/0246567; US 2008/0182332; US 2002/0081614; US 2003/0021776; WO 2002/057308 A2; US 2013/0123484; US 2010/0291048; WO 2011/017293 A2; and Gaj et al. (2013) Trends in Biotechnology 31(7):397-405, each of which is herein incorporated by reference in its entirety for all purposes.

### Viral Particles

In various embodiments, a molecular cargo of the present disclosure can include a viral particle or a viral capsid protein. An FGFR3 binding protein described herein can be conjugated to a viral particle to direct the viral particle to a desired cell type (e.g., astrocyte) in the central nervous system and/or in the eye. In some embodiments, conjugation with an FGFR3 binding protein alters the tropism of the viral particle (“retargeting”) or enhances the ability of the viral particle to target a desired cell type (e.g., astrocyte) in the central nervous system and/or in the eye. In some embodiments, the viral particle described herein is an adeno-associated virus (AAV). In some embodiments, the viral capsid protein described herein is an AAV capsid protein.

An FGFR3 binding protein described herein can be conjugated to the viral particle or viral capsid protein directly, or via, for example, a protein:protein binding pair. In some embodiments, a viral particle or a viral capsid protein conjugated to an FGFR3 binding protein described herein comprises: (i) a first member of a protein:protein binding pair inserted and/or displayed by the viral capsid, (ii) a second member of the protein:protein binding pair, wherein the first member of the protein:protein binding pair and the second member of the protein:protein binding pair are associated, and (iii) an antibody or antigen-binding fragment thereof that binds FGFR3, wherein the antibody or binding portion thereof is fused to the second member of the protein:protein binding pair. In some embodiments, (i) the first member of the protein:protein binding pair, (ii) the second member of the protein:protein binding pair, and (iii) the antibody or antigen-binding fragment thereof together redirect the tropism of the viral capsid to a cell that expresses FGFR3 (e.g., astrocyte).

“Retargeting” or “redirecting” may include a scenario in which the wildtype particle targets several cells within a tissue and/or several organs within an organism, and general targeting of the tissue or organs is reduced or abolished by insertion of the heterologous amino acid, and retargeting to more a specific cell in the tissue or a specific organ in the organism is achieved with the targeting ligand (e.g., via a targeting ligand) that binds a marker expressed by the specific cell. Such retargeting or redirecting may also include a scenario in which the wildtype particle targets a tissue, and targeting of the tissue is reduced to or abolished by insertion of the heterologous amino acid, and retargeting to a completely different tissue is achieved with the targeting ligand.

“Specific binding pair,” “protein:protein binding pair” and the like includes two proteins (e.g., a first member (e.g., a first polypeptide) and a second cognate member (e.g., a second polypeptide)) that interact to form a bond (e.g., a non-covalent bond between a first member epitope and a second member antigen-binding portion of an antibody that recognizes the epitope) or a covalent isopeptide bond under conditions that enable or facilitate bond formation. In some embodiments, the term “cognate” refers to components that function together. Epitopes and cognate antibodies thereto, particularly epitopes that may also act as a detectable label (e.g., c-myc) are well-known in the art. Specific protein:protein binding pairs capable of interacting to form a covalent isopeptide bond are reviewed in Veggiani et al. (2014) Trends Biotechnol. 32:506, and include peptide:peptide binding pairs such as SpyTag:SpyCatcher, SpyTag002:SpyCatcher002, SpyTag:KTag; isopeptag:pilin C, SnoopTag:SnoopCatcher, etc. Generally, a first member of a protein:protein binding pair refers to member of a protein:protein binding pair, which is generally less than 30 amino acids in length, and which forms a covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e. between two peptide/protein molecules, e.g. between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SpyTag:SpyCatcher system is described in U.S. Pat. No. 9,547,003 and Zaveri et al. (2012) PNAS 109: E690-E697, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK (SEQ ID NO: 273) which forms an amide bond to its cognate protein “SpyCatcher,” an 112 amino acid polypeptide having the amino acid sequence VDTLSGLSSEQGQSGDMTI EEDSATHIKFSKRDEDGKELAGA™ ELRDSSGKTISTWIS DGQVKDFYLYPGKYTFVETAAPDGYEVATAITFTVNEQGQVTVNGKATKGDAHI (SEQ ID NO: 274). (Zakeri (2012), supra). An additional specific binding pair derived from CnaB2 domain is SpyTag:KTag, which forms an isopeptide bond in the presence of SpyLigase. (Fierer (2014) PNAS 111: E1176-1181) SpyLigase (MSYYHHHHHHDYDGQSGDGKELAGA™ ELRDSSGKTISTWISDGQVKDFYLYPGKY TFVETAAPDGYEVATAITFTVNEQGQVTVNGKATKGGSGGSGGSGEDSATHI (SEQ ID NO: 275)) was engineered by excising the p strand from SpyCatcher that contains a reactive lysine, resulting in KTag, 10-residue first member of a protein:protein binding pair having the amino acid sequence ATHIKFSKRD (SEQ ID NO: 276). The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK (SEQ ID NO: 277), and binds SpyCatcher002 (VTTLSGLSGEQGPSGDMTTEEDSATHI KFSKRDEDGRELAGA™ ELRDSSGKTISTWI SDGHVKDFYLYPGKYTFVETAAPDGYEVATAITFTVNEQGQVTVNGEATKGDAHT (SEQ ID NO:278)).

The SnoopTag:SnoopCatcher system is described in Veggiani (2016) PNAS 113:1202-07. The D4 Ig-like domain of RrgA, an adhesion from Streptococcus pneumoniae, was split to form SnoopTag (residues 734-745; KLGDIEFIKVNK (SEQ ID NO: 279)) and SnoopCatcher (residues 749-860;

(SEQ ID NO: 280))
MGSSHHHHHHSSGLVPRGSHMKPLRGAVFS
LQKQHPDYPDIYGAIDQNGTYQNVRTGEDGKLTFKNLSDGKYRLFENSE
PAGYKPVQNKP IVAFQIVNGEVRDVTSIVPQDIPATYEFTNGKHYITN
EPIPPK.

Incubation of SnoopTag and SnoopCatcher results in a spontaneous isopeptide bond that is specific between the complementary proteins. Veggiani (2016)), supra.

The isopeptag:pilinC specific binding pair was derived from the major pilin protein Spy0128 from Streptococcus pyogenes. (Zakeir and Howarth (2010) J. Am. Chem. Soc. 132:4526-27). Isopeptag has the amino acid sequence TDKDMTITFTNKKDAE (SEQ ID NO: 281), and binds pilin-C (residues 18-299 of Spy0128). Incubation of SnoopTag and SnoopCatcher results in a spontaneous isopeptide bond that is specific between the complementary proteins. Zakeir and Howarth (2010), supra.

The term “detectable label” includes a polypeptide sequence that is a member of a specific binding pair, e.g., that specifically binds via a non-covalent bond with another polypeptide sequence, e.g., an antibody paratope, with high affinity. Exemplary and non-limiting detectable labels include hexahistidine tag (SEQ ID NO: 235), FLAG tag, Strep II tag, streptavidin-binding peptide (SBP) tag, calmodulin-binding peptide (CBP), glutathione S-transferase (GST), maltose-binding protein (MBP), S-tag, HA tag, and c-myc (EQKLISEEDL (SEQ ID NO: 282)). (Reviewed in Zhao et al. (2013) J. Analytical Meth. Chem. 1-8; incorporated herein by reference). A common detectable label for primate AAV is the B1 epitope (IGTRYLTR; SEQ ID NO: 283). Some AAV capsid proteins described herein, which do not naturally comprise the B1 epitope, may be modified herein to comprise a B1 epitope. Generally, AAV capsid proteins described herein may comprise a sequence with substantial homology to the B1 epitope within the last 10 amino acids of the capsid protein. Accordingly, in some embodiments, a non-primate AAV capsid protein of the invention may be modified with one but less than five point mutations within the last 10 amino acids of the capsid protein such that the AAV capsid protein comprises a B1 epitope.

Adeno-Associated Viruses (AAV)

“AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference. Table 1-2 herein provides sequences of various non-primate AAV.

TABLE 1-2
Example Non-primate AAVs

| | | | | |
|---|---|---|---|---|
| | | | Region | |
| | | | encoding the | |
| | | | non- | Region |
| | | | Structural/rep | encoding the |
| | | | protein | capsid protein |
| | | | (Accession | (Accession |
| | Accession | | Number/ | Number/ |
| | Number/ | | GI Number of | GI Number of |
| Description | GI Number | ITR | protein) | protein) |

Avian adeno-associated virus

| | | | | |
|---|---|---|---|---|
| Avian adeno-associated | GQ368252.1/ | | 243-2237 | 2255-4471 |
| vinis isolate YZ-1, | GI: 255710032 | | (ACU30843.1/ | (ACU30842.1/ |
| complete genome | | | GI: 255710033) | GI: 255710034) |
| Avian adeno-associated | KF937794.1/ | 1-142 | 244-2235 | 2253-4469 |
| virus isolate ZN1, | GI: 588284633 | 4540-4682 | (AHK22792.1/ | (AHK22793.1/ |
| complete genome | | | GI: 588284634) | GI: 588284635) |

Avian adeno-associated virus ATCC VR-865

| | | | | |
|---|---|---|---|---|
| Avian adeno-associated | AY186198.17/ | 1-142 | 244-2232 | 2250-4481 |
| virus ATCC VR-865, | GI: 31414777 | 4552-4694 | (AAO32086.1/ | (AAO32087.1/ |
| complete genome | | | GI: 31414778) | GI: 31414779) |
| Avian adeno-associated | NC_004828.1/ | 1-142 | 244-2232 | 2250-4481 |
| virs ATCC VR-865, | GI: 31543992 | 4552-4694 | (NP_852780.1/ | (NP_852781.1/ |
| complete genome | | | GI: 31543993) | GI: 31543994) |
| Avian adeno-associated | AY629582.1/ | 1-142 | 244-2232 | 2250-4483 |
| virus ATCC VR-865, | GI: 48996102 | 4552-4694 | (AAT48612.3/ | (AAT48613.1/ |
| complete genome | | | GI: 48996103) | GI: 48996104) |

Avian adeno-associated virus strain DA-1

| | | | | |
|---|---|---|---|---|
| Avian adeno-associated | AY629583.3/ | 1-142 | 244-2235 | 2253-4469 |
| virus strain DA-1, | GI: 48996105 | 4540-4682 | (AAT48614.1/ | (AAT48615.1/ |
| complete genome | | | GI: 48996106) | GI: 48996107) |
| Avian adeno-associated | NC_006263.1/ | 1-142 | 244-2235 | 2253-4469 |
| viras strain DA-1, | GI: 51949968 | 4540-4682 | (YP_077182.1/ | (YP_077183.1/ |
| complete genome | | | GI: 51949969) | GI: 51949970) |

Bat adeno-associated virus YNM

| | | | |
|---|---|---|---|
| Bat adeno-associated | GU226971.17/ | 200-2041 | 2059-4233 |
| virus YNM, complete | GI: 289719008 | (ADD17085.1/ | (ADD17086.1/ |
| sequence | | GI: 289719009) | GI: 289719010) |
| Bat adeno-associated | NC_014468.1/ | 200-2041 | 2059-4233 |
| virus YNM, complete | GI: 304422916 | (YP_003838571.1/ | (YP_003858572.1/ |
| genome | | GI: 304422917) | GI: 304422918) |

Bovine adeno-associated virus

| | | | |
|---|---|---|---|
| Bovine adeno- | AY388617.1/ | 368-2200 | 2216-4426 |
| associated virus, | GI: 38679253 | (AAR26464.1/ | (AAR26465.1/ |
| complete genome | | GI: 38679254) | GI: 38679255) |
| Bovine adeno- | NC_005889.1/ | 368-2200 | 2216-4426 |
| associated virus, | GI: 48696557 | (YP_024970.1/ | (YP_024971.1/ |
| complete genome | | GI: 48696558) | GI: 48696559) |

Bovine parvovirus-2

| | | | |
|---|---|---|---|
| Bovine parvovirus 2 | AF406966.1/ | 306-1919 | 2268-5384 |
| putative non-structural | GI: 15825370 | (AAL09671.1/ | (AAL09672.1/ |
| protein and putative | | GI: 15825371) | GI: 15825372) |
| capsid protein genes, | | | |
| complete cds. | | | |
| Bovine parvovirus 2, | NC_006259.1/ | 306-1919 | 2268-5384 |
| complete genome | GI: 51949957 | (YP_077175.1/ | (YP_077176.17 |
| | | GI: 51949958) | GI: 51949959) |

California sea lion adeno-associated virus 1

| | | | |
|---|---|---|---|
| Califomia sea lion | JN420371.1/ | 284-2086 | 2117-4273 |
| adeno-associated virus | GI: 343458966 | (AEM37641.1/ | (ABM37642.1/ |
| 1 isolate 1136 Rep78 | | GI: 343458967) | GI: 343458968) |
| and VP1 genes, | | | |
| complete cds. | | | |
| California sea lion | JN420372.1/ | 291-2093 | 2124-4280 |
| adeno-associated virus | GI: 343458969 | (AEM37643.1/ | (AEM37644.1/ |
| 1 isolate 1187 Rep78 | | GI: 343458970) | GI: 343458971) |
| and VP1 genes, | | | |
| complete cds. | | | |

Goose parvovirus

| | | | | |
|---|---|---|---|---|
| Goose parvovirus | (U25749.1/ | 1-444 | 537-2420 | 2439-4637 |
| virulent B strain, | GI: 1113795) | 4663-5106 | (AAA83229.1/ | (AAA83230.1/ |
| complete genome | | | GI: 1113796) | GI: 1113797) |
| | | | | 2874-4637 |
| | | | | (AAA83231.1/ |
| | | | | GI: 1113798) |
| | | | | 3033-4637 |
| | | | | (AAA83232.1/ |
| | | | | GI: 1113799) |
| Goose parvovirus, | (NC_001701.1/ | 1-444 | 537-2420 | 2439-4637 |
| complete genome | GI: 9628649 | 4663-5106 | (NP_043514.1/ | (NP_043515.1/ |
| | | | GI: 9628650) | GI: 9628651) |
| | | | | 2874-4637 |
| | | | | (NP_043516.1/ |
| | | | | GI: 9628652) |
| | | | | 3033-4637 |
| | | | | (NP_043517.1/ |
| | | | | GI: 9628653) |
| Goose parvovirus strain | (EU583389.1/ | 1-381 | 474-2357 | 2376-4574 |
| 82-0321V, complete | GI: 190888188) | 4600-4980 | (ACE95848.1/ | (ACE95849.1/ |
| genome | | | GI: 190888189) | GI: 190888190) |
| Goose parvovinis strain | (EU583390.1/ | 1-416 | 509-2392 | 2411-4609 |
| 82-0321, complete | GI: 190888191) | 4635-5050 | (ACE95850.1/ | (ACE95851.1/ |
| genome | | | GI: 190888192) | GI: 190888193) |
| Goose parvovirus strain | (EU583391.1/ | 1-418 | 511-2394 | 2413-4611 |
| 06-0329, complete | GI: 190888194) | 4637-5054 | (ACE95852.1/ | ACE95853.1/ |
| genome | | | GI: 190888195) | GI: 190888196) |
| Goose parvovirus strain | (EU583392.1/ | 1-443 | 536-2419 | 2438-4636 |
| VG32/1, complete | GI: 190888197) | 4662-5104 | (ACE95854.1/ | (ACE95855.1/ |
| genome | | | GI: 190888198) | GI: 190888199) |
| Goose parvovirus strain | (JF333590.1/ | 1-444 | 537-2420 | 2439-4637 |
| SH, complete genome | GI: 343113618) | 4663-5106 | (AEL87777.1/ | (AEL87778.1/ |
| | | | GI: 343113619) | GI: 343113620) |
| | | | | 2874-4637 |
| | | | | (AEL87779.1/ |
| | | | | GI: 343113621) |
| | | | | 3033-4637 |
| | | | | (AEL87780.17/ |
| | | | | GI: 343113622) |
| Goose parvovinis strain | (HQ891825.1/ | 1-444 | 537-2420 | 2439-4637 |
| GDaGPV, complete | GI: 359843307) | 4663-5106 | (ABV89789.1/ | (AEV89790.1/ |
| genome | | | GI: 359843308) | GI: 359843309) |
| | | | | 2874-4637 |
| | | | | (AEV89791.1/ |
| | | | | GI: 359843310) |
| | | | | 3033-4637 |
| | | | | (AEV89792.1/ |
| | | | | GI: 369843311) |
| Goose parvovirus strain | (KC478066.1/ | 1-416 | 509-2392 | 2411-4609 |
| SHFX1201, complete | GI: 459360514) | | (AGG56527.1/ | (AGG56328.1/ |
| genome | | | GI: 459360515) | GI: 459360516} |
| Goose parvovirus strain | (KC178571.1/ | 1-444 | 537-2420 | 2439-4637 |
| Y. complete genome | G1:513129761) | | (AGO17637.1/ | (AGO17638.1/ |
| | | | GI: 513129762) | GI: 513129763) |
| Goose parvovirus strain | (KC184133.1/ | 1-443 | 536-2419 | 2438-4636 |
| E. complete genome | GI: 513129764) | | (AGO17639.1/ | (AGO17640.1/ |
| | | | GI: 513129765) | GI: 513129766) |
| Goose parvovirus strain | (KC996729.1/ | 1-442 | 535-2418 | 2437-4635 |
| SYG61v, complete | GI: 531997217) | | (AGT62580.1/ | (AGT62581.1/ |
| genome | | | GI: 531997218) | GI: 531997219) |
| Goose parvovirus strain | (KC996730.1/ | 1-414 | 507-2390 | 2409-4607 |
| YZ99-6, complete | GI: 531997220) | | (AGT62582.1/ | (AGT62583.1/ |
| genome | | | GI: 531997221) | GI: 531997222) |
| Goose parvovinus | (AF416726.1/ | | 1-1884 | 1903-4101 |
| nonstructural protein | GI: 17226299) | | (AAL37721.1/ | (AAL37722.1/ |
| NS (ns) and capsid | | | GI: 17226300) | GI: 17226301) |
| protein VP (vp) genes, | | | 529-1884 | |
| complete cds. | | | (ABP93843.1/ | |
| | | | GI: 145694447) | |
| Goose parvovirus straitt | (EF515837.1/ | | 1-1884 | 1903-4101 |
| DY NS1 (NSD), NS2 | GI: 145694445) | | (ABP93842.1/ | (ABP82770.1/ |
| (NS2), VP1 (VP1). | | | GI: 145694446) | GI: 145573169) |
| VP2 (VP2), and VP3 | | | | 2338-4101 |
| (VP3) genes, complete | | | | (ABP93844.1/ |
| cds. | | | | GI: 145694448) |
| | | | | 2497-4101 |
| | | | | (ABP93845.1/ |
| | | | | GI: 145694449) |
| Goose parvovirus strain | (JF926695.1/ | | 9-1892 | 1911-4109 |
| PT NS1 protein and | GI: 354463162) | | (AER25356.1/ | (AER25357.1/ |
| VP1 protein genes, | | | GI: 354463163) | GI: 354463164) |
| complete cds. | | | | |
| Goose parvovirus strain | (JF926696.1/ | | 6-1889 | 1908-4106 |
| D NS1 protein and VP1 | GI: 354463165) | | (AER25358.1/ | (AER25359.1/ |
| protein genes, complete | | | GI: 354463166) | GI: 354463167) |
| cds. | | | | |

Mouse adeno-associated virus 1

| | | | |
|---|---|---|---|
| Mouse adeno- | DQ100362.1/ | 1-327 | 344-2485 |
| associated virus 1 rep | GI: 73665994 | (AAZ79671.1/ | (AAZ79672.1/ |
| gene, partial cds; and | | GI: 73665995) | GI: 73665996) |
| VP1 capsid, VP2 | | | 731-2485 |
| capsid, and VP3 capsid | | | (AAZ79673.1/ |
| genes, complete cds. | | | GI: 73665997) |
| | | | 911-2485 |
| | | | (AAZ79674.1/ |
| | | | GI: 73665998) |

Muscovy duck parvovirus

| | | | | |
|---|---|---|---|---|
| Barbarie duck | U22967.1/ | 1-457 | 548-2431 | 2450-4648 |
| parvovinis R.EP protein | GI: 1113784 | 4678-5132 | (AAA83224.1/ | (AAA83225.1/ |
| (rep) and three capsid | | | GI: 1113785) | GI: 1113786) |
| protein VP (vp) genes, | | | | 2885-4648 |
| complete cds | | | | (AAA83226.1/ |
| | | | | GI: 1113787) |
| | | | | 3044-4648 |
| | | | | (AAA83227.1/ |
| | | | | GI: 1113788) |
| Muscovy duck | NC_006147.2/ | 1-457 | 548-2431 | 2450-4648 |
| parvovirus, complete | GI: 51593841 | 4678-5132 | (YP_068410.1/ | (YP_068411.1/ |
| genome | | | GI: 51593842) | GI: 51593843) |
| | | | | 2885-4648 |
| | | | | (YP_068412.1/ |
| | | | | GI: 51593844) |
| | | | | 3044-4648 |
| | | | | (YP_068413.1/ |
| | | | | GI: 51593845) |
| Muscovy duck | JF926697.1/ | | 61-1944 | 1963-4161 |
| parvovirus strain P REP | GI: 354463168 | | (AER25360.1/ | (AER25361.1/ |
| protein and VP1 protein | | | GI: 354463169) | GI: 354463170) |
| genes, complete cds. | | | | |
| Muscovy duck | JF926698.1/ | | 61-1944 | 1963-4161 |
| parvovirus strain P1 | GI: 354463171 | | (AER25362.1/ | (AER25363.1/ |
| REP protein and VP1 | | | GI: 354463172) | GI: 354463173) |
| protein genes, complete | | | | |
| cds. | | | | |
| Muscovy duck | KC171936.1/ | 1-381 | 512-2395 | 2414-4612 |
| parvovirus isolate | GI: 459256867 | 4681-5061 | (AGG53766.1/ | (AGG53768.1/ |
| SAAS-SHNH, | | | GI: 459256868) | GI: 459256870) |
| complete genome | | | | 2849-4612 |
| | | | | (AGG53769.1/ |
| | | | | GI: 459256871) |
| | | | | 3008-4612 |
| | | | | (AGG53767.1/ |
| | | | | GI: 459256869) |

Serpentine adeno-associated virus 2

| | | | |
|---|---|---|---|
| Serpentine adeno- | EU872429.1/ | 1-642 | 661-1297 |
| associated vinis 2 non- | GI: 215401981 | (ACJ66590.1/ | (ACJ66591.1/ |
| structural protein 1 and | | GI: 215401982) | GI: 215401983) |
| capsid protein genes, | | | |
| partial cds | | | |

Snake parvovirus 1

| | | | |
|---|---|---|---|
| Snake parvovirus 1 | AY349010.1/ | 324-2012 | 2030-4210 |
| non-structural protein 1 | GI: 38017148 | (AAR07954.1/ | (AAR07955.1/ |
| (NS1) and capsid | | GI: 38017149) | GI: 38017150) |
| protein (VP1) genes, | | | |
| complete cds | | | |
| Snake parvovirus 1, | NC_006148.1/ | 324-2012 | 2030-4210 |
| complete genome | GI: 51555744 | (YP_068093.1/ | (YP_068094.1/ |
| | | GI: 51555745) | GI: 51555746) |

“AAV” encompasses all subtypes and both naturally occurring and modified forms, except where stated otherwise. AAV includes primate AAV (e.g., AAV type 1 (AAV1), primate AAV type 2 (AAV2), primate AAV type 3 (AAV3), primate AAV3B, primate AAV type 4 (AAV4), primate AAV type 5 (AAV5), primate AAV type 6 (AAV6), primate AAV6.2, primate AAV type 7 (AAV7), primate AAV type 8 (AAV8), primate AAV type 9 (AAV9), AAV10, AAV type hull (AAV hull), AAV11, AAV12, AAV13, AAVDJ, Anc80L65, AAV2G9, AAVLK03, AAV type rh32.33 (AAVrh.32.33), AAV retro (AAV retro), AAV PHP.B, AAV PHP.eB, AAV PHP.S, AAVrh.64R1, AAVhu.37, AAVrh.8, AAV2/8, etc.; non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc., “Primate AAV” refers to AAV generally isolated from primates. Similarly, “non-primate animal AAV” refers to AAV isolated from non-primate animals.

As used herein, “of a [specified] AAV” in relation to a gene (e.g., rep, cap, etc.), capsid protein (e.g., a VP1 capsid protein, a VP2 capsid protein, a VP3 capsid protein, etc.), region of a capsid protein of a specified AAV (e.g., PLA 2 region, VP1-u region, VP1/VP2 common region, VP3 region), nucleotide sequence (e.g., ITR sequence), e.g., a cap gene or capsid protein of AAV etc., encompasses, in addition to the gene or the polypeptide respectively comprising a nucleic acid sequence or amino acid sequence set forth herein for the specified AAV, also variants of the gene or polypeptide, including variants comprising the least number of nucleotides or amino acids required to retain one or more biological functions. As used herein, a variant gene or a variant polypeptide comprises a nucleic acid sequence or amino acid sequence that differs from the nucleic acid sequence or amino acid sequence set forth herein for the gene or polypeptide of a specified AAV, wherein the difference(s) does not generally alter at least one biological function of the gene or polypeptide, and/or the phylogenetic characterization of the gene or polypeptide, e.g., where the difference(s) may be due to degeneracy of the genetic code, isolate variations, length of the sequence, etc. For example, rep gene and the cap gene as used here may encompass rep and cap genes that differ from the wildtype gene in that the genes may encode one or more Rep proteins and Cap proteins, respectively. In some embodiments, a Rep gene encodes at least Rep78 and/or Rep68. In some embodiments, cap gene includes those may differ from the wildtype in that one or more alternative start codons or sequences between one or more alternative start codons are removed such that the cap gene encodes only a single Cap protein, e.g., wherein the VP2 and/or VP3 start codons are removed or substituted such that the cap gene encodes a functional VP1 capsid protein but not a VP2 capsid protein or a VP3 capsid protein. Accordingly, as used herein, a rep gene encompasses any sequence that encodes a functional Rep protein. A cap gene encompasses any sequence that encodes at least one functional cap gene.

It is well-known that the wildtype cap gene expresses all three VP1, VP2, and VP3 capsid proteins from a single open reading frame of the cap gene under control of the p40 promoter found in the rep ORF. The term “capsid protein,” “Cap protein” and the like includes a protein that is part of the capsid of the virus. For adeno-associated viruses, the capsid proteins are generally referred to as VP1, VP2 and/or VP3, and may be encoded by the single cap gene. For AAV, the three AAV capsid proteins are produced in nature an overlapping fashion from the cap ORF alternative translational start codon usage, although all three proteins use a common stop codon. The ORF of a wildtype cap gene encodes from 5′ to 3′ three alternative start codons: “the VP1 start codon,” “the VP2 start codon,” and “the VP3 start codon”; and one “common stop codon”. The largest viral protein, VP1, is generally encoded from the VP1 start codon to the “common stop codon.” VP2 is generally encoded from the VP2 start codon to the common stop codon. VP3 is generally encoded from the VP3 start codon to the common stop codon. Accordingly, VP1 comprises at its N-terminus sequence that it does not share with the VP2 or VP3, referred to as the VP1-unique region (VP1-u). The VP1-u region is generally encoded by the sequence of a wildtype cap gene starting from the VP1 start codon to the “VP2 start codon.” VP1-u comprises a phospholipase A2 domain (PLA 2), which may be important for infection, as well as nuclear localization signals which may aid the virus in targeting to the nucleus for uncoating and genome release. The VP1, VP2, and VP3 capsid proteins share the same C-terminal sequence that makes up the entirety of VP3, which may also be referred to herein as the VP3 region. The VP3 region is encoded from the VP3 start codon to the common stop codon. VP2 has an additional˜60 amino acids that it shares with the VP1. This region is called the VP1/VP2 common region.

In some embodiments, one or more of the Cap proteins of the invention may be encoded by one or more cap genes having one or more ORFs. In some embodiments, the VP proteins of the invention may be expressed from more than one ORF comprising nucleotide sequence encoding any combination of VP1, VP2, and/or VP3 by use of separate nucleotide sequences operably linked to at least one expression control sequence for expression in packaging cell, each producing one or more of VP1, VP2, and/or VP3 capsid proteins of the invention. In some embodiments, a VP capsid protein of the invention may be expressed individually from an ORF comprising nucleotide sequence encoding any one of VP1, VP2, or VP3 by use of separate nucleotide sequences operably linked to one expression control sequence for expression in a viral replication cell, each producing only one of VP1, VP2, or VP3 capsid protein. In another embodiment, VP proteins may be expressed from one ORF comprising nucleotide sequences encoding VP1, VP2, and VP3 capsid proteins operably linked to at least one expression control sequence for expression in a viral replication cell, each producing VP1, VP2, and VP3 capsid protein. Accordingly, although amino acid positions provided herein may be provided in relation to the VP1 capsid protein of the referenced AAV, a skilled artisan would be able to respectively and readily determine the position of that same amino acid within the VP2 and/or VP3 capsid protein of the AAV, and the corresponding position of amino acids among different AAV.

Non-limiting examples of wildtype and/or genetically modified nucleic acid sequences of cap genes and cap proteins useful for retargeting viral particles as described herein are set forth in SEQ ID NOs: 322-362.

>pAAV2-CAP wt (DNA, artificial) -
SEQ ID NO: 322
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGAGAGGCAACAGACAAGCAGCTACCG
CAGATGTCAACACACAAGGCGTTCTTCCAGGCATGGTCTGGCAGGACAGAGATGTGTACCTTCAGGGGCCC
ATCTGGGCAAAGATTCCACACACGGACGGACATTTTCACCCCTCTCCCCTCATGGGTGGATTCGGACTTAAA
CACCCTCCTCCACAGATTCTCATCAAGAACACCCCGGTACCTGCGAATCCTTCGACCACCTTCAGTGCGGCA
AAGTTTGCTTCCTTCATCACACAGTACTCCACGGGACAGGTCAGCGTGGAGATCGAGTGGGAGCTGCAGAA
GGAAAACAGCAAACGCTGGAATCCCGAAATTCAGTACACTTCCAACTACAACAAGTCTGTTAATGTGGACTTT
ACTGTGGACACTAATGGCGTGTATTCAGAGCCTCGCCCCATTGGCACCAGATACCTGACTCGTAATCTGTAA
>pAAV2-CAP wt (AA, Adeno-associated virus) -
SEQ ID NO: 323
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQRGN
RQAATADVNTQGVLPGMVWQDRDVYLQGPIWAKIPHTDGHFHPSPLMGGFGLKHPPPQILIKNTPVPANPSTTF
SAAKFASFITQYSTGQVSVEIEWELQKENSKRWNPEIQYTSNYNKSVNVDFTVDTNGVYSEPRPIGTRYLTRNL*
>pAAV2-CAP R585A R588A (HBM) (DNA, Artificial) -
SEQ ID NO: 324
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGGGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGCGCAAGCAGCTACCG
CAGATGTCAACACACAAGGCGTTCTTCCAGGCATGGTCTGGCAGGACAGAGATGTGTACCTTCAGGGGCCC
ATCTGGGCAAAGATTCCACACACGGACGGACATTTTCACCCCTCTCCCCTCATGGGTGGATTCGGACTTAAA
CACCCTCCTCCACAGATTCTCATCAAGAACACCCCGGTACCTGCGAATCCTTCGACCACCTTCAGTGCGGCA
AAGTTTGCTTCCTTCATCACACAGTACTCCACGGGACAGGTCAGCGTGGAGATCGAGTGGGAGCTGCAGAA
GGAAAACAGCAAACGCTGGAATCCCGAAATTCAGTACACTTCCAACTACAACAAGTCTGTTAATGTGGACTTT
ACTGTGGACACTAATGGCGTGTATTCAGAGCCTCGCCCCATTGGCACCAGATACCTGACTCGTAATCTGTAA
>pAAV2-CAP R585A R588A (HBM) (AA, Artificial) -
SEQ ID NO: 325
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
AQAATADVNTQGVLPGMVWQDRDVYLQGPIWAKIPHTDGHFHPSPLMGGFGLKHPPPQILIKNTPVPANPSTTF
SAAKFASFITQYSTGQVSVEIEWELQKENSKRWNPEIQYTSNYNKSVNVDFTVDTNGVYSEPRPIGTRYLTRNL*
>pAAV2-CAP N587 SpyTag HBM (DNA, Artificial) -
SEQ ID NO: 326
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGCCCACATCGTGATGG
TGGACGCCTACAAGCCGACGAAGCAAGCAGCTACCGCAGATGTCAACACACAAGGCGTTCTTCCAGGCATG
GTCTGGCAGGACAGAGATGTGTACCTTCAGGGGCCCATCTGGGCAAAGATTCCACACACGGACGGACATTT
TCACCCCTCTCCCCTCATGGGTGGATTCGGACTTAAACACCCTCCTCCACAGATTCTCATCAAGAACACCCC
GGTACCTGCGAATCCTTCGACCACCTTCAGTGCGGCAAAGTTTGCTTCCTTCATCACACAGTACTCCACGGG
ACAGGTCAGCGTGGAGATCGAGTGGGAGCTGCAGAAGGAAAACAGCAAACGCTGGAATCCCGAAATTCAGT
ACACTTCCAACTACAACAAGTCTGTTAATGTGGACTTTACTGTGGACACTAATGGCGTGTATTCAGAGCCTCG
CCCCATTGGCACCAGATACCTGACTCGTAATCTGTAA
>pAAV2-CAP N587 SpyTag HBM (AA, Artificial)-
SEQ ID NO: 327
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
AHIVMVDAYKPTKQAATADVNTQGVLPGMVWQDRDVYLQGPIWAKIPHTDGHFHPSPLMGGFGLKHPPPQILIK
NTPVPANPSTTFSAAKFASFITQYSTGQVSVEIEWELQKENSKRWNPEIQYTSNYNKSVNVDFTVDTNGVYSEPR
PIGTRYLTRNL*
>pAAV2-CAP N587 Linker 1 SpyTag HBM (DNA, Artificial) -
SEQ ID NO: 328
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGCAACGCGAGCGCCCACATC
GTGATGGTGGACGCCTACAAGCCGACGAAGGCGCAAGCAGCTACCGCAGATGTCAACACACAAGGCGTTC
TTCCAGGCATGGTCTGGCAGGACAGAGATGTGTACCTTCAGGGGCCCATCTGGGCAAAGATTCCACACACG
GACGGACATTTTCACCCCTCTCCCCTCATGGGTGGATTCGGACTTAAACACCCTCCTCCACAGATTCTCATC
AAGAACACCCCGGTACCTGCGAATCCTTCGACCACCTTCAGTGCGGCAAAGTTTGCTTCCTTCATCACACAG
TACTCCACGGGACAGGTCAGCGTGGAGATCGAGTGGGAGCTGCAGAAGGAAAACAGCAAACGCTGGAATC
CCGAAATTCAGTACACTTCCAACTACAACAAGTCTGTTAATGTGGACTTTACTGTGGACACTAATGGCGTGTA
TTCAGAGCCTCGCCCCATTGGCACCAGATACCTGACTCGTAATCTGTAA
>pAAV2-CAP N587 Linker 1 SpyTag HBM (AA, Artificial) -
SEQ ID NO: 329
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
ASAHIVMVDAYKPTKAQAATADVNTQGVLPGMVWQDRDVYLQGPIWAKIPHTDGHFHPSPLMGGFGLKHPPPQI
LIKNTPVPANPSTTFSAAKFASFITQYSTGQVSVEIEWELQKENSKRWNPEIQYTSNYNKSVNVDFTVDTNGVYSE
PRPIGTRYLTRNL*
>pAAV2-CAP N587 Linker 2 SpyTag HBM (DNA, Artificial) -
SEQ ID NO: 330
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGGCAGCGCCCACATC
GTGATGGTGGACGCCTACAAGCCGACGAAGGGCAGCCAAGCAGCTACCGCAGATGTCAACACACAAGGCG
TTCTTCCAGGCATGGTCTGGCAGGACAGAGATGTGTACCTTCAGGGGCCCATCTGGGCAAAGATTCCACAC
ACGGACGGACATTTTCACCCCTCTCCCCTCATGGGTGGATTCGGACTTAAACACCCTCCTCCACAGATTCTC
ATCAAGAACACCCCGGTACCTGCGAATCCTTCGACCACCTTCAGTGCGGCAAAGTTTGCTTCCTTCATCACA
CAGTACTCCACGGGACAGGTCAGCGTGGAGATCGAGTGGGAGCTGCAGAAGGAAAACAGCAAACGCTGGA
ATCCCGAAATTCAGTACACTTCCAACTACAACAAGTCTGTTAATGTGGACTTTACTGTGGACACTAATGGCGT
GTATTCAGAGCCTCGCCCCATTGGCACCAGATACCTGACTCGTAATCTGTAA
>pAAV2-CAP N587 Linker 2 SpyTag HBM (AA, Artificial) -
SEQ ID NO: 331
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
GSAHIVMVDAYKPTKGSQAATADVNTQGVLPGMVWQDRDVYLQGPIWAKIPHTDGHFHPSPLMGGFGLKHPPP
QILIKNTPVPANPSTTFSAAKFASFITQYSTGQVSVEIEWELQKENSKRWNPEIQYTSNYNKSVNVDFTVDTNGVY
SEPRPIGTRYLTRNL*
>pAAV2-CAP N587 Linker 4 SpyTag HBM (DNA, Artificial) -
SEQ ID NO: 332
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGGCCTGAGCGGCGCC
CACATCGTGATGGTGGACGCCTACAAGCCGACGAAGGGCCTGAGCGGCCAAGCAGCTACCGCAGATGTCA
ACACACAAGGCGTTCTTCCAGGCATGGTCTGGCAGGACAGAGATGTGTACCTTCAGGGGCCCATCTGGGCA
AAGATTCCACACACGGACGGACATTTTCACCCCTCTCCCCTCATGGGTGGATTCGGACTTAAACACCCTCCT
CCACAGATTCTCATCAAGAACACCCCGGTACCTGCGAATCCTTCGACCACCTTCAGTGCGGCAAAGTTTGCT
TCCTTCATCACACAGTACTCCACGGGACAGGTCAGCGTGGAGATCGAGTGGGAGCTGCAGAAGGAAAACAG
CAAACGCTGGAATCCCGAAATTCAGTACACTTCCAACTACAACAAGTCTGTTAATGTGGACTTTACTGTGGAC
ACTAATGGCGTGTATTCAGAGCCTCGCCCCATTGGCACCAGATACCTGACTCGTAATCTGTAA
pAAV2-CAP N587 Linker 4 SpyTag HBM (AA, Artificial) -
SEQ ID NO: 333
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
GLSGAHIVMVDAYKPTKGLSGQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNYNKSVNVDFTVDT
NGVYSEPRPIGTRYLTRNL*
>pAAV2-CAP N587 Linker 6 SpyTag HBM (DNA, Artificial) -
SEQ ID NO: 334
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGGGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGGCCTGAGCGGCAGC
GGCGCCCACATCGTGATGGTGGACGCCTACAAGCCGACGAAGGGCCTGAGCGGCAGCGGCCAAGCAGCT
ACCGCAGATGTCAACACACAAGGCGTTCTTCCAGGCATGGTCTGGCAGGACAGAGATGTGTACCTTCAGGG
GCCCATCTGGGCAAAGATTCCACACACGGACGGACATTTTCACCCCTCTCCCCTCATGGGTGGATTCGGAC
TTAAACACCCTCCTCCACAGATTCTCATCAAGAACACCCCGGTACCTGCGAATCCTTCGACCACCTTCAGTG
CGGCAAAGTTTGCTTCCTTCATCACACAGTACTCCACGGGACAGGTCAGCGTGGAGATCGAGTGGGAGCTG
CAGAAGGAAAACAGCAAACGCTGGAATCCCGAAATTCAGTACACTTCCAACTACAACAAGTCTGTTAATGTG
GACTTTACTGTGGACACTAATGGCGTGTATTCAGAGCCTCGCCCCATTGGCACCAGATACCTGACTCGTAAT
CTGTAA
>pAAV2-CAP N587 Linker 6 SpyTag HBM (AA, Artificial) -
SEQ ID NO: 335
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
GLSGSGAHIVMVDAYKPTKGLSGSGQAATADVNTQGVLPGMVWQDRDVYLQGPIWAKIPHTDGHFHPSPLMG
GFGLKHPPPQILIKNTPVPANPSTTFSAAKFASFITQYSTGQVSVEIEWELQKENSKRWNPEIQYTSNYNKSVNVD
FTVDTNGVYSEPRPIGTRYLTRNL*
>pAAV2-CAP N587 Linker 8 SpyTag HBM (DNA, Artificial)-
SEQ ID NO: 336
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGGCCTGAGCGGCCTG
AGCGGCAGCGCCCACATCGTGATGGTGGACGCCTACAAGCCGACGAAGGGCCTGAGCGGCCTGAGCGGC
AGCCAAGCAGCTACCGCAGATGTCAACACACAAGGCGTTCTTCCAGGCATGGTCTGGCAGGACAGAGATGT
GTACCTTCAGGGGCCCATCTGGGCAAAGATTCCACACACGGACGGACATTTTCACCCCTCTCCCCTCATGG
GTGGATTCGGACTTAAACACCCTCCTCCACAGATTCTCATCAAGAACACCCCGGTACCTGCGAATCCTTCGA
CCACCTTCAGTGCGGCAAAGTTTGCTTCCTTCATCACACAGTACTCCACGGGACAGGTCAGCGTGGAGATC
GAGTGGGAGCTGCAGAAGGAAAACAGCAAACGCTGGAATCCCGAAATTCAGTACACTTCCAACTACAACAA
GTCTGTTAATGTGGACTTTACTGTGGACACTAATGGCGTGTATTCAGAGCCTCGCCCCATTGGCACCAGATA
CCTGACTCGTAATCTGTAA
>pAAV2-CAP N587 Linker 8 SpyTag HBM (AA, Artificial) -
SEQ ID NO: 337
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
GLSGLSGSAHIVMVDAYKPTKGLSGLSGSQAATADVNTQGVLPGMVWQDRDVYLQGPIWAKIPHTDGHFHPSP
LMGGFGLKHPPPQILIKNTPVPANPSTTFSAAKFASFITQYSTGQVSVEIEWELQKENSKRWNPEIQYTSNYNKSV
NVDFTVDTNGVYSEPRPIGTRYLTRNL*
>pAAV2-CAP N587 Linker 10 SpyTag HBM (DNA, Artificial) -
SEQ ID NO: 338
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGGGGGGCAACGGCCTGAGCGGCCTG
AGCGGCCTGAGCGGCGCCCACATCGTGATGGTGGACGCCTACAAGCCGACGAAGGGCCTGAGCGGCCTG
AGCGGCCTGAGCGGCCAAGCAGCTACCGCAGATGTCAACACACAAGGCGTTCTTCCAGGCATGGTCTGGC
AGGACAGAGATGTGTACCTTCAGGGGCCCATCTGGGCAAAGATTCCACACACGGACGGACATTTTCACCCC
TCTCCCCTCATGGGTGGATTCGGACTTAAACACCCTCCTCCACAGATTCTCATCAAGAACACCCCGGTACCT
GCGAATCCTTCGACCACCTTCAGTGCGGCAAAGTTTGCTTCCTTCATCACACAGTACTCCACGGGACAGGTC
AGCGTGGAGATCGAGTGGGAGCTGCAGAAGGAAAACAGCAAACGCTGGAATCCCGAAATTCAGTACACTTC
CAACTACAACAAGTCTGTTAATGTGGACTTTACTGTGGACACTAATGGCGTGTATTCAGAGCCTCGCCCCAT
TGGCACCAGATACCTGACTCGTAATCTGTAA
>pAAV2-CAP N587 Linker 10 SpyTag HBM (AA, Artificial) -
SEQ ID NO: 339
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQAGN
GLSGLSGLSGAHIVMVDAYKPTKGLSGLSGLSGQAATADVNTQGVLPGMVWQDRDVYLQGPIWAKIPHTDGHF
HPSPLMGGFGLKHPPPQILIKNTPVPANPSTTFSAAKFASFITQYSTGQVSVEIEWELQKENSKRWNPEIQYTSNY
NKSVNVDFTVDTNGVYSEPRPIGTRYLTRNL*
>pAAV2-CAP G453 SpyTag HBMx5 (DNA, Artificial) -
SEQ ID NO: 340
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAGCCCACATCGTGATGGTGGACGCCTACAAGCCGACGAAGACCACCACGCAGTCAAGGCTTC
AGTTTTCTCAGGCCGGAGCGAGTGACATTCGGGACCAGTCTAGGAACTGGCTTCCTGGACCCTGTTACGCC
CAGCAGGCCGTATCAAAGACATCTGCGGATAACAACAACAGTGAATACTCGTGGACTGGAGCTACCAAGTA
CCACCTCAATGGCAGAGACTCTCTGGTGAATCCGGGCCCGGCCATGGCAAGCCACAAGGACGATGAAGAA
GCCTTTTTTCCTCAGAGCGGGGTTCTCATCTTTGGGAAGCAAGGCTCAGAGAAAACAAATGTGGACATTGAA
AAGGTCATGATTACAGACGAAGAGGAAATCAGGACAACCAATCCCGTGGCTACGGAGCAGTATGGTTCTGT
ATCTACCAACCTCCAGGCCGGCAACGCCCAAGCAGCTACCGCAGATGTCAACACACAAGGCGTTCTTCCAG
GCATGGTCTGGCAGGACAGAGATGTGTACCTTCAGGGGCCCATCTGGGCAAAGATTCCACACACGGACGG
ACATTTTCACCCCTCTCCCCTCATGGGTGGATTCGGACTTAAACACCCTCCTCCACAGATTCTCATCAAGAAC
ACCCCGGTACCTGCGAATCCTTCGACCACCTTCAGTGCGGCAAAGTTTGCTTCCTTCATCACACAGTACTCC
ACGGGACAGGTCAGCGTGGAGATCGAGTGGGAGCTGCAGAAGGAAAACAGCAAACGCTGGAATCCCGAAA
TTCAGTACACTTCCAACTACAACAAGTCTGTTAATGTGGACTTTACTGTGGACACTAATGGCGTGTATTCAGA
GCCTCGCCCCATTGGCACCAGATACCTGACTCGTAATCTGTAA
>pAAV2-CAP G453 SpyTag HBMx5 (AA, Artificial) -
SEQ ID NO: 341
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGAHIVMVDAYKPTKTTTQSRLQFSQAGASDIRDQSRNWLPGPCYAQQAVSKTSADNNNSEY
SWTGATKYHLNGRDSLVNPGPAMASHKDDEEAFFPQSGVLIFGKQGSEKTNVDIEKVMITDEEEIRTTNPVATEQ
YGSVSTNLQAGNAQAATADVNTQGVLPGMVWQDRDVYLQGPIWAKIPHTDGHFHPSPLMGGFGLKHPPPQILIK
NTPVPANPSTTFSAAKFASFITQYSTGQVSVEIEWELQKENSKRWNPEIQYTSNYNKSVNVDFTVDTNGVYSEPR
PIGTRYLTRNL*
>pAAV2-CAP G453 Linker10 SpyTag HBMx5 (DNA, Artificial) -
SEQ ID NO: 342
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAGGCCTGAGCGGCCTGAGCGGCCTGAGCGGCGCCCACATCGTGATGGTGGACGCCTACAAG
CCGACGAAGGGCCTGAGCGGCCTGAGCGGCCTGAGCGGCACCACCACGCAGTCAAGGCTTCAGTTTTCTC
AGGCCGGAGCGAGTGACATTCGGGACCAGTCTAGGAACTGGCTTCCTGGACCCTGTTACGCCCAGCAGGC
CGTATCAAAGACATCTGCGGATAACAACAACAGTGAATACTCGTGGACTGGAGCTACCAAGTACCACCTCAA
TGGCAGAGACTCTCTGGTGAATCCGGGCCCGGCCATGGCAAGCCACAAGGACGATGAAGAAGCCTTTTTTC
CTCAGAGCGGGGTTCTCATCTTTGGGAAGCAAGGCTCAGAGAAAACAAATGTGGACATTGAAAAGGTCATG
ATTACAGACGAAGAGGAAATCAGGACAACCAATCCCGTGGCTACGGAGCAGTATGGTTCTGTATCTACCAAC
CTCCAGGCCGGCAACGCCCAAGCAGCTACCGCAGATGTCAACACACAAGGCGTTCTTCCAGGCATGGTCTG
GCAGGACAGAGATGTGTACCTTCAGGGGCCCATCTGGGCAAAGATTCCACACACGGACGGACATTTTCACC
CCTCTCCCCTCATGGGTGGATTCGGACTTAAACACCCTCCTCCACAGATTCTCATCAAGAACACCCCGGTAC
CTGCGAATCCTTCGACCACCTTCAGTGCGGCAAAGTTTGCTTCCTTCATCACACAGTACTCCACGGGACAGG
TCAGCGTGGAGATCGAGTGGGAGCTGCAGAAGGAAAACAGCAAACGCTGGAATCCCGAAATTCAGTACACT
TCCAACTACAACAAGTCTGTTAATGTGGACTTTACTGTGGACACTAATGGCGTGTATTCAGAGCCTCGCCCC
ATTGGCACCAGATACCTGACTCGTAATCTGTAA
>pAAV2-CAP G453 Linker10 SpyTag HBMx5 (AA, Artificial) -
SEQ ID NO: 343
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGGLSGLSGLSGAHIVMVDAYKPTKGLSGLSGLSGTTTQSRLQFSQAGASDIRDQSRNWLP
GPCYAQQAVSKTSADNNNSEYSWTGATKYHLNGRDSLVNPGPAMASHKDDEEAFFPQSGVLIFGKQGSEKTNV
DIEKVMITDEEEIRTTNPVATEQYGSVSTNLQAGNAQAATADVNTQGVLPGMVWQDRDVYLQGPIWAKIPHTDG
HFHPSPLMGGFGLKHPPPQILIKNTPVPANPSTTFSAAKFASFITQYSTGQVSVEIEWELQKENSKRWNPEIQYTS
NYNKSVNVDFTVDTNGVYSEPRPIGTRYLTRNL*
>pAAV9-CAP wt (DNA Artificial)-
SEQ ID NO: 344
ATGGCTGCCGATGGTTATCTTCCAGATTGGCTCGAGGACAACCTTAGTGAAGGAATTCGCGAGTGGTGGGC
TTTGAAACCTGGAGCCCCTCAACCCAAGGCAAATCAACAACATCAAGACAACGCTCGAGGTCTTGTGCTTCC
GGGTTACAAATACCTTGGACCCGGCAACGGACTCGACAAGGGGGAGCCGGTCAACGCAGCAGACGCGGC
GGCCCTCGAGCACGACAAGGCCTACGACCAGCAGCTCAAGGCCGGAGACAACCCGTACCTCAAGTACAAC
CACGCCGACGCCGAGTTCCAGGAGCGGCTCAAAGAAGATACGTCTTTTGGGGGCAACCTCGGGCGAGCAG
TCTTCCAGGCCAAAAAGAGGCTTCTTGAACCTCTTGGTCTGGTTGAGGAAGCGGCTAAGACGGCTCCTGGA
AAGAAGAGGCCTGTAGAGCAGTCTCCTCAGGAACCGGACTCCTCCGCGGGTATTGGCAAATCGGGTGCAC
AGCCCGCTAAAAAGAGACTCAATTTCGGTCAGACTGGCGACACAGAGTCAGTCCCAGACCCTCAACCAATC
GGAGAACCTCCCGCAGCCCCCTCAGGTGTGGGATCTCTTACAATGGCTTCAGGTGGTGGCGCACCAGTGG
CAGACAATAACGAAGGTGCCGATGGAGTGGGTAGTTCCTCGGGAAATTGGCATTGCGATTCCCAATGGCTG
GGGGACAGAGTCATCACCACCAGCACCCGAACCTGGGCCCTGCCCACCTACAACAATCACCTCTACAAGCA
AATCTCCAACAGCACATCTGGAGGATCTTCAAATGACAACGCCTACTTCGGCTACAGCACCCCCTGGGGGT
ATTTTGACTTCAACAGATTCCACTGCCACTTCTCACCACGTGACTGGCAGCGACTCATCAACAACAACTGGG
GATTCCGGCCTAAGCGACTCAACTTCAAGCTCTTCAACATTCAGGTCAAAGAGGTTACGGACAACAATGGAG
TCAAGACCATCGCCAATAACCTTACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTTTTCATGATTCCTCAGTACGG
GTATCTGACGCTTAATGATGGAAGCCAGGCCGTGGGTCGTTCGTCCTTTTACTGCCTGGAATATTTCCCGTC
GCAAATGCTAAGAACGGGTAACAACTTCCAGTTCAGCTACGAGTTTGAGAACGTACCTTTCCATAGCAGCTA
CGCTCACAGCCAAAGCCTGGACCGACTAATGAATCCACTCATCGACCAATACTTGTACTATCTCTCAAAGAC
TATTAACGGTTCTGGACAGAATCAACAAACGCTAAAATTCAGTGTGGCCGGACCCAGCAACATGGCTGTCCA
GGGAAGAAACTACATACCTGGACCCAGCTACCGACAACAACGTGTCTCAACCACTGTGACTCAAAACAACAA
CAGCGAATTTGCTTGGCCTGGAGCTTCTTCTTGGGCTCTCAATGGACGTAATAGCTTGATGAATCCTGGACC
TGCTATGGCCAGCCACAAAGAAGGAGAGGACCGTTTCTTTCCTTTGTCTGGATCTTTAATTTTTGGCAAACAA
GGAACTGGAAGAGACAACGTGGATGCGGACAAAGTCATGATAACCAACGAAGAAGAAATTAAAACTACTAAC
CCGGTAGCAACGGAGTCCTATGGACAAGTGGCCACAAACCACCAGAGTGCCCAAGCACAGGCGCAGACCG
GCTGGGTTCAAAACCAAGGAATACTTCCGGGTATGGTTTGGCAGGACAGAGATGTGTACCTGCAAGGACCC
ATTTGGGCCAAAATTCCTCACACGGACGGCAACTTTCACCCTTCTCCGCTGATGGGAGGGTTTGGAATGAAG
CACCCGCCTCCTCAGATCCTCATCAAAAACACACCTGTACCTGCGGATCCTCCAACGGCCTTCAACAAGGAC
AAGCTGAACTCTTTCATCACCCAGTATTCTACTGGCCAAGTCAGCGTGGAGATCGAGTGGGAGCTGCAGAA
GGAAAACAGCAAGCGCTGGAACCCGGAGATCCAGTACACTTCCAACTATTACAAGTCTAATAATGTTGAATT
TGCTGTTAATACTGAAGGTGTATATAGTGAACCCCGCCCCATTGGCACCAGATACCTGACTCGTAATCTGTA
A
>pAAV9-CAP wt (AA Adeno-associated virus) -
SEQ ID NO: 345
MAADGYLPDWLEDNLSEGIREWWALKPGAPQPKANQQHQDNARGLVLPGYKYLGPGNGLDKGEPVNAADAAA
LEHDKAYDQQLKAGDNPYLKYNHADAEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAPSGVGSLTMASGGGAPVADNNEGAD
GVGSSSGNWHCDSQWLGDRVITTSTRTWALPTYNNHLYKQISNSTSGGSSNDNAYFGYSTPWGYFDFNRFHC
HFSPRDWQRLINNNWGFRPKRLNFKLFNIQVKEVTDNNGVKTIANNLTSTVQVFTDSDYQLPYVLGSAHEGCLPP
FPADVFMIPQYGYLTLNDGSQAVGRSSFYCLEYFPSQMLRTGNNFQFSYEFENVPFHSSYAHSQSLDRLMNPLI
DQYLYYLSKTINGSGQNQQTLKFSVAGPSNMAVQGRNYIPGPSYRQQRVSTTVTQNNNSEFAWPGASSWALNG
RNSLMNPGPAMASHKEGEDRFFPLSGSLIFGKQGTGRDNVDADKVMITNEEEIKTTNPVATESYGQVATNHQSA
QAQAQTGWVQNQGILPGMVWQDRDVYLQGPIWAKIPHTDGNFHPSPLMGGFGMKHPPPQILIKNTPVPADPPT
AFNKDKLNSFITQYSTGQVSVEIEWELQKENSKRWNPEIQYTSNYYKSNNVEFAVNTEGVYSEPRPIGTRYLTRN
L*
>pAAV9-CAP W503A (DNA, Artificial) -
SEQ ID NO: 346
ATGGCTGCCGATGGTTATCTTCCAGATTGGCTCGAGGACAACCTTAGTGAAGGAATTCGCGAGTGGTGGGC
TTTGAAACCTGGAGCCCCTCAACCCAAGGCAAATCAACAACATCAAGACAACGCTCGAGGTCTTGTGCTTCC
GGGTTACAAATACCTTGGACCCGGCAACGGACTCGACAAGGGGGAGCCGGTCAACGCAGCAGACGCGGC
GGCCCTCGAGCACGACAAGGCCTACGACCAGCAGCTCAAGGCCGGAGACAACCCGTACCTCAAGTACAAC
CACGCCGACGCCGAGTTCCAGGAGCGGCTCAAAGAAGATACGTCTTTTGGGGGCAACCTCGGGCGAGCAG
TCTTCCAGGCCAAAAAGAGGCTTCTTGAACCTCTTGGTCTGGTTGAGGAAGCGGCTAAGACGGCTCCTGGA
AAGAAGAGGCCTGTAGAGCAGTCTCCTCAGGAACCGGACTCCTCCGCGGGTATTGGCAAATCGGGTGCAC
AGCCCGCTAAAAAGAGACTCAATTTCGGTCAGACTGGCGACACAGAGTCAGTCCCAGACCCTCAACCAATC
GGAGAACCTCCCGCAGCCCCCTCAGGTGTGGGATCTCTTACAATGGCTTCAGGTGGTGGCGCACCAGTGG
CAGACAATAACGAAGGTGCCGATGGAGTGGGTAGTTCCTCGGGAAATTGGCATTGCGATTCCCAATGGCTG
GGGGACAGAGTCATCACCACCAGCACCCGAACCTGGGCCCTGCCCACCTACAACAATCACCTCTACAAGCA
AATCTCCAACAGCACATCTGGAGGATCTTCAAATGACAACGCCTACTTCGGCTACAGCACCCCCTGGGGGT
ATTTTGACTTCAACAGATTCCACTGCCACTTCTCACCACGTGACTGGCAGCGACTCATCAACAACAACTGGG
GATTCCGGCCTAAGCGACTCAACTTCAAGCTCTTCAACATTCAGGTCAAAGAGGTTACGGACAACAATGGAG
TCAAGACCATCGCCAATAACCTTACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTTTTCATGATTCCTCAGTACGG
GTATCTGACGCTTAATGATGGAAGCCAGGCCGTGGGTCGTTCGTCCTTTTACTGCCTGGAATATTTCCCGTC
GCAAATGCTAAGAACGGGTAACAACTTCCAGTTCAGCTACGAGTTTGAGAACGTACCTTTCCATAGCAGCTA
CGCTCACAGCCAAAGCCTGGACCGACTAATGAATCCACTCATCGACCAATACTTGTACTATCTCTCAAAGAC
TATTAACGGTTCTGGACAGAATCAACAAACGCTAAAATTCAGTGTGGCCGGACCCAGCAACATGGCTGTCCA
GGGAAGAAACTACATACCTGGACCCAGCTACCGACAACAACGTGTCTCAACCACTGTGACTCAAAACAACAA
CAGCGAATTTGCTGCTCCTGGAGCTTCTTCTTGGGCTCTCAATGGACGTAATAGCTTGATGAATCCTGGACC
TGCTATGGCCAGCCACAAAGAAGGAGAGGACCGTTTCTTTCCTTTGTCTGGATCTTTAATTTTTGGCAAACAA
GGAACTGGAAGAGACAACGTGGATGCGGACAAAGTCATGATAACCAACGAAGAAGAAATTAAAACTACTAAC
CCGGTAGCAACGGAGTCCTATGGACAAGTGGCCACAAACCACCAGAGTGCCCAAGCACAGGCGCAGACCG
GCTGGGTTCAAAACCAAGGAATACTTCCGGGTATGGTTTGGCAGGACAGAGATGTGTACCTGCAAGGACCC
ATTTGGGCCAAAATTCCTCACACGGACGGCAACTTTCACCCTTCTCCGCTGATGGGAGGGTTTGGAATGAAG
CACCCGCCTCCTCAGATCCTCATCAAAAACACACCTGTACCTGCGGATCCTCCAACGGCCTTCAACAAGGAC
AAGCTGAACTCTTTCATCACCCAGTATTCTACTGGCCAAGTCAGCGTGGAGATCGAGTGGGAGCTGCAGAA
GGAAAACAGCAAGCGCTGGAACCCGGAGATCCAGTACACTTCCAACTATTACAAGTCTAATAATGTTGAATT
TGCTGTTAATACTGAAGGTGTATATAGTGAACCCCGCCCCATTGGCACCAGATACCTGACTCGTAATCTGTA
A
>pAAV9-CAP W503A (AA, Artificial) -
SEQ ID NO: 347
MAADGYLPDWLEDNLSEGIREWWALKPGAPQPKANQQHQDNARGLVLPGYKYLGPGNGLDKGEPVNAADAAA
LEHDKAYDQQLKAGDNPYLKYNHADAEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAPSGVGSLTMASGGGAPVADNNEGAD
GVGSSSGNWHCDSQWLGDRVITTSTRTWALPTYNNHLYKQISNSTSGGSSNDNAYFGYSTPWGYFDFNRFHC
HFSPRDWQRLINNNWGFRPKRLNFKLFNIQVKEVTDNNGVKTIANNLTSTVQVFTDSDYQLPYVLGSAHEGCLPP
FPADVFMIPQYGYLTLNDGSQAVGRSSFYCLEYFPSQMLRTGNNFQFSYEFENVPFHSSYAHSQSLDRLMNPLI
DQYLYYLSKTINGSGQNQQTLKFSVAGPSNMAVQGRNYIPGPSYRQQRVSTTVTQNNNSEFAAPGASSWALNG
RNSLMNPGPAMASHKEGEDRFFPLSGSLIFGKQGTGRDNVDADKVMITNEEEIKTTNPVATESYGQVATNHQSA
QAQAQTGWVQNQGILPGMVWQDRDVYLQGPIWAKIPHTDGNFHPSPLMGGFGMKHPPPQILIKNTPVPADPPT
AFNKDKLNSFITQYSTGQVSVEIEWELQKENSKRWNPEIQYTSNYYKSNNVEFAVNTEGVYSEPRPIGTRYLTRN
L*
>pAAV9-CAP N272A (DNA, Artificial) -
SEQ ID NO: 348
ATGGCTGCCGATGGTTATCTTCCAGATTGGCTCGAGGACAACCTTAGTGAAGGAATTCGCGAGTGGTGGGC
TTTGAAACCTGGAGCCCCTCAACCCAAGGCAAATCAACAACATCAAGACAACGCTCGAGGTCTTGTGCTTCC
GGGTTACAAATACCTTGGACCCGGCAACGGACTCGACAAGGGGGAGCCGGTCAACGCAGCAGACGCGGC
GGCCCTCGAGCACGACAAGGCCTACGACCAGCAGCTCAAGGCCGGAGACAACCCGTACCTCAAGTACAAC
CACGCCGACGCCGAGTTCCAGGAGCGGCTCAAAGAAGATACGTCTTTTGGGGGCAACCTCGGGCGAGCAG
TCTTCCAGGCCAAAAAGAGGCTTCTTGAACCTCTTGGTCTGGTTGAGGAAGCGGCTAAGACGGCTCCTGGA
AAGAAGAGGCCTGTAGAGCAGTCTCCTCAGGAACCGGACTCCTCCGCGGGTATTGGCAAATCGGGTGCAC
AGCCCGCTAAAAAGAGACTCAATTTCGGTCAGACTGGCGACACAGAGTCAGTCCCAGACCCTCAACCAATC
GGAGAACCTCCCGCAGCCCCCTCAGGTGTGGGATCTCTTACAATGGCTTCAGGTGGTGGCGCACCAGTGG
CAGACAATAACGAAGGTGCCGATGGAGTGGGTAGTTCCTCGGGAAATTGGCATTGCGATTCCCAATGGCTG
GGGGACAGAGTCATCACCACCAGCACCCGAACCTGGGCCCTGCCCACCTACAACAATCACCTCTACAAGCA
AATCTCCAACAGCACATCTGGAGGATCTTCAAATGACGCCGCCTACTTCGGCTACAGCACCCCCTGGGGGT
ATTTTGACTTCAACAGATTCCACTGCCACTTCTCACCACGTGACTGGCAGCGACTCATCAACAACAACTGGG
GATTCCGGCCTAAGCGACTCAACTTCAAGCTCTTCAACATTCAGGTCAAAGAGGTTACGGACAACAATGGAG
TCAAGACCATCGCCAATAACCTTACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTTTTCATGATTCCTCAGTACGG
GTATCTGACGCTTAATGATGGAAGCCAGGCCGTGGGTCGTTCGTCCTTTTACTGCCTGGAATATTTCCCGTC
GCAAATGCTAAGAACGGGTAACAACTTCCAGTTCAGCTACGAGTTTGAGAACGTACCTTTCCATAGCAGCTA
CGCTCACAGCCAAAGCCTGGACCGACTAATGAATCCACTCATCGACCAATACTTGTACTATCTCTCAAAGAC
TATTAACGGTTCTGGACAGAATCAACAAACGCTAAAATTCAGTGTGGCCGGACCCAGCAACATGGCTGTCCA
GGGAAGAAACTACATACCTGGACCCAGCTACCGACAACAACGTGTCTCAACCACTGTGACTCAAAACAACAA
CAGCGAATTTGCTTGGCCTGGAGCTTCTTCTTGGGCTCTCAATGGACGTAATAGCTTGATGAATCCTGGACC
TGCTATGGCCAGCCACAAAGAAGGAGAGGACCGTTTCTTTCCTTTGTCTGGATCTTTAATTTTTGGCAAACAA
GGAACTGGAAGAGACAACGTGGATGCGGACAAAGTCATGATAACCAACGAAGAAGAAATTAAAACTACTAAC
CCGGTAGCAACGGAGTCCTATGGACAAGTGGCCACAAACCACCAGAGTGCCCAAGCACAGGCGCAGACCG
GCTGGGTTCAAAACCAAGGAATACTTCCGGGTATGGTTTGGCAGGACAGAGATGTGTACCTGCAAGGACCC
ATTTGGGCCAAAATTCCTCACACGGACGGCAACTTTCACCCTTCTCCGCTGATGGGAGGGTTTGGAATGAAG
CACCCGCCTCCTCAGATCCTCATCAAAAACACACCTGTACCTGCGGATCCTCCAACGGCCTTCAACAAGGAC
AAGCTGAACTCTTTCATCACCCAGTATTCTACTGGCCAAGTCAGCGTGGAGATCGAGTGGGAGCTGCAGAA
GGAAAACAGCAAGCGCTGGAACCCGGAGATCCAGTACACTTCCAACTATTACAAGTCTAATAATGTTGAATT
TGCTGTTAATACTGAAGGTGTATATAGTGAACCCCGCCCCATTGGCACCAGATACCTGACTCGTAATCTGTA
A
>pAAV9-CAP N272A (AA, Artificial) -
SEQ ID NO: 349
MAADGYLPDWLEDNLSEGIREWWALKPGAPQPKANQQHQDNARGLVLPGYKYLGPGNGLDKGEPVNAADAAA
LEHDKAYDQQLKAGDNPYLKYNHADAEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAPSGVGSLTMASGGGAPVADNNEGAD
GVGSSSGNWHCDSQWLGDRVITTSTRTWALPTYNNHLYKQISNSTSGGSSNDAAYFGYSTPWGYFDFNRFHC
HFSPRDWQRLINNNWGFRPKRLNFKLFNIQVKEVTDNNGVKTIANNLTSTVQVFTDSDYQLPYVLGSAHEGCLPP
FPADVFMIPQYGYLTLNDGSQAVGRSSFYCLEYFPSQMLRTGNNFQFSYEFENVPFHSSYAHSQSLDRLMNPLI
DQYLYYLSKTINGSGQNQQTLKFSVAGPSNMAVQGRNYIPGPSYRQQRVSTTVTQNNNSEFAWPGASSWALNG
RNSLMNPGPAMASHKEGEDRFFPLSGSLIFGKQGTGRDNVDADKVMITNEEEIKTTNPVATESYGQVATNHQSA
QAQAQTGWVQNQGILPGMVWQDRDVYLQGPIWAKIPHTDGNFHPSPLMGGFGMKHPPPQILIKNTPVPADPPT
AFNKDKLNSFITQYSTGQVSVEIEWELQKENSKRWNPEIQYTSNYYKSNNVEFAVNTEGVYSEPRPIGTRYLTRN
L*
>pAAV9-CAP A589 SpyTag W503A (DNA, Artificial) -
SEQ ID NO: 350
ATGGCTGCCGATGGTTATCTTCCAGATTGGCTCGAGGACAACCTTAGTGAAGGAATTCGCGAGTGGTGGGC
TTTGAAACCTGGAGCCCCTCAACCCAAGGCAAATCAACAACATCAAGACAACGCTCGAGGTCTTGTGCTTCC
GGGTTACAAATACCTTGGACCCGGCAACGGACTCGACAAGGGGGAGCCGGTCAACGCAGCAGACGCGGC
GGCCCTCGAGCACGACAAGGCCTACGACCAGCAGCTCAAGGCCGGAGACAACCCGTACCTCAAGTACAAC
CACGCCGACGCCGAGTTCCAGGAGCGGCTCAAAGAAGATACGTCTTTTGGGGGCAACCTCGGGCGAGCAG
TCTTCCAGGCCAAAAAGAGGCTTCTTGAACCTCTTGGTCTGGTTGAGGAAGCGGCTAAGACGGCTCCTGGA
AAGAAGAGGCCTGTAGAGCAGTCTCCTCAGGAACCGGACTCCTCCGCGGGTATTGGCAAATCGGGTGCAC
AGCCCGCTAAAAAGAGACTCAATTTCGGTCAGACTGGCGACACAGAGTCAGTCCCAGACCCTCAACCAATC
GGAGAACCTCCCGCAGCCCCCTCAGGTGTGGGATCTCTTACAATGGCTTCAGGTGGTGGCGCACCAGTGG
CAGACAATAACGAAGGTGCCGATGGAGTGGGTAGTTCCTCGGGAAATTGGCATTGCGATTCCCAATGGCTG
GGGGACAGAGTCATCACCACCAGCACCCGAACCTGGGCCCTGCCCACCTACAACAATCACCTCTACAAGCA
AATCTCCAACAGCACATCTGGAGGATCTTCAAATGACAACGCCTACTTCGGCTACAGCACCCCCTGGGGGT
ATTTTGACTTCAACAGATTCCACTGCCACTTCTCACCACGTGACTGGCAGCGACTCATCAACAACAACTGGG
GATTCCGGCCTAAGCGACTCAACTTCAAGCTCTTCAACATTCAGGTCAAAGAGGTTACGGACAACAATGGAG
TCAAGACCATCGCCAATAACCTTACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTTTTCATGATTCCTCAGTACGG
GTATCTGACGCTTAATGATGGAAGCCAGGCCGTGGGTCGTTCGTCCTTTTACTGCCTGGAATATTTCCCGTC
GCAAATGCTAAGAACGGGTAACAACTTCCAGTTCAGCTACGAGTTTGAGAACGTACCTTTCCATAGCAGCTA
CGCTCACAGCCAAAGCCTGGACCGACTAATGAATCCACTCATCGACCAATACTTGTACTATCTCTCAAAGAC
TATTAACGGTTCTGGACAGAATCAACAAACGCTAAAATTCAGTGTGGCCGGACCCAGCAACATGGCTGTCCA
GGGAAGAAACTACATACCTGGACCCAGCTACCGACAACAACGTGTCTCAACCACTGTGACTCAAAACAACAA
CAGCGAATTTGCTGCTCCTGGAGCTTCTTCTTGGGCTCTCAATGGACGTAATAGCTTGATGAATCCTGGACC
TGCTATGGCCAGCCACAAAGAAGGAGAGGACCGTTTCTTTCCTTTGTCTGGATCTTTAATTTTTGGCAAACAA
GGAACTGGAAGAGACAACGTGGATGCGGACAAAGTCATGATAACCAACGAAGAAGAAATTAAAACTACTAAC
CCGGTAGCAACGGAGTCCTATGGACAAGTGGCCACAAACCACCAGAGTGCCCAAGCAGCCCACATCGTGAT
GGTGGACGCCTACAAGCCGACGAAGCAGGCGCAGACCGGCTGGGTTCAAAACCAAGGAATACTTCCGGGT
ATGGTTTGGCAGGACAGAGATGTGTACCTGCAAGGACCCATTTGGGCCAAAATTCCTCACACGGACGGCAA
CTTTCACCCTTCTCCGCTGATGGGAGGGTTTGGAATGAAGCACCCGCCTCCTCAGATCCTCATCAAAAACAC
ACCTGTACCTGCGGATCCTCCAACGGCCTTCAACAAGGACAAGCTGAACTCTTTCATCACCCAGTATTCTAC
TGGCCAAGTCAGCGTGGAGATCGAGTGGGAGCTGCAGAAGGAAAACAGCAAGCGCTGGAACCCGGAGATC
CAGTACACTTCCAACTATTACAAGTCTAATAATGTTGAATTTGCTGTTAATACTGAAGGTGTATATAGTGAACC
CCGCCCCATTGGCACCAGATACCTGACTCGTAATCTGTAA
>pAAV9-CAP A589 SpyTag W503A (AA, Artificial) -
SEQ ID NO: 351
MAADGYLPDWLEDNLSEGIREWWALKPGAPQPKANQQHQDNARGLVLPGYKYLGPGNGLDKGEPVNAADAAA
LEHDKAYDQQLKAGDNPYLKYNHADAEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAPSGVGSLTMASGGGAPVADNNEGAD
GVGSSSGNWHCDSQWLGDRVITTSTRTWALPTYNNHLYKQISNSTSGGSSNDNAYFGYSTPWGYFDFNRFHC
HFSPRDWQRLINNNWGFRPKRLNFKLFNIQVKEVTDNNGVKTIANNLTSTVQVFTDSDYQLPYVLGSAHEGCLPP
FPADVFMIPQYGYLTLNDGSQAVGRSSFYCLEYFPSQMLRTGNNFQFSYEFENVPFHSSYAHSQSLDRLMNPLI
DQYLYYLSKTINGSGQNQQTLKFSVAGPSNMAVQGRNYIPGPSYRQQRVSTTVTQNNNSEFAAPGASSWALNG
RNSLMNPGPAMASHKEGEDRFFPLSGSLIFGKQGTGRDNVDADKVMITNEEEIKTTNPVATESYGQVATNHQSA
QAAHIVMVDAYKPTKQAQTGWVQNQGILPGMVWQDRDVYLQGPIWAKIPHTDGNFHPSPLMGGFGMKHPPPQI
LIKNTPVPADPPTAFNKDKLNSFITQYSTGQVSVEIEWELQKENSKRWNPEIQYTSNYYKSNNVEFAVNTEGVYS
EPRPIGTRYLTRNL*
>pAAV9-CAP A589 Linker10 SpyTag W503A (DNA, Artificial) -
SEQ ID NO: 352
ATGGCTGCCGATGGTTATCTTCCAGATTGGCTCGAGGACAACCTTAGTGAAGGAATTCGCGAGTGGTGGGC
TTTGAAACCTGGAGCCCCTCAACCCAAGGCAAATCAACAACATCAAGACAACGCTCGAGGTCTTGTGCTTCC
GGGTTACAAATACCTTGGACCCGGCAACGGACTCGACAAGGGGGAGCCGGTCAACGCAGCAGACGCGGC
GGCCCTCGAGCACGACAAGGCCTACGACCAGCAGCTCAAGGCCGGAGACAACCCGTACCTCAAGTACAAC
CACGCCGACGCCGAGTTCCAGGAGCGGCTCAAAGAAGATACGTCTTTTGGGGGCAACCTCGGGCGAGCAG
TCTTCCAGGCCAAAAAGAGGCTTCTTGAACCTCTTGGTCTGGTTGAGGAAGCGGCTAAGACGGCTCCTGGA
AAGAAGAGGCCTGTAGAGCAGTCTCCTCAGGAACCGGACTCCTCCGCGGGTATTGGCAAATCGGGTGCAC
AGCCCGCTAAAAAGAGACTCAATTTCGGTCAGACTGGCGACACAGAGTCAGTCCCAGACCCTCAACCAATC
GGAGAACCTCCCGCAGCCCCCTCAGGTGTGGGATCTCTTACAATGGCTTCAGGTGGTGGCGCACCAGTGG
CAGACAATAACGAAGGTGCCGATGGAGTGGGTAGTTCCTCGGGAAATTGGCATTGCGATTCCCAATGGCTG
GGGGACAGAGTCATCACCACCAGCACCCGAACCTGGGCCCTGCCCACCTACAACAATCACCTCTACAAGCA
AATCTCCAACAGCACATCTGGAGGATCTTCAAATGACAACGCCTACTTCGGCTACAGCACCCCCTGGGGGT
ATTTTGACTTCAACAGATTCCACTGCCACTTCTCACCACGTGACTGGCAGCGACTCATCAACAACAACTGGG
GATTCCGGCCTAAGCGACTCAACTTCAAGCTCTTCAACATTCAGGTCAAAGAGGTTACGGACAACAATGGAG
TCAAGACCATCGCCAATAACCTTACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTTTTCATGATTCCTCAGTACGG
GTATCTGACGCTTAATGATGGAAGCCAGGCCGTGGGTCGTTCGTCCTTTTACTGCCTGGAATATTTCCCGTC
GCAAATGCTAAGAACGGGTAACAACTTCCAGTTCAGCTACGAGTTTGAGAACGTACCTTTCCATAGCAGCTA
CGCTCACAGCCAAAGCCTGGACCGACTAATGAATCCACTCATCGACCAATACTTGTACTATCTCTCAAAGAC
TATTAACGGTTCTGGACAGAATCAACAAACGCTAAAATTCAGTGTGGCCGGACCCAGCAACATGGCTGTCCA
GGGAAGAAACTACATACCTGGACCCAGCTACCGACAACAACGTGTCTCAACCACTGTGACTCAAAACAACAA
CAGCGAATTTGCTGCTCCTGGAGCTTCTTCTTGGGCTCTCAATGGACGTAATAGCTTGATGAATCCTGGACC
TGCTATGGCCAGCCACAAAGAAGGAGAGGACCGTTTCTTTCCTTTGTCTGGATCTTTAATTTTTGGCAAACAA
GGAACTGGAAGAGACAACGTGGATGCGGACAAAGTCATGATAACCAACGAAGAAGAAATTAAAACTACTAAC
CCGGTAGCAACGGAGTCCTATGGACAAGTGGCCACAAACCACCAGAGTGCCCAAGCAGGCCTGAGCAGCG
GCAGCGGCCTGAGCGGCGCCCACATCGTGATGGTGGACGCCTACAAGCCGACGAAGGGCCTGAGCGGCG
GCAGCGGCCTGAGCGGCCAGGCGCAGACCGGCTGGGTTCAAAACCAAGGAATACTTCCGGGTATGGTTTG
GCAGGACAGAGATGTGTACCTGCAAGGACCCATTTGGGCCAAAATTCCTCACACGGACGGCAACTTTCACC
CTTCTCCGCTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTACTGGCCAAG
TCAGCGTGGAGATCGAGTGGGAGCTGCAGAAGGAAAACAGCAAGCGCTGGAACCCGGAGATCCAGTACAC
TTCCAACTATTACAAGTCTAATAATGTTGAATTTGCTGTTAATACTGAAGGTGTATATAGTGAACCCCGCCCC
ATTGGCACCAGATACCTGACTCGTAATCTGTAA
>pAAV9-CAP A589 Linker10 SpyTag W503A (AA, Artificial) -
SEQ ID NO: 353
MAADGYLPDWLEDNLSEGIREWWALKPGAPQPKANQQHQDNARGLVLPGYKYLGPGNGLDKGEPVNAADAAA
LEHDKAYDQQLKAGDNPYLKYNHADAEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAPSGVGSLTMASGGGAPVADNNEGAD
GVGSSSGNWHCDSQWLGDRVITTSTRTWALPTYNNHLYKQISNSTSGGSSNDNAYFGYSTPWGYFDFNRFHC
HFSPRDWQRLINNNWGFRPKRLNFKLFNIQVKEVTDNNGVKTIANNLTSTVQVFTDSDYQLPYVLGSAHEGCLPP
FPADVFMIPQYGYLTLNDGSQAVGRSSFYCLEYFPSQMLRTGNNFQFSYEFENVPFHSSYAHSQSLDRLMNPLI
DQYLYYLSKTINGSGQNQQTLKFSVAGPSNMAVQGRNYIPGPSYRQQRVSTTVTQNNNSEFAAPGASSWALNG
RNSLMNPGPAMASHKEGEDRFFPLSGSLIFGKQGTGRDNVDADKVMITNEEEIKTTNPVATESYGQVATNHQSA
QAGLSSGSGLSGAHIVMVDAYKPTKGLSGGSGLSGQAQTGWVQNQGILPGMVWQDRDVYLQGPIWAKIPHTD
GNFHPSPLMGGFGMKHPPPQILIKNTPVPADPPTAFNKDKLNSFITQYSTGQVSVEIEWELQKENSKRWNPEIQY
TSNYYKSNNVEFAVNTEGVYSEPRPIGTRYLTRNL*
>pAAV9-CAP G453 Linker10 SpyTag W503A (DNA, Artificial) -
SEQ ID NO: 354
ATGGCTGCCGATGGTTATCTTCCAGATTGGCTCGAGGACAACCTTAGTGAAGGAATTCGCGAGTGGTGGGC
TTTGAAACCTGGAGCCCCTCAACCCAAGGCAAATCAACAACATCAAGACAACGCTCGAGGTCTTGTGCTTCC
GGGTTACAAATACCTTGGACCCGGCAACGGACTCGACAAGGGGGAGCCGGTCAACGCAGCAGACGCGGC
GGCCCTCGAGCACGACAAGGCCTACGACCAGCAGCTCAAGGCCGGAGACAACCCGTACCTCAAGTACAAC
CACGCCGACGCCGAGTTCCAGGAGCGGCTCAAAGAAGATACGTCTTTTGGGGGCAACCTCGGGCGAGCAG
TCTTCCAGGCCAAAAAGAGGCTTCTTGAACCTCTTGGTCTGGTTGAGGAAGCGGCTAAGACGGCTCCTGGA
AAGAAGAGGCCTGTAGAGCAGTCTCCTCAGGAACCGGACTCCTCCGCGGGTATTGGCAAATCGGGTGCAC
AGCCCGCTAAAAAGAGACTCAATTTCGGTCAGACTGGCGACACAGAGTCAGTCCCAGACCCTCAACCAATC
GGAGAACCTCCCGCAGCCCCCTCAGGTGTGGGATCTCTTACAATGGCTTCAGGTGGTGGCGCACCAGTGG
CAGACAATAACGAAGGTGCCGATGGAGTGGGTAGTTCCTCGGGAAATTGGCATTGCGATTCCCAATGGCTG
GGGGACAGAGTCATCACCACCAGCACCCGAACCTGGGCCCTGCCCACCTACAACAATCACCTCTACAAGCA
AATCTCCAACAGCACATCTGGAGGATCTTCAAATGACAACGCCTACTTCGGCTACAGCACCCCCTGGGGGT
ATTTTGACTTCAACAGATTCCACTGCCACTTCTCACCACGTGACTGGCAGCGACTCATCAACAACAACTGGG
GATTCCGGCCTAAGCGACTCAACTTCAAGCTCTTCAACATTCAGGTCAAAGAGGTTACGGACAACAATGGAG
TCAAGACCATCGCCAATAACCTTACCAGCACGGTCCAGGTCTTCACGGACTCAGACTATCAGCTCCCGTACG
TGCTCGGGTCGGCTCACGAGGGCTGCCTCCCGCCGTTCCCAGCGGACGTTTTCATGATTCCTCAGTACGG
GTATCTGACGCTTAATGATGGAAGCCAGGCCGTGGGTCGTTCGTCCTTTTACTGCCTGGAATATTTCCCGTC
GCAAATGCTAAGAACGGGTAACAACTTCCAGTTCAGCTACGAGTTTGAGAACGTACCTTTCCATAGCAGCTA
CGCTCACAGCCAAAGCCTGGACCGACTAATGAATCCACTCATCGACCAATACTTGTACTATCTCTCAAAGAC
TATTAACGGTGGCCTGAGCAGCGGCAGCGGCCTGAGCGGCGCCCACATCGTGATGGTGGACGCCTACAAG
CCGACGAAGGGCCTGAGCGGCGGCAGCGGCCTGAGCGGCTCTGGACAGAATCAACAAACGCTAAAATTCA
GTGTGGCCGGACCCAGCAACATGGCTGTCCAGGGAAGAAACTACATACCTGGACCCAGCTACCGACAACAA
CGTGTCTCAACCACTGTGACTCAAAACAACAACAGCGAATTTGCTGCTCCTGGAGCTTCTTCTTGGGCTCTC
AATGGACGTAATAGCTTGATGAATCCTGGACCTGCTATGGCCAGCCACAAAGAAGGAGAGGACCGTTTCTTT
CCTTTGTCTGGATCTTTAATTTTTGGCAAACAAGGAACTGGAAGAGACAACGTGGATGCGGACAAAGTCATG
ATAACCAACGAAGAAGAAATTAAAACTACTAACCCGGTAGCAACGGAGTCCTATGGACAAGTGGCCACAAAC
CACCAGAGTGCCCAAGCACAGGCGCAGACCGGCTGGGTTCAAAACCAAGGAATACTTCCGGGTATGGTTTG
GCAGGACAGAGATGTGTACCTGCAAGGACCCATTTGGGCCAAAATTCCTCACACGGACGGCAACTTTCACC
CTTCTCCGCTGATGGGAGGGTTTGGAATGAAGCACCCGCCTCCTCAGATCCTCATCAAAAACACACCTGTAC
CTGCGGATCCTCCAACGGCCTTCAACAAGGACAAGCTGAACTCTTTCATCACCCAGTATTCTACTGGCCAAG
TCAGCGTGGAGATCGAGTGGGAGCTGCAGAAGGAAAACAGCAAGCGCTGGAACCCGGAGATCCAGTACAC
TTCCAACTATTACAAGTCTAATAATGTTGAATTTGCTGTTAATACTGAAGGTGTATATAGTGAACCCCGCCCC
ATTGGCACCAGATACCTGACTCGTAATCTGTAA
>pAAV9-RC G453 Linker10 SpyTag W503A (AA, Artificial) -
SEQ ID NO: 355
MAADGYLPDWLEDNLSEGIREWWALKPGAPQPKANQQHQDNARGLVLPGYKYLGPGNGLDKGEPVNAADAAA
LEHDKAYDQQLKAGDNPYLKYNHADAEFQERLKEDTSFGGNLGRAVFQAKKRLLEPLGLVEEAAKTAPGKKRPV
EQSPQEPDSSAGIGKSGAQPAKKRLNFGQTGDTESVPDPQPIGEPPAAPSGVGSLTMASGGGAPVADNNEGAD
GVGSSSGNWHCDSQWLGDRVITTSTRTWALPTYNNHLYKQISNSTSGGSSNDNAYFGYSTPWGYFDFNRFHC
HFSPRDWQRLINNNWGFRPKRLNFKLFNIQVKEVTDNNGVKTIANNLTSTVQVFTDSDYQLPYVLGSAHEGCLPP
FPADVFMIPQYGYLTLNDGSQAVGRSSFYCLEYFPSQMLRTGNNFQFSYEFENVPFHSSYAHSQSLDRLMNPLI
DQYLYYLSKTINGGLSSGSGLSGAHIVMVDAYKPTKGLSGGSGLSGSGQNQQTLKFSVAGPSNMAVQGRNYIP
GPSYRQQRVSTTVTQNNNSEFAAPGASSWALNGRNSLMNPGPAMASHKEGEDRFFPLSGSLIFGKQGTGRDN
VDADKVMITNEEEIKTTNPVATESYGQVATNHQSAQAQAQTGWVQNQGILPGMVWQDRDVYLQGPIWAKIPHT
DGNFHPSPLMGGFGMKHPPPQILIKNTPVPADPPTAFNKDKLNSFITQYSTGQVSVEIEWELQKENSKRWNPEIQ
YTSNYYKSNNVEFAVNTEGVYSEPRPIGTRYLTRNL*
>pAAV8-CAP wt (DNA Artificial)-
SEQ ID NO: 356
ATGGCTGCCGATGGTTATCTTCCAGATTGGCTCGAGGACAACCTCTCTGAGGGCATTCGCGAGTGGTGGGC
GCTGAAACCTGGAGCCCCGAAGCCCAAAGCCAACCAGCAAAAGCAGGACGACGGCCGGGGTCTGGTGCTT
CCTGGCTACAAGTACCTCGGACCCTTCAACGGACTCGACAAGGGGGAGCCCGTCAACGCGGCGGACGCAG
CGGCCCTCGAGCACGACAAGGCCTACGACCAGCAGCTGCAGGGGGGTGACAATCCGTACCTGCGGTATAA
CCACGCCGACGCCGAGTTTCAGGAGCGTCTGCAAGAAGATACGTCTTTTGGGGGCAACCTCGGGCGAGCA
GTCTTCCAGGCCAAGAAGCGGGTTCTCGAACCTCTCGGTCTGGTTGAGGAAGGCGCTAAGACGGCTCCTG
GAAAGAAGAGACCGGTAGAGCCATCACCCCAGCGTTCTCCAGACTCCTCTACGGGCATCGGCAAGAAAGG
CCAACAGCCCGCCAGAAAAAGACTCAATTTTGGTCAGACTGGCGACTCAGAGTCAGTTCCAGACCCTCAAC
CTCTCGGAGAACCTCCAGCAGCGCCCTCTGGTGTGGGACCTAATACAATGGCTGCAGGCGGTGGCGCACC
AATGGCAGACAATAACGAAGGCGCCGACGGAGTGGGTAGTTCCTCGGGAAATTGGCATTGCGATTCCACAT
GGCTGGGCGACAGAGTCATCACCACCAGCACCCGAACCTGGGCCCTGCCCACCTACAACAACCACCTCTA
CAAGCAAATCTCCAACGGGACATCGGGAGGAGCCACCAACGACAACACCTACTTCGGCTACAGCACCCCCT
GGGGGTATTTTGACTTTAACAGATTCCACTGCCACTTTTCACCACGTGACTGGCAGCGACTCATCAACAACA
ACTGGGGATTCCGGCCCAAGAGACTCAGCTTCAAGCTCTTCAACATCCAGGTCAAGGAGGTCACGCAGAAT
GAAGGCACCAAGACCATCGCCAATAACCTCACCAGCACCATCCAGGTGTTTACGGACTCGGAGTACCAGCT
GCCGTACGTTCTCGGCTCTGCCCACCAGGGCTGCCTGCCTCCGTTCCCGGCGGACGTGTTCATGATTCCCC
AGTACGGCTACCTAACACTCAACAACGGTAGTCAGGCCGTGGGACGCTCCTCCTTCTACTGCCTGGAATAC
TTTCCTTCGCAGATGCTGAGAACCGGCAACAACTTCCAGTTTACTTACACCTTCGAGGACGTGCCTTTCCAC
AGCAGCTACGCCCACAGCCAGAGCTTGGACCGGCTGATGAATCCTCTGATTGACCAGTACCTGTACTACTT
GTCTCGGACTCAAACAACAGGAGGCACGGCAAATACGCAGACTCTGGGCTTCAGCCAAGGTGGGCCTAATA
CAATGGCCAATCAGGCAAAGAACTGGCTGCCAGGACCCTGTTACCGCCAACAACGCGTCTCAACGACAACC
GGGCAAAACAACAATAGCAACTTTGCCTGGACTGCTGGGACCAAATACCATCTGAATGGAAGAAATTCATTG
GCTAATCCTGGCATCGCTATGGCAACACACAAAGACGACGAGGAGCGTTTTTTTCCCAGTAACGGGATCCT
GATTTTTGGCAAACAAAATGCTGCCAGAGACAATGCGGATTACAGCGATGTCATGCTCACCAGCGAGGAAG
AAATCAAAACCACTAACCCTGTGGCTACAGAGGAATACGGTATCGTGGCAGATAACTTGCAGCAGCAAAACA
CGGCTCCTCAAATTGGAACTGTCAACAGCCAGGGGGCCTTACCCGGTATGGTCTGGCAGAACCGGGACGT
GTACCTGCAGGGTCCCATCTGGGCCAAGATTCCTCACACGGACGGCAACTTCCACCCGTCTCCGCTGATGG
GCGGCTTTGGCCTGAAACATCCTCCGCCTCAGATCCTGATCAAGAACACGCCTGTACCTGCGGATCCTCCG
ACCACCTTCAACCAGTCAAAGCTGAACTCTTTCATCACGCAATACAGCACCGGACAGGTCAGCGTGGAAATT
GAATGGGAGCTGCAGAAGGAAAACAGCAAGCGCTGGAACCCCGAGATCCAGTACACCTCCAACTACTACAA
ATCTACAAGTGTGGACTTTGCTGTTAATACAGAAGGCGTGTACTCTGAACCCCGCCCCATTGGCACCCGTTA
CCTCACCCGTAATCTGTAA
>pAAV8-CAP wt (AA Adeno-associated virus) -
SEQ ID NO: 357
MAADGYLPDWLEDNLSEGIREWWALKPGAPKPKANQQKQDDGRGLVLPGYKYLGPFNGLDKGEPVNAADAAA
LEHDKAYDQQLQAGDNPYLRYNHADAEFQERLQEDTSFGGNLGRAVFQAKKRVLEPLGLVEEGAKTAPGKKRP
VEPSPQRSPDSSTGIGKKGQQPARKRLNFGQTGDSESVPDPQPLGEPPAAPSGVGPNTMAAGGGAPMADNNE
GADGVGSSSGNWHCDSTWLGDRVITTSTRTWALPTYNNHLYKQISNGTSGGATNDNTYFGYSTPWGYFDFNRF
HCHFSPRDWQRLINNNWGFRPKRLSFKLFNIQVKEVTQNEGTKTIANNLTSTIQVFTDSEYQLPYVLGSAHQGCL
PPFPADVFMIPQYGYLTLNNGSQAVGRSSFYCLEYFPSQMLRTGNNFQFTYTFEDVPFHSSYAHSQSLDRLMNP
LIDQYLYYLSRTQTTGGTANTQTLGFSQGGPNTMANQAKNWLPGPCYRQQRVSTTTGQNNNSNFAWTAGTKY
HLNGRNSLANPGIAMATHKDDEERFFPSNGILIFGKQNAARDNADYSDVMLTSEEEIKTTNPVATEEYGIVADNLQ
QQNTAPQIGTVNSQGALPGMVWQNRDVYLQGPIWAKIPHTDGNFHPSPLMGGFGLKHPPPQILIKNTPVPADPP
TTFNQSKLNSFITQYSTGQVSVEIEWELQKENSKRWNPEIQYTSNYYKSTSVDFAVNTEGVYSEPRPIGTRYLTR
NL*
>pAAV1-CAP wt (DNA Artificial)-
SEQ ID NO: 358
ATGGCTGCCGATGGTTATCTTCCAGATTGGCTCGAGGACAACCTCTCTGAGGGCATTCGCGAGTGGTGGGA
CTTGAAACCTGGAGCCCCGAAGCCCAAAGCCAACCAGCAAAAGCAGGACGACGGCCGGGGTCTGGTGCTT
CCTGGCTACAAGTACCTCGGACCCTTCAACGGACTCGACAAGGGGGAGCCCGTCAACGCGGCGGACGCAG
CGGCCCTCGAGCACGACAAGGCCTACGACCAGCAGCTCAAAGCGGGTGACAATCCGTACCTTCGGTATAAC
CACGCCGACGCCGAGTTTCAGGAGCGTCTGCAAGAAGATACGTCTTTTGGGGGCAACCTCGGGCGAGCAG
TCTTCCAGGCCAAGAAGCGGGTTCTCGAACCTCTCGGTCTGGTTGAGGAAGGCGCTAAGACGGCTCCTGGA
AAGAAACGTCCGGTAGAGCAGTCGCCACAAGAGCCAGACTCCTCCTCGGGCATCGGCAAGACAGGCCAGC
AGCCCGCTAAAAAGAGACTCAATTTTGGTCAGACTGGCGACTCAGAGTCAGTCCCCGACCCACAACCTCTC
GGAGAACCTCCAGCAACCCCCGCTGCTGTGGGACCTACTACAATGGCTTCAGGCGGTGGCGCACCAATGG
CAGACAATAACGAAGGCGCCGACGGAGTGGGTAATGCCTCAGGAAATTGGCATTGCGATTCCACATGGCTG
GGCGACAGAGTCATCACCACCAGCACCCGCACCTGGGCCTTGCCCACCTACAATAACCACCTCTACAAGCA
AATCTCCAGTGCTTCAACGGGGGCCAGCAACGACAACCACTACTTCGGCTACAGCACCCCCTGGGGGTATT
TTGATTTCAACAGATTCCACTGCCACTTTTCACCACGTGACTGGCAGCGACTCATCAACAACAATTGGGGATT
CCGGCCCAAGAGACTCAACTTCAAACTCTTCAACATCCAAGTCAAGGAGGTCACGACGAATGATGGCGTCA
CAACCATCGCTAATAACCTTACCAGCACGGTTCAAGTCTTCTCGGACTCGGAGTACCAGCTTCCGTACGTCC
TCGGCTCTGCGCACCAGGGCTGCCTCCCTCCGTTCCCGGCGGACGTGTTCATGATTCCGCAATACGGCTAC
CTGACGCTCAACAATGGCAGCCAAGCCGTGGGACGTTCATCCTTTTACTGCCTGGAATATTTCCCTTCTCAG
ATGCTGAGAACGGGCAACAACTTTACCTTCAGCTACACCTTTGAGGAAGTGCCTTTCCACAGCAGCTACGCG
CACAGCCAGAGCCTGGACCGGCTGATGAATCCTCTCATCGACCAATACCTGTATTACCTGAACAGAACTCAA
AATCAGTCCGGAAGTGCCCAAAACAAGGACTTGCTGTTTAGCCGTGGGTCTCCAGCTGGCATGTCTGTTCA
GCCCAAAAACTGGCTACCTGGACCCTGTTATCGGCAGCAGCGCGTTTCTAAAACAAAAACAGACAACAACAA
CAGCAATTTTACCTGGACTGGTGCTTCAAAATATAACCTCAATGGGCGTGAATCCATCATCAACCCTGGCAC
TGCTATGGCCTCACACAAAGACGACGAAGACAAGTTCTTTCCCATGAGCGGTGTCATGATTTTTGGAAAAGA
GAGCGCCGGAGCTTCAAACACTGCATTGGACAATGTCATGATTACAGACGAAGAGGAAATTAAAGCCACTAA
CCCTGTGGCCACCGAAAGATTTGGGACCGTGGCAGTCAATTTCCAGAGCAGCAGCACAGACCCTGCGACC
GGAGATGTGCATGCTATGGGAGCATTACCTGGCATGGTGTGGCAAGATAGAGACGTGTACCTGCAGGGTCC
CATTTGGGCCAAAATTCCTCACACAGATGGACACTTTCACCCGTCTCCTCTTATGGGCGGCTTTGGACTCAA
GAACCCGCCTCCTCAGATCCTCATCAAAAACACGCCTGTTCCTGCGAATCCTCCGGCGGAGTTTTCAGCTAC
AAAGTTTGCTTCATTCATCACCCAATACTCCACAGGACAAGTGAGTGTGGAAATTGAATGGGAGCTGCAGAA
AGAAAACAGCAAGCGCTGGAATCCCGAAGTGCAGTACACATCCAATTATGCAAAATCTGCCAACGTTGATTT
TACTGTGGACAACAATGGACTTTATACTGAGCCTCGCCCCATTGGCACCCGTTACCTTACCCGTCCCCTGTA
A
>pAAV1-CAP wt (AA Adeno-associated virus) -
SEQ ID NO: 359
MAADGYLPDWLEDNLSEGIREWWDLKPGAPKPKANQQKQDDGRGLVLPGYKYLGPFNGLDKGEPVNAADAAA
LEHDKAYDQQLKAGDNPYLRYNHADAEFQERLQEDTSFGGNLGRAVFQAKKRVLEPLGLVEEGAKTAPGKKRP
VEQSPQEPDSSSGIGKTGQQPAKKRLNFGQTGDSESVPDPQPLGEPPATPAAVGPTTMASGGGAPMADNNEG
ADGVGNASGNWHCDSTWLGDRVITTSTRTWALPTYNNHLYKQISSASTGASNDNHYFGYSTPWGYFDFNRFHC
HFSPRDWQRLINNNWGFRPKRLNFKLFNIQVKEVTTNDGVTTIANNLTSTVQVFSDSEYQLPYVLGSAHQGCLPP
FPADVFMIPQYGYLTLNNGSQAVGRSSFYCLEYFPSQMLRTGNNFTFSYTFEEVPFHSSYAHSQSLDRLMNPLID
QYLYYLNRTQNQSSAQNKDLLFSRGSPAGMSVQPKNWLPGPCYRQQRVSKTKTDNNNSNFTWTGASKYNLNG
RESIINPGTAMASHKDDEDKFFPMSGVMIFGKESAGASNTALDNVMITDEEEIKATNPVATERFGTVAVNFQSSST
DPATGDVHAMGALPGMVWQDRDVYLQGPIWAKIPHTDGHFHPSPLMGGFGLKNPPPQILIKNTPVPANPPAEFS
ATKFASFITQYSTGQVSVEIEWELQKENSKRWNPEVQYTSNYAKSANVDFTVDNNGLYTEPRPIGTRYLTRPL*
AAV6 VP1 (AA - Adeno-associated virus) -
SEQ ID NO: 360
MAADGYLPDW LEDNLSEGIR EWWDLKPGAP KPKANQQKQD DGRGLVLPGY KYLGPFNGLDKGEPVNAADA
AALEHDKAYD QQLKAGDNPY LRYNHADAEF QERLQEDTSF GGNLGRAVFQ AKKRVLEPFG LVEEGAKTAP
GKKRPVEQSP QEPDSSSGIG KTGQQPAKKR LNFGQTGDSE SVPDPQPLGE PPATPAAVGP TTMASGGGAP
MADNNEGADG VGNASGNWHC DSTWLGDRVI TTSTRTWALP TYNNHLYKQI SSASTGASND NHYFGYSTPW
GYFDFNRFHC HFSPRDWQRL INNNWGFRPK RLNFKLFNIQ VKEVTTNDGV TTIANNLTST VQVFSDSEYQ
LPYVLGSAHQ GCLPPFPADV FMIPQYGYLT LNNGSQAVGR SSFYCLEYFP SQMLRTGNNF TFSYTFEDVP
FHSSYAHSQS LDRLMNPLID QYLYYLNRTQ NQSGSAQNKD LLFSRGSPAG MSVQPKNWLP GPCYRQQRVS
KTKTDNNNSN FTWTGASKYN LNGRESIINP GTAMASHKDD KDKFFPMSGV MIFGKESAGA SNTALDNVMI
TDEEEIKATN PVATERFGTV AVNLQSSSTD PATGDVHVMG ALPGMVWQDR DVYLQGPIWA KIPHTDGHFH
PSPLMGGFGL KHPPPQILIK NTPVPANPPA EFSATKFASF ITQYSTGQVS VEIEWELQKE NSKRWNPEVQ
YTSNYAKSAN VDFTVDNNGL YTEPRPIGTR YLTRPL
>pAAV2-CAP N587 Myc (DNA, Artificial)-
SEQ ID NO: 361
ATGGCTGCCGATGGTTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCAGGGGTCTTGTGCTT
CCTGGGTACAAGTACCTCGGACCCTTCAACGGACTCGACAAGGGAGAGCCGGTCAACGAGGCAGACGCCG
CGGCCCTCGAGCACGACAAAGCCTACGACCGGCAGCTCGACAGCGGAGACAACCCGTACCTCAAGTACAA
CCACGCCGACGCGGAGTTTCAGGAGCGCCTTAAAGAAGATACGTCTTTTGGGGGCAACCTCGGACGAGCA
GTCTTCCAGGCGAAAAAGAGGGTTCTTGAACCTCTGGGCCTGGTTGAGGAACCTGTTAAGACGGCTCCGGG
AAAAAAGAGGCCGGTAGAGCACTCTCCTGTGGAGCCAGACTCCTCCTCGGGAACCGGAAAGGCGGGCCAG
CAGCCTGCAAGAAAAAGATTGAATTTTGGTCAGACTGGAGACGCAGACTCAGTACCTGACCCCCAGCCTCT
CGGACAGCCACCAGCAGCCCCCTCTGGTCTGGGAACTAATACGATGGCTACAGGCAGTGGCGCACCAATG
GCAGACAATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTCTACAAAC
AAATTTCCAGCCAATCAGGAGCCTCGAACGACAATCACTACTTTGGCTACAGCACCCCTTGGGGGTATTTTG
ACTTCAACAGATTCCACTGCCACTTTTCACCACGTGACTGGCAAAGACTCATCAACAACAACTGGGGATTCC
GACCCAAGAGACTCAACTTCAAGCTCTTTAACATTCAAGTCAAAGAGGTCACGCAGAATGACGGTACGACGA
CGATTGCCAATAACCTTACCAGCACGGTTCAGGTGTTTACTGACTCGGAGTACCAGCTCCCGTACGTCCTCG
GCTCGGCGCATCAAGGATGCCTCCCGCCGTTCCCAGCAGACGTCTTCATGGTGCCACAGTATGGATACCTC
ACCCTGAACAACGGGAGTCAGGCAGTAGGACGCTCTTCATTTTACTGCCTGGAGTACTTTCCTTCTCAGATG
CTGCGTACCGGAAACAACTTTACCTTCAGCTACACTTTTGAGGACGTTCCTTTCCACAGCAGCTACGCTCAC
AGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTC
TAGGAACTGGCTTCCTGGACCCTGTTACCGCCAGCAGCGAGTATCAAAGACATCTGCGGATAACAACAACA
GTGAATACTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTGGGAAGCA
AGGCTCAGAGAAAACAAATGTGGACATTGAAAAGGTCATGATTACAGACGAAGAGGAAATCAGGACAACCAA
TCCCGTGGCTACGGAGCAGTATGGTTCTGTATCTACCAACCTCCAGAGAGGCAACGAACAAAAACTCATCTC
AGAAGAGGATCTGAGACAAGCAGCTACCGCAGATGTCAACACACAAGGCGTTCTTCCAGGCATGGTCTGGC
AGGACAGAGATGTGTACCTTCAGGGGCCCATCTGGGCAAAGATTCCACACACGGACGGACATTTTCACCCC
TCTCCCCTCATGGGTGGATTCGGACTTAAACACCCTCCTCCACAGATTCTCATCAAGAACACCCCGGTACCT
GCGAATCCTTCGACCACCTTCAGTGCGGCAAAGTTTGCTTCCTTCATCACACAGTACTCCACGGGACAGGTC
AGCGTGGAGATCGAGTGGGAGCTGCAGAAGGAAAACAGCAAACGCTGGAATCCCGAAATTCAGTACACTTC
CAACTACAACAAGTCTGTTAATGTGGACTTTACTGTGGACACTAATGGCGTGTATTCAGAGCCTCGCCCCAT
TGGCACCAGATACCTGACTCGTAATCTGTAA
>pAAV2-CAP N587 Myc (AA, Artificial)-
SEQ ID NO: 362
MAADGYLPDWLEDTLSEGIRQWWKLKPGPPPPKPAERHKDDSRGLVLPGYKYLGPFNGLDKGEPVNEADAAAL
EHDKAYDRQLDSGDNPYLKYNHADAEFQERLKEDTSFGGNLGRAVFQAKKRVLEPLGLVEEPVKTAPGKKRPV
EHSPVEPDSSSGTGKAGQQPARKRLNFGQTGDADSVPDPQPLGQPPAAPSGLGTNTMATGSGAPMADNNEGA
DGVGNSSGNWHCDSTWMGDRVITTSTRTWALPTYNNHLYKQISSQSGASNDNHYFGYSTPWGYFDFNRFHCH
FSPRDWQRLINNNWGFRPKRLNFKLFNIQVKEVTQNDGTTTIANNLTSTVQVFTDSEYQLPYVLGSAHQGCLPPF
PADVFMVPQYGYLTLNNGSQAVGRSSFYCLEYFPSQMLRTGNNFTFSYTFEDVPFHSSYAHSQSLDRLMNPLID
QYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQRGN
EQKLISEEDLRQAATADVNTQGVLPGMVWQDRDVYLQGPIWAKIPHTDGHFHPSPLMGGFGLKHPPPQILIKNTP
VPANPSTTFSAAKFASFITQYSTGQVSVEIEWELQKENSKRWNPEIQYTSNYNKSVNVDFTVDTNGVYSEPRPIG
TRYLTRNL

The phrase “Inverted terminal repeat” or “ITR” includes symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV particles, e.g., packaging into AAV particles.

AAV ITR comprise recognition sites for replication proteins Rep78 or Rep68. A″D″ region of the ITR comprises the DNA nick site where DNA replication initiates and provides directionality to the nucleic acid replication step. An AAV replicating in a mammalian cell typically comprises two ITR sequences.

A single ITR may be engineered with Rep binding sites on both strands of the “A” regions and two symmetrical D regions on each side of the ITR palindrome. Such an engineered construct on a double-stranded circular DNA template allows Rep78 or Rep68 initiated nucleic acid replication that proceeds in both directions. A single ITR is sufficient for AAV replication of a circular particle. In methods of producing an AAV viral particle of the invention, the rep encoding sequence encodes a Rep protein or Rep protein equivalent that is capable of binding an ITR comprised on the transfer plasmid.

The Cap proteins of the invention, when expressed with appropriate Rep proteins by a packaging cell, may encapsidate a transfer plasmid comprising a nucleotide of interest and an even number of two or more ITR sequences. In some embodiments, a transfer plasmid comprises one ITR sequence. In some embodiments, a transfer plasmid comprises two ITR sequences.

Either Rep78 and/or Rep68 bind to unique and known sites on the sequence of the ITR hairpin, and act to break and unwind the hairpin structures on the end of an AAV genome, thereby providing access to replication machinery of the viral replication cell. As is well-known, Rep proteins may be expressed from more than one ORF comprising nucleotide sequence encoding any combination of Rep78, Rep68, Rep 52 and/or Rep40 by use of separate nucleotide sequences operably linked to at least one expression control sequence for expression in a viral replication cell, each producing one or more of Rep78, Rep68, Rep52 and/or Rep40 Rep proteins. Alternatively, Rep proteins may be expressed individually from an ORF comprising a nucleotide sequence encoding any one of Rep78, Rep68, Rep52, or Rep40 by use of separate nucleotide sequences operably linked to one expression control sequence for expression in a packaging cell, each producing only one Rep78, Rep68, Rep52, or Rep40 Rep protein. In another embodiment, Rep proteins may be expressed from one ORF comprising nucleotide sequences encoding Rep78 and Rep52 Rep proteins operably linked to at least one expression control sequence for expression in a viral replication cell each producing Rep78 and Rep52 Rep protein.

In a method of producing an AAV virion, e.g., viral particle, of the invention, a rep encoding sequence and a cap gene of the invention may be provided a single packaging plasmid. However, a skilled artisan will recognize that such proviso is not necessary. Such viral particles may or may not include a genome.

A “chimeric AAV capsid protein” includes an AAV capsid protein that comprises amino acid sequences, e.g., portions, from two or more different AAV and that is capable of forming and/or forms an AAV viral capsid/viral particle. A chimeric AAV capsid protein is encoded by a chimeric AAV capsid gene, e.g., a chimeric nucleotide comprising a plurality, e.g., at least two, nucleic acid sequences, each of which plurality is identical to a portion of a capsid gene encoding a capsid protein of distinct AAV, and which plurality together encodes a functional chimeric AAV capsid protein. Association of a chimeric capsid protein to a specific AAV indicates that the capsid protein comprises one or more portions from a capsid protein of that AAV and one or more portions from a capsid protein of a different AAV. For example, a chimeric AAV2 capsid protein includes a capsid protein comprising one or more portions of a VP1, VP2, and/or VP3 capsid protein of AAV2 and one or more portions of a VP1, VP2, and/or VP3 capsid protein of a different AAV.

The term “portion” refers to at least 5 amino acids or at least 15 nucleotides, but less than the full-length polypeptide or nucleic acid molecule, with 100% identity to a sequence from which the portion is derived, see Penzes (2015) J. General Virol. 2769. A “portion” encompasses any contiguous segment of amino acids or nucleotides sufficient to determine that the polypeptide or nucleic acid molecule form which the portion is derived is “of a [specified] AAV” or has “significant identity” to a particular AAV, e.g., a non-primate animal AAV or remote AAV. In some embodiments, a portion comprises at least 5 amino acids or 15 nucleotides with 100% identity to a sequence associated with the specified AAV. In some embodiments, a portion comprises at least 10 amino acids or 30 nucleotides with 100% identity to a sequence associated with the specified AAV. In some embodiments, a portion comprises at least 15 amino acids or 45 nucleotides with 100% identity to a sequence associated with the specified AAV. In some embodiments, a portion comprises at least 20 amino acids or 60 nucleotides with 100% identity to a sequence associated with the specified AAV. In some embodiments, a portion comprises at least 25 amino acids or 75 nucleotides with 100% identity to a sequence associated with the specified AAV. In some embodiments, a portion comprises at least 30 amino acids or 90 nucleotides with 100% identity to a sequence associated with the specified AAV. In some embodiments, a portion comprises at least 35 amino acids or 105 nucleotides with 100% identity to a sequence associated with the specified AAV. In some embodiments, a portion comprises at least 40 amino acids or 120 nucleotides with 100% identity to a sequence associated with the specified AAV. In some embodiments, a portion comprises at least 45 amino acids or 135 nucleotides with 100% identity to a sequence associated with the specified AAV. In some embodiments, a portion comprises at least 50 amino acids or 150 nucleotides with 100% identity to a sequence associated with the specified AAV. In some embodiments, a portion comprises at least 60 amino acids or 180 nucleotides with 100% identity to a sequence associated with the specified AAV. In some embodiments, a portion comprises at least 70 amino acids or 210 nucleotides with 100% identity to a sequence associated with the specified AAV. In some embodiments, a portion comprises at least 80 amino acids or 240 nucleotides with 100% identity to a sequence associated with the specified AAV. In some embodiments, a portion comprises at least 90 amino acids or 270 nucleotides with 100% identity to a sequence associated with the specified AAV. In some embodiments, a portion comprises at least 100 amino acids or 300 nucleotides with 100% identity to a sequence associated with the specified AAV.

Modified Virus Capsid Proteins, Viral Particles, Nucleic Acids

In some embodiments, a Cap protein, e.g., a VP1 capsid protein as described herein, a VP2 capsid protein as described herein, and/or a VP3 capsid protein as described herein, is modified to comprise e.g., a first member of a protein:protein binding pair, a detectable label, point mutation, etc.

Chimerism is a type of modification as described herein. Generally, modification of gene or a polypeptide of a specified AAV, or variants thereof, results in nucleic acid sequence or an amino acid sequence that differs from the nucleic acid sequence or amino acid sequence set forth herein for the specified AAV, wherein the modification alters, confers, or removes one or more biological functions, but does not change the phylogenetic characterization of, the gene or polypeptide. A modification may include an insertion of, e.g., a first member of a protein:protein binding pair and a point mutation, e.g., such that the natural tropism of the capsid protein is reduced to abolished and/or such that the capsid protein comprises a detectable label. Preferred modifications include those that do not alter and preferably decrease the low to no recognition of the modified capsid by pre-existing antibodies found in the general population that were produced during the course of infection with another AAV, e.g., infection with serotypes such as AAV1, AAV2, AAV3, AAV4, AAV5, AAV6, AAV7, AAV8, AAV9, AAV10, AAV11, AAV12, AAV13, AAVDJ, Anc80L65, AAV2G9, AAV-LK03, virions based on such serotypes, virions from currently used AAV gene therapy modalities, or a combination thereof. Other modifications as described herein include modification of a capsid protein such that it comprises a first member of a protein:protein binding pair, a detectable label, etc., which modifications generally result from modifications at the genetic level, e.g., via modification of a cap gene.

In some embodiments a viral capsid comprising a modified viral capsid protein as described herein is a mosaic capsid, e.g., comprises at least two sets of VP1, VP2, and/or VP3 proteins, each set of which is encoded by a different cap gene. A mosaic capsid herein generally refers to a mosaic of a first viral capsid protein modified to comprise a first member of a protein:protein binding pair and a second corresponding viral capsid protein lacking the first member of a protein:protein binding pair. In relation to a mosaic capsid, the second viral capsid protein lacking the first member of a protein:protein binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a protein:protein binding pair, except that the reference capsid protein lacks the first member of a protein:protein binding pair. In some mosaic capsid embodiments, a VP1, VP2, and/or VP3 reference capsid protein corresponds to the viral VP1, VP2, and/or VP3 capsid protein modified with a first member of a protein:protein binding pair, except that the reference capsid protein lacks the first member of a protein:protein binding pair. In some embodiments, a VP1 reference capsid protein corresponds to the viral VP1 capsid protein modified with a first member of a protein:protein binding pair, except that the reference capsid protein lacks the first member of a protein:protein binding pair. In some embodiments, a VP2 reference capsid protein corresponds to the viral VP2 capsid protein modified with a first member of a protein:protein binding pair, except that the reference capsid protein lacks the first member of a protein:protein binding pair. In some embodiments, a VP3 reference capsid protein corresponds to the viral VP3 capsid protein modified with a first member of a protein:protein binding pair, except that the reference capsid protein lacks the first member of a protein:protein binding pair. In some mosaic capsid embodiments comprising a chimeric VP1, VP2, and/or VP3 capsid protein further modified to comprise a first member of a protein:protein binding pair, a reference protein may be a corresponding capsid protein from which portions thereof form part of the chimeric capsid protein. As a non-limiting example in some embodiments, mosaic capsid comprising a chimeric AAV2/AAAV VP1 capsid protein modified to comprise a first member of a protein:protein binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the protein:protein binding pair and is able to form a capsid with the first capsid protein modified with the first member of a protein:protein binding pair.

Generally mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a protein:protein binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1. In some mosaic viral particle embodiments, the protein subunit is at least about 1:1 (e.g., the mosaic viral particle comprises about 30 modified capsid proteins and about 30 reference capsid protein). In some mosaic viral particle embodiments, the protein subunit ratio is at least about 1:2 (e.g., the mosaic viral particle comprises about 20 modified capsid proteins and about 40 reference capsid proteins). In some mosaic viral particle embodiments, the protein subunit ratio is at least about 3:5. In some mosaic viral particle embodiments, the protein subunit ratio is at least about 1:3 (e.g., the mosaic viral particle comprises about 15 modified capsid proteins and about 45 reference capsid proteins). In some mosaic viral particle embodiments, the protein subunit ratio is at least about 1:4 (e.g., the mosaic viral particle comprises about 12 modified capsid proteins and 48 reference capsid proteins). In some mosaic viral particle embodiments, the protein subunit ratio is at least about 1:5 (e.g., the mosaic viral particle comprises 10 modified capsid proteins and 50 reference capsid proteins). In some mosaic viral particle embodiments, the protein subunit ratio is at least about 1:6. In some mosaic viral particle embodiments, the protein subunit ratio is at least about 1:7. In some mosaic viral particle embodiments, the protein subunit ratio is at least about 1:8. In some mosaic viral particle embodiments, the protein subunit ratio is at least about 1:9 (e.g., the mosaic viral particle comprises about 6 modified capsid proteins and about 54 reference capsid proteins). In some mosaic viral particle embodiments, the protein subunit ratio is at least about 1:10. In some mosaic viral particle embodiments, the protein subunit ratio is at least about 1:11 (e.g., the mosaic viral particle comprises about 5 modified capsid proteins and about 55 reference capsid proteins). In some mosaic viral particle embodiments, the protein subunit ratio is at least about 1:12. In some mosaic viral particle embodiments, the protein subunit ratio is at least about 1:13. In some mosaic viral particle embodiments, the protein subunit ratio is at least about 1:14 (e.g., the mosaic viral particle comprises about 4 modified capsid proteins and about 56 reference capsid proteins). In some mosaic viral particle embodiments, the protein subunit ratio is at least about 1:15. In some mosaic viral particle embodiments, the protein subunit ratio is at least about 1:19 (e.g., the mosaic viral particle comprises about 3 modified capsid proteins and about 57 reference capsid proteins). In some mosaic viral particle embodiments, the protein subunit ratio is at least about 1:29 (e.g., the mosaic viral particle comprises about 2 modified capsid proteins and about 58 reference capsid proteins). In some mosaic viral particle embodiments, the protein subunit ratio is at least about 1:59. In some mosaic viral particle embodiments, the protein subunit ratio is at least about 2:1 (e.g., the mosaic viral particle comprises about 40 modified capsid proteins and about 20 reference capsid proteins). In some mosaic viral particle embodiments, the protein subunit ratio is at least about 5:3. In some mosaic viral particle embodiments, the protein subunit ratio is at least about 3:1 (e.g., the mosaic viral particle comprises about 45 modified capsid proteins and about 15 reference capsid proteins). In some mosaic viral particle embodiments, the protein subunit ratio is at least about 4:1 (e.g., the mosaic viral particle comprises about 48 modified capsid proteins and 12 reference capsid proteins). In some mosaic viral particle embodiments, the protein subunit ratio is at least about 5:1 (e.g., the mosaic viral particle comprises 50 modified capsid proteins and 10 reference capsid proteins). In some mosaic viral particle embodiments, the protein subunit ratio is at least about 6:1. In some mosaic viral particle embodiments, the protein subunit ratio is at least about 7:1. In some mosaic viral particle embodiments, the protein subunit ratio is at least about 8:1. In some mosaic viral particle embodiments, the protein subunit ratio is at least about 9:1 (e.g., the mosaic viral particle comprises about 54 modified capsid proteins and about 6 reference capsid proteins). In some mosaic viral particle embodiments, the protein subunit ratio is at least about 10:1. In some mosaic viral particle embodiments, the protein subunit ratio is at least about 11:1 (e.g., the mosaic viral particle comprises about 55 modified capsid proteins and about 5 reference capsid proteins). In some mosaic viral particle embodiments, the protein subunit ratio is at least about 12:1. In some mosaic viral particle embodiments, the protein subunit ratio is at least about 13:1. In some mosaic viral particle embodiments, the protein subunit ratio is at least about 14:1 (e.g., the mosaic viral particle comprises about 56 modified capsid proteins and about 4 reference capsid proteins). In some mosaic viral particle embodiments, the protein subunit ratio is at least about 15:1. In some mosaic viral particle embodiments, the protein subunit ratio is at least about 19:1 (e.g., the mosaic viral particle comprises about 57 modified capsid proteins and about 3 reference capsid proteins). In some mosaic viral particle embodiments, the protein subunit ratio is at least about 29:1 (e.g., the mosaic viral particle comprises about 58 modified capsid proteins and about 2 reference capsid proteins). In some mosaic viral particle embodiments, the protein subunit ratio is at least about 59:1.

In some non-mosaic viral particle embodiments, the protein subunit ratio may be 1:0 wherein each capsid protein of the non-mosaic viral particle is modified with a first member of a protein:protein binding pair. In some non-mosaic viral particle embodiments, the protein subunit ratio may be 0:1 wherein each capsid protein of the non-mosaic viral particle is not modified with a first member of a protein:protein binding pair.

In some embodiments, a capsid protein of the invention is modified to comprise a detectable label. Many detectable labels are known in the art. (See, e.g.: Nilsson et al. (1997) “Affinity fusion strategies for detection, purification, and immobilization of modified proteins” Protein Expression and Purification 11: 1-16, Terpe et al. (2003) “Overview of tag protein fusions: From molecular and biochemical fundamentals to commercial systems” Applied Microbiology and Biotechnology 60:523-533, and references therein). Detectable labels include, but are not limited to, a polyhistidine detectable labels (e.g., a His-6 (SEQ ID NO: 235), His-8 (SEQ ID NO: 392), or His-10 (SEQ ID NO: 393)) that binds immobilized divalent cations (e.g., Ni 2+), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high detectable label that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary detectable label is a SNAP-tag, commercially available from Covalys (www.covalys.com). In some embodiments, a detectable label disclosed herein comprises a detectable label recognized only by an antibody paratope. In some embodiments, a detectable label disclosed herein comprises a detectable label recognized by an antibody paratope and other specific binding pairs.

In some embodiments, the detectable label forms a binding pair with an immunoglobulin constant domain. In some embodiments, the detectable label and/or detectable label does form a binding pair with a metal ion, e.g., Ni²⁺, Co²⁺, Cu²⁺, Zn²⁺, Fe³⁺, etc. In some embodiments, the detectable label is selected from the group consisting of Streptavidin, Strep II, HA, L14, 4C-RGD, LH, and Protein A.

In some embodiments, the detectable label is selected from the group consisting of FLAG, HA and c-myc (EQKLISEEDL; SEQ ID NO: 282). In some embodiments, the detectable label is c-myc (SEQ ID NO: 282).

In some embodiments, a detectable label is a B cell epitope, e.g., is between about 1 amino acid and about 35 amino acids in length, and forms a binding pair with an antibody paratope, e.g., an immunoglobulin variable domain. In some embodiments, the detectable label comprises a B1 epitope (SEQ ID NO: 283). In some embodiments, a capsid protein is modified to comprise a B1 epitope in the VP3 region.

In some embodiments, a capsid protein of the invention comprises at least a first member of a peptide:peptide binding pair.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc (SEQ ID NO: 282). Use of a detectable label as a first member of a protein:protein binding pair is described in, e.g., WO2019006043, incorporated herein in its entirety by reference.

In some embodiments, a capsid protein comprises a first member of a protein:protein binding pair, wherein the protein:protein binding pair forms a covalent isopeptide bond. In some embodiments, the first member of a peptide:peptide binding pair is covalently bound via an isopeptide bond to a cognate second member of the peptide:peptide binding pair, and optionally wherein the cognate second member of the peptide:peptide binding pair is fused with a targeting ligand, which targeting ligand binds a target expressed by a cell of interest. In some embodiments, the protein:protein binding pair may be selected from the group consisting of SpyTag:SpyCatcher, SpyTag002:SpyCatcher002, SpyTag:KTag, I sopeptag:pilinC, and SnoopTag:SnoopCatcher. In some embodiments, wherein the first member is SpyTag (or a biologically active portion thereof) and the protein (second cognate member) is SpyCatcher (or a biologically active portion thereof). In some embodiments, wherein the first member is SpyTag (or a biologically active portion thereof) and the protein (second cognate member) is KTag (or a biologically active portion thereof). In some embodiments, wherein the first member is KTag (or a biologically active portion thereof) and the protein (second cognate member) is SpyTag (or a biologically active portion thereof). In some embodiments, wherein the first member is SnoopTag (or a biologically active portion thereof) and the protein (second cognate member) is SnoopCatcher (or a biologically active portion thereof). In some embodiments, wherein the first member is Isopeptag (or a biologically active portion thereof) and the protein (second cognate member) is Pilin-C (or a biologically active portion thereof). In some embodiments, wherein the first member is SpyTag002 (or a biologically active portion thereof) and the protein (second cognate member) is SpyCatcher002 (or a biologically active portion thereof). In some embodiments, a Cap protein of the invention comprises a SpyTag. Use of a first member of a protein:protein binding pair is described in WO2019006046, incorporated herein in its entirety.

In some embodiments, a first member of a protein:protein binding pair and/or detectable label is operably linked to (translated in frame with, chemically attached to, and/or displayed by) a Cap protein of the invention via a first or second linker, e.g., an amino acid spacer that is at least one amino acid in length. In some embodiments, the first member of a protein:protein binding pair is flanked by a first and/or second linker, e.g., a first and/or second amino acid spacer, each of which spacer is at least one amino acid in length.

In some embodiments, the first and/or second linkers are not identical. In some embodiments, the first and/or second linker is each independently one or two amino acids in length. In some embodiments, the first and/or second linker is each independently one, two or three amino acids in length. In some embodiments, the first and/or second linker is each independently one, two, three, or four amino acids in length. In some embodiments, the first and/or second linker is each independently one, two, three, four, or five amino acids in length. In some embodiments, the first and/or second linker are each independently one, two, three, four, or five amino acids in length. In some embodiments, the first and/or second linker is each independently one, two, three, four, five, or six amino acids in length. In some embodiments, the first and/or second linker is each independently one, two, three, four, five, six, or seven amino acids in length. In some embodiments, the first and/or second linker is each independently one, two, three, four, five, six, seven, or eight amino acids in length. In some embodiments, the first and/or second linker is each independently one, two, three, four, five, six, seven, eight or nine amino acids in length. In some embodiments, the first and or second linker is each independently one, two, three, four, five, six, seven, eight, nine, or ten amino acids in length. In some embodiments, the first and or second linker is each independently one, two, three, four, five, six, seven, eight, nine, ten, or more amino acids in length.

In some embodiments, the first and second linkers are identical in sequence and/or in length and are each one amino acid in length. In some embodiments, the first and second linkers are identical in length, and are each one amino acid in length. In some embodiments, the first and second linkers are identical in length, and are each two amino acids in length. In some embodiments, the first and second linkers are identical in length, and are each three amino acids in length. In some embodiments, the first and second linkers are identical in length, and are each four amino acids in length, e.g., the linker is GLSG (SEQ ID NO: 284). In some embodiments, the first and second linkers are identical in length, and are each five amino acids in length. In some embodiments, the first and second linkers are identical in length, and are each six amino acids in length, e.g., the first and second linkers each comprise a sequence of GLSGSG (SEQ ID NO: 285). In some embodiments, the first and second linkers are identical in length, and are each seven amino acids in length. In some embodiments, the first and second linkers are identical in length, and are each eight amino acids in length, e.g., the first and second linkers each comprise a sequence of GLSGLSGS (SEQ ID NO: 286). In some embodiments, the first and second linkers are identical in length, and are each nine amino acids in length. In some embodiments, the first and second linkers are identical in length, and are each ten amino acids in length, e.g., the first and second linkers each comprise a sequence of GLSGLSGLSG (SEQ ID NO: 287) or GLSGGSGLSG (SEQ ID NO: 288). In some embodiments, the first and second linkers are identical in length, and are each more than ten amino acids in length.

Generally, a first member of a protein:protein binding pair amino acid sequence as described herein, e.g., comprising a first member of a specific binding pair by itself or in combination with one or more linkers, is between about 5 amino acids to about 50 amino acids in length. In some embodiments, the first member of a protein:protein binding pair amino acid sequence is at least 5 amino acids in length. In some embodiments, the first member of a protein:protein binding pair amino acid sequence is 6 amino acids in length. In some embodiments, the first member of a protein:protein binding pair amino acid sequence is 7 amino acids in length. In some embodiments, the first member of a protein:protein binding pair amino acid sequence is 8 amino acids in length. In some embodiments, the first member of a protein:protein binding pair amino acid sequence is 9 amino acids in length. In some embodiments, the first member of a protein:protein binding pair amino acid sequence is 10 amino acids in length. In some embodiments, the first member of a protein:protein binding pair amino acid sequence is 11 amino acids in length. In some embodiments, the first member of a protein:protein binding pair amino acid sequence is 12 amino acids in length. In some embodiments, the first member of a protein:protein binding pair amino acid sequence is 13 amino acids in length. In some embodiments, the first member of a protein:protein binding pair amino acid sequence is 14 amino acids in length. In some embodiments, the first member of a protein:protein binding pair amino acid sequence is 15 amino acids in length. In some embodiments, the first member of a protein:protein binding pair amino acid sequence is 16 amino acids in length. In some embodiments, the first member of a protein:protein binding pair amino acid sequence is 17 amino acids in length. In some embodiments, the first member of a protein:protein binding pair amino acid sequence is 18 amino acids in length. In some embodiments, the first member of a protein:protein binding pair amino acid sequence is 19 amino acids in length. In some embodiments, the first member of a protein:protein binding pair amino acid sequence is 20 amino acids in length. In some embodiments, the first member of a protein:protein binding pair amino acid sequence is 21 amino acids in length. In some embodiments, the first member of a protein:protein binding pair amino acid sequence is 22 amino acids in length. In some embodiments, the first member of a protein:protein binding pair amino acid sequence is 23 amino acids in length. In some embodiments, the first member of a protein:protein binding pair amino acid sequence is 24 amino acids in length. In some embodiments, the first member of a protein:protein binding pair amino acid sequence is 25 amino acids in length. In some embodiments, the first member of a protein:protein binding pair amino acid sequence is 26 amino acids in length. In some embodiments, the first member of a protein:protein binding pair amino acid sequence is 27 amino acids in length. In some embodiments, the first member of a protein:protein binding pair amino acid sequence is 28 amino acids in length. In some embodiments, the first member of a protein:protein binding pair amino acid sequence is 29 amino acids in length. In some embodiments, the first member of a protein:protein binding pair amino acid sequence is 30 amino acids in length. In some embodiments, the first member of a protein:protein binding pair amino acid sequence is 31 amino acids in length. In some embodiments, the first member of a protein:protein binding pair amino acid sequence is 32 amino acids in length. In some embodiments, the first member of a protein:protein binding pair amino acid sequence is 33 amino acids in length. In some embodiments, the first member of a protein:protein binding pair amino acid sequence is 34 amino acids in length. In some embodiments, the first member of a protein:protein binding pair amino acid sequence is 35 amino acids in length. In some embodiments, the first member of a protein:protein binding pair amino acid sequence is 36 amino acids in length. In some embodiments, the first member of a protein:protein binding pair amino acid sequence is 37 amino acids in length. In some embodiments, the first member of a protein:protein binding pair amino acid sequence is 38 amino acids in length. In some embodiments, the first member of a protein:protein binding pair amino acid sequence is 39 amino acids in length. In some embodiments, the first member of a protein:protein binding pair amino acid sequence is 40 amino acids in length. In some embodiments, the first member of a protein:protein binding pair amino acid sequence is 41 amino acids in length. In some embodiments, the first member of a protein:protein binding pair amino acid sequence is 42 amino acids in length. In some embodiments, the first member of a protein:protein binding pair amino acid sequence is 43 amino acids in length. In some embodiments, the first member of a protein:protein binding pair amino acid sequence is 44 amino acids in length. In some embodiments, the first member of a protein:protein binding pair amino acid sequence is 45 amino acids in length. In some embodiments, the first member of a protein:protein binding pair amino acid sequence is 46 amino acids in length. In some embodiments, the first member of a protein:protein binding pair amino acid sequence is 47 amino acids in length. In some embodiments, the first member of a protein:protein binding pair amino acid sequence is 48 amino acids in length. In some embodiments, the first member of a protein:protein binding pair amino acid sequence is 49 amino acids in length. In some embodiments, the first member of a protein:protein binding pair amino acid sequence is 50 amino acids in length.

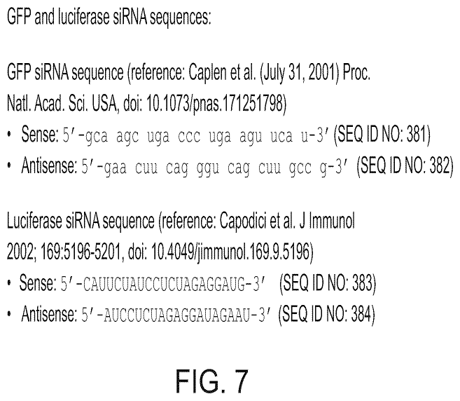

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzcsh et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of a first member of a protein:protein binding pair and/or detectable label.

Accordingly, in some embodiments, the first member of a protein:protein binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a protein:protein binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, I-261, I-266, I-381, I-453, I-447, I-448, I-459, I-471, I-520, I-534, I-570, I-573, I-584, I-587, I-588, I-591, I-657, I-664, I-713 and I-716 of the VP1 capsid protein of AAV2 (Wu et al. (2000) J. Virol. 74:8635-8647). In some embodiments, an insertion site of a non-primate animal VP1 capsid protein corresponds to 1-453. A modified virus capsid protein as described herein may be a non-primate animal capsid protein comprising a first member of a protein:protein binding pair and/or detectable label inserted into a position corresponding with a position of an AAV2 capsid protein selected from the group consisting of I-1, I-34, I-138, I-139, I-161, I-261, I-266, I-381, I-447, I-448, I-453, I-459, I-471, I-520, I-534, I-570, I-573, I-584, I-587, I-588, 1591, 1-657, I-664, I-713, I-716, and a combination thereof. In some embodiments, an insertion site of a non-primate animal VP1 capsid protein corresponds to 1-453. Additional suitable insertion sites of a non-primate animal AAV that include those corresponding to 1-587 of AAV1, I-589 of AAV1, I-585 of AAV3, I-585 of AAV4, and I-585 of AAV5. In some embodiments, a modified virus capsid protein as described herein may be a non-primate animal capsid protein comprising a first member of a protein:protein binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a protein:protein binding pair and/or detectable label is inserted in a VP1 capsid protein of a non-primate animal AAV after an amino acid position corresponding with an amino acid position selected from the group consisting of I444 of an avian AAV capsid protein VP1, I580 of an avian AAV capsid protein VP1, I573 of a bearded dragon AAV capsid protein VP1, I436 of a bearded dragon AAV capsid protein VP1, I429 of a sea lion AAV capsid protein VP1, I430 of a sea lion AAV capsid protein VP1, I431 of a sea lion AAV capsid protein VP1, I432 of a sea lion AAV capsid protein VP1, I433 of a sea lion AAV capsid protein VP1, I434 of a sea lion AAV capsid protein VP1, I436 of a sea lion AAV capsid protein VP1, I437 of a sea lion AAV capsid protein VP1, and 1565 of a sea lion AAV capsid protein VP1.

The nomenclature I-###, I #or the like herein refers to the insertion site (1) with ###naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s)N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site 1-587. Therefore, the present invention encompasses structural genes of AAV with corresponding insertions in the VP1, VP2 and/or VP3 proteins.

Also provided herein are nucleic acids that encode a VP3 capsid protein of the invention. AAV capsid proteins may be, but are not necessarily, encoded by overlapping reading frames of the same gene with staggered start codons. In some embodiments, a nucleic acid that encodes a VP3 capsid protein of the invention does not also encode a VP2 capsid protein or VP1 capsid protein of the invention. In some embodiments, a nucleic acid that encodes a VP3 capsid protein of the invention may also encode a VP2 capsid protein of the invention but does not also encode a VP1 capsid of the invention. In some embodiments, a nucleic acid that encodes a VP3 capsid protein of the invention may also encode a VP2 capsid protein of the invention and a VP1 capsid of the invention.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 1.5-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 2-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 3-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 4-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 5-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 6-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 7-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 8-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 9-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 10-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 20-fold greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is at least 30-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 40-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 50-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 60-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 70-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 80-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 90-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 100-fold greater than the transduction efficiency of a control viral capsid In some embodiments, a viral particle of the invention comprising a viral capsid protein comprising an amino acid sequence of a capsid protein of a non-primate animal AAV, a remote AAV, or a combination thereof, and optionally comprising a first and second members of a protein:protein binding pair (e.g., wherein the second member is operably linked to a targeting ligand, comprises a multispecific binding protein, etc.) is better able to evade neutralization by pre-existing antibodies in serum isolated from a human patient compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a protein:protein binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC₅₀ value that is at least 2-fold that of a control virus particle).

Targeting Ligands

A viral particle described herein may further comprise a targeting ligand.

In some embodiments of the invention comprising a detectable label, a targeting ligand comprises a multispecific binding molecule comprising (i) an antibody paratope that specifically binds the detectable label and (ii) a second binding domain that specifically binds a receptor (e.g., FGFR3), which may be conjugated to the surface of a bead (e.g., for purification) or expressed by a target cell (e.g., astrocyte). Accordingly, a multispecific binding molecule includes those binding moleucles comprising (i) an antibody paratope that specifically binds the detectable label and (ii) a second binding domain that specifically binds a receptor (e.g., FGFR3).

In some embodiments of the invention, a viral vector comprises a protein:protein binding pair associated by an isopeptide bond as described herein, wherein the second member of the protein:protein binding pair is fused to a targeting ligand. In some embodiments, a targeting ligand fused to a second member of a protein:protein binding pair associated by an isopeptide bond comprises an antibody, or binding portion thereof, e.g., an antibody paratope.

An antibody paratope as described herein generally comprises at a minimum a complementarity determining region (CDR) that is involved in the specific recognition of a target (e.g., a detectable label, a cell surface receptor, etc.) e.g., a CDR3 region of a heavy and/or light chain variable domain. In some embodiments, a multispecific binding molecule comprises an antibody (or portion thereof) that comprises the antibody paratope that specifically binds the detectable label.

One embodiment of the present invention is a multimeric structure comprising a modified viral capsid protein of the present invention. A multimeric structure comprises at least 5, preferably at least 10, more preferably at least 30, most preferably at least 60 modified viral capsid proteins comprising a first member of a specific binding pair as described herein. They can form regular viral capsids (empty viral particles) or viral particles (capsids encapsidating a nucleotide of interest). The formation of viral particles comprising a viral genome is a preferred feature for use of the modified viral capsids described herein.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell (e.g., astrocyte).

### Conjugation of Molecular Cargo to Antigen-Binding Protein

In some embodiments, a molecular cargo described herein, e.g., a polynucleotide molecule described herein, or a liposome or LNP, is conjugated to an FGFR3 binding protein for delivery to a site of interest (e.g., brain or spinal cord). In some embodiments, the FGFR3 binding protein is conjugated to at least one molecular cargo (e.g., polynucleotide molecule, polypeptide molecule, liposome or LNP, and/or viral particle or viral capsid protein).

In some embodiments, an FGFR3 binding protein is conjugated to the 5′ terminus of a polynucleotide molecule, the 3′ terminus of a polynucleotide molecule, an internal site on a polynucleotide molecule, or in any combinations thereof.

In some embodiments, an FGFR3 binding protein is conjugated to the N terminus of a polypeptide molecule, the C terminus of a polypeptide molecule, an internal site on a polypeptide molecule, or in any combinations thereof.

In some embodiments, the FGFR3 binding protein is conjugated to at least one molecular cargo (e.g., at least one polynucleotide molecule, polypeptide molecule, liposome or LNP, and/or viral particle or viral capsid protein). In some embodiments, the FGFR3 binding protein is conjugated to at least 2, 3, 4, 5, 6, 7, 8, 10, 12, 16, 20, 24, 30 or more molecular cargoes described herein (e.g., at least 2, 3, 4, 5, 6, 7, 8, 10, 12, 16, 20, 24, 30 or more polynucleotide molecules, polypeptide molecules, and/or liposomes or LNPs).

In some embodiments, the FGFR3 In some embodiments, the FGFR3 binding protein is conjugated to a molecular cargo (e.g., polynucleotide molecule, polypeptide molecule, liposome or LNP, and/or viral particle or viral capsid protein) non-specifically. In some embodiments, the FGFR3 binding protein is conjugated to a molecular cargo (e.g., polynucleotide molecule, polypeptide molecule, liposome or LNP, and/or viral particle or viral capsid protein) via a lysine residue or a cysteine residue, in a non-site-specific manner. In some embodiments, the FGFR3 binding protein is conjugated to a molecular cargo (e.g., polynucleotide molecule, polypeptide molecule, liposome or LNP, and/or viral particle or viral capsid protein) via a lysine residue (e.g., lysine residue present in the FGFR3 binding protein) in a non-site-specific manner. In some cases, the FGFR3 binding protein is conjugated to a molecular cargo (e.g., polynucleotide molecule, or liposome or LNP) via a cysteine residue (e.g., cysteine residue present in the FGFR3 binding protein) in a non-site-specific manner.

In some embodiments, the FGFR3 binding protein is conjugated to a molecular cargo (e.g., polynucleotide molecule, polypeptide molecule, liposome or LNP, and/or viral particle or viral capsid protein) in a site-specific manner. In some embodiments, the FGFR3 binding protein is conjugated to a molecular cargo (e.g., polynucleotide molecule, polypeptide molecule, liposome or LNP, and/or viral particle or viral capsid protein) through a lysine residue, a cysteine residue, at the N-terminus, at the C-terminus, an unnatural amino acid, or an enzyme-modified or enzyme-catalyzed residue, via a site-specific manner. In some embodiments, the FGFR3 binding protein is conjugated to a molecular cargo (e.g., polynucleotide molecule, polypeptide molecule, liposome or LNP, and/or viral particle or viral capsid protein) through a lysine residue (e.g., lysine residue present in the FGFR3 binding protein) via a site-specific manner. In some embodiments, the FGFR3 binding protein is conjugated to a molecular cargo (e.g., polynucleotide molecule, polypeptide molecule, liposome or LNP, and/or viral particle or viral capsid protein) through a cysteine residue (e.g., cysteine residue present in the FGFR3 binding protein) via a site-specific manner. In some embodiments, the FGFR3 binding protein is conjugated to a molecular cargo (e.g., polynucleotide molecule, polypeptide molecule, liposome or LNP, and/or viral particle or viral capsid protein) at the N-terminus via a site-specific manner. In some embodiments, the FGFR3 binding protein is conjugated to a molecular cargo (e.g., polynucleotide molecule, polypeptide molecule, liposome or LNP, and/or viral particle or viral capsid protein) at the C-terminus via a site-specific manner. In some embodiments, the FGFR3 binding protein is conjugated to a molecular cargo (e.g., polynucleotide molecule, polypeptide molecule, liposome or LNP, and/or viral particle or viral capsid protein) through an unnatural amino acid via a site-specific manner. In some embodiments, the FGFR3 binding protein is conjugated to a molecular cargo (e.g., polynucleotide molecule, polypeptide molecule, liposome or LNP, and/or viral particle or viral capsid protein) through an enzyme-modified or enzyme-catalyzed residue via a site-specific manner.

In some embodiments, one or more molecular cargoes (e.g., polynucleotide molecule, polypeptide molecule, liposome or LNP, and/or viral particle or viral capsid protein) is conjugated to an FGFR3 binding protein. In some embodiments, about 1, 2, 3, 4, 5, 6, 7, 8, 9, 10, 11, 12, 13, 14, 15, 16, 20, 24, 30, 36 or more molecular cargoes (e.g., polynucleotide molecule, polypeptide molecule, liposome or LNP, and/or viral particle or viral capsid protein) are conjugated to one FGFR3 binding protein. In some embodiments, 1 molecular cargo is conjugated to one FGFR3 binding protein. In some embodiments, 2 molecular cargoes are conjugated to one FGFR3 binding protein. In some embodiments, 3 molecular cargoes are conjugated to one FGFR3 binding protein. In some embodiments, 4 molecular cargoes are conjugated to one FGFR3 binding protein. In some embodiments, 5 molecular cargoes are conjugated to one FGFR3 binding protein. In some embodiments, 6 molecular cargoes are conjugated to one FGFR3 binding protein. In some embodiments, 7 molecular cargoes are conjugated to one FGFR3 binding protein. In some embodiments, 8 molecular cargoes are conjugated to one FGFR3 binding protein. In some embodiments, 9 molecular cargoes are conjugated to one FGFR3 binding protein. In some embodiments, 10 molecular cargoes are conjugated to one FGFR3 binding protein. In some embodiments, 11 molecular cargoes are conjugated to one FGFR3 binding protein. In some embodiments, 12 molecular cargoes are conjugated to one FGFR3 binding protein. In some embodiments, 13 molecular cargoes are conjugated to one FGFR3 binding protein. In some embodiments, 14 molecular cargoes are conjugated to one FGFR3 binding protein. In some embodiments, 15 molecular cargoes are conjugated to one FGFR3 binding protein. In some embodiments, 16 molecular cargoes are conjugated to one FGFR3 binding protein. In some cases, the one or more molecular cargoes are the same. In other cases, the one or more molecular cargoes are different.

In some embodiments, the number of molecular cargoes conjugated to an FGFR3 binding protein forms a ratio. In some embodiments, the ratio is referred to as a DAR (drug-to-antibody) ratio, in which the drug as referred to herein is a molecular cargo described herein (e.g., polynucleotide molecule, polypeptide molecule, liposome or LNP, and/or viral particle or viral capsid protein). In some embodiments, the DAR ratio of the molecular cargo to FGFR3 binding protein is about 1, 2, 3, 4, 5, 6, 7, 8, 9, 10, 11, 12, 13, 14, 15, 16, 20, 24, 30, 36 or greater. In some embodiments, the DAR ratio of the molecular cargo to FGFR3 binding protein is about 1 or greater. In some embodiments, the DAR ratio of the molecular cargo to FGFR3 binding protein is about 2 or greater. In some embodiments, the DAR ratio of the molecular cargo to FGFR3 binding protein is about 3 or greater. In some embodiments, the DAR ratio of the molecular cargo to FGFR3 binding protein is about 4 or greater. In some embodiments, the DAR ratio of the molecular cargo to FGFR3 binding protein is about 5 or greater. In some embodiments, the DAR ratio of the molecular cargo to FGFR3 binding protein is about 6 or greater. In some embodiments, the DAR ratio of the molecular cargo to FGFR3 binding protein is about 7 or greater. In some embodiments, the DAR ratio of the molecular cargo to FGFR3 binding protein is about 8 or greater. In some embodiments, the DAR ratio of the molecular cargo to FGFR3 binding protein is about 9 or greater. In some embodiments, the DAR ratio of the molecular cargo to FGFR3 binding protein is about 10 or greater. In some embodiments, the DAR ratio of the molecular cargo to FGFR3 binding protein is about 11 or greater. In some embodiments, the DAR ratio of the molecular cargo to FGFR3 binding protein is about 12 or greater. In some embodiments, the DAR ratio of the molecular cargo to FGFR3 binding protein is about 16 or greater. In some embodiments, the DAR ratio of the molecular cargo to FGFR3 binding protein is about 20 or greater. In some embodiments, the DAR ratio of the molecular cargo to FGFR3 binding protein is about 24 or greater.

In some embodiments, the DAR ratio of the molecular cargo to FGFR3 binding protein is about 1, 2, 3, 4, 5, 6, 7, 8, 9, 10, 11, 12, 13, 14, 15, 16, 20, 24, 30, or 36. In some embodiments, the DAR ratio of the molecular cargo to FGFR3 binding protein is about 1. In some embodiments, the DAR ratio of the molecular cargo to FGFR3 binding protein is about 2. In some embodiments, the DAR ratio of the molecular cargo to FGFR3 binding protein is about 3. In some embodiments, the DAR ratio of the molecular cargo to FGFR3 binding protein is about 4. In some embodiments, the DAR ratio of the molecular cargo to FGFR3 binding protein is about 5. In some embodiments, the DAR ratio of the molecular cargo to FGFR3 binding protein is about 6. In some embodiments, the DAR ratio of the molecular cargo to FGFR3 binding protein is about 7. In some embodiments, the DAR ratio of the molecular cargo to FGFR3 binding protein is about 8. In some embodiments, the DAR ratio of the molecular cargo to FGFR3 binding protein is about 9. In some embodiments, the DAR ratio of the molecular cargo to FGFR3 binding protein is about 10. In some embodiments, the DAR ratio of the molecular cargo to FGFR3 binding protein is about 11. In some embodiments, the DAR ratio of the molecular cargo to FGFR3 binding protein is about 12. In some embodiments, the DAR ratio of the molecular cargo to FGFR3 binding protein is about 13. In some embodiments, the DAR ratio of the molecular cargo to FGFR3 binding protein is about 14. In some embodiments, the DAR ratio of the molecular cargo to FGFR3 binding protein is about 15. In some embodiments, the DAR ratio of the molecular cargo to FGFR3 binding protein is about 16.

In some embodiments, liposome or LNP functionalization with binding moieties is carried out via the adsorption phenomenon, covalent-nature binding, or binding by the use of adapter molecules or linkers.

### Adsorption

This phenomenon is a non-covalent immobilization strategy that comprises physical adsorption and ionic binding. Physical adsorption occurs via weak interactions such as hydrogen bonding, electrostatic, hydrophobic and Van der Weals attractive forces, while ionic binding occurs between the opposite charges of the FGFR3 binding protein and liposome or LNP surfaces. However, when compared to other methodologies such as covalent binding, adsorption provides less stability. On the other hand, the fact that the interaction is non-covalent may allow easier release of the cargo in the tumor tissue.

Covalent Strategies

Covalent binding requires prior activation of the LNPs. In some embodiments, covalent strategies occur via carbodiimide chemistry, maleimide chemistry or “click chemistry”, as discussed in detail below.

### Conjugation Chemistry

In some embodiments, a molecular cargo described herein (e.g., a polynucleotide molecule or polypeptide molecule described herein, or a liposome or LNP, or a viral particle or viral capsid protein) is conjugated to an FGFR3 binding protein. In some embodiments, a molecular cargo described herein (e.g., a polynucleotide molecule or polypeptide molecule described herein, or a liposome or LNP, or a viral particle or viral capsid protein) is conjugated to an FGFR3 binding protein directly. In some embodiments, a molecular cargo described herein (e.g., a polynucleotide molecule or polypeptide molecule described herein, or a liposome or LNP, or a viral particle or viral capsid protein) is conjugated to an FGFR3 binding protein via a linker covalently connecting the FGFR3 binding protein with the molecular cargo. In some embodiments, the FGFR3 binding protein is an antibody or antigen binding fragment thereof (e.g., scFv, Fab, or one-armed antibody).

In some embodiments, the molecular cargo described herein (e.g., a polynucleotide molecule or polypeptide molecule described herein, or a liposome or LNP, or a viral particle or viral capsid protein) is conjugated to the FGFR3 binding protein by a chemical ligation process. In some embodiments, the molecular cargo described herein (e.g., a polynucleotide molecule or polypeptide molecule described herein, or a liposome or LNP, or a viral particle or viral capsid protein) is conjugated to the FGFR3 binding protein by a native ligation. In some embodiments, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some embodiments, the conjugation is as described in U.S. Pat. No. 8,936,910. In some embodiments, the molecular cargo described herein (e.g., a polynucleotide molecule or polypeptide molecule, described herein, or a liposome or LNP, or a viral particle or viral capsid protein) is conjugated to the FGFR3 binding protein either site-specifically or non-specifically via native ligation chemistry.

In some embodiments, the molecular cargo described herein (e.g., a polynucleotide molecule or polypeptide molecule described herein, or a liposome or LNP, or a viral particle or viral capsid protein) is conjugated to the FGFR3 binding protein by a site-directed method utilizing a “traceless” coupling technology (Philochem). In some embodiments, the “traceless” coupling technology utilizes an N-terminal 1,2-aminothiol group on the FGFR3 binding protein which is then conjugated with a molecular cargo described herein (e.g., a polynucleotide molecule or polypeptide molecule described herein, or a liposome or LNP, or a viral particle or viral capsid protein) containing an aldehyde group. (see Casi et al., “Site-specific traceless coupling of potent cytotoxic drugs to recombinant antibodies for pharmacodelivery,” JACS 134(13): 5887-5892 (2012)).

In some embodiments, the molecular cargo described herein (e.g., a polynucleotide molecule or polypeptide molecule described herein, or a liposome or LNP, or a viral particle or viral capsid protein) is conjugated to the FGFR3 binding protein by a site-directed method utilizing an unnatural amino acid incorporated into the FGFR3 binding protein. In some embodiments, the unnatural amino acid comprises p-acetylphenylalanine (pAcPhe). In some embodiments, the keto group of pAcPhe is selectively coupled to an alkoxy-amine derivatived conjugating moiety to form an oxime bond. (see Axup et al., “Synthesis of site-specific antibody-drug conjugates using unnatural amino acids, “PNAS 109(40): 16101-16106 (2012)).

In some embodiments, the molecular cargo described herein (e.g., a polynucleotide molecule or polypeptide molecule described herein, or a liposome or LNP, or a viral particle or viral capsid protein) is conjugated to the FGFR3 binding protein by a site-directed method utilizing an enzyme-catalyzed process. In some embodiments, the site-directed method utilizes SMARTag™ technology (Catalent, Inc.). In some embodiments, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized molecular cargo described herein (e.g., a polynucleotide molecule or polypeptide molecule described herein, or a liposome or LNP, or a viral particle or viral capsid protein) via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013))

In some embodiments, the enzyme-catalyzed process comprises transglutaminase (TG), e.g., microbial transglutaminase (mTG). In some cases, the molecular cargo described herein (e.g., a polynucleotide molecule or polypeptide molecule described herein, or a liposome or LNP, or a viral particle or viral capsid protein) is conjugated to the FGFR3 binding protein utilizing a microbial transglutaminase-catalyzed process. In some embodiments, mTG catalyzes the formation of a covalent bond between the amide side chain of a glutamine within the recognition sequence and a primary amine of a functionalized molecular cargo described herein (e.g., a polynucleotide molecule or polypeptide molecule described herein, or a liposome or LNP, or a viral particle or viral capsid protein). In some embodiments, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some embodiments, a sequence of amino acids comprising an acceptor glutamine residue are incorporated into (e.g., appended to) a polypeptide sequence, under suitable conditions, for recognition by a TG. This sequence leads to cross-linking by the TG through a reaction between an amino acid side chain within the sequence of amino acids and a reaction partner. The recognition tag may be a peptide sequence that is not naturally present in the polypeptide comprising the TG recognition tag. In some embodiments, the TG recognition tag comprises at least one Gln. In some embodiments, the TGase recognition tag comprises an amino acid sequence XXQX, wherein X is any amino acid (e.g., conventional amino acid Leu, Ala, Gly, Ser, Val, Phe, Tyr, His, Arg, Asn, Glu, Asp, Cys, Gln, Ile, Met, Pro, Thr, Lys, or Trp or nonconventional amino acid). In some embodiments, the acyl donor glutamine-containing tag comprises an amino acid sequence selected from the group consisting of LLQGG (SEQ ID NO: 290), LLQG (SEQ ID NO: 291), LSLSQG (SEQ ID NO: 292), GGGLLQGG (SEQ ID NO: 293), GLLQG (SEQ ID NO: 294), LLQ, GSPLAQSHGG (SEQ ID NO: 295), GLLQGGG (SEQ ID NO: 296), GLLQGG (SEQ ID NO: 297), GLLQ (SEQ ID NO: 298), LLQLLQGA (SEQ ID NO: 299), LLQGA (SEQ ID NO: 300), LLQYQGA (SEQ ID NO: 301), LLQGSG (SEQ ID NO: 302), LLQYQG (SEQ ID NO: 303), LLQLLQG (SEQ ID NO: 304), SLLQG (SEQ ID NO: 305), LLQLQ (SEQ ID NO: 306), LLQLLQ (SEQ ID NO: 307), and LLQGR (SEQ ID NO: 308). See, e.g., PCT Publication No. WO2012/059882. In some embodiments, the acyl donor glutamine-containing tag is present at the N-terminus of the antigen-binding protein. In some embodiments, the acyl donor glutamine-containing tag is present at the C-terminus of the antigen-binding protein. In some embodiments, the acyl donor glutamine-containing tag is present both at the N-terminus and the C-terminus of the antigen-binding protein.

In some embodiments, the molecular cargo described herein (e.g., a polynucleotide molecule or polypeptide molecule described herein, or a liposome or LNP, or a viral particle or viral capsid protein) is conjugated to the FGFR3 binding protein by a method as described in PCT Publication No. WO2014/140317, which utilizes a sequence-specific transpeptidase. In some embodiments, the molecular cargo described herein (e.g., a polynucleotide molecule or polypeptide molecule described herein, or a liposome or LNP, or a viral particle or viral capsid protein) is conjugated to the FGFR3 binding protein by a method as described in U.S. Patent Publication Nos. 2015/0105539 and 2015/0105540.

In some embodiments, the molecular cargo described herein (e.g., a polynucleotide molecule or polypeptide molecule described herein, or a liposome or LNP, or a viral particle or viral capsid protein) is conjugated to the FGFR3 binding protein utilizing Azide-Alkyne Cycloaddition (CuAAC) click chemistry. Azides and alkynes can undergo catalyst free [3+2] cycloaddition by a using the reaction of activated alkynes with azides. Such catalyst-free [3+2] cycloaddition can be used in the methods described herein to conjugate an FGFR3 binding protein and the molecular cargo described herein (e.g., a polynucleotide molecule or polypeptide molecule described herein, or a liposome or LNP, or a viral particle or viral capsid protein). Alkynes can be activated by ring strain such as, by way of example only, eight-membered ring structures, or nine-membered, appending electron-withdrawing groups to such alkyne rings, or alkynes can be activated by the addition of a Lewis acid such as, by way of example only, Au(I) or Au(III).

Alkynes activated by ring strain have been described and used in “copperless” [3+2] cycloaddition. For example, the cyclooctynes and difluorocyclooctynes described by Agard et al., J. Am. Chem. Soc., 126 (46):15046-15047 (2004), the dibenzocyclooctynes described by Boons et al., PCT International Publication No. WO 2009/067663 A1 (2009), the aza-dibenzocyclooctynes described by Debets et al., Chem. Comm., 46:97-99 (2010), and the cyclononynes described by Dommerholt et al., Angew. Chem. 122:9612-9615 (2010)). In some embodiments, a tetrazine (Tzn)-activated FGFR3 binding protein may be cross-linked to a trans-cyclooctene (TCO)-activated molecular cargo described herein (e.g., a polynucleotide molecule or polypeptide molecule described herein, or a liposome or LNP, or a viral particle or viral capsid protein). In some embodiments, a TCO-activated FGFR3 binding protein may be crosslinked to a Tzn-activated molecular cargo described herein (e.g., a polynucleotide molecule or polypeptide molecule described herein, or a liposome or LNP, or a viral particle or viral capsid protein).

### Linkers

Complexes described herein generally comprise a linker that connects a binding agent to a molecular cargo (e.g., a polynucleotide molecule, polypeptide molecule, a liposome or an LNP). A linker comprises at least one covalent bond. In some embodiments, a linker may be a single bond, e.g., a disulfide bond or disulfide bridge, that connects a binding agent to a polynucleotide molecule, a polypeptide molecule, a liposome or LNP, or a viral particle or viral capsid protein. However, in some embodiments, a linker may connect a binding agent to a polynucleotide molecule, a polypeptide molecule, a liposome or LNP, or a viral particle or viral capsid protein through multiple covalent bonds. A linker is generally stable in vitro and in vivo, and may be stable in certain cellular environments. Additionally, generally a linker does not negatively impact the functional properties of either the binding agent or the polynucleotide molecule, polypeptide molecule, liposome or LNP, or viral particle or viral capsid protein. Examples and methods of synthesis of linkers are known in the art (see, e.g. Kline, T. et al. “Methods to Make Homogenous Antibody Drug Conjugates.” Pharmaceutical Research, 2015, 32:11, 3480-3493; Jain, N. et al. “Current ADC Linker Chemistry” Pharm Res. 2015, 32:11, 3526-3540; McCombs, J. R. and Owen, S. C., “Antibody Drug Conjugates: Design and Selection of Linker, Payload and Conjugation Chemistry” AAPS J. 2015, 17:2, 339-351).

A precursor to a linker typically will contain two different reactive species that allow for attachment to both the binding agent and a polynucleotide molecule, a polypeptide molecule, a liposome or LNP, ora viral particle or viral capsid protein. In some embodiments, the two different reactive species may be a nucleophile and/or an electrophile. In some embodiments, a linker is connected to a binding agent via conjugation to a lysine residue or a cysteine residue of the binding agent. In some embodiments, a linker is connected to a cysteine residue of a muscle-targeting agent via a maleimide-containing linker, wherein optionally the maleimide-containing linker comprises a maleimidocaproyl or maleimidomethyl cyclohexane-1-carboxylate group. In some embodiments, a linker is connected to a cysteine residue of a muscle-targeting agent or thiol functionalized molecular cargo via a 3-arylpropionitrile functional group. In some embodiments, a linker is connected to a binding agent and/or a polynucleotide molecule, polypeptide molecule or an LNP via an amide bond, a hydrazide, a triazole, a thioether or a disulfide bond.

In some embodiments, a linker described herein is a cleavable linker or a non-cleavable linker. In some embodiments, the linker is a cleavable linker. In other embodiments, the linker is a non-cleavable linker.

### Cleavable Linkers

A cleavable linker may be a protease-sensitive linker, a pH-sensitive linker, or a glutathione-sensitive linker. These linkers are generally cleavable only intracellularly and are preferably stable in extracellular environments.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline dipeptide sequence. In some embodiments, a protease-sensitive linker can be cleaved by a lysosomal protease, e.g. cathepsin B, and/or an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g. a cysteine residue.

### Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyneazide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation will be utilized to covalently link a muscle-targeting agent comprising a LPXT sequence to a molecular cargo comprising a (G), sequence (see, e.g. Proft T. Sortase-mediated protein ligation: an emerging biotechnology tool for protein modification and immobilization. Biotechnol Lett. 2010, 32(1):1-10).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S; an imino, an optionally substituted nitrogen species, an optionally substituted oxygen species, an optionally substituted sulfur species, or a poly(alkylene oxide), e.g. polyethylene oxide or polypropylene oxide.

In some cases, the linker is a non-polymeric linker. A non-polymeric linker refers to a linker that does not contain a repeating unit of monomers generated by a polymerization process. Exemplary non-polymeric linkers include, but are not limited to, C1-C30 alkyl group (e.g., a C5, C4, C3, C2, or C1 alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C1-C30 alkyl group (e.g., a C5, C4, C3, C2, or C1 alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups. In one embodiment, the linker has a structure

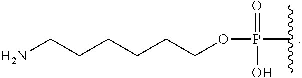 [SMILES: *P(=O)(O)OCCCCCCN]

In some cases, the non-polymeric linker does not encompass a polyalkylene oxide (e.g., PEG). In some cases, the non-polymeric linker does not encompass a PEG.

In some embodiments, the linker comprises a homobifunctional linker. Exemplary homobifunctional linkers include, but are not limited to, organoazide, organoalkyne, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DM P), dimethyl suberimidate (DMS), dimethyl-3,3′-dithiobispropionimidate (DTBP), 1,4-di-3′-(2′-pyridyldithio)propionamido) butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-113-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, a,a′-p-diaminodiphenyl, diiodo-p-xylene sulfonic acid, N,N′-ethylene-bis(iodoacetamide), or N, N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio) propionate (sPDP), long-chain N-succinimidyl 3-(2-pyridyldithio) propionate (LC-sPDP), water-soluble-long-chain N-succinimidyl 3-(2-pyridyldithio) propionate (sulfo-LCsPDP), succinimidyloxycarbonyl-a-methyl-α-(2-pyridyldithio) toluene (sMPT), sulfosuccinimidyl-64a-methyl-α-(2-pyridyldithio)toluamidoThexanoate (sulfo-LC-sMPT), succinimidyl-4-(N-maleimidomethyl) cyclohexane-1-car-boxylate (sMCC), sulfosuccinimidyl-4-(N-maleimidomethyl) cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MB s), N-succinimidyl (4-iodoacteyl)aminobenzoate (sIAB), sulfosuccinimidyl (4-iodoacteyl)aminobenzoate (sulfo-sIAB), succinimidyl-4-(p-maleimidophenyl)butyrate (sM PB), sulfosuccinimidyl-4-(p-maleimidophenyl)butyrate (sulfo-sM PB), N-(y-maleimidobutyryloxy)succinimide ester

(GM Bs), N-(y-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino) hexanoyl)amino]hexanoate (sIAXX), succinimidyl 4-(((iodoacetyl) amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydrylreactive cross-linkers such as 4-(4-N-maleimidophenyl) butyric acid hydrazide (MPBH), 4-(N-maleimidomethyl) cyclohexane-1-carboxyl-hydrazide-8 (M2C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-N Hs-AsA), sulfosuccinimidyl-(4-azidosalicylamido)hexanoate (sulfo-N Hs-LC-AsA), sulfosuccinimidyl-2-(p-azidosalicylamido)ethyl 1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido2′-nitrophenylamino)hexanoate (sANPAH), sulfo succinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate

(sADP), N-sulfosuccinimidyl(4-azidophenyl)-1,3′-dithiopropionate (sulfo-sADP), sulfosuccinimidyl 4-(p-azidophenyl) butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), sulfhydryl-reactive and photoreactive crosslinkers such as 1-(p-Azidosalicylamido)-4-(iodoacetamido) butane (AsIB), N44-(p-azidosalicylamido)butyl]-3′-(2′-pyridyldithio)propionamide (APDP), benzophenone-4-iodoacetamide, benzophenone-4-maleimide carbonylreactive and photoreactive cross-linkers such as p-azidobenzoyl hydrazide (ABH), carboxylate-reactive and photoreactive cross-linkers such as 4-(p-azidosalicylamido) butylamine (AsBA), and arginine-reactive and photoreactive cross-linkers such as p-azidophenyl glyoxal (APG).

In some embodiments, the linker comprises a reactive functional group. In some cases, the reactive functional group comprises a nucleophilic group that is reactive to an electrophilic group present on an FGFR3 binding protein. Exemplary electrophilic groups include carbonyl groups such as aldehyde, ketone, carboxylic acid, ester, amide, enone, acyl halide or acid anhydride. In some embodiments, the reactive functional group is aldehyde. Exemplary nucleophilic groups include hydrazide, oxime, amino, hydrazine, thiosemicarbazone, hydrazine carboxylate, and arylhydrazide.

In some embodiments, the linker comprises a maleimide group. In some embodiments, the maleimide group is also referred to as a maleimide spacer. In some embodiments, the maleimide group further encompasses a caproic acid, forming maleimidocaproyl (mc). In some cases, the linker comprises maleimidocaproyl (mc). In some cases, the linker is maleimidocaproyl (mc). In other embodiments, the maleimide group comprises a maleimidomethyl group, such as succinimidyl-4-(N-maleimidomethyl)cyclohexane-1-carboxylate (sMCC) or sulfosuccinimidyl-4-(N-maleimidomethyl)cyclohexane-1-carboxylate (sulfo-sMCC) described above.

In some embodiments, the maleimide group is a self-stabilizing maleimide. In some embodiments, the self-stabilizing maleimide utilizes diaminopropionic acid (DPR) to incorporate a basic amino group adjacent to the maleimide to provide intramolecular catalysis of tiosuccinimide ring hydrolysis, thereby eliminating maleimide from undergoing an elimination reaction through a retro-Michael reaction. In some embodiments, the self-stabilizing maleimide is a maleimide group described in Lyon, et al., “Self-hydrolyzing maleimides improve the stability and pharmacological properties of antibody-drug conjugates,” Nat. Biotechnol. 32(10):1059-1062 (2014). In some embodiments, the linker comprises a self-stabilizing maleimide. In some embodiments, the linker is a self-stabilizing maleimide.

In some embodiments, the linker comprises at least one azide moiety, e.g., as part of an organoazide moiety. In some embodiments, the linker comprises at least one alkyne moiety, e.g., as part of an organoalkyne moiety. In one embodiment, the alkyne is an activated alkyne. In some embodiments, the linker comprises a trizole (e.g., formed via a 1,3-cycloaddition reaction of an azide and an alkyne). In some embodiments, the linker comprises a Diels-Alder adduct.

In some embodiments, the linker comprises a peptide moiety. In some embodiments, the peptide moiety comprises at least 2, 3, 4, 5, or 6 more amino acid residues. In some embodiments, the peptide moiety comprises at most 2, 3, 4, 5, 6, 7, or 8 amino acid residues. In some embodiments, the peptide moiety comprises about 2, about 3, about 4, about 5, or about 6 amino acid residues. In some embodiments, the peptide moiety is a cleavable peptide moiety (e.g., either enzymatically or chemically). In some embodiments, the peptide moiety is a non-cleavable peptide moiety. In some embodiments, the peptide moiety comprises Val-Cit (valine-citrulline), Gly-Gly-Phe-Gly (SEQ ID NO: 394), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 395), or Gly-Phe-Leu-Gly (SEQ ID NO: 396). In some embodiments, the linker comprises a peptide moiety such as: Val-Cit (valine-citrulline), Gly-Gly-Phe-Gly (SEQ ID NO: 394), Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu (SEQ ID NO: 395), or Gly-Phe-Leu-Gly (SEQ ID NO: 396). In some cases, the linker comprises Val-Cit. In some cases, the linker is Val-Cit.

In some embodiments, the linker comprises a benzoic acid group, or its derivatives thereof. In some embodiments, the benzoic acid group or its derivatives thereof comprise paraaminobenzoic acid (PABA). In some embodiments, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some embodiments, the maleimide group is maleimidocaproyl (mc). In some embodiments, the peptide group is val-cit. In some embodiments, the benzoic acid group is PABA. In some embodiments, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some embodiments, the linker comprises a linker described in U.S. Pat. No. 9,089,614 or PCT Publication No. WO 2015/038426.

In some embodiments, the linker is a dendritic type linker. In some embodiments, the dendritic type linker comprises a branching, multifunctional linker moiety. In some embodiments, the dendritic type linker is used to increase the molar ratio of polynucleotide B to the FGFR3 binding protein. In some embodiments, the dendritic type linker comprises PAMAM dendrimers. In some embodiments, the dendritic type linker comprises triazoles. In some embodiments, the triazoles are connected by PEG links. In some embodiments, the linkers are as described in WO 2022/015656.

In some embodiments, the linker is a traceless linker or a linker in which after cleavage does not leave behind a linker moiety (e.g., an atom or a linker group) to an FGFR3 binding protein or a polynucleotide B. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some embodiments, the linker is a traceless linker described in Blaney, et al., “Traceless solid-phase organic synthesis,” Chem. Rev. 102: 2607-2024 (2002). In some embodiments, a linker is a traceless linker as described in U.S. Pat. No. 6,821,783.

In some embodiments, the linker is a linker described in U.S. Pat. Nos. 6,884,869; 7,498,298; 8,288,352; 8,609,105; or 8,697,688; U.S. Patent Publication Nos. US2014/0127239, US2013/028919, US2014/286970, US2013/0309256, US2015/037360, and US2014/0294851, or International Application Publication Nos. WO2015/057699; WO2014/080251; WO2014/197854; WO2014/145090; WO2014/177042, WO2022/015656.

In some embodiments, a linker is a bond, i.e., a linker is absent. In some cases, a linker is a non-polymeric linker. In some cases, a linker is a polymeric linker.

In some embodiments, the linker comprises an alkyl group. In some embodiments, the linker comprises a C1-C30 alkyl group, or a C1-C24 alkyl group, or a C1-C20 alkyl group, or a C1-C16 alkyl group, or a C1-C12 alkyl group, or a C1-C10 alkyl group, or a C1-C8 alkyl group, or a C1-C6 alkyl group, or a C1-C4 alkyl group. In some cases, a linker is a C1-C6 alkyl group, such as for example, a C3, C4, C3, C2, or C1 alkyl group. In some cases, the C1-C6 alkyl group is an unsubstituted C1-C6 alkyl group. As used in the context of a linker, alkyl means a saturated straight or branched hydrocarbon radical containing up to six carbon atoms. In some embodiments, the linker comprises a homobifunctional linker or a heterobifunctional linker described supra.

In some cases, a linker is an oligomeric or a polymeric linker. In some embodiments, a linker is a natural or synthetic oligomer or polymer, consisting of branched or unbranched monomers, and/or cross-linked network of monomers in two or three dimensions. In some embodiments, the linker comprises a polysaccharide, lignin, rubber, or polyalkylen oxide (e.g., polyethylene glycol).

In some embodiments, the at least one polymeric linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethylene terephthalate (also known as poly(ethylene terephthalate), PET, PETG, or PETE), polytetramethylene glycol (PTG), or polyurethane as well as mixtures thereof. In some embodiments, the linker comprises polyalkylene oxide. In some embodiments, the linker comprises PEG. In some embodiments, the linker comprises polyethylene imide (PEI) or hydroxy ethyl starch (HES).

In some embodiments, the linker comprises a polyalkylene oxide (e.g., PEG) comprising discrete ethylene oxide units. In some cases, the linker comprises between about 2 and about 48 ethylene oxide units. In some cases, the polymer moiety C comprises about 2, about 3, about 4, about 5, about 6, about 7, about 8, about 9, about 10, about 11, about 12, about 13, about 14, about 15, about 16, about 17, about 18, about 19, about 20, about 24, about 30, about 36, about 42, or about 48 ethylene oxide units.

In some embodiments, the FGFR3 binding protein is conjugated to the molecular cargo described herein (e.g., a polynucleotide molecule, a polypeptide molecule, a liposome or LNP, or a viral particle or viral capsid protein described herein) using a protamine linker, as disclosed in the U.S. Patent Application Publication Nos. US2002/0132990, US2004/0023902, US2007/012152, and US2010/0209440. In some embodiments, a protamine linker encompassed for use in the present disclosure comprises a sequence disclosed in US 2010/0209440.

Acid cleavable linkers can also be used with the present disclosure and include, but are not limited to, bismaleimideothoxy propane, adipic acid dihydrazide linkers (see, e.g., Fattom et al., Infection & Immun. 60:584 589, 1992) and acid labile transferrin conjugates that contain a sufficient portion of transferrin to permit entry into the intracellular transferrin cycling pathway (see, e.g., Welhoner et al., J. Biol. Chem. 266:4309 4314, 1991). Conjugates linked via acid cleavable linkers should be preferentially cleaved in acidic intracellular compartments, such as the endosome.

Photocleavable linkers can also be used with the protein-drug conjugates described herein. Photocleavable linkers are cleaved upon exposure to light (see, e.g., Goldmacher et al., Bioconj. Chem. 3:104-107, 1992), thereby releasing the targeted agent upon exposure to light. (Hazum et al., Proc. Eur. Pept. Symp., 16th, Brunfeldt, K (Ed), pp. 105 110, 1981; nitrobenzyl group as a photocleavable protective group for cysteine; Yen et al., Makromol. Chem 190:69 82, 1989; water soluble photocleavable copolymers, including hydroxypropylmethacrylamide copolymer, glycine copolymer, fluorescein copolymer and methylrhodamine copolymer; and Senter et al., Photochem. Photobiol. 42:231 237, 1985; nitrobenzyloxy carbonyl chloride cross linking reagents that produce photocleavable linkages), relevant portions incorporated herein by reference. Such linkers are particularly useful in treating dermatological or ophthalmic conditions. In addition, other tissues, such as blood vessels that can be exposed to light using fiber-optics during angioplasty in the prevention or treatment of restenosis may benefit from the use of photocleavable linkers. After administration of the conjugate, the body part is exposed to light, resulting in release of the targeted moiety from the conjugate. Heat sensitive linkers would also have similar applicability.

In one embodiment, the linker has a structure

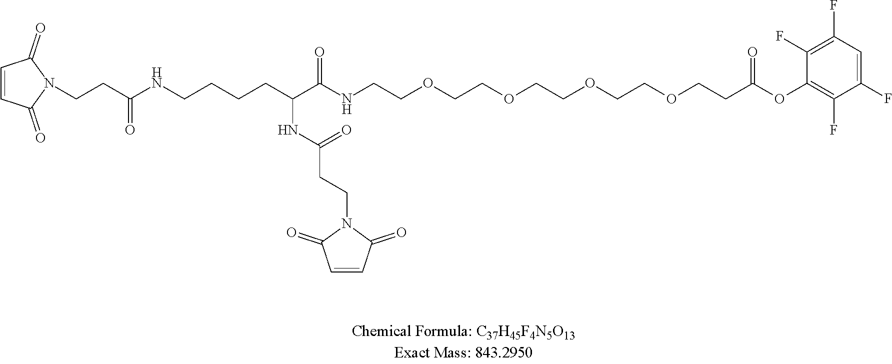 [SMILES: O=C(CCN1C(=O)C=CC1=O)NCCCCC(NC(=O)CCN1C(=O)C=CC1=O)C(=O)NCCOCCOCCOCCOCCC(=O)Oc1c(F)c(F)cc(F)c1F]

### Polynucleotides and Methods of Making

The present disclosure includes any polynucleotide described herein, for example, encoding an immunoglobulin V_{H}, V_{L}, CDR-H, CDR-L, HC or LC of H4H30063P; H4H30089P2; H4H30071P, H4H30066P; H4H30102P2; H4H30076P; H4H30105P2; H4H30108P2; H4H30117P2, H4H30045P; H4H30061P, H4H30095P2; and/or H4H30093P2, optionally, which is operably linked to a promoter or other expression control sequence. For example, the present disclosure provides any polynucleotide (e.g., DNA) that includes a nucleotide sequence set forth in SEQ ID NO: 1; 3; 5; 7; 9; 11; 13; 15; 17; 19; 21; 23; 25; 27; 29; 31; 33; 35; 37; 39; 41; 43; 45; 47; 49; 51; 53; 55; 57; 59; 61; 63; 65; 67; 69; 71; 73; 75; 77; 79; 81; 83; 85; 87; 89; 91; 93; 95; 97; 99; 101; 103; 105; 107; 109; 111; 113; 115; 117; 119; 121; 123; 125; 127; 129; 131; 133; 135; 137; 139; 141; 143; 145; 147; 149; 151; 152; 154; 156; 158; 160; 162; 164; 166; 168; 170; 172; 174; 176; 178; 180; 182; 184; 186; 188; 190; 192; 194; 196; 198; 200; 202; 204; 206; 208; 210; 212; 214; 216; 218; 220; 222; 224; 226; 228; or 230. In an embodiment, a polynucleotide of the present disclosure is fused to a secretion signal sequence. Polypeptides encoded by such polynucleotides are also within the scope of the present disclosure. A polynucleotide described herein can be DNA or RNA.

Nucleotide sequences of HCVRs and LCVRs of anti-FGFR3 protein-drug conjugates set forth herein are summarized below in Table 1-3. Polynucleotides encoding an anti-FGFR3 protein-drug conjugates, or polypeptide portion(s) thereof, that include one or more of the HCVRs and/or LCVRs set forth in Table 1-3 form part of the present disclosure.

TABLE 1-3
Nucleotide Sequences encoding Domains in Antibodies, Antigen-binding Fragments (e.g.,
Fabs or scFv Molecules) in Protein-drug Conjugates of the Present Disclosure.

| | anti-FGFR3 | | | | | | | | | | |
|---|---|---|---|---|---|---|---|---|---|---|---|
| # | Molecule | HCVR | HCDR1 | HCDR2 | HCDR3 | LCVR | LCDR1 | LCDR2 | LCDR3 | HC** | LC |
| 1 | H4H30063P | 1 | 3 | 5 | 7 | 9 | 11 | 13 | 15 | 17 | 19 |
| 2 | H4H30066P | 21 | 23 | 25 | 27 | 29 | 31 | 33 | 35 | 37 | 39 |
| 3 | H4H30071P | 41 | 43 | 45 | 47 | 49 | 51 | 53 | 55 | 57 | 59 |
| 4 | H4H30089P2 | 61 | 63 | 65 | 67 | 69 | 71 | 73 | 75 | 77 | 79 |
| 5 | H4H30093P2 | 81 | 83 | 85 | 87 | 89 | 91 | 93 | 95 | 97 | 99 |
| 6 | H4H30102P2 | 101 | 103 | 105 | 107 | 109 | 111 | 113 | 115 | 117 | 119 |
| 7 | H4H30076P | 121 | 123 | 125 | 127 | 129 | 131 | 33 | 133 | 135 | 137 |
| 8 | H4H30105P2 | 139 | 141 | 143 | 145 | 147 | 149 | 151 | 152 | 154 | 156 |
| 9 | H4H30108P2 | 158 | 160 | 162 | 164 | 147 | 149 | 151 | 152 | 166 | 156 |
| 10 | H4H30117P2 | 168 | 170 | 172 | 174 | 147 | 149 | 151 | 152 | 176 | 156 |
| 11 | H4H30045P | 178 | 180 | 182 | 184 | 186 | 188 | 190 | 192 | 194 | 196 |
| 12 | H4H30061P | 198 | 200 | 202 | 204 | 206 | 208 | 210 | 212 | 214 | 216 |
| 13 | H4H30095P2 | 218 | 220 | 222 | 224 | 226 | 31 | 33 | 75 | 228 | 230 |

**In an embodiment of the disclosure, the heavy chain lacks a codon encoding the C-terminal Lysine.

For example, the present disclosure includes a polynucleotide encoding an anti-FGFR3 protein-drug conjugate, or polypeptide portion(s) thereof, that includes:
a polynucleotide encoding a HCVR that comprises the nucleotide sequence set forth in SEQ ID NO: 1, and a LCVR that comprises the nucleotide sequence set forth in SEQ ID NO: 9;
a polynucleotide encoding a HCVR that comprises the nucleotide sequence set forth in SEQ ID NO: 21, and a LCVR that comprises the nucleotide sequence set forth in SEQ ID NO: 29;
a polynucleotide encoding a HCVR that comprises the nucleotide sequence set forth in SEQ ID NO: 41, and a LCVR that comprises the nucleotide sequence set forth in SEQ ID NO: 49;
a polynucleotide encoding a HCVR that comprises the nucleotide sequence set forth in SEQ ID NO: 61, and a LCVR that comprises the nucleotide sequence set forth in SEQ ID NO: 69;
a polynucleotide encoding a HCVR that comprises the nucleotide sequence set forth in SEQ ID NO: 81, and a LCVR that comprises the nucleotide sequence set forth in SEQ ID NO: 89;
a polynucleotide encoding a HCVR that comprises the nucleotide sequence set forth in SEQ ID NO: 101, and a LCVR that comprises the nucleotide sequence set forth in SEQ ID NO: 109;
a polynucleotide encoding a HCVR that comprises the nucleotide sequence set forth in SEQ ID NO: 121, and a LCVR that comprises the nucleotide sequence set forth in SEQ ID NO: 129;
a polynucleotide encoding a HCVR that comprises the nucleotide sequence set forth in SEQ ID NO: 139, and a LCVR that comprises the nucleotide sequence set forth in SEQ ID NO:147;
a polynucleotide encoding a HCVR that comprises the nucleotide sequence set forth in SEQ ID NO: 158, and a LCVR that comprises the nucleotide sequence set forth in SEQ ID NO:147;
a polynucleotide encoding a HCVR that comprises the nucleotide sequence set forth in SEQ ID NO: 168, and a LCVR that comprises the nucleotide sequence set forth in SEQ ID NO: 147;
a polynucleotide encoding a HCVR that comprises the nucleotide sequence set forth in SEQ ID NO: 178, and a LCVR that comprises the nucleotide sequence set forth in SEQ ID NO: 186;
a polynucleotide encoding a HCVR that comprises the nucleotide sequence set forth in SEQ ID NO: 198, and a LCVR that comprises the nucleotide sequence set forth in SEQ ID NO: 206; or
a polynucleotide encoding a HCVR that comprises the nucleotide sequence set forth in SEQ ID NO: 218, and a LCVR that comprises the nucleotide sequence set forth in SEQ ID NO: 226;
wherein the HCVR and LCVR are in either order.

In some embodiments, a nucleic acid molecule as described herein comprises a nucleic acid sequence encoding any of the HCVR amino acid sequences listed in Table 1-3; in certain embodiments the nucleic acid molecule comprises a polynucleotide sequence selected from any of the HCVR nucleic acid sequences listed in Table 1-3, or a substantially similar sequence thereof having at least 90%, at least 95%, at least 98% or at least 99% sequence identity thereto.

In some embodiments, the nucleic acid molecule comprises a polynucleotide sequence selected from any of the LCVR nucleic acid sequences listed in Table 1-3, or a substantially similar sequence thereof having at least 90%, at least 95%, at least 98% or at least 99% sequence identity thereto.

In some embodiments the nucleic acid molecule comprises a polynucleotide sequence selected from any of the HCDR1 nucleic acid sequences listed in Table 1-3, or a substantially similar sequence thereof having at least 90%, at least 95%, at least 98% or at least 99% sequence identity thereto.

In some embodiments the nucleic acid molecule comprises a polynucleotide sequence selected from any of the HCDR2 nucleic acid sequences listed in Table 1-3, or a substantially similar sequence thereof having at least 90%, at least 95%, at least 98% or at least 99% sequence identity thereto.

In some embodiments the nucleic acid molecule comprises a polynucleotide sequence selected from any of the HCDR3 nucleic acid sequences listed in Table 1-3, or a substantially similar sequence thereof having at least 90%, at least 95%, at least 98% or at least 99% sequence identity thereto.

In some embodiments the nucleic acid molecule comprises a polynucleotide sequence selected from any of the LCDR1 nucleic acid sequences listed in Table 1-3, or a substantially similar sequence thereof having at least 90%, at least 95%, at least 98% or at least 99% sequence identity thereto.

In some embodiments the nucleic acid molecule comprises a polynucleotide sequence selected from any of the LCDR2 nucleic acid sequences listed in Table 1-3, or a substantially similar sequence thereof having at least 90%, at least 95%, at least 98% or at least 99% sequence identity thereto.

In some embodiments the nucleic acid molecule comprises a polynucleotide sequence selected from any of the LCDR3 nucleic acid sequences listed in Table 1-3, or a substantially similar sequence thereof having at least 90%, at least 95%, at least 98% or at least 99% sequence identity thereto.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide described herein. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VI Ila-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present disclosure includes a polynucleotide comprising the following polynucleotide pairs encoding a V_{H} and V_{L}:
SEQ ID NO: 1 and SEQ ID NO: 9;
SEQ ID NO: 21 and SEQ ID NO: 29;
SEQ ID NO: 41 and SEQ ID NO: 49;
SEQ ID NO: 61 and SEQ ID NO: 69;
SEQ ID NO: 81 and SEQ ID NO: 89;
SEQ ID NO: 101 and SEQ ID NO: 109;
SEQ ID NO: 121 and SEQ ID NO: 129;
SEQ ID NO: 139 and SEQ ID NO:147;
SEQ ID NO: 158 and SEQ ID NO:147;
SEQ ID NO: 168 and SEQ ID NO: 147;
SEQ ID NO: 178 and SEQ ID NO: 186;
SEQ ID NO: 198 and SEQ ID NO: 206; and/or
SEQ ID NO: 218 and SEQ ID NO: 226;
   or two separate polynucleotides, each including one of the sequences
SEQ ID NO: 1 and SEQ ID NO: 9;
SEQ ID NO: 21 and SEQ ID NO: 29;
SEQ ID NO: 41 and SEQ ID NO: 49;
SEQ ID NO: 61 and SEQ ID NO: 69;
SEQ ID NO: 81 and SEQ ID NO: 89;
SEQ ID NO: 101 and SEQ ID NO: 109
SEQ ID NO: 121 and SEQ ID NO: 129;
SEQ ID NO: 139 and SEQ ID NO:147;
SEQ ID NO: 158 and SEQ ID NO:147;
SEQ ID NO: 168 and SEQ ID NO: 147;
SEQ ID NO: 178 and SEQ ID NO: 186;
SEQ ID NO: 198 and SEQ ID NO: 206; or
SEQ ID NO: 218 and SEQ ID NO: 226.

The present disclosure includes a polynucleotide comprising the following polynucleotide sets which encode a CDR-H1, CDR-H2, CDR-H3, CDR-L1, CDR-L2 and CDR-L3:
SEQ ID NOs: 3, 5, 7, 11, 13 and 15;
SEQ ID NOs: 23, 25, 27, 31, 33 and 35;
SEQ ID NOs: 43, 45, 47, 51, 53 and 55;
SEQ ID NOs: 63, 65, 67, 71, 73 and 75;
SEQ ID NOs: 83, 85, 87, 91, 93 and 95;
SEQ ID NOs: 103, 105, 107, 111, 113 and 115;
SEQ ID NOs: 123, 125, 127, 131, 33 and 133;
SEQ ID NOs: 141, 143, 415, 149, 151 and 152;
SEQ ID NOs: 160, 162, 164, 149, 151 and 152;
SEQ ID NOs: 170, 172, 174, 149, 151 and 152;
SEQ ID NOs: 180, 182, 184, 188, 190 and 192;
SEQ ID NOs: 200, 202, 204, 208, 210 and 212; and/or
SEQ ID NOs: 220, 222, 224, 31, 33 and 75;
   or two separate polynucleotides, each including one of the sequences
SEQ ID NOs: 3, 5 and 7; and 11, 13 and 15;
SEQ ID NOs: 23, 25 and 27; and 31, 33 and 35;
SEQ ID NOs: 43, 45 and 47; and 51, 53 and 55;
SEQ ID NOs: 63, 65 and 67; and 71, 73 and 75;
SEQ ID NOs: 83, 85 and 87; and 91, 93 and 95;
SEQ ID NOs: 103, 105 and 107; and 111, 113 and 115
SEQ ID NOs: 123, 125, and 127; 131, 33 and 133;
SEQ ID NOs: 141, 143, and 415; 149, 151 and 152;
SEQ ID NOs: 160, 162, and 164; 149, 151 and 152;
SEQ ID NOs: 170, 172, and 174; 149, 151 and 152;
SEQ ID NOs: 180, 182, and 184; 188, 190 and 192;
SEQ ID NOs: 200, 202, and 204; 208, 210 and 212; or
SEQ ID NOs: 220, 222, and 224; 31, 33 and 75.

The present disclosure includes a polynucleotide comprising the following polynucleotide pairs encoding a HC and LC:
SEQ ID NO: 17 and SEQ ID NO: 19;
SEQ ID NO: 37 and SEQ ID NO: 39;
SEQ ID NO: 57 and SEQ ID NO: 59;
SEQ ID NO: 77 and SEQ ID NO: 79;
SEQ ID NO: 97 and SEQ ID NO: 99;
SEQ ID NO: 117 and SEQ ID NO: 119;
SEQ ID NO: 135 and SEQ ID NO: 137;
SEQ ID NO: 154 and SEQ ID NO: 156;
SEQ ID NO: 166 and SEQ ID NO: 156;
SEQ ID NO: 176 and SEQ ID NO: 156;
SEQ ID NO: 194 and SEQ ID NO: 196;
SEQ ID NO: 214 and SEQ ID NO: 216; and/or
SEQ ID NO: 228 and SEQ ID NO: 230
   or two separate polynucleotides, each including one of the sequences
SEQ ID NO: 17 and SEQ ID NO: 19;
SEQ ID NO: 37 and SEQ ID NO: 39;
SEQ ID NO: 57 and SEQ ID NO: 59;
SEQ ID NO: 77 and SEQ ID NO: 79;
SEQ ID NO: 97 and SEQ ID NO: 99;
SEQ ID NO: 117 and SEQ ID NO: 119
SEQ ID NO: 135 and SEQ ID NO: 137;
SEQ ID NO: 154 and SEQ ID NO: 156;
SEQ ID NO: 166 and SEQ ID NO: 156;
SEQ ID NO: 176 and SEQ ID NO: 156;
SEQ ID NO: 194 and SEQ ID NO: 196;
SEQ ID NO: 214 and SEQ ID NO: 216; and/or
SEQ ID NO: 228 and SEQ ID NO: 230.

Host cells including the two separate polynucleotides as discussed above, each integrated into chromosomal DNA of the host cell at different loci or ectopic, wherein such polynucleotide are maintained in separate genetic elements, are within the scope of the present disclosure.

The present disclosure includes polynucleotides encoding immunoglobulin polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, -2; gap costs: linear). In an embodiment, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment, be missense or nonsense mutations. In an embodiment, such a variant polynucleotide encodes an immunoglobulin polypeptide chain which can be incorporated into an FGFR3 binding protein, i.e., such that the protein retains specific binding to FGFR3.

Eukaryotic and prokaryotic host cells, including mammalian cells, may be used as hosts for expression of an FGFR3 binding protein (e.g., antibody or antigen-binding fragment thereof). Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanofica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present disclosure includes an isolated host cell (e.g., a CHO cell or any type of host cell set forth above) comprising an antigen-binding protein, a V_{H}, V_{L}, HC, LC or CDRs thereof (or variant thereof), such as H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2; and/or a polynucleotide encoding one or more immunoglobulin chains thereof (e.g., as discussed herein). In an embodiment, a host cell includes two separate polynucleotides, one encoding a V_{H} and the other encoding a V_{L}; or one encoding a HC and the other encoding a LC.

The present disclosure also includes a cell which is expressing FGFR3 or an antigenic fragment or fusion thereof (e.g., His₆ (SEQ ID NO: 235), Fc (e.g., mouse Fc (mFc)), myc, or mycmycHis6 (mmh)) which is bound by an antigen-binding protein of the present disclosure (e.g., an antibody or antigen-binding fragment thereof), for example, H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2, for example, wherein the cell is in the body of a subject or is in vitro. In addition, the present disclosure also provides a complex comprising an FGFR3 binding protein, e.g., antibody or antigen-binding fragment thereof, as discussed herein complexed with FGFR3 polypeptide or an antigenic fragment thereof or fusion thereof and/or with a secondary antibody or antigen-binding fragment thereof (e.g., detectably labeled secondary antibody) that binds specifically to the anti-FGFR3 antibody or fragment. In an embodiment of the disclosure, the complex is in vitro (e.g., is immobilized to a solid substrate) or is in the body of a subject.

In an embodiment, a myc tag has the amino acid sequence EQKLISEEDLGG (SEQ ID NO: 234), a His₆ (SEQ ID NO: 235) or hexahis (SEQ ID NO: 235) or hexahistidine (SEQ ID NO: 235) tag has the amino acid sequence HHHHHH (SEQ ID NO: 235), an mmh tag has the amino acid sequence EQKLISEEDLGGEQKLISEEDLHHHHHH (SEQ ID NO: 236) and a mouse Fc tag has the amino acid sequence

(SEQ ID NO: 237)
EPRGPTIKPCPPCKCPAPNLLGGPSVFIFPPKIKDVLMISLSPIV
TCVVVDVSEDDPDVQISWFVNNVEVHTAQTQTHREDYNSTLRVVS
ALPIQHQDWMSGKEFKCKVNNKDLPAPIERTISKPKGSVRAPQVY
VLPPPEEEMTKKQVTLTCMVTDFMPEDIYVEWTNNGKTELNYKNT
EPVLDSDGSYFMYSKLRVEKKNWVERNSYSCSVVHEGLHNHHTTK
SFSRTPGK.

In addition, the present disclosure also provides a complex comprising an anti-FGFR3 protein-drug conjugate, as discussed herein complexed with a FGFR3 polypeptide or an antigenic fragment thereof or fusion thereof and/or with a secondary antibody or antigen-binding fragment thereof (e.g., detectably labeled secondary antibody) that binds specifically to the anti-FGFR3 protein-drug conjugate. In an embodiment, the complex is in vitro (e.g., is immobilized to a solid substrate) or is in the body of a subject.

Recombinant FGFR3 binding proteins, e.g., antibodies and antigen-binding fragments, or anti-FGFR3 fusion proteins disclosed herein may also be produced in an E. coli/T7 expression system. In this embodiment, polynucleotides encoding the anti-FGFR3 antibody immunoglobulin molecules described herein (e.g., HC, LC, V_{H} and/or V_{L} or CDRs thereof of (H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2)) may be inserted into a pET-based plasmid and expressed in the E. coli/T7 system. For example, the present disclosure includes methods for expressing an antibody or antigen-binding fragment thereof or immunoglobulin chain thereof in a host cell (e.g., bacterial host cell such as E. coli such as BL21 or BL21DE3) comprising expressing T7 RNA polymerase in the cell which also includes a polynucleotide encoding an immunoglobulin chain (e.g., including the nucleotide sequence in any one or more of SEQ ID NOs: 1, 3, 5, 7, 9, 11, 13, 15, 17 or 19; ora variant thereof) that is operably linked to a T7 promoter. For example, in an embodiment, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5; 189(1): 113-30.

There are several methods by which to produce recombinant antibodies which are known in the art. One example of a method for recombinant production of antibodies is disclosed in U.S. Pat. No. 4,816,567.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, polynucleotides may be introduced into mammalian cells by viral vectors. Methods of transforming cells are well known in the art. See, for example, U.S. Pat. Nos. 4,399,216; 4,912,040; 4,740,461 and 4,959,455. Thus, recombinant methods for making an anti-FGFR3 (e.g., monomeric or dimeric FGFR3b and/or FGFR3c) antigen-binding protein, such as an antibody or antigen-binding fragment thereof of the present disclosure, or an immunoglobulin chain thereof, described herein may comprise (i) introducing, into a host cell, one or more polynucleotides (e.g., including the nucleotide sequence in any one or more of SEQ ID NOs: 1, 9, 17 and/or 19; or a variant thereof) encoding light and/or heavy immunoglobulin chains of the antigen-binding protein, e.g., H4H30063P; H4H30089P2; H4H30071P, H4H30066P; H4H30102P2; H4H30076P; H4H30105P2; H4H30108P2; H4H30117P2; H4H30045P; H4H30061P, H4H30095P2; or H4H30093P2, for example, wherein the polynucleotide is in a vector; and/or integrates into the host cell chromosome and/or is operably linked to a promoter; (ii) culturing the host cell (e.g., CHO or Pichia or Pichia pastoris) under conditions favorable to expression of the polynucleotide and, (iii) optionally, isolating the antigen-binding protein (e.g., antibody or antigen-binding fragment) or chain from the host cell and/or medium in which the host cell is grown. When making an antigen-binding protein (e.g., antibody or antigen-binding fragment) comprising more than one immunoglobulin chain, e.g., an antibody that comprises two heavy immunoglobulin chains and two light immunoglobulin chains, co-expression of the chains in a single host cell leads to association of the chains, e.g., in the cell or on the cell surface or outside the cell if such chains are secreted, so as to form the antigen-binding protein (e.g., antibody or antigen-binding fragment). The methods described herein include those wherein only a heavy immunoglobulin chain or only a light immunoglobulin chain or both (e.g., any of those discussed herein including mature fragments and/or variable domains thereof) are expressed in a cell. Such single chains are useful, for example, as intermediates in the expression of an antibody or antigen-binding fragment that includes such a chain. For example, the present disclosure also includes FGFR3 binding proteins, such as antibodies and antigen-binding fragments thereof which are the product of the production methods set forth herein, and, optionally, the purification methods set forth herein.

In an embodiment of the disclosure, a method for making an anti-FGFR3 (e.g., monomeric or dimeric FGFR3b and/or FGFR3c) antigen-binding protein, e.g., antibody or antigen-binding fragment thereof, includes a method of purifying the antigen-binding protein, e.g., by column chromatography, precipitation and/or filtration. As discussed, the product of such a method also forms part of the present disclosure.

### Preparation of Human Antibodies

The anti-FGFR3 (e.g., monomeric or dimeric FGFR3b and/or FGFR3c) antibodies and antigen-binding fragments described herein (e.g., H4H30063P; H4H30089P2; H4H30071P, H4H30066P; H4H30102P2; H4H30076P; H4H30105P2; H4H30108P2, H4H30117P2, H4H30045P; H4H30061P, H4H30095P2; or H4H30093P2) can be fully human antibodies and fragments. Methods for generating monoclonal antibodies, including fully human monoclonal antibodies are known in the art. Any such known methods can be used in the methods described herein to make human antibodies that specifically bind to FGFR3.

Using VELOCIMMUNE™ technology, for example, or any other similar known method for generating fully human monoclonal antibodies, high affinity chimeric antibodies to FGFR3 are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate a fully human anti-FGFR3 antibody. While the constant region selected may vary according to specific use, high affinity antigen-binding and target specificity characteristics reside in the variable region. In certain instances, fully human anti-FGFR3 antibodies are isolated directly from antigen-positive B cells. See, for example, U.S. Pat. No. 6,596,541, Regeneron Pharmaceuticals, VELOCIMMUNE®.

### Treatment and Administration

The present disclosure provides anti-FGFR3 protein-drug conjugates which can be used, for example, for delivering a molecular cargo to the body of a subject (e.g., the nervous system including the brain and the spinal cord, or eye, and, in particular, astrocytes residing therein), for treating or preventing a disease or disorder (e.g., neurological disease or disorder), in the body of the subject.

In some embodiments, the disease or disorder being treated here can be an FGFR3-mediated condition. An FGFR3-mediated condition is any condition that is mediated at least in part by the activity of FGFR3, for example, tyrosine kinase activity of FGFR3 or activity of molecules downstream of FGFR3 (e.g., CD73 or the MEK pathway in a tumor cell expressing FGFR3). An FGFR3-mediated condition can also include T-cell suppression mediated by a tumor cell expressing FGFR3, e.g., adenosine-mediated suppression of T-cells via the A2A receptor, for example, wherein the CD73 catalyzes conversion of AMP to adenosine. CD73 (NTSE, ecto-5′-nucleotidase) is a glycosylphosphatidylinositol-(GPI-)anchored cell-surface enzyme that plays a crucial role in the purinergic signaling pathway by dephosphorylating AMP (adenosine monophosphate) into adenosine. Extracellular adenosine itself is involved in tumor immunoescape and invasion of tumor cells, while nonenzymatic functions of CD73 are related to cell adhesion and migration of tumor cells.

In some embodiments, the disease or disorder being treated here can be a condition that is not mediated by the activity of FGFR3.

In one aspect, the disclosure provides a pharmaceutical composition comprising protein-drug conjugate together with a pharmaceutically acceptable carrier and/or excipient. The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The pharmaceutical compositions of the disclosure may be in any suitable form (depending upon the desired method of administering to a patient). Suitable compositions and methods of administration are known to those skilled in the art, for example see, Johnson et al., Blood. 2009; 114(3):535-46.

The pharmaceutical compositions may comprise the protein-drug conjugates described herein either in the free form or in the form of a pharmaceutically acceptable salt. The term “pharmaceutically acceptable salt” as used herein refers to a derivative of the disclosed protein-drug conjugates wherein the protein-drug conjugates is modified by making acid or base salts of the agent. For example, acid salts are prepared from the free base (typically wherein the neutral form of the drug has a neutral —NH2 group) involving reaction with a suitable acid. Suitable acids for preparing acid salts include both organic acids, e.g., acetic acid, benzoic acid, citric acid, propionic acid, glycolic acid, trifluoroacetic acid, pyruvic acid, oxalic acid, malic acid, malonic acid, maleic acid, succinic acid, fumaric acid, tartaric acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a protein-drug conjugates are prepared using a pharmaceutically acceptable base such as sodium hydroxide, potassium hydroxide, ammonium hydroxide, calcium hydroxide, trimethylamine or the like.

In some embodiments, the protein-drug conjugates described herein may be present in a solution at a concentration of about 1 μg/mL to 50 mg/mL, for example, about 0.1 mg/mL to 10 mg/mL, about 0.2 mg/mL to 5 mg/mL, about 0.5 mg/mL to 8 mg/mL, about 0.8 mg/mL to 12 mg/mL, about 1 mg/mL to 15 mg/mL, about 2 mg/mL to 20 mg/mL, or about 5 mg/mL to 25 mg/mL, or about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.25 mg/mL, 1.5 mg/mL, 1.75 mg/mL, 2 mg/mL, 2.25 mg/mL, 2.5 mg/mL, 2.75 mg/mL, 3 mg/mL, 3.25 mg/mL, 3.5 mg/mL, 3.75 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL or 20 mg/mL.

The pharmaceutical composition may be adapted for administration by any appropriate route such as, e.g., parenteral (including subcutaneous, intramuscular, or intravenous), intrathecal, intracerebroventricular, intracisternal (e.g., cisterna magna), intraparenchymal injections into the central nervous system, enteral (including oral or rectal), inhalation, or intranasal routes.

Such compositions may be prepared by any method known in the art of pharmacy, for example, by mixing the active ingredient with the carrier(s) or excipient(s) under sterile conditions.

In addition, disclosed herein are pharmaceutical dosage forms comprising the protein-drug conjugates described herein.

Pharmaceutical compositions based on the protein-drug conjugates disclosed herein can be formulated in any conventional manner using one or more physiologically acceptable carriers and/or excipients. The protein-drug conjugates may be formulated for administration by, for example, injection, inhalation, or insulation (either through the mouth or the nose) or by oral, buccal, parenteral or rectal administration, or by administration directly to an organ or tissue.

The pharmaceutical compositions can be formulated for a variety of modes of administration, including systemic, topical, or localized administration. Techniques and formulations can be found in, for example, Remington's Pharmaceutical Sciences, Meade Publishing Co., Easton, Pa. In some embodiments, localized injection is used, including intrathecal, intracisternal (e.g., cisterna magna), intracerebroventricular, intraparenchymal injection. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

In some embodiments, the pharmaceutical compositions of the present disclosure may be lyophilized. As a non-limiting example, the obtained lyophilizate can be reconstituted into a hydrous composition by adding a hydrous solvent. In some embodiments, the hydrous composition may be able to be directly administered parenterally (e.g., via intrathecal, intracisternal (e.g., cisterna magna), intracerebroventricular, or intraparenchymal injection) to a patient. Therefore, a further embodiment of the present disclosure is a hydrous pharmaceutical composition, obtainable via reconstitution of the lyophilizate with a hydrous solvent.

In some embodiments, the pharmaceutical composition disclosed herein may comprise a lyophilized formulation. As a non-limiting example, the lyophilization formulation may comprise protein-drug conjugates described herein, mannitol, and/or TWEEN 80®. As another non-limiting example, the lyophilization formulation may comprise the protein-drug conjugates disclosed herein, mannitol and poloxamer 188. In some embodiments, the pharmaceutical composition may comprise a lyophilization formulation comprising a reconstituted-liquid composition.

In some embodiments, pharmaceutical compositions described herein may provide a formulation with an enhanced solubility and/or moistening of the lyophilizate over previously known compositions. As a non-limiting example, enhanced solubility and/or moistening of the lyophilizate may be achieved using an appropriate composition of excipients. In this way, pharmaceutical compositions described herein comprising protein-drug conjugates described herein may be developed to show a desired shelf stability at (e.g., at −20° C., +5° C., or +25° C.) and can be easily resolubilized such that the lyophilizate can be completely dissolved through the use of a buffer or other excipients from seconds up to two or more minutes, with or without the use of an of ultrasonic homogenizer. Furthermore, the composition can be easily provided to a patient in need of treatment via any appropriate delivery route disclosed herein, e.g., parenteral (including intrathecal, intracisternal (e.g., cisterna magna), intracerebroventricular, or intraparenchymal injections), enteral (including oral or rectal), inhalation, or intranasal routes. As a non-limiting example, the pH-value of the resulting solution may be between pH 2.7 and pH 9.

In some embodiments, the pharmaceutical compositions of the present disclosure may be desiccated, e.g., freeze-dried, or a pharmaceutical formulation thereof that includes a pharmaceutically acceptable carrier but substantially lacks water.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. ationd oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long-acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is then slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the vector particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

A protein-drug conjugate described herein can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutically acceptable carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow-release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered the protein-drug conjugates described herein on a daily or weekly basis for a time period or on a monthly, bi-yearly or yearly basis.

In addition to the compounds formulated for parenteral administration, such as intrathecal, intracerebroventricular, intracisternal (e.g., cisterna magna), or intraparenchymal injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compounds sucrose as a sweetening agent methyl and propylparabens as preservatives, a dye and flavoring, such as cherry or orange flavor.

The mode of administration of the anti-FGFR3 protein-drug conjugates or composition thereof can vary. Routes of administration include parenteral, non-parenteral, oral, rectal, transmucosal, intestinal, parenteral; intramuscular, subcutaneous, intradermal, intramedullary, intrathecal, intracisternal (e.g., cisterna magna), intracerebroventricular, direct intraventricular, intravenous, intraperitoneal, intranasal, intraocular, inhalation, insufflation, topical, cutaneous, intraocular, intravitreal, transdermal or intra-arterial.

Compositions may be administered to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, intracisternally, intracerebroventricularly, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. Compositions and methods as disclosed herein can also include adjuvant therapy.

The pharmaceutical compositions described herein may be administered directly into the patient, into the affected organ or systemically, or applied ex vivo to cells derived from the patient or a human cell line which are subsequently administered to the patient, or used in vitro to select a subpopulation of cells derived from the patient, which are then re-administered to the patient.

The present disclosure provides a vessel (e.g., a plastic or glass vial, e.g., with a cap or a chromatography column, hollow bore needle or a syringe cylinder) comprising any of the anti-FGFR3 protein-drug conjugates, or a pharmaceutical formulation comprising a pharmaceutically acceptable carrier thereof.

In some embodiments, anti-FGFR3 protein-drug conjugates described herein are used for treating or preventing a disease or disorder, such as but not limited to, a central nervous system (CNS) disease or disorder (e.g., a brain disease or disorder or a spinal cord disease or disorder), or an eye disease or disorder.

In some embodiments, anti-FGFR3 protein-drug conjugates described herein are used for treating or preventing a neurological disease or disorder. Non-limiting examples of neurological diseases or disorders include, but are not limited to, lysosomal storage diseases, amyloidosis, neuropathy, neurodegenerative diseases, leukodystrophy, neuropsychiatric diseases, traumatic brain injury, neurodevelopmental diseases, and neuromuscular diseases, seizure, behavioral disorders, ocular diseases or disorders, viral or microbial infections, inflammation, ischemia, and cancer. Specific examples of neurological disorders include, but are not limited to, neurodegenerative diseases (e.g., Lewy body disease, olivopontocerebellar atrophy, multiple system atrophy, postpoliomyelitis syndrome, Parkinson's disease, striatonigral degeneration, Shy-Draeger syndrome, tauopathies (e.g., Alzheimer disease and supranuclear palsy)), prion diseases (e.g., bovine spongiform encephalopathy, kuru, Creutzfeldt-Jakob syndrome, Gerstmann-Straussler-Scheinker disease, chronic wasting disease, fatal familial insomnia, and scrapie), bulbar palsy, motor neuron disease, and nervous system heterodegenerative disorders (e.g., Alexanders disease, Cockayne syndrome, Canavan disease, hepatolenticular degeneration, Huntington's disease, Halervorden-Spatz syndrome, neuronal ceroid-lipofuscinosis, Turette's syndrome, Menkes kinky hair syndrome, lafora disease, Rett syndrome, Lesch-Nyhan syndrome, and Unverricht-Lundborg syndrome), dementia (e.g., Pick's disease and spinocerebellar ataxia), cancer (e.g., central nervous system (CNS) cancers, including brain metastases resulting from cancer elsewhere in the body).

Exemplary neurological diseases that can be treated or prevented using the anti-FGFR3 protein-drug conjugates described herein are disclosed in Table 1-4. Corresponding genes that can be targeted for knockdown/knockout or knock-in replacement using, e.g., anti-FGFR3 protein-drug conjugates comprising interfering nucleic acids disclosed herein, or including a gene editing system or components of such systems disclosed herein, as well as other broad targeting options (e.g., using neuroprotective molecules), are also included.

TABLE 1-4
Exemplary Neurological Diseases and/or Disorders for Treatment or Prevention with Anti-FGFR3 Protein-
Drug Conjugates and Corresponding Genes that can be targeted

| | | Knockdown/Knockout | | | |
|---|---|---|---|---|---|
| | | | Multi- | Knock-in/Replacement | |
| | | Specific for | disease/indication | | Multi-disease/indication |
| | Disease | disease | targeting | Specific for disease | targeting |
| Neuro- | Alzheimer's | ApoE risk alleles | Stat3 | Protective ApoE | Neuroprotective molecules such |
| degenerative | disease | (e.g., ApoE4) | (PMID: 30322407) | isoforms or gene | as but not limited to below: |
| Disease | | | | variants (e.g., ApoE2, | a. Neurotrophic factors (CNTF, |
| | | | | ApoE Christchurch, | BDNF, NGF, GDNF, IGF, etc) |
| | | | | ApoE Jacksonville) | PMID: 24340223; |
| | Parkinson's | | | Atp13a2 (PMID: | b. Antibodies and antibody |
| | disease | | | 33046546) | fragments/derivatives (e.g., full |
| | Huntington's | | | | antibodies, ScFv, F(ab), F(ab′)2 |
| | disease | | | | targeting pathological proteins, |
| | Amyotrophic lateral | | | | such as misfolded or aggregated |
| | sclerosis (ALS) | | | | Abeta or Tau in Alzheimer's |
| | Prion disease | | | | disease; alpha synuclein in |
| | (transmissible | | | | Parkinson's disease; huntingtin in |
| | spongiform | | | | Huntington's disease; SOD1, Fus, |
| | encephalopathies) | | | | TDP-43 in ALS; prion protein in |
| Neuro- | Alexander disease | Gfap (PMID: | | | Prion disease); |
| developmental | | 34826654) | | | c. Antibody receptor fusion |
| disease | Multiple sulfatase | | | Sumf1 | proteins (e.g., anti-amyloid beta |
| | deficiency | | | (PMID: 22826245) | Gas6 fusion protein for |
| | Autism | | | Fmr1 (PMID: 22549954) | Alzheimer's:PMID: 35927581); |
| | Epilepsy | | | Glt1 (PMID: 34246771) | also envisioned for other |
| | Rett syndrome | MeCp2 (PMID: | | | antibodies for other |
| | | 29445033) | | | neurodegenerative diseases; or |
| | Fragile X | | | Fmr1 (PMID: 22549954) | d. Socs3 (inhibition of Jak-Stat3 |
| Physical | Traumatic brain | | | | pathway a signaling pathway that |
| injury | injury | | | | leads to astrocyte activation) |
| | Spinal cord injury | | | | |
| | Stroke | | | | |
| | Brain edema | Aqp4 (PMID: | | | |
| | | 16564496) | | | |

In some embodiments, the FGFR3 protein-drug conjugates described herein are used for treating or preventing, for example, without limitation, Alzheimer's disease, Alexander's disease, Parkinson's disease, Huntington's disease, prion diseases (transmissible spongiform encephalopathies), amyotrophic lateral sclerosis, Rett syndrome, fragile X mental retardation, multiple sulfatase deficiency (MSD), and stroke.

In some embodiments, the FGFR3 protein-drug conjugates described herein are used for treating or preventing a neuropsychiatric disease or disorder. Non-limiting examples of neuropsychiatric disease or disorders that can be treated with FGFR3 protein-drug conjugates of the present disclosure include major depressive disorder, anxiety disorders, and bipolar disorder by targeting proteins that modulate glioneuronal signaling and synaptic transmission, including but not limited to serotonin receptors (i.e., 5HT1a and 5HT7), noradrenaline receptors (i.e., ADRA1, ADRA2, ADRB1, ADRB2), serotonin transporter (SERT), noradrenaline transporter (NET), glutamine transporter (GLT1) and glutamine synthetase (GLUL), astrocytic gap junction proteins (i.e., GJB6 and GJA1), purinoceptor channels (i.e., P2RX family proteins), synaptic proteins (i.e., SNAP23, VAMP3).

Additional diseases or disorders are described in Zhao, et al. Front Mol Neurosci. 2019 Jun. 5; 12:136, which is incorporated herein by reference in its entity. As an example, astrocyte numbers or density have been reported to be affected in neuropsychiatric disorders. Either too little or too many astrocytes could be modulating by targeting astrocytes with an FGFR3 protein conjugated with a proliferative agent (if too few astrocytes), or a cytotoxic agent (if too many).

In some embodiments, anti-FGFR3 protein-drug conjugates of the present disclosure are used for treating or preventing a cancer, e.g., brain cancer. In some embodiments, anti-FGFR3 protein-drug conjugates of the present disclosure are used for treating or preventing a glioma (e.g., astrocytoma, glioblastoma) where dysregulation of FGFR3 expression is implicated (Lasorella et al., Neuro Oncol. 2017 Apr. 1; 19(4):475-483, which is incorporated herein by reference in its entity).

In some embodiments, anti-FGFR3 protein-drug conjugates described herein are used for treating or preventing glioblastoma multiforme (GBM). Glioblastoma multiforme (GBM) is a rapidly-growing type of tumor typically of the brain or spinal cord. It is the most common type of primary malignant brain tumor in adults. Only 25% of glioblastoma patients survive more than one year and 5% of patients survive more than five years. Symptoms of can depend on the brain region where the glioblastoma is located. For instance, the tumor can grow in areas of the brain that lead to difficulties in forming words or in moving limbs. Expanding tumors can increase pressure within the skull, leading to headaches. Other symptoms include impaired vision, nausea and vomiting, loss of appetite; mood swings; loss of balance, problems with memory or concentration, problems speaking and/or seizures.

As used herein, the term “subject” refers to a mammal (e.g., rat, mouse, cat, dog, cow, sheep, horse, goat, rabbit), preferably a human, for example, in need of prevention and/or treatment of a disease or disorder described herein.

The present disclosure includes combinations including an anti-FGFR3 protein-drug conjugate described herein, in association with one or more further therapeutic agents. A further therapeutic agent that is administered to a subject in association with an anti-FGFR3 protein-drug conjugate is administered to the subject in accordance with the Physicians' Desk Reference 2003 (Thomson Healthcare; 57th edition (Nov. 1, 2002)). The anti-FGFR3 protein-drug conjugate and the further therapeutic agent can be in a single composition or in separate compositions.

Methods for treating or preventing a disease or disorder in a subject in need of said treatment or prevention by administering an anti-FGFR3 protein-drug conjugate, in association with a further therapeutic agent are part of the present disclosure. Compositions comprising the anti-FGFR3 protein-drug conjugate in association with one or more further therapeutic agents also form part of the present disclosure.

The term “in association with” indicates that components, an FGFR3 binding protein, e.g., antibody or antigen-binding fragment thereof of the present disclosure, along with another agent such as methotrexate, can be formulated into a single composition, e.g., for simultaneous delivery, or formulated separately into two or more compositions (e.g., a kit including each component). Components administered in association with each another can be administered to a subject at a different time than when the other component is administered; for example, each administration may be given non-simultaneously (e.g., separately or sequentially) at intervals over a given period of time. Separate components administered in association with each another may also be administered sequentially, though essentially simultaneously, during the same administration session. Moreover, the separate components administered in association with each another may be administered to a subject by the same or by a different route.

In some embodiments, an anti-FGFR3 protein-drug conjugate may be administered in association with a further therapeutic agent such as Aducanumab (Aduhelm™), Donepezil (Aricept®), Galantamine (Razadyne®), Lecanemab, Memantine (Namenda®), Rivastigmine (Exelon®), Donepezil and memantine (Namzaric®), Orexin receptor antagonist (Belsomra®), or Suvorexant (Belsomra®) for the treatment of Alzheimer's disease.

In some embodiments, an anti-FGFR3 protein-drug conjugate may be administered in association with a further therapeutic agent such as Carbidopa-levodopa (Sinemet), Carbidopa-levodopa (controlled release) (Sinemet CR), Carbidopa-levodopa (orally disintegrating tablet) (Parcopa), Carbidopa-levodopa (extended release capsules) (Rytary), Carbidopa-levodopa-entacapone (enteral suspension) (Duopa), Levodopa Inhalation powder (Inbrija), Entacapone (Comtan), Tolcapone (Tasmar), Opicapone (Ongentys), Carbidopa/Levodopa Entacapone (Stalevo), Pramipexole (Mirapex), Pramipexole (extended release) (Mirapex ER), Ropinirole (Requip), Ropinirole (extended release) (Requip XL), Apomorphine (injection) (Apokyn), Apomorphine sublingual film (Kynmobi), Rotigotine (transdermal patch) (Neupro), Selegiline (Eldepryl), Selegiline (orally disintegrating tablet) (Zelapar), Rasagiline (Azilect), Safinamide (Xadago), Amantadine (Symmetrel), Amantadine (extended release) (Gocovri), Amantadine (extended release) (Osmolex), Istradefylline (Nourianz), Trihexyphenidyl (Artane), or Benztropine (Cogentin) for the treatment of Parkinson's disease.

In some embodiments, an anti-FGFR3 protein-drug conjugate may be administered in association with a further therapeutic agent such as Austedo, Baclofen, Citalopram, Creatine, Deutetrabenazine, Fluoxetine, Fluphenazine, Haloperidol, Mirtazapine, Olanzapine, Pimozide, Risperidone, Sertraline, Tetrabenazine, Tetrabenazine, or Xenazine for the treatment of Huntington's disease.

In some embodiments, an anti-FGFR3 protein-drug conjugate may be administered in association with a further therapeutic agent such as RELYVRIO (AMX0035), Radicava™ (edaravone), Riluzole, Tiglutik (thickened riluzole), Exservan™ (riluzole oral film), Nuedexta®, Diazepam, Gabapentin, Trihexyphenidyl, or amitriptyline for the treatment of Huntington's disease.

In some embodiments, an anti-FGFR3 protein-drug conjugate may be administered in association with a further therapeutic agent such as Acetazolamide, Brivaracetam, Cannabidiol, Carbamazepine, Cenobamate, Clobazam, Clonazepam, Eslicarbazepine acetate, Ethosuximide, Everolimus, Fenfluramine, Gabapentin, Lacosamide, Lamotrigine, Levetiracetam, Oxcarbazepine, Perampanel, Phenobarbital, Phenytoin, Piracetam, Pregabalin, Primidone, Rufinamide, Sodium valproate, Chrono, Epilim Chronosphere, Episenta, Epival, Dyzantil, Stiripentol, Tiagabine, Topiramate, Valproic acid, Vigabatrin, or Zonisamide for the treatment of Alexander disease or Epilepsy.

In some embodiments, an anti-FGFR3 protein-drug conjugate may be administered in association with a further therapeutic agent such as Abilify, Risperidone, Risperdal, Aripiprazole, Effexor, Venlafaxine, Effexor XR, Geodon, Memantine, Ziprasidone, Paliperidone, or Bumetanide for the treatment of Autism.

In some embodiments, an anti-FGFR3 protein-drug conjugate may be administered in association with a further therapeutic agent such as ANAVEX2-73, Trofinetide, Sarizotan, Ketamine, Cannabidiol, AMO-04, AVXS-201, mecasermin or INCRELEX for the treatment of Rett syndrome.

In some embodiments, an anti-FGFR3 protein-drug conjugate may be administered in association with a further therapeutic agent such as Aripiprazole (Abilify), Risperidone (Risperdal), Olanzapine (Zyprexa), Quetiapine (Seroquel), Pimavanserin (Nuplazid), Clozapine, Ziprasidone (Geodon), Lurasidone (Latuda), Brexpiprazole (Rexulti), Valproic Acid (Depakote), Carbamazepine (Tegretol), Lamotrigine (Lamictal), Risperidone (Risperdal M-TAB), Olanzapine (Zyprexa Zydis), Sertraline (Zoloft), Citalopram (Celexa), Escitalopram (Lexapro), Duloxetine (Cymbalta), Venlafaxine (Effexor), Desvenlafaxine (Pristiq), Trazodone (Desyrel), Bupropion (Wellbutrin), Buspirone (Buspar), Clonazepam (Klonopin), Lorazepam (Ativan), Propranolol (Inderal), Imipramine (Tofranil), Clomipramine (Anafranil), Nortriptyline (Pamelor), Methylphenidate (Ritalin, Concerta), Dexmethylphenidate (Focalin, Focalin XR), Adderall, Lisdexamfetamine (Vyvanse), Adzenys XR-ODT, Clonidine (Catapres), Guanfacine (Tenex), Atomoxetine (Strattera), Lithium (Eskalith, Eskalith-CR, Lithobid), Lisdexamfetamine (Vyvanse), Topiramate (Topamax), Naltrexone/Bupropion (Contrave), or Benztropine (Cogentin) for the treatment of Fragile X disease.

In some embodiments, an anti-FGFR3 protein-drug conjugate may be administered in association with a further therapeutic agent such as Mannitol (Osmitrol, Resectisol), carbamazepine, Phenytoin (Dilantin, Phenytek), Valproate sodium, Gabapentin (Neurontin), Topiramate (Topamax), Carbamazepine (Equetro), Magnesium sulfate (NMDA), Potassium, Phosphate, Dextromethorphan/quinidine (Nuedexta), Pentobarbital sodium (Nembutal sodium), Nimodipine (Nymalize), Methylphenidate hydrochloride (Ritalin, Daytrana), Modafinil (Provigil), Levodopa, Sertraline hydrochloride (Zoloft), Citalopram hydrobromide (Celexa), Paroxetine hydrochloride (Paxil), Quetiapine fumarate (Seroquel), Tizanidine hydrochloride (Zanaflex), Baclofen (Lioresal), Dantrolene sodium (Dantrium), Diazepam (Valium, Diazepam Intensol), Cyclobenzaprine hydrochloride (Amrix), Acetaminophen (Tylenol), Ibuprofen (Advil, Motrin), or Naproxen sodium (Naprosyn, Aleve, Anaprox DS) for the treatment of Traumatic brain injury.

In some embodiments, an anti-FGFR3 protein-drug conjugate may be administered in association with a further therapeutic agent such as Methylprednisolone, Tirilazad mesylate, Lyrica (pregabalin), Riluzole, GM1 ganglioside, Gacyclidine, or naloxone for the treatment of Spinal cord injury.

In some embodiments, an anti-FGFR3 protein-drug conjugate may be administered in association with a further therapeutic agent such as Alteplase, Astaxanthin, Aspirin, Aspirin/dipyridamole (Aggrenox), Clopidogrel (Plavix), Dabigatran, Ticagrelor (Brilinta), Warfarin (Coumadin, Jantoven), Dabigatran (Pradaxa), Apixaban (Eliquis), Rivaroxaban (Xarelto), Thiazide diuretics (water pills), such as hydrochlorothiazide (Microzide), Angiotensin-converting enzyme (ACE) inhibitors, such as lisinopril (Prinivil, Zestril), Angiotensin II receptor blockers (ARBs), such as losartan (Cozaar) and valsartan (Diovan), Losartan, Nimodipine, Policosanol, Rivaroxaban, or Ticlopidine for the treatment of stroke.

In some embodiments, an anti-FGFR3 protein-drug conjugate may be administered in association with a further therapeutic agent such as Dexamethasone, Mannitol, Dexamethasone Intensol, Osmitrol, De-Sone LA, Dxevo, HiDex, or Zcort for the treatment of brain edema.

An effective or therapeutically effective amout of FGFR3 binding protein, e.g., antibody or antigen-binding fragment, for treating or preventing an FGFR3-mediated condition refers to the amount of the antigen-binding protein sufficient to alleviate one or more signs and/or symptoms of the disease or condition in the treated subject, whether by inducing the regression or elimination of such signs and/or symptoms or by inhibiting the progression of such signs and/or symptoms. In an embodiment of the disclosure, an effective or therapeutically effective amount of FGFR3 binding protein is about 2-30 mg/kg. This dose may be administered, for example, about once a month. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks or months.

A symptom is a manifestation of disease apparent to the patient himself, while a sign is a manifestation of disease that the physician perceives. Reduction, fully or in part, of a sign or symptom may be referred to as alleviation of the sign or symptom.

In an embodiment, an effective or therapeutically effective dose of anti-FGFR3 protein-drug conjugate is about 1 mg/kg and 50 mg/kg body weight. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks.

In some embodiments, the anti-FGFR3 protein-drug conjugate, or pharmaceutical composition thereof, may be administered in accordance with a repeat dosing regimen wherein the anti-FGFR3 protein-drug conjugate may be administered a first time (e.g., in an initial dose) and then re-administered any number of subsequent times thereafter at any amount over the time course of treatment of a subject. For example, the anti-FGFR3 protein-drug conjugate may be re-administered once, twice, three times, four times, five times, six times, seven times, eight times, nine times, ten times, or more, over the time course of the treatment of a subject which can occur over any number of days, weeks, or years.

In some embodiments, the anti-FGFR3 protein-drug conjugate, or pharmaceutical composition thereof, may be administered in accordance with a stepwise dosing regimen. Stepwise dosing of a composition can refer to breaking up (i.e., dividing) dosing of the same composition over multiple administrations. In some embodiments, the dosing of the same composition is broken up once, twice, three times, four times, five times, six times, seven times, eight times, nine times, ten times, or more, over the time course of the treatment of a subject which can occur over any number of days, weeks, or years. In some embodiments, when a stepwise dose regimen is used in the administration of an anti-FGFR3 protein-drug conjugate, the stepwise dosing regimen may result in a gradual increase in therapeutic transgene levels with each administration of the anti-FGFR3 protein-drug conjugate

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Expression of Total FGFR3, and FGFR3b and FGFR3c Isoforms in Mouse Brain, Primary Mouse Astrocytes, and Primary Human Astrocytes

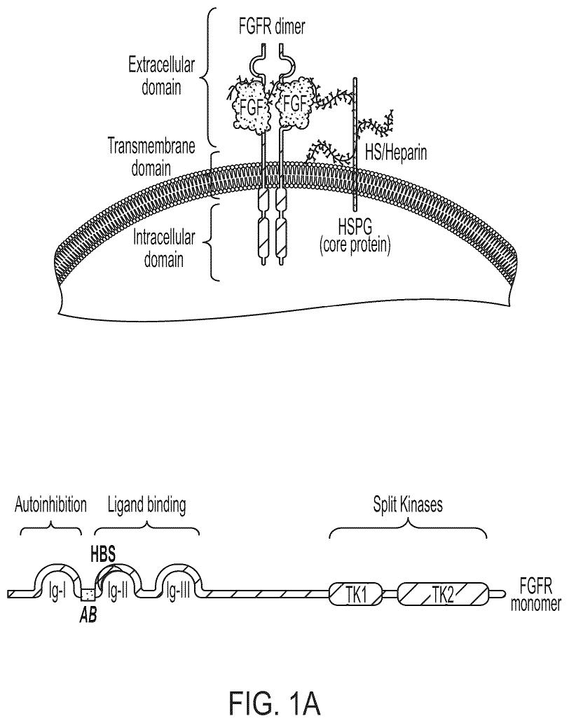

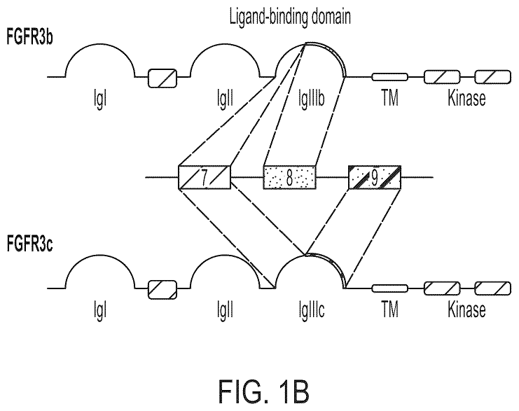

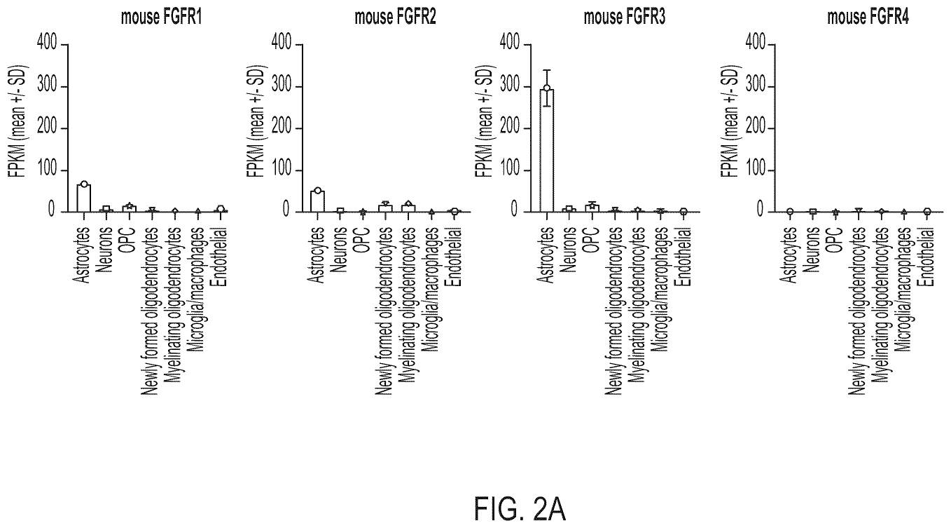

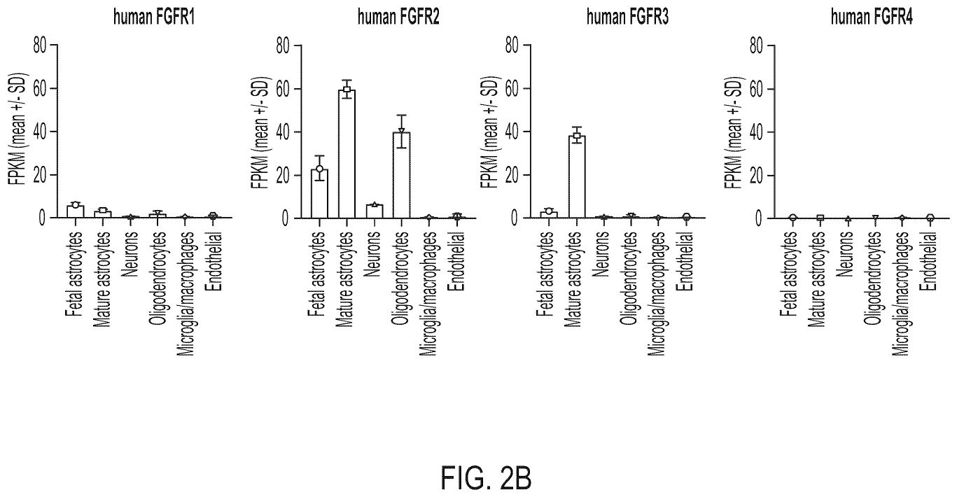

The present Example investigated the expression levels of FGFR3, and FGFR3b and FGFR3c in mouse brain, primary mouse astrocytes, and human astrocytes. Schematic representations of the general architecture of fibroblast growth factor receptor 3 (FGFR3), and isoforms FGFR3b and FGFR3c are depicted in FIGS. 1A-1B. RNA sequencing data showing expression of FGFR1-FGFR4 transcript in mouse and human nervous and supporting cells have shown that FGFR3 is highly expressed on mouse (FIG. 2A) and human astrocytes (FIG. 2B). This finding provides support for FGFR3 as attractive target since it is both largely surface-abundant, e.g., for internalization with FGFR3 antibody-molecular cargo conjugates described herein, and is present in cell types of both species.

Astrocytes were isolated either from mouse brain, or from human fetal brain using an ATPase Na+/K+ Transporting Subunit Beta 2 (ATP1B2) selection kit (Miltenyi Biosciences). Following isolation, the cells were plated onto poly-D-lysine coated cell culture dishes. Cells were grown in 10% fetal bovine serum (FBS) in complete Dulbecco's Modified Eagle Medium (DMEM) media, and allowed to proliferate for 7-14 days. After reaching 80% confluency in 6-well plates, the cells were collected with Trizol reagent (Life Technologies). Trizol-collected samples were then flash frozen on dry ice and stored at −80° C. until RNA extraction.

For total brain TaqMan analysis of FGFR3, FGFR3b, and FGFR3c expression, 057131/6 mice were euthanized by CO₂ and cervical dislocation, followed by transcardial perfusion with ice-cold phosphate buffered saline (PBS). Brains were removed and collected in RNALater solution (I nvitrogen), and total RNA extracted using MagMAX-96 for Microarrays Total RNA Isolation Kit (ThermoFisher) with an additional DNAse (Qiagen) step added between the first and second washes. Gene expression was determined via TaqMan reverse transcription-polymerase chain reaction (RT-PCR) assays using the following mouse- and human-specific FGFR3 primers and probes:

Human FGFR3c
Forward primer:
(SEQ ID NO: 309)
TTGCACAACGTCACCTTTGAG
Reverse primer:
(SEQ ID NO: 310)
CAGCCACGCAGAGTGATGAG
Probe:
(SEQ ID NO: 311)
AGTACACCTGCCTGGCGGGCAATT
Human FGFR3b
Forward primer:
(SEQ ID NO: 312)
CAAGTCCTGGATCAGTGAGAGTG
Reverse primer:
(SEQ ID NO: 313)
TCCCGCTCCGACACATTG
Probe:
(SEQ ID NO: 314)
AGGCCGACGTGCGCCTCC
Mouse FGFR3c
Forward primer:
(SEQ ID NO: 315)
CACCGACAAGGAGCTAGAGGTT
Reverse primer:
(SEQ ID NO: 316)
ACCAGCCACGCAGAGTGATG
Probe:
(SEQ ID NO: 317)
TGTCCTTGCACAATGTCACCTTTGA
Mouse FGFR3b
Former primer:
(SEQ ID NO: 318)
CCTGGATCAGTGAGAATGTGGA
Reverse primer:
(SEQ ID NO: 319)
GGTGGCTCGACAGAGGTACT
Probe:
(SEQ ID NO: 320)
ACGCCTCCGCCTGGCCAATG

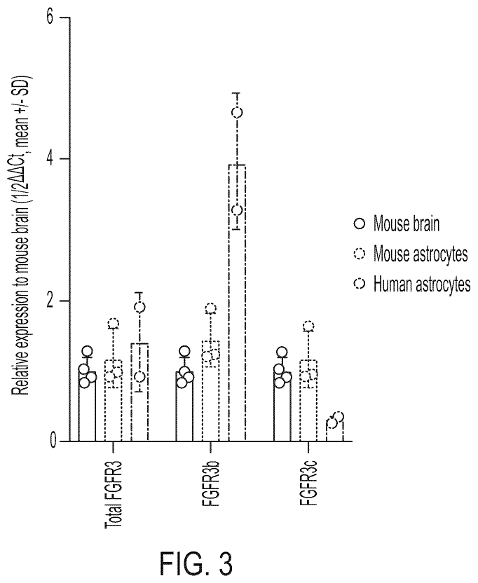

As shown in FIG. 3, mouse astrocytes expressed equal levels of FGFR3b and FGFR3c isoforms, whereas human astrocytes expressed approximately 8 times more FGFR3b than FGFR3c, although both isoforms were expressed at appreciable levels on human cells.

### Example 2. FGFR3 Antibodies are Internalized into Live Astrocytes as Evidenced by Perinuclear Speckles

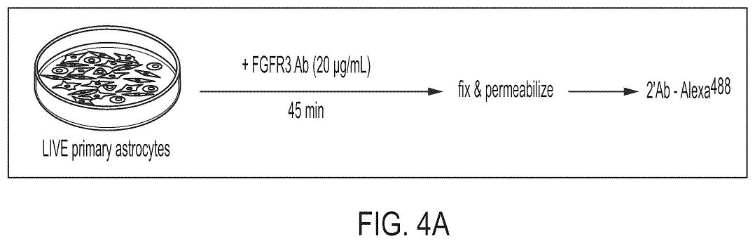

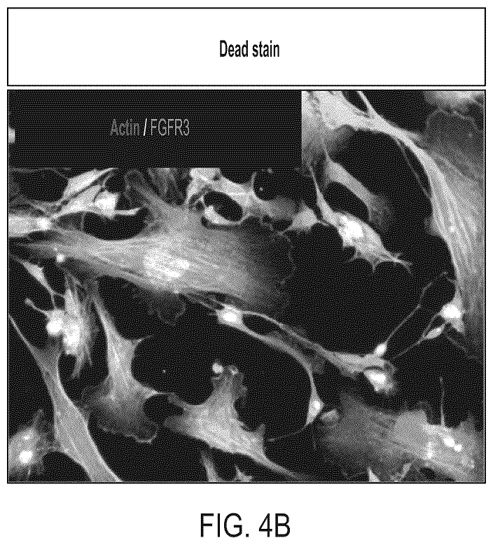

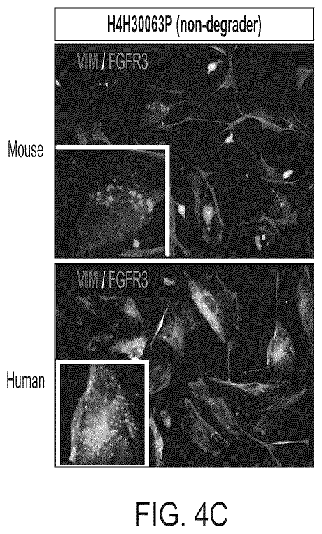

This Example was designed to interrogate internalization of FGFR3 antibodies described herein into live astrocytes. DEAD and LIVE immunofluorescence staining approaches were used for visualization of cytoskeletal proteins (e.g., actin and vimentin), as well as FGFR3 in primary human and mouse astrocytes. A schematic depicting an exemplary experimental timeline used in the present Example is displayed in FIG. 4A.

Primary human and mouse astrocytes were isolated in accordance with procedures as described herein (see, e.g., Example 1). For DEAD staining, primary astrocytes were grown to 70-80% confluency on glass coverslips coated with poly-L-lysine in normal growth media (DMEM+10% FBS+Penicillin/streptomycin [Pen/strep]+GlutaMax) before media was exchanged to serum-free media (DMEM (Life Technologies)+N2 supplement (Gibco)+Pen/strep+GlutaMax) for 4 hours. Media was then removed and cells were washed with 1×PBS and fixed with 4% paraformaldehyde (PFA) in 1×PBS for 10 minutes at room temperature. Non-specific binding was blocked using PBGA blocking buffer (1×PBS, pH7.2 (Gibco), 10% normal goat serum (Vector Laboratories), 0.01% Triton-X 100 (Sigma), 0.2% porcine fish skin gelatin (Sigma)) for 30 minutes at room temperature. Fixed cells were treated with anti-human FGFR3 antibodies at 4 μg/mL in blocking buffer overnight at 4° C. Cells were washed 3 times with 1×PBS and treated with Alexa-labeled goat anti-human IgG secondary antibodies (Life Technologies; 1:400) and DAPI (4′,6-diamidino-2-phenylindole (Life Technologies); 1:20,000) for 20 minutes at room temperature. Cells were washed with 1×PBS and imaged on Zeiss Axio Observer microscope.

For LIVE staining, primary astrocytes were grown to 60% confluency on glass coverslips coated with poly-L-lysine in normal growth media (DMEM+10% FBS+Pen/strep+GlutaMax) before media was exchanged to serum-free media (DMEM (Life Technologies)+N2 supplement (Gibco)+Pen/strep+GlutaMax) 4 hours prior to antibody treatment. Cells were treated with anti-human FGFR3 antibodies at 20 μg/mL for 45 minutes at 37° C. Media was then removed and cells were washed with 1×PBS and fixed with 4% paraformaldehyde in 1×PBS for 10 minutes at room temperature. Non-specific binding was blocked using PBGA blocking buffer for 30 minutes at room temperature, and treated with Alexa-labeled goat anti-human IgG secondary antibodies (Life Technologies; 1:400) and DAPI (4′,6-diamidino-2-phenylindole (Life Technologies); 1:20,000) for 20 minutes at room temperature. Cells were washed with 1×PBS and imaged on Zeiss Axio Observer microscope.

Immunoffluorescence images revealed FGFR3 antibodies were internalized into live astrocytes as evidenced by perinuclear speckles (puncta) visualized using the above-described DEAD (FIG. 4B) and LIVE staining approaches (FIG. 4C). For the LIVE staining, receptor non-degrading FGFR3 antibodies (e.g., H4H30063P), i.e., FGFR3 antibodies which do not promote degradation of the FGFR3 receptor, showed robust internalization (FIG. 4C).

### Example 3. Validation of FGFR3 Expression and Internalization in the U87-FGFR3b-FLuc-GFP Line

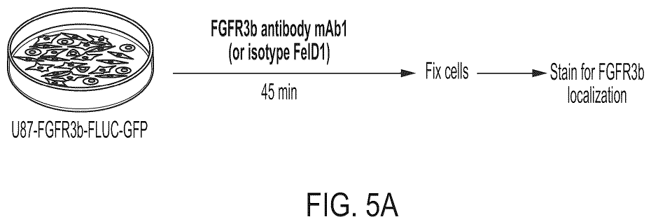

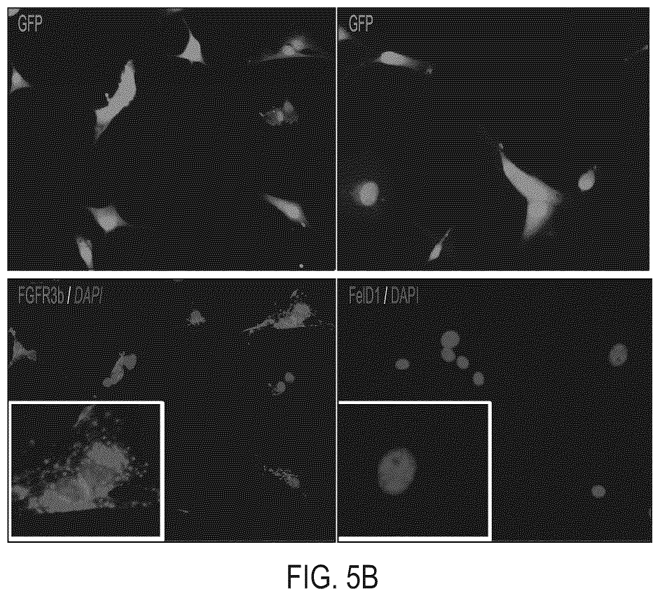

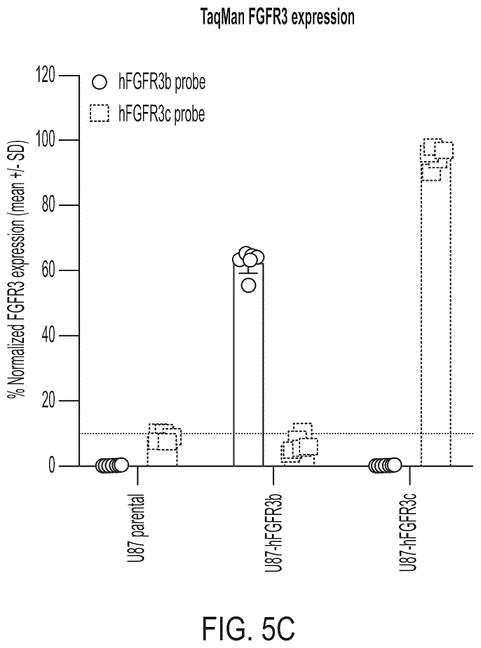

The present Example was designed to validate expression and internalization of FGFR3 using the U87-FGFR3b-FLuc-GFP line. A schematic depicting an exemplary experimental timeline used in the present Example is displayed in FIG. 5A.

For generation of U87 cell lines expressing FGFR3b or FGFR3c plus green fluorescent protein (GFP)-luciferase (Luc), U87 cells were grown in a 24-well plate until reaching 80% confluence in normal growth media (Eagle's Minimum Essential Medium [EMEM]+10% FBS+non-essential amino acids+Pen/Strep+GlutaMax). For viral transduction, lentiviruses containing the human FGFR3b (hFGFR3b) or human FGFR3c (hFGFR3c) isoforms were obtained from ViGene Bioscieneces and transduced for 48 hours with 5 multiplicity of infection (MOI) of virus. After 48 hours, the cells were switched to growth medium containing 1 μg/mL puromycin, where non-transduced cells were killed off, and transduced cells were allowed to expand. Cells were then treated with lentivirus containing GFP-P2A-luciferase (1 MOI) for 48 hours. Green cells were sorted by fluorescence-activated cell sorting (FACS) into single wells of 96-well plates and colonies were allowed to expand.

For validation of human FGFR3b or human FGFR3c expression and internalization, U87-FGFR3b-GFP-P2A-Fluc or U87-FGFR3c-GFP-P2A-Fluc were grown to 60% confluency on glass coverslips coated with poly-L-lysine in normal growth media before media was exchanged to serum-free media (EMEM+non-essential amino acids+Pen/strep+GlutaMax) 4 hours prior to antibody treatment. Cells were treated with anti-human FGFR3 antibodies at 20 μg/mL for 45 minutes at 37° C. Media was then removed and cells were washed with 1×PBS and fixed with 4% paraformaldehyde (PFA) in 1×PBS for 10 minutes at room temperature. Non-specific binding was blocked using PBGA blocking buffer for 30 minutes at room temperature, followed by treatment with Alexa-labeled goat anti-human IgG secondary antibodies (Life Technologies; 1:400) and DAPI (4′,6-diamidino-2-phenylindole (Life Technologies; 1:20,000) for 20 minutes at room temperature. Cells were washed with 1×PBS and imaged on a Leica microscope. Using the above-described protocol, FGFR3 were visualized as prevalent fluorescent puncta surrounding the nucleus (DAPI straining), thereby confirming FGFR3 expression and internalization in the U87-FGFR3b-FLuc-GFP line (FIG. 5B).

Human FGFR3 expression was determined using human-specific FGFR3b and FGFR3c TaqMan assays described herein. Briefly, cells were grown to 80% confluency in 6-well plates in growth media and collected using TriZol reagent (Life Technologies). Trizol samples were flash frozen on dry ice and total RNA was isolated using MagMAX-96 for Microarrays Total RNA Isolation Kit (ThermoFisher). Data were plotted as 1/Ct value and normalized to the highest and lowest values in the data set. A graph showing percent normalized TaqMan FGFR3 expression (mean±standard deviation [SD]) in U87 parental cells, and in U87 cell lines expressing human FGFR3b (U87-hFGFR3b), and human FGFR3c (U87-hFGFR3c) isoforms using hFGFR3b and hFGFR3c probes is shown in FIG. 5C. hFGFR3b and hFGFR3c probe groups revealed a high percentage FGFR3 expression levels in U87-hFGFR3b and U87-hFGFR3c groups, respectively.

### Example 4. GFP and Luciferase siRNA Knockdown of their Respective Targets in U87-FGFR3b/c-GFP-Luciferase Cells

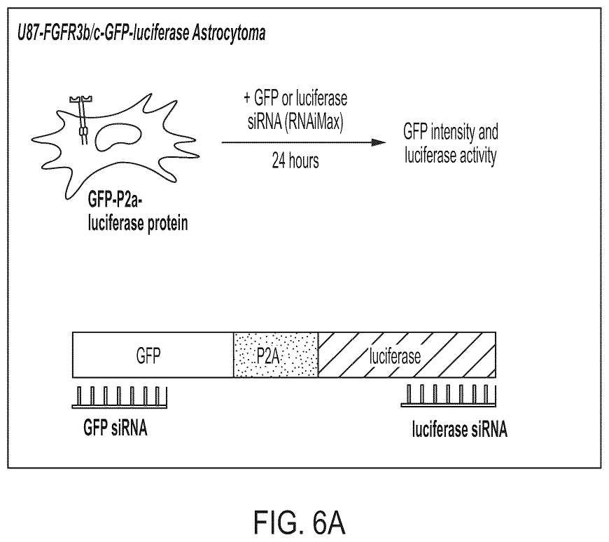

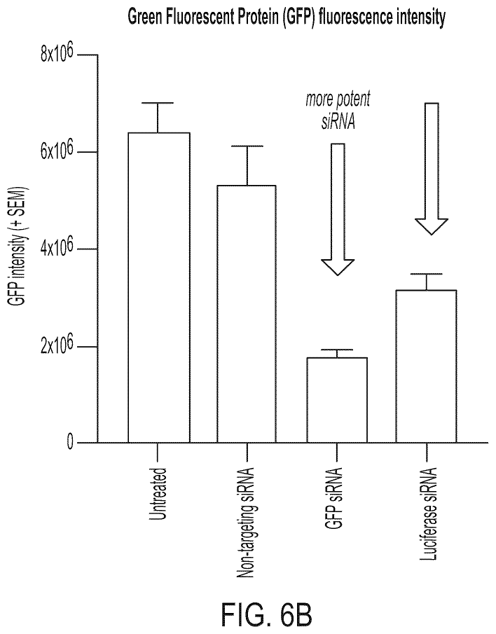

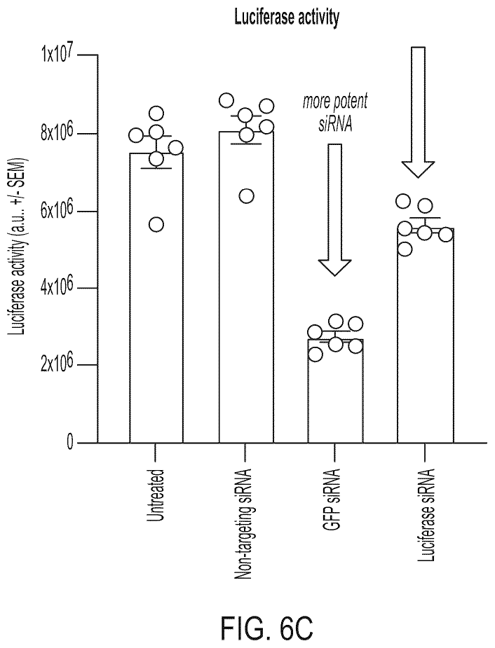

A schematic depicting an exemplary treatment paradigm of the U87-FGFR3b or U87-FGFR3c cell lines used in the present Example, and including a schematic of the GFP-P2A-Fluc mRNA, is shown in FIG. 6A. Luciferase and GFP are present within one contiguous mRNA, and as such, a GFP siRNA may knockdown luciferase expression and, conversely, a luciferase siRNA may knockdown GFP expression. Sequences corresponding to the GFP siRNA and luciferase siRNA molecules (commercially sourced from Horizon Discovery Biosciences) tested in the present Example are shown in FIG. 7.

For measuring GFP fluorescence, U87-FGFR3-GFP-P2A-Fluc cells were grown to 60% confluency in normal growth media in 96 well black wall glass bottom plate (manufacturer). Media was replaced with fresh growth media and treated with non-targeting, GFP-targeting, or luciferase-targeting siRNA using Lipofectamine RNAiMax (Life Technologies) following the manufacturer's suggested protocol. Briefly, 50 pmol of siRNA was diluted in 25 μL OptiMEM media (Life Technologies) and 1.5 μL of RNAiMax reagent was diluted in 25 μL OptiMEM. The diluted siRNA and RNAiMax reagent were mixed and allowed to incubate at room temperature for 5-10 minutes. 10 μL of siRNA-RNAiMax complexes were added per well (10 pmol siRNA per well). Cells were incubated at 37° C. for 24-48 hours and GFP fluorescence was monitored using Incucyte live cell imaging system.

For measuring luciferase activity, U87-FGFR3-GFP-P2A-Fluc cells were grown to 60% confluency in normal growth media in 96 well white plates (Corning). Cells were treated with siRNA molecules as described above for 24 hours. At which point, luciferase activity was assessed using Firefly Luc one-step glow assay kit (Pierce) using the suggested manufacturer's protocol. Briefly, luciferase substrate was diluted 1:100 in provided buffer and 100 uL of reagent was added per well, covered, placed on shaker at 300 rpm for 3 minutes followed by a 10-minute incubation period at room temperature. Luciferase activity was read on i3M plate reader (Molecular Devices) for detection of all wavelengths with 1000 ms integration time.

Graphs showing mean GFP fluorescence intensity and luciferase activity within the two different siRNA (GFP siRNA and luciferase siRNA) versus non-targeting siRNA, and as compared to untreated controls, are shown in FIG. 6B and FIG. 6C, respectively. GFP siRNA was more potent than luciferase siRNA (arrows), a finding which served as an impetus to advance the GFP siRNA via introduction of truncation modifications to this molecule described herein. This Example showed parental siRNA were validated in the U87 cell lines expressing FGFR3b or FGFR3c plus FLuc-GFP.

### Example 5. Modified GFP-Targeting siRNA Knockdown in U87-FGFR3b-GFP-Luciferase Cells

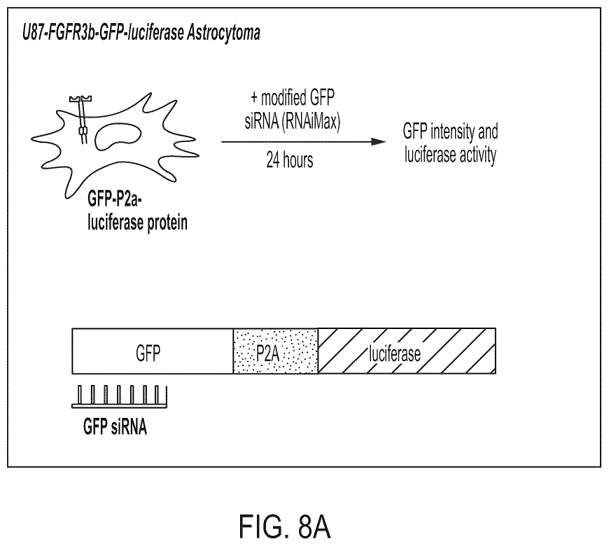

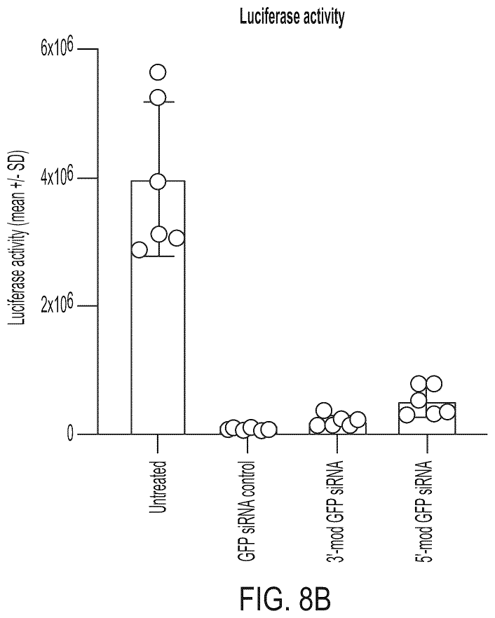

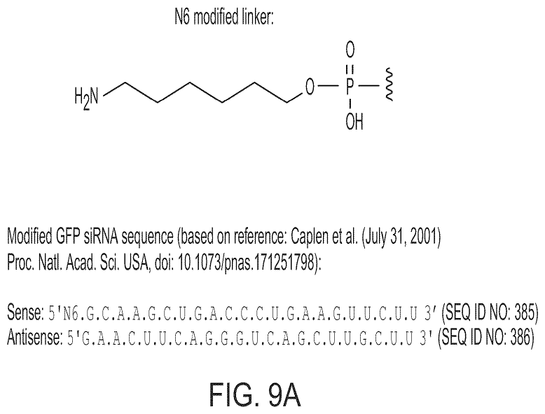

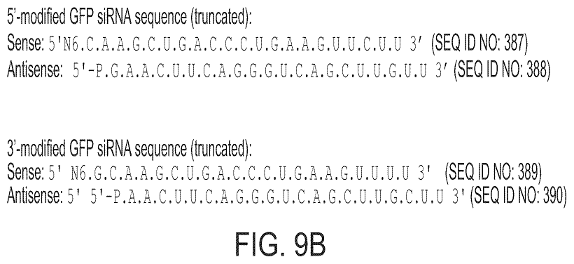

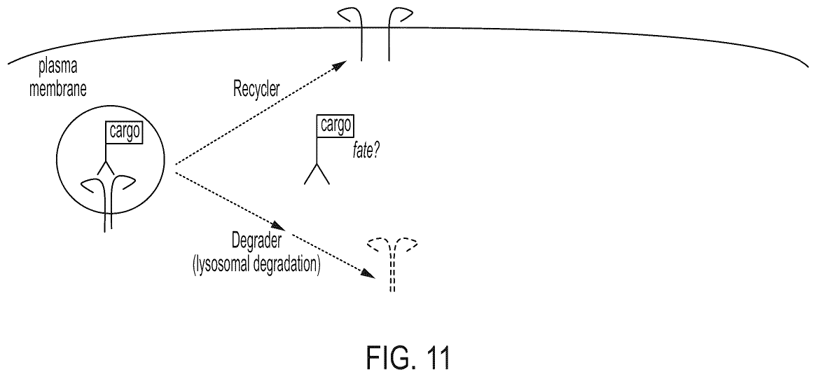

A schematic depicting an exemplary treatment paradigm of the U87-FGFR3b or U87-FGFR3c cell lines used in the present Example, and including a schematic of the GFP-P2A-Fluc mRNA, is shown in FIG. 8A. Sequences corresponding to the GFP siRNA molecules tested in the present Example are shown in FIGS. 9A-9B. GFP siRNA sequences shown in FIG. 9A (bottom) are identical to those depicted in FIG. 7, but include an N6 modification (see, e.g., FIG. 9A, top) at the 5′ end of the siRNA. A modified (truncated) parent sequence where the GFP siRNA has been shortened by one nucleotide on the 5′ end, and combined with the additional 5′ N6 modification (5′-modified GFP siRNA sequence [truncated]), is presented in FIG. 9B, top. A modified (truncated) parent sequence where the GFP siRNA has been shorted by one nucleotide on the 3′ end, and combined with the 5′ N6 modification (5′-modified GFP siRNA sequence [truncated]) is presented in FIG. 9B, bottom. Shortened siRNA molecules allowed for incorporation of siSTABLE technology (Horizon Discovery). siSTABLE-modified siRNA molecules, as described by the manufacturer, are chemical modifications that provided extended stability and resistance from extracellular and intracellular nucleases. As FGFR3 antibody-conjugates could potentially shuttle to intracellular compartments that may ultimately degrade some of the siRNA, for example, via lysosomal degradation (as illustrated in FIG. 11), siRNA designs were selected with maximal possible half-life and stable modifications to permit a more sustained effect.

For measuring luciferase activity, U87-FGFR3-GFP-P2A-Fluc cells were grown to 60% confluency in normal growth media in 96 well white plates (Corning). Cells were treated with modified GFP-targeting siRNA molecules as described above for 24 hours. Luciferase activity was then assessed using Firefly Luc one-step glow assay kit (Pierce) in accordance with the suggested manufacturer's protocol. Briefly, luciferase substrate was diluted 1:100 in provided buffer and 100 μL of reagent was added per well. The plate was then covered and placed on a shaker at 300 rpm for 3 minutes, followed by 10-minute incubation at room temperature. Luciferase activity was read on i3M plate reader (Molecular Devices) for detection of all wavelengths with 1000 ms integration time. Shortened siRNA were still potent for knockdown of their target, and these data are shown in FIG. 8B. Thus, truncation of the GFP siRNA molecules allowing for their formulation in the siSTABLE format did not affect potency or knockdown ability of the siRNA.

### Example 6. Internalized Anti-FGFR3b H4H30105P2 Co-Localizes with Early and Late Endosomes, but not Rab4 Recycling Endosomes, and Internalized Anti-FGFR3b Antibody H4H30063P Co-Localizes with Early and Late Endosomes, Lysosomes, as Well as Rab-4 Positive Recycling Endosomes

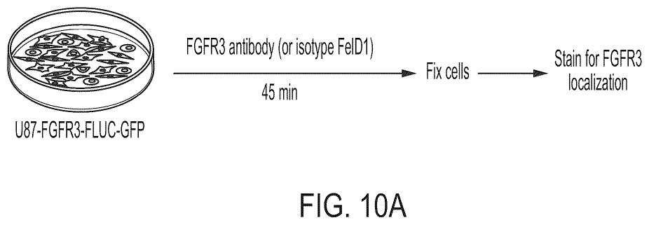

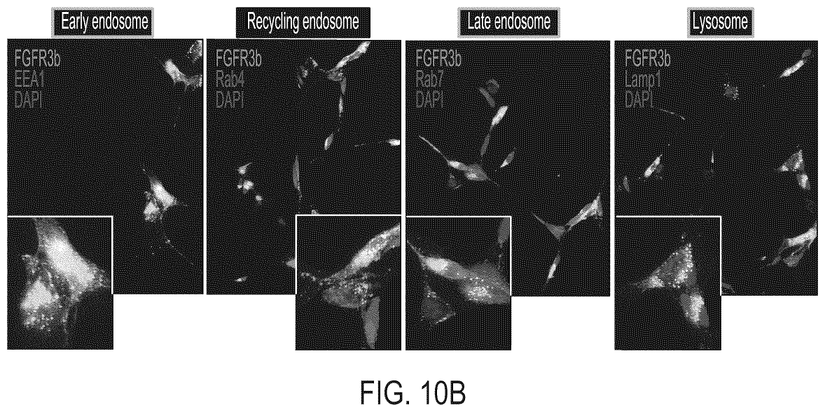

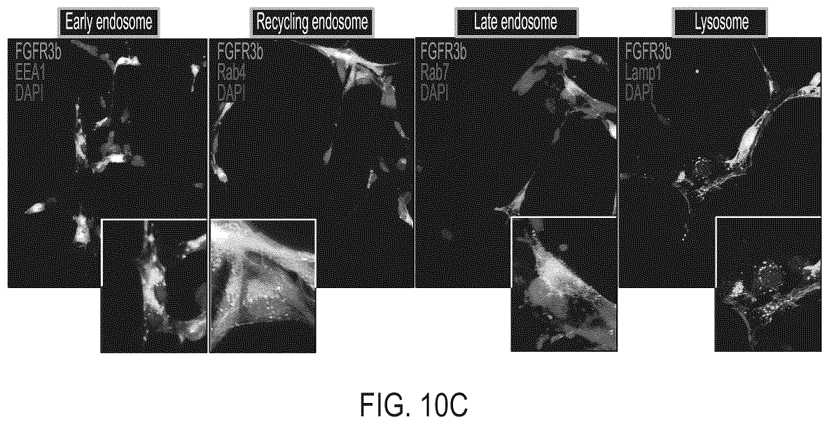

The present Example investigated the endosomal fate of internalized FGFR3b antibodies H4H30105P2 and H4H30063P. A schematic depicting treatment of the U-87-FGFR3b cell line is depicted in FIG. 10A.

U87-FGFR3b-GFP-P2A-Fluc cells were grown to 60% confluency on glass coverslips coated with poly-L-lysine in normal growth media (EMEM+10% FBS+Pen/strep+GlutaMax+1 μg/mL puromycin) before media was exchanged to serum-free media (EMEM+Pen/strep+GlutaMax+1 μg/mL puromycin) 4 hours prior to antibody treatment. Cells were treated with humanized anti-human FGFR3 antibodies at 20 μg/mL for 45 minutes at 37° C. Media was then removed and cells were washed with 1×PBS, and subsequently fixed with 4% paraformaldehyde in 1×PBS for 10 minutes at room temperature. Non-specific binding was blocked using blocking buffer as described above for 30 minutes at room temperature. Fixed cells were treated with endolysosomal markers as follows: Early Endosome Antigen 1 (EEA1) (Invitrogen at 1:100), Ras-Related Protein Rab-4A (Rab4) (Invitrogen at 1:100), Ras-Related Protein Rab-7a (Rab7) (Invitrogen at 1:100), or Lysosomal Associated Membrane Protein 1 (LAMP1) (Abcam at 1:100) and incubated overnight at 4° C. Cells were washed with 1×PBS and treated with Alexa-labeled goat anti-human IgG secondary antibodies (Life Technologies; 1:400) and DAPI (4′,6-diamidino-2-phenylindole (Life Technologies); 1:20,000) for 20 minutes at room temperature. Cells were washed with 1×PBS and imaged on a Leica microscope. These experiments support robust internalization of FGFR3 in the engineered cell lines described herein. Specifically, endosomal trafficking of FGFR3b antibodies H4H30105P2 (non-conjugated) and H4H30063P (non-conjugated) illustrated achievement of robust internalization as evidenced by perinuclear speckles (puncta). H4H30105P2 co-localized with the early endosomal marker EEA1 and the late endosomal marker Rab7, but not with the recycling endosomal marker Rab4 (FIG. 10B). H4H30063P co-localized with the early endosomal marker EEA1, the late endosomal marker Rab7, and the lysosomal marker Lamp1, as well as with the recycling endosomal marker Rab4 (FIG. 10C).

### Example 7. Screening of FGFR3 Antibodies in U87-FGFR3b-FLUC-GFP Cell Lines

This Example relates to screening of FGFR3 antibodies with various internalization and binding properties in U87-FGFR3b-Fluc-GFP versus U87-FGFR3c-GFP-P2A-Fluc cell lines. A schematic depicting treatment of the U87-FGFR3b-Fluc-GFP cell line is depicted in FIG. 10A. The same treatment paradigm was also implemented using the U87-FGFR3c cell line for certain experiments.

For validation of human FGFR3b or human FGFR3c expression and internalization, U87-FGFR3b-GFP-P2A-Fluc or U87-FGFR3c-GFP-P2A-Fluc were grown to 60% confluency on glass coverslips coated with poly-L-lysine in normal growth media before media was exchanged to serum-free media (EMEM+non-essential amino acids+Pen/strep+GlutaMax) 4 hours prior to antibody treatment. Cells were treated with anti-human FGFR3 antibodies at 20 μg/mL for 45 minutes at 37° C. Media was then removed and cells were washed with 1×PBS and fixed with 4% paraformaldehyde (PFA) in 1×PBS for 10 minutes at room temperature. Non-specific binding was blocked using PBGA blocking buffer for 30 minutes at room temperature, followed by treatment with Alexa-labeled goat anti-human IgG secondary antibodies (Life Technologies; 1:400) and DAPI (4′,6-diamidino-2-phenylindole (Life Technologies; 1:20,000) for 20 minutes at room temperature. Cells were washed with 1×PBS and imaged on a Leica microscope. Using the above-described protocol, FGFR3 were visualized as prevalent fluorescent puncta surrounding the nucleus (DAPI straining), thereby confirming FGFR3 expression and internalization in the U87-FGFR3b-GFP-P2A-FLuc line.

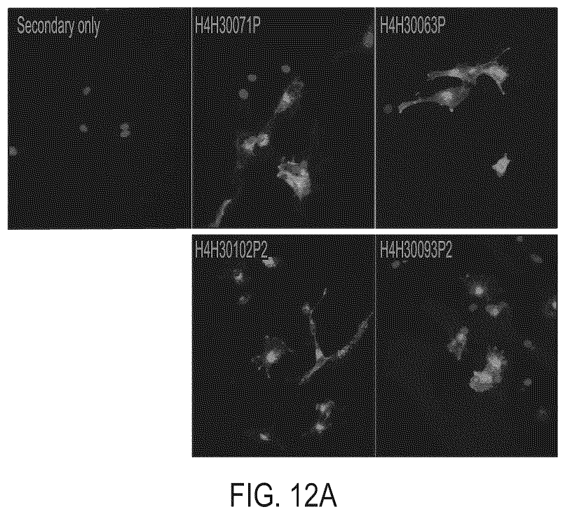

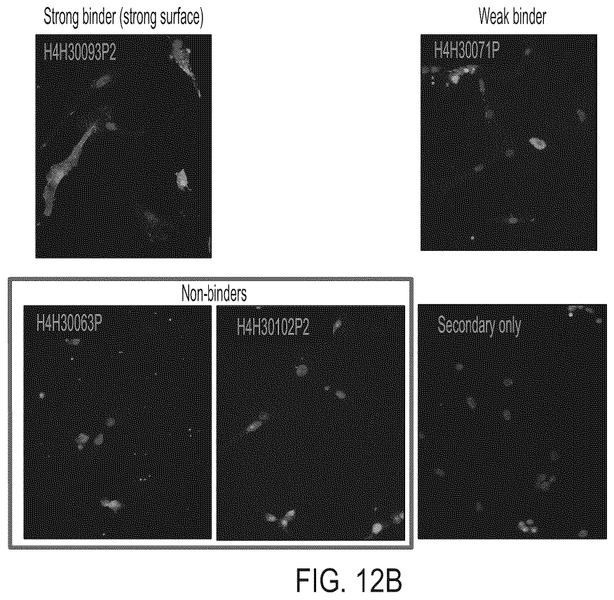

The engineered U87-FGFR3b-GFP-P2A-FLuc cell line was utilized to identify FGFR3 antibodies that could be robustly internalized. As described above, the cell line (e.g., U87-FGFR3b-GFP-P2A-FLuc) was live treated with FGFR3 antibodies described herein. The cells were then fixed and subsequently stained with anti-human IgG to identify intracellular localization of the human FGFR3 antibody-receptor complex. Observation of intracellular puncta (speckles) was indicative of FGFR3 internalization. FIG. 12A shows FGFR3 antibodies demonstrating robust internalization in the U87-FGFR3b-GFP-P2A-FLuc cell line as evidenced by prevalent intracellular puncta. FGFR3 antibodies and corresponding binding strength (as determined by measuring antibody binding to recombinant human FGFR3 protein) for strong binders are displayed in Table 2-1. For generation of the binding affinity data, a surface Plasmon Resonance (SPR) assay (Biacore) was used to immobilize recombinant human FGFR3 protein on a surface for specific testing of binding affinity to the various antibodies described herein. Internalization status, i.e., whether the antibody was internalized in the FGF3b cell line, is also included. K_{D}, equilibrium dissociation constant.

TABLE 2-1
FGFR3 antibodies and corresponding binding affinities

| | | | Internalization in |
|---|---|---|---|
| | Antibody | hFGFR3b K_{D} (M) | FGFR3b line? |
| Strong binders | H4H30071P | 1.37E−10 | Yes |
| | H4H30063P | 2.22E−10 | Yes |
| | H4H30102P2 | 1.44E−09 | Yes |
| | H4H30093P2 | 9.06E−09 | Yes |

A summary of the internalization and binding properties of the FGFR3 antibodies in FGFR3b versus FGFR3c cell lines is provided in Table 2-2. FIG. 12B illustrates non-limiting examples of immunostaining patterns observed for FGFR3 antibodies exhibiting strong binding and non-binder properties.

TABLE 2-2
Summary of internalization and binding properties of FGFR3 antibodies

| | | | Internalization | | Internalization |
|---|---|---|---|---|---|
| | | | in FGFR3b | | in |
| | Antibody | hFGFR3b K_{D} (M) | line? | hFGFR3c KD (M) | FGFR3c line? |
| Strong | H4H30093P2 | 5.14E−09 | Yes | 9.20E−09 | Yes |
| binder | | | | | |
| (strong | | | | | |
| surface) | | | | | |
| Weak | H4H30071P | 1.37E−10 | Yes | NB | Yes |
| binders | | | | | |
| Non- | H4H30063P | 2.22E−10 | Yes | NB | No |
| binders | H4H30102P2 | 1.44E−09 | Yes | NB | No |

Data depicted in FIGS. 12A-12B and Tables 2-1 and 2-2 demonstrate that differential properties (e.g., binding affinity and internalization properties) of the FGFR3 antibodies described herein could be identified when the antibodies were tested head-to-head in the U87-FGFR3b-GFP-P2A-FLuc versus the U87-FGFR3c-Fluc-GFP-P2A-FLuc cell lines. For example, H4H30063P strongly internalized in the FGFR3b line whereas no staining was observed in the FGFR3c line, in alignment with corresponding binding affinity data (see, e.g., FIG. 12A and FIG. 12B, Tables 2-1 and 2-2). In contrast, H4H30071P showed no binding via Biacore, but in the cell line screening showed weak binding with some evidence of internalization (FIG. 12A and FIG. 12B, Tables 2-1 and 2-2).

The cell line screening tool described in the present Example can be used as a complimentary technique to assess binding and internalization properties of the FGFR3 antibodies described herein. The cellular assay, in particular, can offer a conformationally-relevant FGFR3 approach since it occurs in a natural cell environment as compared to the Biacore assay which uses a recombinant protein immobilized to a surface to test for antibody binding strength.

### Example 8. Binding Kinetics

Biacore binding kinetics of anti-FGFR3 antibodies, in an antibody capture format, to monomeric human FGFR3b, cynomolgus FGFR3b, murine FGFR3b, human FGFR3c, and dimeric human FGFR3b ecto domain recombinant proteins at 25° C. were analyzed.

Equilibrium dissociation constants (K_{D} values) for human FGFR3b expressed with a C-terminal myc-myc-hexahistidine tag (hFGFR3b.mmH, REGN3152) or cynomolgus FGFR3b expressed with a C-terminal myc-myc-hexahistidine tag (mfFGFR3b.mmH, REGN3521), or murine FGFR3b expressed with a C-terminal myc-myc-hexahistidine tag (mFGFR3b.mmH, REGN3215) or human FGFR3c expressed with a C-terminal myc-myc-hexahistidine tag (hFGFR3c.mmH, REGN3155) or human FGFR3b expressed with an C-terminal murine Fc tag (hFGFR3b.mFc, REGN3153) binding to purified anti-FGFR3 antibodies were determined using a real-time surface plasmon resonance biosensor technology using a Biacore 3000 or Biacore 4000 instrument. The CM5 Biacore sensor surface was derivatized by amine coupling with a monoclonal mouse anti-human Fc monoclonal antibody (REGN2567). All Biacore binding studies were performed in a buffer composed of 0.01M HEPES pH 7.4, 0.15 M NaCl, 3 mM EDTA, 0.05% v/v Surfactant P20 (HBS-EP running buffer). Different concentrations of monomeric proteins prepared in HBS-EP running buffer (ranging from 90 nM to 3.33 nM in 3-fold serial dilutions) or dimeric protein (hFGFR3b.mFc, REGN3153) prepared in HBS-EP running buffer (ranging from 30 to 10 nM in 3-fold serial dilution) were injected over the captured anti-FGFR3 antibodies at a flow rate of 30 μL/minute. Antibody-reagent association was monitored for 5 minutes while dissociation in HBS-EP running buffer was monitored for 10 minutes. At the end of each cycle, the anti-FGFR3 antibody capture surface was regenerated using a 10 sec injection of 20 mM phosphoric acid. All binding kinetics experiments were performed at 25° C.

The specific SPR-Biacore sensorgrams were obtained by a double referencing procedure. The double referencing was performed by first subtracting the signal of each injection over a reference surface (anti-hFc) from the signal over the experimental surface (anti-hFc-captured anti-FGFR3 antibodies) thereby removing contributions from refractive index changes. In addition, running buffer injections were performed to allow subtraction of the signal changes resulting from the dissociation of captured antibodies from the coupled anti-hFc surface. Kinetic association (kₐ) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{kd}{ka} ,$$

$$and$$

$$t \frac{1}{2} ( min ) = \frac{ln ( 2 )}{60 * kd}$$

Monomeric kinetics results are presented in Tables 3-1 through 3-4. Dimeric kinetic results are presented in Table 3-5.

REGN3152 (human FGFR3b expressed with a
C-terminal myc-myc-hexahistidine tag
(hFGFR3b.mmH))
(SEQ ID NO: 238)
ESLGTEQRVVGRAAEVPGPEPGQQEQLVFGSGDAVELSCPPPGGG
PMGPTVWVKDGTGLVPSERVLVGPORLOVLNASHEDSGAYSCRQR
LTQRVLCHFSVRVTDAPSSGDDEDGEDEAEDTGVDTGAPYWTRPE
RMDKKLLAVPAANTVRFRCPAAGNPTPSISWLKNGREFRGEHRIG
GIKLRHQQWSLVMESVVPSDRGNYTCVVENKFGSIRQTYTLDVLE
RSPHRPILQAGLPANQTAVLGSDVEFHCKVYSDAQPHIQWLKHVE
VNGSKVGPDGTPYVTVLKSWISESVEADVRLRLANVSERDGGEYL
CRATNFIGVAEKAFWLSVHGPRAAEEELVEADEAGSVYAGEQKLI
SEEDLGGEQKLISEEDLHHHHHH
REGN3153 (human FGFR3b expressed with a
C-terminal murine Fc tag (hFGFR3b.mFc))
(SEQ ID NO: 239)
ESLGTEQRVVGRAAEVPGPEPGQQEQLVFGSGDAVELSCPPPGGG
PMGPTVWVKDGTGLVPSERVLVGPQRLQVLNASHEDSGAYSCRQR
LTQRVLCHFSVRVTDAPSSGDDEDGEDEAEDTGVDTGAPYWTRPE
RMDKKLLAVPAANTVRFRCPAAGNPTPSISWLKNGREFRGEHRIG
GIKLRHQQWSLVMESVVPSDRGNYTCVVENKFGSIRQTYTLDVLE
RSPHRPILQAGLPANQTAVLGSDVEFHCKVYSDAQPHIQWLKHVE
VNGSKVGPDGTPYVTVLKSWISESVEADVRLRLANVSERDGGEYL
CRATNFIGVAEKAFWLSVHGPRAAEEELVEADEAGSVYAGEPRGP
TIKPCPPCKCPAPNLLGGPSVFIFPPKIKDVLMISLSPIVTCVVV
DVSEDDPDVQISWFVNNVEVHTAQTQTHREDYNSTLRVVSALPIQ
HQDWMSGKEFKCKVNNKDLPAPIERTISKPKGSVRAPQVYVLPPP
EEEMTKKQVTLTCMVTDFMPEDIYVEWTNNGKTELNYKNTEPVLD
SDGSYFMYSKLRVEKKNWVERNSYSCSVVHEGLHNHHTTKSFSRT
PGK
REGN3215 (murine FGFR3b expressed with a
C-terminal myc-myc-hexahistidine tag
(mFGFR3b.mmH))
(SEQ ID NO: 240)
EPPGPEQRVVRRAAEVPGPEPSQQEQVAFGSGDTVELSCHPPGGA
PTGPTVWAKDGTGLVASHRILVGPQRLQVLNASHEDAGVYSCQHR
LTRRVLCHFSVRVTDAPSSGDDEDGEDVAEDTGAPYWTRPERMDK
KLLAVPAANTVRERCPAAGNPTPSISWLKNGKEFRGEHRIGGIKL
RHQQWSLVMESVVPSDRGNYTCVVENKFGSIRQTYTLDVLERSPH
RPILQAGLPANQTAILGSDVEFHCKVYSDAQPHIQWLKHVEVNGS
KVGPDGTPYVTVLKSWISENVEADARLRLANVSERDGGEYLCRAT
NFIGVAEKAFWLRVHGPQAAEEELMETDEAGSVYAGEQKLISEED
LGGEQKLISEEDLHHHHHH
REGN3155 (human FGFR3c expressed with a
C-terminal myc-myc-hexahistidine tag
(hFGFR3c.mmH))
(SEQ ID NO: 241)
ESLGTEQRVVGRAAEVPGPEPGQQEQLVFGSGDAVELSCPPPGGG
PMGPTVWVKDGTGLVPSERVLVGPQRLQVLNASHEDSGAYSCRQR
LTQRVLCHFSVRVTDAPSSGDDEDGEDEAEDTGVDTGAPYWTRPE
RMDKKLLAVPAANTVRFRCPAAGNPTPSISWLKNGREFRGEHRIG
GIKLRHQQWSLVMESVVPSDRGNYTCVVENKFGSIRQTYTLDVLE
RSPHRPILQAGLPANQTAVLGSDVEFHCKVYSDAQPHIQWLKHVE
VNGSKVGPDGTPYVTVLKTAGANTTDKELEVLSLHNVTFEDAGEY
TCLAGNSIGFSHHSAWLVVLPAEEELVEADEAGSVYAGEQKLISE
EDLGGEQKLISEEDLHHHHHH
REGN3521 (mf FGFR3b ecto (R357G)
(E23-G377;R357G).mmH)
(SEQ ID NO: 242)
ESLGTEQRVVGRVAEVSGPEPSQQEQLVFGSGDAVELSCPPPGGG
PMGPTVWVKDGAGLVPSERVLVGPQRLQVLNASHEDSGAYSCRQR
LTQLVLCHFSVRVTDAPSSGDDEDGEDEAEDTGVDTGAPYWTRPE
RMDKKLLAVPAANTVRFRCPAAGNPTPSISWLKNGKEFRGEHRIG
GIKLRHQQWSLVMESVVPSDRGNYTCVVENKFGSIRQTYTLDVLE
RSPHRPILOAGLPANQTAVLGSDVEFHCKVYSDAQPHIQWLKHVE
VNGSKVGPDGTPYVTVLKSWISESVEADVRLRLANVSERDGGEYL
CRATNFIGVAEKAFWLSVHGPRAAEEELVEADEAGSVYAGEQKLI
SEEDLGGEQKLISEEDLHHHHHH

TABLE 3-1
Kinetic and Equilibrium Binding Parameters of Monomeric Human
FGFR3b to Surface-captured anti-FGFR3 Antibodies at 25° C.

| | mAb | 90 nM | | | | |
|---|---|---|---|---|---|---|
| | Capture | hFGFR3b.mmH | ka | kd | KD | t½ |
| | (RU) | Bind (RU) | (1/Ms) | (1/s) | (M) | (min) |
| H4H30045P | 584.3 ± 7.5 | 124.1 | 3.85E+05 | 6.24E−03 | 1.62E−08 | 1.9 |
| H4H30061P | 561.3 ± 8.2 | 249.2 | 1.73E+05 | 8.74E−05 | 5.06E−10 | 132.2 |
| H4H30063P | 577.0 ± 5.6 | 240.5 | 1.72E+05 | 3.83E−05 | 2.22E−10 | 301.9 |
| H4H30066P | 486.8 ± 2.9 | 224.5 | 4.96E+05 | 6.35E−04 | 1.28E−09 | 18.2 |
| H4H30071P | 515.2 ± 7.9 | 253.0 | 2.16E+05 | 2.96E−05 | 1.37E−10 | 390.5 |
| H4H30076P | 436.8 ± 13.8 | 198.0 | 2.36E+05 | 4.39E−05 | 1.86E−10 | 263.3 |
| H4H30089P2 | 479.7 ± 3.5 | 152.1 | 2.76E+05 | 2.82E−03 | 1.02E−08 | 4.1 |
| H4H30093P2 | 496.4 ± 5.2 | 217.0 | 4.06E+05 | 2.09E−03 | 5.14E−09 | 5.5 |
| H4H30095P2 | 506.5 ± 1.1 | 239.4 | 6.98E+05 | 5.00E−03 | 7.16E−09 | 2.3 |
| H4H30102P2 | 438.3 ± 2.7 | 134.8 | 1.68E+05 | 2.43E−04 | 1.44E−09 | 47.5 |
| H4H30105P2 | 467.9 ± 0.3 | 157.7 | 4.38E+05 | 5.11E−03 | 1.17E−08 | 2.3 |
| H4H30108P2 | 544.3 ± 3.1 | 227.2 | 1.76E+05 | 1.59E−04 | 9.05E−10 | 72.6 |
| H4H30117P2 | 412.2 ± 0.8 | 177.5 | 6.56E+05 | 6.30E−03 | 9.60E−09 | 1.8 |
| COMP6331 | 145.4 ± 3.4 | 58.38 | 8.83E+05 | 6.16E−04 | 6.97E−10 | 18.76 |

* See V_{H} and V_{L} set forth in WO2016/134234

REGN6331 Heavy Chain
(SEQ ID NO: 243)
EVQLVESGGGLVQPGGSLRLSCAASGFTFTSTGISWVRQAPGKGL
EWVGRIYPTSGSTNYADSVKGRFTISADTSKNTAYLQMNSLRAED
TAVYYCARTYGIYDLYVDYTEYVMDYWGQGTLVTVSSASTKGPSV
FPLAPSSKSTSGGTAALGCLVKDYFPEPVTVSWNSGALTSGVHTF
PAVLQSSGLYSLSSVVTVPSSSLGTQTYICNVNHKPSNTKVDKKV
EPKSCDKTHTCPPCPAPELLGGPSVFLFPPKPKDTLMISRTPEVT
CVVVDVSHEDPEVKENWYVDGVEVHNAKTKPREEQYNSTYRVVSV
LTVLHQDWLNGKEYKCKVSNKALPAPIEKTISKAKGQPREPQVYT
LPPSRDELTKNQVSLTCLVKGFYPSDIAVEWESNGQPENNYKTTP
PVLDSDGSFFLYSKLTVDKSRWQQGNVFSCSVMHEALHNHYTQKS
LSLSPGK
REGN6331 Light Chain
(SEQ ID NO: 244)
DIQMTQSPSSLSASVGDRVTITCRASQDVDTSLAWYKQKPGKAPK
LLIYSASFLYSGVPSRFSGSGSGTDFTLTISSLQPEDFATYYCQQ
STGHPQTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

TABLE 3-2
Kinetic and Equilibrium Binding Parameters of Monomeric Cynomolgus
FGFR3b to Surface-captured anti-FGFR3 Antibodies at 25° C.

| | mAb | 90 nM | | | | |
|---|---|---|---|---|---|---|
| | Capture | mfFGFR3b.mmH | ka | kd | KD | t½ |
| Ab PID # | (RU) | Bind (RU) | (1/Ms) | (1/s) | (M) | (min) |
| H4H30045P | 587.4 ± 1.6 | 71.9 | 4.29E+05 | 8.82E−03 | 2.06E−08 | 1.3 |
| H4H30061P | 555.8 ± 4.2 | 197.3 | 1.51E+05 | 9.83E−05 | 6.50E−10 | 117.5 |
| H4H30063P | 574.1 ± 4.8 | 212.5 | 1.53E+05 | 4.21E−05 | 2.75E−10 | 274.7 |
| H4H30066P | 487.2 ± 3.8 | 203.2 | 3.91E+05 | 7.79E−04 | 1.99E−09 | 14.8 |
| H4H30071P | 507.6 ± 6.8 | 233.1 | 1.80E+05 | 2.22E−05 | 1.23E−10 | 520.3 |
| H4H30076P | 437.5 ± 6.9 | 179.7 | 2.00E+05 | 3.30E−05 | 1.65E−10 | 350.2 |
| H4H30089P2 | 486.8 ± 4.6 | 147.3 | 2.16E+05 | 2.21E−03 | 1.03E−08 | 5.2 |
| H4H30093P2 | 499.6 ± 3.1 | 3.4 | NB* | NB* | NB* | NB* |
| H4H30095P2 | 509.2 ± 1.7 | 205.9 | 5.27E+05 | 4.37E−03 | 8.29E−09 | 2.6 |
| H4H30102P2 | 442.5 ± 2.0 | 125.9 | 1.31E+05 | 2.30E−04 | 1.75E−09 | 50.2 |
| H4H30105P2 | 468.6 ± 2.4 | 67.8 | 5.47E+05 | 8.02E−03 | 1.47E−08 | 1.4 |
| H4H30108P2 | 543.7 ± 3.1 | 211.5 | 1.49E+05 | 1.36E−04 | 9.09E−10 | 85.2 |
| H4H30117P2 | 413.8 ± 1.4 | 147.6 | 5.50E+05 | 8.70E−03 | 1.58E−08 | 1.3 |
| COMP6331 | NT** | NT** | NT** | NT** | NT** | NT** |

*NB = Non-Binding
**NT = Not Tested

TABLE 3-3
Kinetic and Equilibrium Binding Parameters of Monomeric Murine
FGFR3b to Surface-captured anti-FGFR3 Antibodies at 25° C.

| | mAb | 90 nM | | | | |
|---|---|---|---|---|---|---|
| | Capture | mFGFR3b.mmH | ka | kd | KD | t½ |
| Ab PID # | (RU) | Bind (RU) | (1/Ms) | (1/s) | (M) | (min) |
| H4H30045P | 586.0 ± 2.6 | 2.4 | NB* | NB** | NB** | NB** |
| H4H30061P | 561.0 ± 1.5 | −8.5 | NB** | NB** | NB** | NB** |
| H4H30063P | 570.0 ± 6.5 | 259.2 | 2.04E+05 | 2.78E−05 | 1.37E−10 | 415.5 |
| H4H30066P | 486.0 ± 2.9 | 64.6 | 2.88E+05 | 4.87E−03 | 1.69E−08 | 2.4 |
| H4H30071P | 511.0 ± 3.2 | 174.3 | 2.20E+05 | 1.69E−03 | 7.65E−09 | 6.9 |
| H4H30076P | 438.1 ± 14.9 | 201.1 | 2.56E+05 | 3.61E−05 | 1.41E−10 | 319.7 |
| H4H30089P2 | 484.0 ± 7.0 | 60.0 | 2.81E+05 | 2.38E−03 | 8.45E−09 | 4.9 |
| H4H30093P2 | 503.8 ± 4.9 | 11.2 | 1.36E+05 | 9.67E−03 | 7.09E−08 | 1.2 |
| H4H30095P2 | NA* | NA* | NA* | NA* | NA* | NA* |
| H4H30102P2 | NA* | NA* | NA* | NA* | NA* | NA* |
| H4H30105P2 | 469.9 ± 0.9 | 8.3 | 1.49E+05 | 1.22E−03 | 8.19E−09 | 9.5 |
| H4H30108P2 | 541.4 ± 3.4 | 121.1 | 1.62E+05 | 1.99E−03 | 1.23E−08 | 5.8 |
| H4H30117P2 | 414.0 ± 1.0 | 0.9 | NB** | NB** | NB** | NB** |
| COMP6331 | NT*** | NT*** | NT*** | NT*** | NT*** | NT*** |

*Not Acquired. Technical issue prevented data acquisition and analysis.
**NB = Non-Binding
***NT: Not Tested

TABLE 3-4
Kinetic and Equilibrium Binding Parameters of Monomeric Human
FGFR3c to Surface-captured anti-FGFR3 Antibodies at 25° C.

| | mAb | 90 nM | | | | |
|---|---|---|---|---|---|---|
| | Capture | hFGFR3c.mmH | ka | kd | KD | t½ |
| Ab PID # | (RU) | Bind (RU) | (1/Ms) | (1/s) | (M) | (min) |
| H4H30045P | 583.4 ± 1.4 | 77.1 | 3.81E+05 | 7.46E−03 | 1.96E−08 | 1.5 |
| H4H30061P | 555.4 ± 4.1 | 203.8 | 1.51E+05 | 7.92E−05 | 5.26E−10 | 145.8 |
| H4H30063P | 577.8 ± 1.9 | −19.6 | NB* | NB* | NB* | NB* |
| H4H30066P | 485.8 ± 2.4 | 0.5 | NB* | NB* | NB* | NB* |
| H4H30071P | 519.0 ± 0.5 | −4.8 | NB* | NB* | NB* | NB* |
| H4H30076P | 435.7 ± 9.9 | −9.7 | NB* | NB* | NB* | NB* |
| H4H30089P2 | 484.6 ± 5.0 | −6.7 | NB* | NB* | NB* | NB* |
| H4H30093P2 | 503.2 ± 4.1 | 182.1 | 3.63E+05 | 3.34E−03 | 9.20E−09 | 3.5 |
| H4H30095P2 | 508.5 ± 0.9 | 221.8 | 5.75E+05 | 4.54E−03 | 7.88E−09 | 2.5 |
| H4H30102P2 | 448.0 ± 0.7 | −11.7 | NB* | NB* | NB* | NB* |
| H4H30105P2 | 469.7 ± 0.8 | 130.6 | 3.87E+05 | 6.52E−03 | 1.68E−08 | 1.8 |
| H4H30108P2 | 541.2 ± 1.9 | −2.5 | NB* | NB* | NB* | NB* |
| H4H30117P2 | 413.7 ± 0.6 | 165.5 | 5.95E+05 | 7.32E−03 | 1.23E−08 | 1.6 |
| COMP6331 | 141.5 ± 3.2 | 39.56 | 5.44E+05 | 1.32E−03 | 2.42E−09 | 8.77 |

*NB = Non-Binding

TABLE 3-5
Kinetic and Equilibrium Binding Parameters of Dimeric Human
FGFR3b to Surface-captured anti-FGFR3 Antibodies at 25° C.

| | mAb | 30 nM | | | | |
|---|---|---|---|---|---|---|
| | Capture | hFGFR3b.mFc | ka | kd | KD | t½ |
| Ab PID # | (RU) | Bind (RU) | (1/Ms) | (1/s) | (M) | (min) |
| H4H30045P | 573.0 ± 4.6 | 265.5 | 2.77E+05 | 1.61E−04 | 5.81E−10 | 71.8 |
| H4H30061P | 544.3 ± 4.8 | 259.7 | 2.31E+05 | ≤1E−5 | 4.33E−11 | ≥1155 |
| H4H30063P | 567.0 ± 7.1 | 296.1 | 4.42E+05 | ≤1E−5 | 2.26E−11 | ≥1155 |
| H4H30066P | 484.6 ± 2.2 | 282.3 | 6.32E+05 | ≤1E−5 | 1.58E−11 | ≥1155 |
| H4H30071P | 502.2 ± 2.5 | 235.8 | 3.22E+05 | ≤1E−5 | 3.10E−11 | ≥1155 |
| H4H30076P | 435.0 ± 2.6 | 204.1 | 4.07E+05 | ≤1E−5 | 2.46E−11 | ≥1155 |
| H4H30089P2 | 485.6 ± 4.1 | 213.9 | 3.12E+05 | 1.90E−05 | 6.10E−11 | 607.9 |
| H4H30093P2 | 497.8 ± 8.2 | 316.9 | 5.85E+05 | 1.55E−05 | 2.65E−11 | 744.7 |
| H4H30095P2 | 504.5 ± 2.0 | 404.9 | 8.67E+05 | 9.88E−05 | 1.14E−10 | 117.0 |
| H4H30102P2 | 443.7 ± 1.7 | 131.4 | 2.97E+05 | ≤1E−5 | 3.37E−11 | ≥1155 |
| H4H30105P2 | NA* | NA* | NA* | NA* | NA* | NA* |
| H4H30108P2 | 532.1 ± 3.5 | 213.0 | 3.11E+05 | ≤1E−5 | 3.22E−11 | ≥1155 |
| H4H30117P2 | 411.4 ± 1.5 | 337.0 | 7.87E+05 | 1.33E−04 | 1.69E−10 | 87.0 |
| COMP6331 | NT** | NT** | NT** | NT** | NT** | NT** |

*NA = Not Acquired. Technical issue prevented data acquisition and analysis
**NT = Not Tested

### Example 9. Blocking Assays

Anti-FGFR3 antibodies blocking dimeric human FGFR3b or FGFR3c binding to human FGF acidic or FGF basic by ELISA was analyzed.

TABLE 4-1
Reagents used

| Reagent | Source | Identification |
|---|---|---|
| hFGF acidic | R & D Systems | 231-BC-025/CF |
| hFGF basic | Biolegend | 571508 |
| hFGFR3c-hFc | R & D Systems | 766-FR-050 |
| hFGFR3b-mFc | | REGN3153 |
| Human IgG1 | | REGN1932 |
| negative control | | |
| antibody (specific for | | |
| cat allergy antigen- | | |
| Fel d1) | | |
| Human lgG4 | | REGN1945 |
| negative control | | |
| antibody (specific for | | |
| cat allergy antigen- | | |
| Fel d1) | | |
| anti-FGFR3-hlgG1 | | REGN6331 |
| comparator | | |
| Peroxidase | Jackson | 109-035-098 |
| AffiniPure Goat Anti- | ImmunoResearch | |
| Human IgG, Fcγ | | |
| fragment specific | | |
| Peroxidase | Jackson | 115-035-164 |
| AffiniPure Goat Anti- | ImmunoResearch | |
| Mouse IgG | | |
| (subclasses | | |
| 1 + 2a + 2b + 3), Fcγ | | |
| Fragment Specific | | |
| 1X PBS | Irvine Scientific | 9236 |
| 1X PBST (0.05% | Sigma | P3563 |
| tween-20 in PBS) | | |
| BSA:albumin | Sigma | A8577-1L |
| solution from bovine | | |
| serum, 30% | | |
| Heparin | Sigma | H4784-1G |
| 3-3′, 5-5′- | BD Biosciences | 51-2606KC |
| tetramethylbenzidine | | |
| (TMB) Substrate | | |
| Reagent Set- | | |
| Substrate Reagent | | |
| A | | |
| 3-3′, 5-5′- | BD Biosciences | 51-2607KC |
| tetramethylbenzidine | | |
| (TMB) Substrate | | |
| Reagent Set- | | |
| Substrate Reagent | | |
| B | | |
| 2N Sulfuric Acid | VWR | BDH7500-1 |
| Reacti-Bind 96-well | ThermoFisher | P15041 |
| plates | Scientific | |
| 96-well deep well | VWR | 82006-448, |
| plates | | 82007-292 |
| 96-Well | Fisher Scientific | Fisher 12- |
| Polypropylene | | 565-369 |
| storage microplates | | |
| Aquamax Plate | Molecular Devices | Aquamax 2000 |
| Washer 2000 | | |
| VICTOR TM X5 | PerkinElmer | Model #2030 |
| Multilabel Plate | | |
| Reader | | |
| GraphPad Prism | GraphPad Software | Version 9 |

An ELISA-based blocking assay was developed to determine the ability of anti-FGFR3 antibodies to block the binding of human fibroblast growth factor receptor 3 isoform b (hFGFR3b) or human fibroblast growth factor receptor 3 isoform c (hFGFR3c) to human fibroblast growth factor acidic (hFGF acidic) or basic (hFGF basic) ligands.

The human FGFR3b recombinant protein used in the experiments had the hFGFR3b extracellular domain (amino acids E23-G377) expressed with the Fc portion of the mouse IgG2a at the C-terminus (amino acids E98-K330) (hFGFR3b-mFc, accession #NM 001163213.1). The hFGFR3c and hFGF acidic proteins were purchased commercially. The human FGFR3c protein had the hFGFR3c extracellular domain (amino acids Glu23-Gly375) expressed with Fc portion of the human IgG1 at the C-terminus (amino acids Pro100-Lys330) (hFGFR3c-hFc, accession #P22607) and the hFGF acidic protein was expressed with amino acids Ala2-Asp155 (accession #P05230.1). The hFGF basic protein was purchased commercially. It was expressed with amino acids Ala144-Ser288 (accession #NM 002006).

In the blocking assay, a 96-well microtiter plate was coated with either hFGF acidic or hFGF basic proteins at 2 mg/ml in PBS+10 mg/ml heparin overnight at 4° C. Nonspecific binding sites were subsequently blocked using a 0.5% (w/v) solution of BSA+10 μg/ml heparin in PBS. In other 96-well microtiter plates, a fixed amount of 4 nM hFGFR3b-mFc, 0.4 nM or 7 nM hFGFR3c-hFc was bound for one hour with anti-FGFR3, anti-FGFR3 comparator, or irrelevant human IgG1 or IgG4 isotype antibody at dilutions from 3.4 μM to 200 nM in PBS+0.5% BSA+10 μg/ml heparin. The fixed concentration of hFGFR3b or hFGFR3c proteins was selected to be near the concentration that generated 50% of the maximal binding (EC 50 value) to plate-adhered hFGF acidic or hFGF basic protein. The antibody complexes with 4 nM hFGFR3b-mFc or 0.4 nM hFGFR3c-hFc were transferred to microtiter plates coated with hFGF acidic protein. In parallel, antibody complexes with 7 nM hFGFR3c-hFc were added to the microtiter plates coated with hFGF basic protein. After a 1 hour incubation at room temperature, plates were washed, and plate-bound hFGFR3b-mFc or hFGFR3c-hFc proteins were detected with horseradish peroxidase (HRP) conjugated goat anti-mouse or goat anti-human Fcγ fragment specific antibodies. The plates were then developed using TMB substrate solution (BD Biosciences) according to the manufacturer's recommended procedure and absorbance at 450 nm was measured on a Victor X5 plate reader.

Binding data were analyzed using a sigmoidal (four-parameter logistic) dose-response model using GraphPad Prism software. The calculated IC₅₀ value, defined as the concentration of antibody required to block 50% of hFGFR3b-mFc or hFGFR3c-hFc binding to plate-coated hFGF acidic or hFGF basic protein, was used as an indicator of blocking potency. Percent blocking of FGFR3 antibody at a given concentration was calculated based on the formula shown below.

$$\% Blocking = 100 - ( \frac{\begin{matrix}[ Experimental {Signal}_{( highest Ab conc )} - \\ Background {Signal}_{( buffer )}\end{matrix}}{\begin{matrix}[ Maximum {Signal}_{( hFGFR 3 b - mFc or hFGF 3 c - hFc )} - \\ Background {Signal}_{( buffer )}\end{matrix}} ) \times 100$$

Antibodies that blocked binding more than 50% at the highest concentration tested were classified as blockers and IC 50 values were reported.

The ability of anti-FGFR3 antibodies to block human FGFR3b binding to hFGF acidic protein or hFGFR3c binding to hFGF acidic or hFGF basic proteins was evaluated using a sandwich ELISA-based blocking assay. In this assay, a fixed concentration of the hFGFR3b-mFc or hFGFR3c-hFc was pre-incubated with a wide concentration range of anti-FGFR3 antibodies before binding to plate immobilized hFGF acidic or basic proteins, and the plate-bound hFGFR3b-mFc or hFGFR3c-hFc was detected with HRP-conjugated goat anti-mouse or goat anti-human Fcγ fragment specific antibodies, respectively. Six anti-FGFR3 antibodies were evaluated for inhibition of hFGFR3b-mFc binding to hFGF acidic protein. Antibody H4H30093P2, which also binds hFGFR3c, was additionally tested for inhibition of hFGFR3c-hFc binding to hFGF acidic or hFGF basic proteins. The IC 50 values and maximum blocking at the highest tested concentrations of the FGFR3 antibodies are summarized in Table 4-2.

All six anti-FGFR3 antibodies (H4H30063P, H4H30066P, H4H30071P, H4H30089P2, H4H30093P2 and H4H30102P2) displayed concentration-dependent blocking of hFGFR3b-mFc binding to hFGF acidic protein with the extent of block ranging from 68% to 95% at the highest antibody concentration tested (200 nM). The IC₅₀ values for these blocking antibodies ranged from 2 nM to 15 nM. Antibody H4H30093P2 displayed less than 50% blocking of the binding of hFGFR3c to hFGF acidic or hFGF basic at the highest antibody concentration tested and was classified as a non-blocker for hFGFR3c. The anti-FGFR3 comparator antibody blocked the binding of hFGFR3b to hFGF acidic with an IC₅₀ of 1.5 nM, and binding of hFGFR3c binding to hFGF acidic or hFGF basic with an IC₅₀ of 0.1 nM and 8.9 nM, respectively. The human IgG1 or IgG4 isotype control antibodies did not block in any assays.

TABLE 4-2
IC50 values and maximum blocking at the highest
tested concentrations of the FGFR3 antibodies

| | Antibody blocking 4 nM | | Antibody blocking 0.4 nM | | Antibody blocking 7 nM | |
|---|---|---|---|---|---|---|
| | hFGFR3b-mFc binding | | hFGFR3c-hFc binding to | | hFGFR3c-hFc binding to | |
| | to hFGF acidic protein | | hFGF acidic protein | | hFGF basic protein | |
| | | % Blocking | | % Blocking | | % Blocking |
| | | at 200 nM | | at 200 nM | | at 200 nM |
| Antibody | IC₅₀, M | mAb | IC₅₀, M | mAb | IC₅₀, M | mAb |
| H4H30063P | 2.0E−09 | 95 | ND | ND | ND | ND |
| H4H30066P | 3.2E−09 | 90 | ND | ND | ND | ND |
| H4H30071P | 3.6E−09 | 88 | ND | ND | ND | ND |
| H4H30089P2 | 1.5E−08 | 68 | ND | ND | ND | ND |
| H4H30093P2 | 2.5E−09 | 91 | Nbl | −7 | Nbl | −15 |
| H4H30102P2 | 2.5E−09 | 92 | ND | ND | ND | ND |
| hIgG4 | Nbl | 14 | Nbl | 16 | Nbl | −5 |
| isotype | | | | | | |
| control | | | | | | |
| anti-FGFR3- | 1.5E−09 | 99 | 1.0E−10 | 86 | 8.9E−09 | 87 |
| hIgG1 | | | | | | |
| comparator | | | | | | |
| (*) | | | | | | |
| hIgG1 | Nbl | −7 | Nbl | 14 | Nbl | −20 |
| isotype | | | | | | |
| control (*) | | | | | | |

Nbl: Non-blocking, % blocking is less than or equal to 50
ND: Not determined
(*) anti-FGFR3-hIgG1 comparator and hIgG1 isotype control were tested in a separate experiment for blocking of hFGFR3b binding to hFGF acidic protein or hFGFR3c binding to hFGF basic protein

### Example 10. Octet Cross-Competition Analysis of Anti-FGFR3 Antibodies

Binding competition between anti-FGFR3 monoclonal antibodies that had been previously determined to bind to hFGFR3b.mmH was determined using a real time, label-free bio-layer interferometry (BLI) assay on an Octet HTX biosensor (ForteBio Corp., A Division of Pall Life Sciences). The entire experiment was performed at 25° C. in buffer comprised of 0.01 M HEPES pH7.4, 0.15 M NaCl, 3 mM EDTA, 0.05% v/v Surfactant P20, 0.1 mg/mL BSA (Octet HBS-EP buffer) with the plate shaking at a speed of 1000 rpm. To assess whether two antibodies were able to compete with one another for binding to hFGFR3b ecto domain expressed with a C-terminal myc-myc-histidine tag (hFGFR3b.mmH, REGN3152) approximately 0.2 nm of hFGFR3b.mmH was first captured onto anti-penta-His antibody coated Octet biosensors (Fortebio Inc, #18-5079) by submerging the biosensors for 23 seconds into wells containing a 20 μg/mL solution of hFGFR3b.mmH. The antigen-captured biosensors were then saturated with the first anti-FGFR3 monoclonal antibody (subsequently referred to as mAb-1) by immersion into wells containing a 50 μg/mL solution of mAb-1 for 5 minutes. The biosensors were then subsequently submerged into wells containing a 50 μg/mL solution of a second anti-FGFR3 monoclonal antibody (subsequently referred to as mAb-2) for 3 minutes. The real-time binding response was monitored during the course of the experiment and the binding response at the end of every step was recorded. The response of mAb-2 binding to hFGFR3b.mmH pre-complexed with mAb-1 was compared and the competitive or non-competitive property of an anti-FGFR3 monoclonal antibody toward another antibody was determined using a 50% inhibition threshold. Table 5-1 summarizes cross-competing antibodies which competed binding to hFGFR3b.mmH independent of the order of the sequential binding of mAb-1 and mAb-2.

TABLE 5-1
Cross-competition of anti-FGFR3 antibodies for
binding to hFGFR3b.mmH.

| First mAb (mAb-1) | mAb-2 Antibodies |
|---|---|
| captured using AHC | Shown to Compete |
| Octet Biosensors | with mAb-1 |
| H4H30045P | H4H30045P |
| | H4H30095P2 |
| H4H30061P | H4H30093P2 |
| | H4H30061P |
| H4H30063P | H4H30063P |
| | H4H30076P |
| H4H30066P | H4H30089P2 |
| | H4H30108P2 |
| | H4H30071P |
| | H4H30102P2 |
| | H4H30066P |
| H4H30071P | H4H30089P2 |
| | H4H30108P2 |
| | H4H30071P |
| | H4H30102P2 |
| | H4H30066P |
| H4H30076P | H4H30063P |
| | H4H30076P |
| | H4H30089P2 |
| H4H30089P2 | H4H30076P |
| | H4H30089P2 |
| | H4H30108P2 |
| | H4H30071P |
| | H4H30102P2 |
| | H4H30066P |
| H4H30093P2 | H4H30093P2 |
| | H4H30061P |
| H4H30095P2 | H4H30045P |
| | H4H30095P2 |
| | H4H30105P2 |
| | H4H30117P2 |
| H4H30102P2 | H4H30089P2 |
| | H4H30108P2 |
| | H4H30071P |
| | H4H30102P2 |
| | H4H30066P |
| H4H30105P2 | H4H30095P2 |
| | H4H30105P2 |
| | H4H30117P2 |
| H4H30108P2 | H4H30089P2 |
| | H4H30108P2 |
| | H4H30071P |
| | H4H30102P2 |
| | H4H30066P |
| H4H30117P2 | H4H30095P2 |
| | H4H30105P2 |
| | H4H30117P2 |

### Example 11: Hydrogen/Deuterium Exchange (HDX) Epitope Mapping

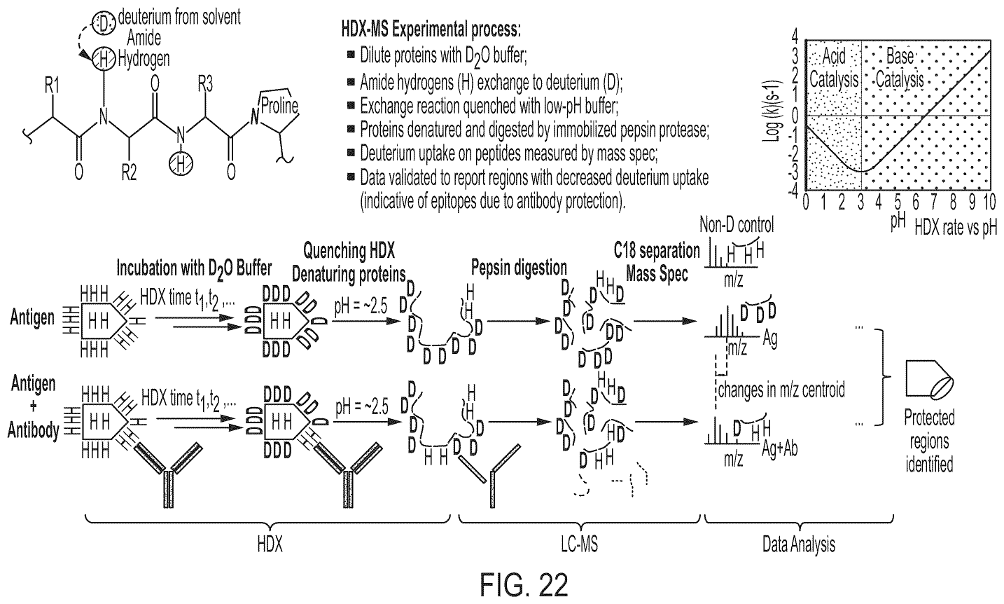

HDX experiment was performed using a customized HDX automation system (NovaBioAssays, MA) coupled to a Q Exactive HF mass spectrometer (Thermo Fisher Scientific, MA). FIG. 22 shows an exemplary HDX-Mass Spectrometry experimental process.

To initiate deuterium exchange, 10 μL of protein sample (hFGFR3b.mmh alone or hFGFR3b.mmh in mixture with H4H30117P2, H4H30063P, H4H30045P, or H4H30108P2 at 2:1 ratio) was diluted with 90 μL PBS-D20 buffer (10 mM, pH 7.4 at 25° C.). After 5 or 10 min, deuterium exchange was quenched by adding 100 μL quenching buffer (0.5 M TCEP, 4 M guanidine hydrochloride, pH 2.08) followed by 90 seconds incubation at 20° C. The quenched samples were digested by online pepsin/protease XIII column at room temperature (NovaBioAssays, MA) with 100 μL/min 0.1% formic acid in water. Peptic peptides were trapped by an ACQUITY UPLC Peptide BEH C18 VanGuard Pre-column (2.0×5 mm, Waters, MA) and further separated by an ACQUITY UPLC Peptide BEH C18 column (2.0×50 mm, Waters, MA) at −5° C., using a 15-min gradient with 0.1% formic acid in water and 0.1% formic acid in acetonitrile as mobile phases at 200 μL/min. Eluted peptides were analyzed by the Q Exactive HF mass spectrometry in LC-MS/MS or LC-MS mode.

Deuterium uptake percentage (D %) of individual peptides was calculated. Differences in deuterium uptake were calculated as ΔD %=D % of hFGFR3b-antibody−D % of hFGFR3b. Differences were considered significant if |ΔD|>5% (averaged from 2 replicates). Mass spectra of peptides showing significant differences were confirmed manually.

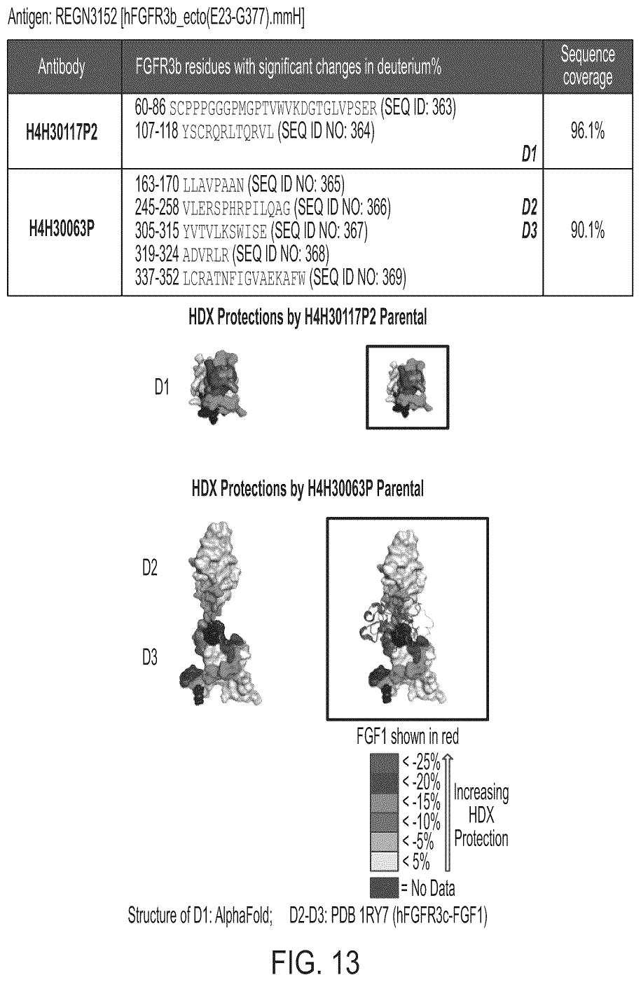

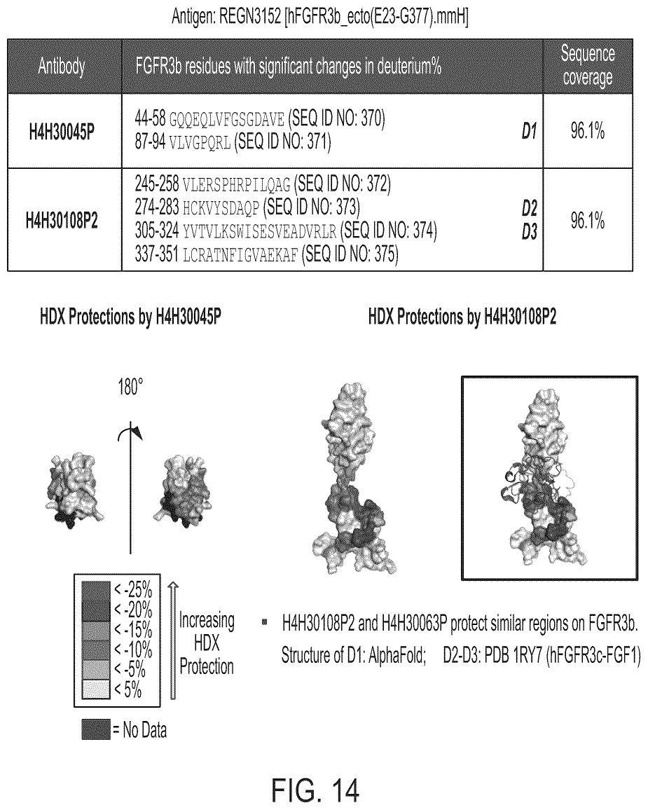

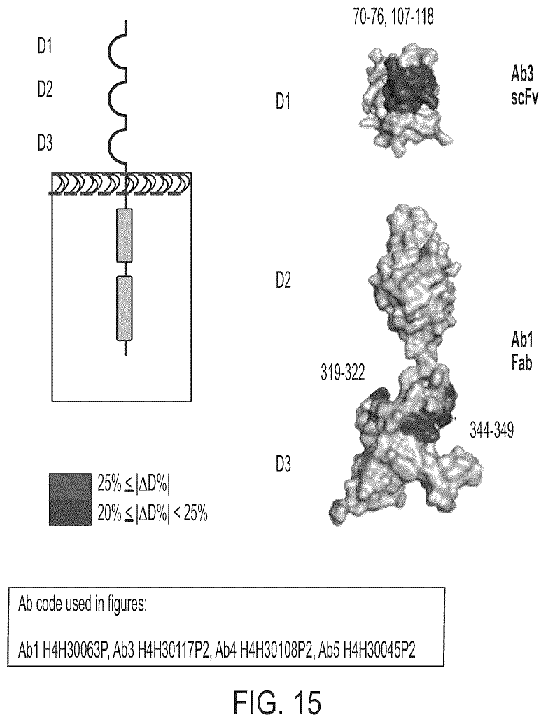

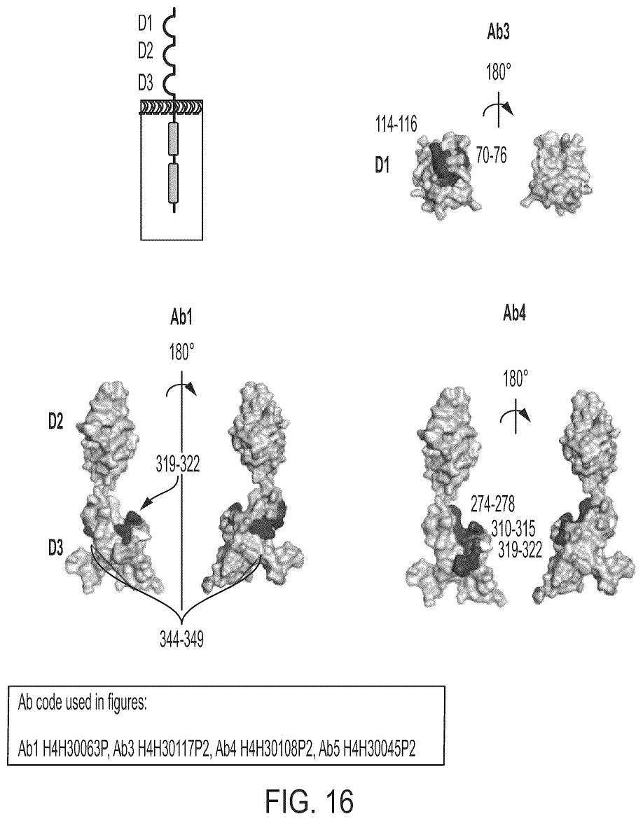

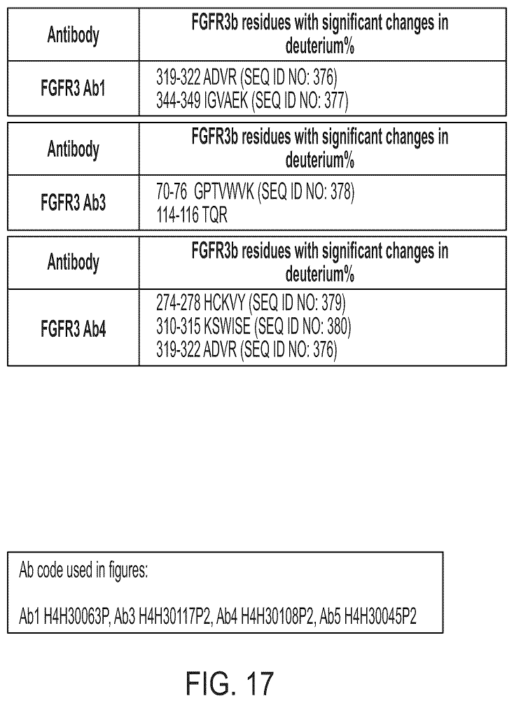

HDX epitope mapping results for anti-FGFR3b antibodies H4H30117P2, H4H30063P are shown in FIG. 13. HDX epitope mapping results for anti-FGFR3b antibodies H4H30045P and H4H30108P2 are shown in FIG. 14. HDX protection of FGFR3 by anti-FGFR3b antibodies H4H30063P Fab and H4H30117P2 scFv are shown in FIG. 15. HDX epitope mapping of FGFR3 antibodies is shown in FIGS. 16 and 17. As shown in the structures, the epitopes comprise segments of peptides, forming a continuous surface patch for interaction. The epitopes are likely conformational epitopes.

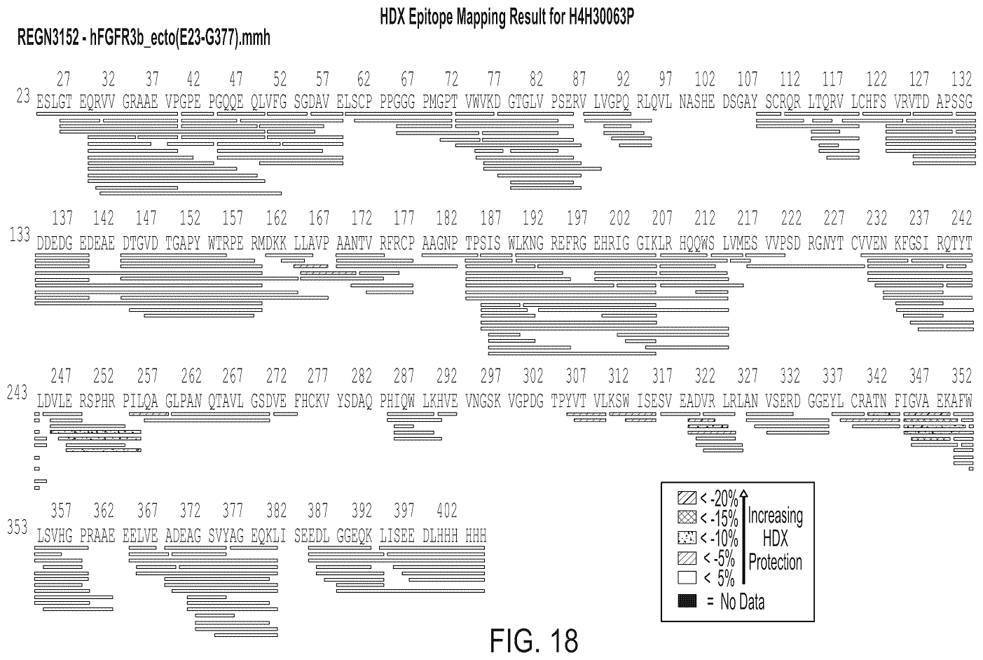

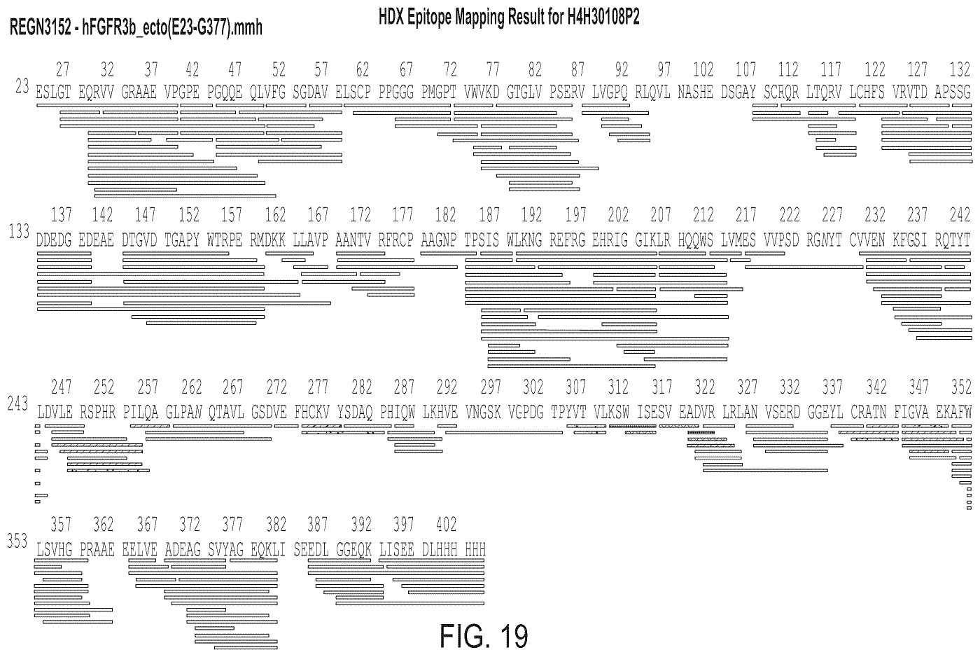

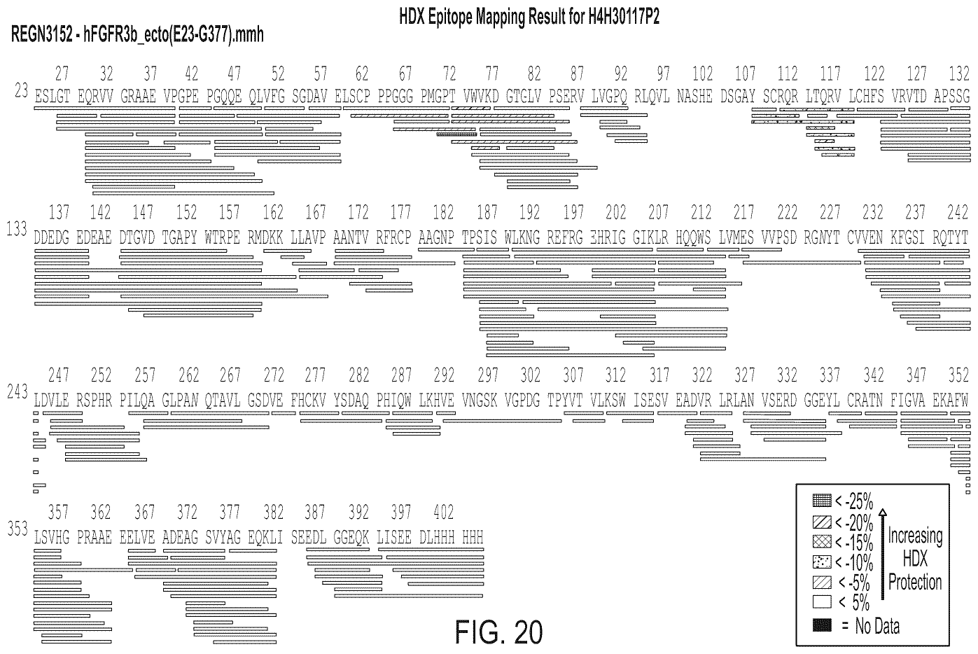

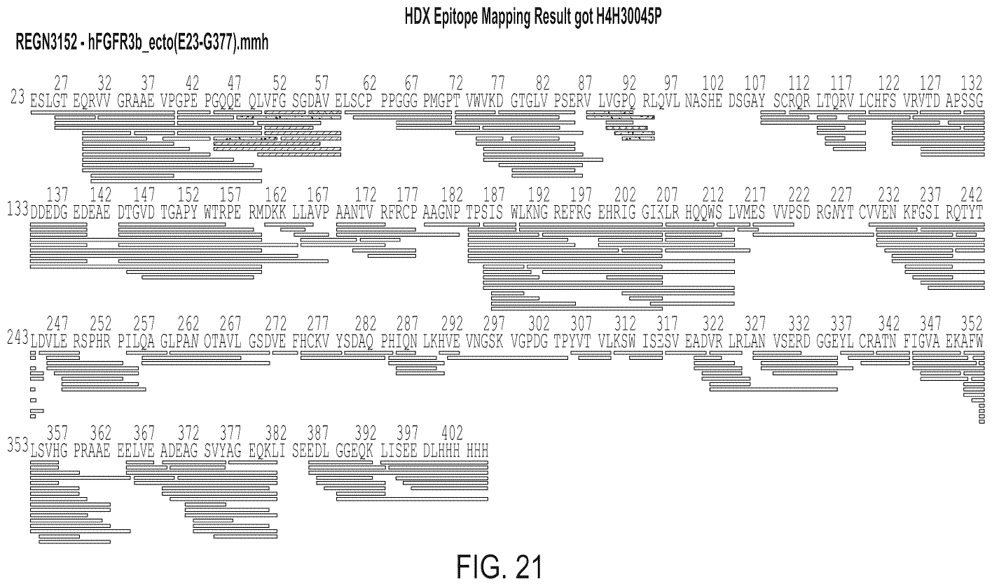

HDX epitope mapping results for H4H30063P are shown in FIG. 18. HDX epitope mapping results for H4H30108P2 are shown in FIG. 19 HDX epitope mapping results for H4H30117P2 are shown in FIG. 20. HDX epitope mapping results for H4H30045P are shown in FIG. 21.

All references cited herein are incorporated by reference to the same extent as if each individual publication, database entry (e.g., Genbank sequences or GenelD entries), patent application, or patent, was specifically and individually indicated to be incorporated by reference. This statement of incorporation by reference is intended by Applicants to relate to each and every individual publication, database entry (e.g., Genbank sequences or GenelD entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

We claim:

1. A protein-drug conjugate comprising an antigen-binding protein that binds specifically to fibroblast growth factor receptor 3 (FGFR3) and is conjugated to a molecular cargo.

2. The protein-drug conjugate of claim 1 wherein the antigen binding protein comprises an antibody or antigen-binding fragment thereof.

3. The protein-drug conjugate of claim 1 or claim 2, wherein the protein drug conjugate comprises a heavy chain variable region (HCVR or V_{H}) and/or a light chain variable region (LCVR or V_{L}).

4. The protein-drug conjugate of claim 3, wherein the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BITE), trispecific antibody, or chemically modified derivatives thereof.

5. The protein-drug conjugate of claim 3, wherein the antigen-binding protein comprises a fragment antigen-binding region (Fab).

6. The protein-drug conjugate of claim 3, wherein the antigen-binding protein comprises a single chain fragment variable (scFv).

7. The protein-drug conjugate of claim 6, wherein the scFv comprises domains arranged in the following orientation from N-terminus to C-terminus: HCVR-LCVR.

8. The protein-drug conjugate of claim 6, wherein the scFv comprises domains arranged in the following orientation from N-terminus to C-terminus: LCVR-HCVR.

9. The protein-drug conjugate of any one of claims 6-8, wherein the scFv variable regions are connected by a linker.

10. The protein-drug conjugate of claim 9, wherein the linker is a peptide linker.

11. The protein-drug conjugate of claim 10, wherein the peptide linker is -(GGGGS)ₙ—(SEQ ID NO: 321); wherein n is 1-10.

12. The protein-drug conjugate of any one of claims 1-11 which comprises:
(i) a HCVR that comprises the HCDR1, HCDR2 and HCDR3 of a HCVR comprising the amino acid sequence set forth in SEQ ID NO: 2, 22, 42, 62, 82, 102, 122, 140, 159, 169, 179, 199 or 219 (or a variant thereof);
and/or
(ii) a LCVR that comprises the LCDR1, LCDR2 and LCDR3 of a LCVR comprising the amino acid sequence set forth in SEQ ID NO: 10, 30, 50, 70, 90, 110, 130, 148, 187, 207, or 227 (or a variant thereof).

13. The protein-drug conjugate of any one of claims 1-12 which comprises:
(1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 10 (or a variant thereof);
(2) a HCVR comprising the HCDR1, HCDR2 and HCDR3 of a HCVR that comprises the amino acid sequence set forth in SEQ ID NO: 22; and a LCVR comprising the LCDR1, LCDR2 and LCDR3 of a LCVR that comprises the amino acid sequence set forth in SEQ ID NO: 30 (or a variant thereof);
(3) a HCVR comprising the HCDR1, HCDR2 and HCDR3 of a HCVR that comprises the amino acid sequence set forth in SEQ ID NO: 42; and a LCVR comprising the LCDR1, LCDR2 and LCDR3 of a LCVR that comprises the amino acid sequence set forth in SEQ ID NO: 50 (or a variant thereof);
(4) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 70 (or a variant thereof);
(5) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 90 (or a variant thereof);
(6) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 110 (or a variant thereof);
(7) a HCVR comprising the HCDR1, HCDR2 and HCDR3 of a HCVR that comprises the amino acid sequence set forth in SEQ ID NO: 122 (or a variant thereof); and LCVR comprising the LCDR1, LCDR2 and LCDR3 of a LCVR that comprises the amino acid sequence set forth in SEQ ID NO: 130 (or a variant thereof);
(8) a HCVR comprising the HCDR1, HCDR2 and HCDR3 of a HCVR that comprises the amino acid sequence set forth in SEQ ID NO: 140 (or a variant thereof); and a LCVR comprising the LCDR1, LCDR2 and LCDR3 of a LCVR that comprises the amino acid sequence set forth in SEQ ID NO: 148 (or a variant thereof);
(9) a HCVR comprising the HCDR1, HCDR2 and HCDR3 of a HCVR that comprises the amino acid sequence set forth in SEQ ID NO: 159 (or a variant thereof); and LCVR comprising the LCDR1, LCDR2 and LCDR3 of a LCVR that comprises the amino acid sequence set forth in SEQ ID NO: 148 (or a variant thereof);
(10) a HCVR comprising the HCDR1, HCDR2 and HCDR3 of a HCVR that comprises the amino acid sequence set forth in SEQ ID NO: 169 (or a variant thereof); and LCVR comprising the LCDR1, LCDR2 and LCDR3 of a LCVR that comprises the amino acid sequence set forth in SEQ ID NO: 148 (or a variant thereof);
(11) a HCVR comprising the HCDR1, HCDR2 and HCDR3 of a HCVR that comprises the amino acid sequence set forth in SEQ ID NO: 179 (or a variant thereof); and LCVR comprising the LCDR1, LCDR2 and LCDR3 of a LCVR that comprises the amino acid sequence set forth in SEQ ID NO: 187 (or a variant thereof);
(12) a HCVR comprising the HCDR1, HCDR2 and HCDR3 of a HCVR that comprises the amino acid sequence set forth in SEQ ID NO: 199 (or a variant thereof); and LCVR comprising the LCDR1, LCDR2 and LCDR3 of a LCVR that comprises the amino acid sequence set forth in SEQ ID NO: 207 (or a variant thereof); or
(13) a HCVR comprising the HCDR1, HCDR2 and HCDR3 of a HCVR that comprises the amino acid sequence set forth in SEQ ID NO: 219 (or a variant thereof); and LCVR comprising the LCDR1, LCDR2 and LCDR3 of a LCVR that comprises the amino acid sequence set forth in SEQ ID NO: 227 (or a variant thereof).

14. The protein-drug conjugate of any one of claims 1-13 which comprises:
(a) a HCVR that comprises:
an HCDR1 comprising the amino acid sequence set forth in SEQ ID NO: 4 (or a variant thereof),
an HCDR2 comprising the amino acid sequence set forth in SEQ ID NO: 6 (or a variant thereof), and
an HCDR3 comprising the amino acid sequence set forth in SEQ ID NO: 8 (or a variant thereof); and
a LCVR that comprises:
an LCDR1 comprising the amino acid sequence set forth in SEQ ID NO: 12 (or a variant thereof),
an LCDR2 comprising the amino acid sequence set forth in SEQ ID NO: 14 (or a variant thereof), and
an LCDR3 comprising the amino acid sequence set forth in SEQ ID NO: 16 (or a variant thereof);
(b) a HCVR that comprises:
an HCDR1 comprising the amino acid sequence set forth in SEQ ID NO: 24 (or a variant thereof),
an HCDR2 comprising the amino acid sequence set forth in SEQ ID NO: 26 (or a variant thereof), and
an HCDR3 comprising the amino acid sequence set forth in SEQ ID NO: 28 (or a variant thereof); and
a LCVR that comprises:
an LCDR1 comprising the amino acid sequence set forth in SEQ ID NO: 32 (or a variant thereof),
an LCDR2 comprising the amino acid sequence set forth in SEQ ID NO: 34 (or a variant thereof), and
an LCDR3 comprising the amino acid sequence set forth in SEQ ID NO: 36 (or a variant thereof);
(c) a HCVR that comprises:
an HCDR1 comprising the amino acid sequence set forth in SEQ ID NO: 44 (or a variant thereof),
an HCDR2 comprising the amino acid sequence set forth in SEQ ID NO: 46 (or a variant thereof), and
an HCDR3 comprising the amino acid sequence set forth in SEQ ID NO: 48 (or a variant thereof); and
a LCVR that comprises:
an LCDR1 comprising the amino acid sequence set forth in SEQ ID NO: 52 (or a variant thereof),
an LCDR2 comprising the amino acid sequence set forth in SEQ ID NO: 54 (or a variant thereof), and
an LCDR3 comprising the amino acid sequence set forth in SEQ ID NO: 56 (or a variant thereof);
(d) a HCVR that comprises:
an HCDR1 comprising the amino acid sequence set forth in SEQ ID NO: 64 (or a variant thereof),
an HCDR2 comprising the amino acid sequence set forth in SEQ ID NO: 66 (or a variant thereof), and
an HCDR3 comprising the amino acid sequence set forth in SEQ ID NO: 68 (or a variant thereof); and
a LCVR that comprises:
an LCDR1 comprising the amino acid sequence set forth in SEQ ID NO: 72 (or a variant thereof),
an LCDR2 comprising the amino acid sequence set forth in SEQ ID NO: 74 (or a variant thereof), and
an LCDR3 comprising the amino acid sequence set forth in SEQ ID NO: 76 (or a variant thereof);
(e) a HCVR that comprises:
an HCDR1 comprising the amino acid sequence set forth in SEQ ID NO: 84 (or a variant thereof),
an HCDR2 comprising the amino acid sequence set forth in SEQ ID NO: 86 (or a variant thereof), and
an HCDR3 comprising the amino acid sequence set forth in SEQ ID NO: 88 (or a variant thereof); and
a LCVR that comprises:
an LCDR1 comprising the amino acid sequence set forth in SEQ ID NO: 92 (or a variant thereof),
an LCDR2 comprising the amino acid sequence set forth in SEQ ID NO: 94 (or a variant thereof), and
an LCDR3 comprising the amino acid sequence set forth in SEQ ID NO: 96 (or a variant thereof);
(f) a HCVR that comprises:
an HCDR1 comprising the amino acid sequence set forth in SEQ ID NO: 104 (or a variant thereof),
an HCDR2 comprising the amino acid sequence set forth in SEQ ID NO: 106 (or a variant thereof), and
an HCDR3 comprising the amino acid sequence set forth in SEQ ID NO: 108 (or a variant thereof); and
a LCVR that comprises:
an LCDR1 comprising the amino acid sequence set forth in SEQ ID NO: 112 (or a variant thereof),
an LCDR2 comprising the amino acid sequence set forth in SEQ ID NO: 114 (or a variant thereof), and
an LCDR3 comprising the amino acid sequence set forth in SEQ ID NO: 116 (or a variant thereof);
(g) a HCVR that comprises:
an HCDR1 comprising the amino acid sequence set forth in SEQ ID NO: 124 (or a variant thereof),
an HCDR2 comprising the amino acid sequence set forth in SEQ ID NO: 126 (or a variant thereof), and
an HCDR3 comprising the amino acid sequence set forth in SEQ ID NO: 128 (or a variant thereof), and
a LCVR that comprises:
an LCDR1 comprising the amino acid sequence set forth in SEQ ID NO: 132 (or a variant thereof),
an LCDR2 comprising the amino acid sequence set forth in SEQ ID NO: 34 (or a variant thereof), and
an LCDR3 comprising the amino acid sequence set forth in SEQ ID NO: 134; (or a variant thereof)
(h) a HCVR that comprises:
an HCDR1 comprising the amino acid sequence set forth in SEQ ID NO: 142 (or a variant thereof),
an HCDR2 comprising the amino acid sequence set forth in SEQ ID NO: 144 (or a variant thereof), and
an HCDR3 comprising the amino acid sequence set forth in SEQ ID NO: 146 (or a variant thereof), and
a LCVR that comprises:
an LCDR1 comprising the amino acid sequence set forth in SEQ ID NO: 150 (or a variant thereof),
an LCDR2 comprising the amino acid sequence set forth in SEQ ID NO: 14 (or a variant thereof), and
an LCDR3 comprising the amino acid sequence set forth in SEQ ID NO: 153 (or a variant thereof);
(i) a HCVR that comprises:
an HCDR1 comprising the amino acid sequence set forth in SEQ ID NO: 161 (or a variant thereof),
an HCDR2 comprising the amino acid sequence set forth in SEQ ID NO: 163 (or a variant thereof), and
an HCDR3 comprising the amino acid sequence set forth in SEQ ID NO: 165 (or a variant thereof), and
a LCVR that comprises:
an LCDR1 comprising the amino acid sequence set forth in SEQ ID NO: 150 (or a variant thereof),
an LCDR2 comprising the amino acid sequence set forth in SEQ ID NO: 14 (or a variant thereof), and
an LCDR3 comprising the amino acid sequence set forth in SEQ ID NO: 153 (or a variant thereof);
(j) a HCVR that comprises:
an HCDR1 comprising the amino acid sequence set forth in SEQ ID NO: 171 (or a variant thereof),
an HCDR2 comprising the amino acid sequence set forth in SEQ ID NO: 173 (or a variant thereof), and
an HCDR3 comprising the amino acid sequence set forth in SEQ ID NO: 175 (or a variant thereof), and
a LCVR that comprises:
an LCDR1 comprising the amino acid sequence set forth in SEQ ID NO: 150 (or a variant thereof),
an LCDR2 comprising the amino acid sequence set forth in SEQ ID NO: 14 (or a variant thereof), and
an LCDR3 comprising the amino acid sequence set forth in SEQ ID NO: 153 (or a variant thereof);
(k) a HCVR that comprises:
an HCDR1 comprising the amino acid sequence set forth in SEQ ID NO: 181 (or a variant thereof),
an HCDR2 comprising the amino acid sequence set forth in SEQ ID NO: 183 (or a variant thereof), and
an HCDR3 comprising the amino acid sequence set forth in SEQ ID NO: 185 (or a variant thereof), and
a LCVR that comprises:
an LCDR1 comprising the amino acid sequence set forth in SEQ ID NO: 189 (or a variant thereof),
an LCDR2 comprising the amino acid sequence set forth in SEQ ID NO: 191 (or a variant thereof), and
an LCDR3 comprising the amino acid sequence set forth in SEQ ID NO: 193 (or a variant thereof);
(l) a HCVR that comprises:
an HCDR1 comprising the amino acid sequence set forth in SEQ ID NO: 201 (or a variant thereof),
an HCDR2 comprising the amino acid sequence set forth in SEQ ID NO: 203 (or a variant thereof), and
an HCDR3 comprising the amino acid sequence set forth in SEQ ID NO: 205 (or a variant thereof), and
a LCVR that comprises:
an LCDR1 comprising the amino acid sequence set forth in SEQ ID NO: 209 (or a variant thereof),
an LCDR2 comprising the amino acid sequence set forth in SEQ ID NO: 211 (or a variant thereof), and
an LCDR3 comprising the amino acid sequence set forth in SEQ ID NO: 213 (or a variant thereof);
and/or (m) a HCVR that comprises:
an HCDR1 comprising the amino acid sequence set forth in SEQ ID NO: 221 (or a variant thereof),
an HCDR2 comprising the amino acid sequence set forth in SEQ ID NO: 223 (or a variant thereof), and
an HCDR3 comprising the amino acid sequence set forth in SEQ ID NO: 225 (or a variant thereof), and
a LCVR that comprises:
an LCDR1 comprising the amino acid sequence set forth in SEQ ID NO: 32 (or a variant thereof),
an LCDR2 comprising the amino acid sequence set forth in SEQ ID NO: 34 (or a variant thereof), and
an LCDR3 comprising the amino acid sequence set forth in SEQ ID NO: 76 (or a variant thereof).

15. The protein-drug conjugate of any one of claims 1-14 which comprises:
(i) a HCVR that comprises the amino acid sequence set forth in SEQ ID NO: 2 (or a variant thereof); and a LCVR that comprises the amino acid sequence set forth in SEQ ID NO: 10 (or a variant thereof);
(ii) a HCVR that comprises the amino acid sequence set forth in SEQ ID NO: 22 (or a variant thereof); and a LCVR that comprises the amino acid sequence set forth in SEQ ID NO: 30 (or a variant thereof);
(iii) a HCVR that comprises the amino acid sequence set forth in SEQ ID NO: 42 (or a variant thereof); and a LCVR that comprises the amino acid sequence set forth in SEQ ID NO: 50 (or a variant thereof);
(iv) a HCVR that comprises the amino acid sequence set forth in SEQ ID NO: 62 (or a variant thereof); and a LCVR that comprises the amino acid sequence set forth in SEQ ID NO: 70 (or a variant thereof);
(v) a HCVR that comprises the amino acid sequence set forth in SEQ ID NO: 82 (or a variant thereof); and a LCVR that comprises the amino acid sequence set forth in SEQ ID NO: 90 (or a variant thereof);
(vi) a HCVR that comprises the amino acid sequence set forth in SEQ ID NO: 102 (or a variant thereof); and a LCVR that comprises the amino acid sequence set forth in SEQ ID NO: 110 (or a variant thereof);
(vii) a HCVR that comprises the amino acid sequence set forth in SEQ ID NO: 122 (or a variant thereof); and a LCVR that comprises the amino acid sequence set forth in SEQ ID NO: 130 (or a variant thereof);
(viii) a HCVR that comprises the amino acid sequence set forth in SEQ ID NO: 140 (or a variant thereof); and a LCVR that the amino acid sequence set forth in SEQ ID NO: 148 (or a variant thereof);
(ix) a HCVR that comprises the amino acid sequence set forth in SEQ ID NO: 159 (or a variant thereof); and a LCVR that comprises the amino acid sequence set forth in SEQ ID NO: 148 (or a variant thereof);
(x) a HCVR that comprises the amino acid sequence set forth in SEQ ID NO: 169 (or a variant thereof); and LCVR that comprises the amino acid sequence set forth in SEQ ID NO: 148 (or a variant thereof);
(xi) a HCVR that comprises the amino acid sequence set forth in SEQ ID NO: 179 (or a variant thereof); and a LCVR that comprises the amino acid sequence set forth in SEQ ID NO: 187 (or a variant thereof);
(xii) a HCVR that comprises the amino acid sequence set forth in SEQ ID NO: 199 (or a variant thereof); and LCVR that comprises the amino acid sequence set forth in SEQ ID NO: 207 (or a variant thereof);
or
(xiii) a HCVR that comprises the amino acid sequence set forth in SEQ ID NO: 219 (or a variant thereof); and LCVR that comprises the amino acid sequence set forth in SEQ ID NO: 227 (or a variant thereof).

16. The protein-drug conjugate of any one of claims 1-3, and 12-15, which comprises:
(a) a heavy chain that comprises the amino acid sequence set forth in SEQ ID NO: 18, and a light chain that comprises the amino acid sequence set forth in SEQ ID NO: 20;
(b) a heavy chain that comprises the amino acid sequence set forth in SEQ ID NO: 38, and a light chain that comprises the amino acid sequence set forth in SEQ ID NO: 40;
(c) a heavy chain that comprises the amino acid sequence set forth in SEQ ID NO: 58, and a light chain that comprises the amino acid sequence set forth in SEQ ID NO: 60;
(d) a heavy chain that comprises the amino acid sequence set forth in SEQ ID NO: 78, and a light chain that comprises the amino acid sequence set forth in SEQ ID NO: 80;
(e) a heavy chain that comprises the amino acid sequence set forth in SEQ ID NO: 98, and a light chain that comprises the amino acid sequence set forth in SEQ ID NO: 100;
(f) a heavy chain that comprises the amino acid sequence set forth in SEQ ID NO: 118, and a light chain that comprises the amino acid sequence set forth in SEQ ID NO: 120;
(g) a heavy chain that comprises the amino acid sequence set forth in SEQ ID NO: 136, and a light chain that comprises the amino acid sequence set forth in SEQ ID NO: 138;
(h) a heavy chain that comprises the amino acid sequence set forth in SEQ ID NO: 155, and a light chain that comprises the amino acid sequence set forth in SEQ ID NO: 157;
(i) a heavy chain that comprises the amino acid sequence set forth in SEQ ID NO: 167, and a light chain that comprises the amino acid sequence set forth in SEQ ID NO: 157;
(j) a heavy chain that comprises the amino acid sequence set forth in SEQ ID NO: 177, and a light chain that comprises the amino acid sequence set forth in SEQ ID NO: 157;
(k) a heavy chain that comprises the amino acid sequence set forth in SEQ ID NO: 195, and a light chain that comprises the amino acid sequence set forth in SEQ ID NO: 197;
(I) a heavy chain that comprises the amino acid sequence set forth in SEQ ID NO: 215, and a light chain that comprises the amino acid sequence set forth in SEQ ID NO: 217;
and/or
(m) a heavy chain that comprises the amino acid sequence set forth in SEQ ID NO: 229, and a light chain that comprises the amino acid sequence set forth in SEQ ID NO: 231.

17. The protein-drug conjugate of any one of claims 1-16, wherein the antigen-binding protein binds specifically to FGFR3b and/or FGFR3c.

18. The protein-drug conjugate of any one of claims 1-17, wherein the antigen-binding protein that binds specifically to human FGFR3b and/or FGFR3c.

19. The protein-drug conjugate of any one of claims 1-18, wherein the antigen-binding protein binds specifically to FGFR3b.

20. The protein-drug conjugate of any one of claims 17-19, wherein the FGFR3b is monomeric and/or dimeric FGFR3b.

21. The protein-drug conjugate of any one of claims 17-20, wherein the FGFR3b is monomeric FGFR3b.

22. The protein-drug conjugate of any one of claims 17-20, wherein the FGFR3b is dimeric FGFR3b.

23. The protein-drug conjugate of claim 22, which comprises:
(a) a heavy chain that comprises the amino acid sequence set forth in SEQ ID NO: 18, and a light chain that comprises the amino acid sequence set forth in SEQ ID NO: 20;
(b) a heavy chain that comprises the amino acid sequence set forth in SEQ ID NO: 38, and a light chain that comprises the amino acid sequence set forth in SEQ ID NO: 40;
(c) a heavy chain that comprises the amino acid sequence set forth in SEQ ID NO: 58, and a light chain that comprises the amino acid sequence set forth in SEQ ID NO: 60;
(d) a heavy chain that comprises the amino acid sequence set forth in SEQ ID NO: 78, and a light chain that comprises the amino acid sequence set forth in SEQ ID NO: 80;
(e) a heavy chain that comprises the amino acid sequence set forth in SEQ ID NO: 98, and a light chain that comprises the amino acid sequence set forth in SEQ ID NO: 100;
(f) a heavy chain that comprises the amino acid sequence set forth in SEQ ID NO: 118, and a light chain that comprises the amino acid sequence set forth in SEQ ID NO: 120;
(g) a heavy chain that comprises the amino acid sequence set forth in SEQ ID NO: 136, and a light chain that comprises the amino acid sequence set forth in SEQ ID NO: 138;
(h) a heavy chain that comprises the amino acid sequence set forth in SEQ ID NO: 167, and a light chain that comprises the amino acid sequence set forth in SEQ ID NO: 157;
(i) a heavy chain that comprises the amino acid sequence set forth in SEQ ID NO: 177, and a light chain that comprises the amino acid sequence set forth in SEQ ID NO: 157;
(j) a heavy chain that comprises the amino acid sequence set forth in SEQ ID NO: 195, and a light chain that comprises the amino acid sequence set forth in SEQ ID NO: 197;
(k) a heavy chain that comprises the amino acid sequence set forth in SEQ ID NO: 215, and a light chain that comprises the amino acid sequence set forth in SEQ ID NO: 217;
and/or
(l) a heavy chain that comprises the amino acid sequence set forth in SEQ ID NO: 229, and a light chain that comprises the amino acid sequence set forth in SEQ ID NO: 231.

24. The protein-drug conjugate of any one of claims 17-18, wherein the FGFR3c is monomeric and/or dimeric FGFR3c.

25. The protein-drug conjugate of any one of claim 24, wherein the FGFR3c is monomeric FGFR3c.

26. The protein-drug conjugate of claim 25, which comprises:
(a) a heavy chain that comprises the amino acid sequence set forth in SEQ ID NO: 98, and a light chain that comprises the amino acid sequence set forth in SEQ ID NO: 100;
(b) a heavy chain that comprises the amino acid sequence set forth in SEQ ID NO: 155, and a light chain that comprises the amino acid sequence set forth in SEQ ID NO: 157;
(c) a heavy chain that comprises the amino acid sequence set forth in SEQ ID NO: 177, and a light chain that comprises the amino acid sequence set forth in SEQ ID NO: 157;
(d) a heavy chain that comprises the amino acid sequence set forth in SEQ ID NO: 195, and a light chain that comprises the amino acid sequence set forth in SEQ ID NO: 197;
(e) a heavy chain that comprises the amino acid sequence set forth in SEQ ID NO: 215, and a light chain that comprises the amino acid sequence set forth in SEQ ID NO: 217;
and/or
(f) a heavy chain that comprises the amino acid sequence set forth in SEQ ID NO: 229, and a light chain that comprises the amino acid sequence set forth in SEQ ID NO: 231.

27. The protein-drug conjugate of any one of claims 1-26, wherein the antigen-binding protein binds specifically to both FGFR3b and FGFR3c.

28. The protein-drug conjugate of any one of claims 18-23 and 27, wherein the antigen-binding protein binds to human FGFR3b with a K_{D} of about 1λ10⁻⁷ M or a stronger affinity.

29. The protein-drug conjugate of claim 28, wherein the antigen-binding protein binds to human FGFR3b with a K_{D} of about 1×10⁻⁸ M or a stronger affinity.

30. The protein-drug conjugate of any one of claims 18 and 24-27, wherein the antigen-binding protein binds to human FGFR3c with a K_{D} of about 1×10⁻⁷ M or a stronger affinity.

31. The protein-drug conjugate of claim 30, wherein the antigen-binding protein binds to human FGFR3c with a K_{D} of about 1×10⁻⁸ M or a stronger affinity.

32. The protein-drug conjugate of any one of claims 1-20, which comprises a heavy chain that comprises the amino acid sequence set forth in SEQ ID NO: 18, and a light chain that comprises the amino acid sequence set forth in SEQ ID NO: 20.

33. The protein-drug conjugate of any one of claims 1-11, wherein the antigen-binding protein binds to the same epitope on FGFR3 as an antibody comprising an HCVR/LCVR amino acid sequence pair as set forth in Table 1-1.

34. The protein-drug conjugate of any one of claims 1-11, wherein the antigen-binding protein competes for binding to FGFR3 with an antibody comprising an HCVR/LCVR amino acid sequence pair as set forth in Table 1-1.

35. A protein-drug conjugate comprising an antigen-binding protein that binds specifically to fibroblast growth factor receptor 3 (FGFR3), wherein the antigen-binding protein is conjugated to a molecular cargo and comprises an antibody or antigen-binding fragment thereof, wherein the antibody or antigen-binding fragment thereof binds to one or more epitopes of FGFR3 selected from:
a. an epitope comprising the sequence GPTVVVVK (SEQ ID NO: 378) and/or an epitope comprising the sequence TQR;
b. an epitope comprising the sequence ADVR (SEQ ID NO: 376) and/or an epitope comprising the sequence IGVAEK (SEQ ID NO: 377);
c. an epitope comprising the sequence HCKVY (SEQ ID NO: 379), and/or an epitope comprising the sequence KSWISE (SEQ ID NO: 380), and/or an epitope comprising the sequence ADVR (SEQ ID NO: 376);
d. an epitope comprised within or overlapping with the sequence GPTVVVVK (SEQ ID NO: 378) and/or an epitope comprised within or overlapping with the sequence TQR;
e. an epitope comprised within or overlapping with the sequence ADVR (SEQ ID NO: 376) and/or an epitope comprised within or overlapping with the sequence IGVAEK (SEQ ID NO: 377); and
f. an epitope comprised within or overlapping with the sequence HCKVY (SEQ ID NO: 379), and/or an epitope comprised within or overlapping with the sequence KSWISE (SEQ ID NO: 380), and/or an epitope comprised within or overlapping with the sequence ADVR (SEQ ID NO: 376).

36. The protein-drug conjugate of claim 35, wherein the antibody or antigen-binding fragment thereof binds to one or more epitopes of FGFR3 selected from:
a. an epitope consisting of the sequence GPTVWVK (SEQ ID NO: 378) and/or an epitope consisting of the sequence TQR;
b. an epitope consisting of the sequence ADVR (SEQ ID NO: 376) and/or an epitope consisting of the sequence IGVAEK (SEQ ID NO: 377); and
c. an epitope consisting of the sequence HCKVY (SEQ ID NO: 379), and/or an epitope consisting of the sequence KSWISE (SEQ ID NO: 380), and/or an epitope consisting of the sequence ADVR (SEQ ID NO: 376).

37. A protein-drug conjugate comprising an antigen-binding protein that binds specifically to fibroblast growth factor receptor 3 (FGFR3), wherein the antigen-binding protein is conjugated to a molecular cargo and comprises an antibody or antigen-binding fragment thereof, wherein the antibody or antigen-binding fragment thereof binds to one or more epitopes of FGFR3 selected from:
a. an epitope comprising the sequence SCPPPGGGPMGPTVVVVKDGTGLVPSER (SEQ ID NO: 363) and/or an epitope comprising the sequence YSCRQRLTQRVL (SEQ ID NO: 364;
b. an epitope comprising the sequence LLAVPAAN (SEQ ID NO: 365), and/or an epitope comprising the sequence VLERSPHRPILQAG (SEQ ID NO: 366) and/or an epitope comprising the sequence YVTVLKSWISE (SEQ ID NO: 367) and/or or an epitope comprising the sequence ADVRLR (SEQ ID NO: 368) and/or an epitope comprising the sequence LCRATNFIGVAEKAFW (SEQ ID NO: 369);
c. an epitope comprising the sequence GQQEQLVFGSGDAVE (SEQ ID NO: 370) and/or an epitope comprising the sequence VLVGPQRL (SEQ ID NO: 371);
d. an epitope comprising the sequence VLERSPHRPILQAG (SEQ ID NO: 372) and/or an epitope comprising the sequence HCKVYSDAQP (SEQ ID NO: 373) and/or an epitope comprising the sequence YVTVLKSWISESVEADVRLR (SEQ ID NO: 374) and/or an epitope comprising the sequence LCRATNFIGVAEKAF (SEQ ID NO: 375);
e. an epitope comprised within or overlapping with the sequence SCPPPGGGPMGPTVWVKDGTGLVPSER (SEQ ID NO: 363) and/or an epitope comprised within or overlapping with the sequence YSCRQRLTQRVL (SEQ ID NO: 364);
f. an epitope comprised within or overlapping with the sequence LLAVPAAN (SEQ ID NO: 365, and/or an epitope comprised within or overlapping with the sequence VLERSPHRPILQAG (SEQ ID NO: 366) and/or an epitope comprised within or overlapping with the sequence YVTVLKSWISE (SEQ ID NO: 367) and/or or an epitope comprised within or overlapping with the sequence ADVRLR (SEQ ID NO: 368) and/or an epitope comprised within or overlapping with the sequence LCRATNFIGVAEKAFW (SEQ ID NO: 369;
g. an epitope comprised within or overlapping with the sequence GQQEQLVFGSGDAVE (SEQ ID NO: 370) and/or an epitope comprised within or overlapping with the sequence VLVGPQRL (SEQ ID NO: 371); and
h. an epitope comprised within or overlapping with the sequence VLERSPHRPILQAG (SEQ ID NO: 372) and/or an epitope comprised within or overlapping with the sequence HCKVYSDAQP (SEQ ID NO: 373) and/or an epitope comprised within or overlapping with the sequence YVTVLKSWISESVEADVRLR (SEQ ID NO: 374) and/or an epitope comprised within or overlapping with the sequence LCRATNFIGVAEKAF (SEQ ID NO: 375.

38. The protein-drug conjugate of claim 37, wherein the antibody or antigen-binding fragment thereof binds to one or more epitopes of FGFR3 selected from:
a. an epitope consisting of the sequence SCPPPGGGPMGPTVVVVKDGTGLVPSER (SEQ ID NO: 363) and/or an epitope consisting of the sequence YSCRQRLTQRVL (SEQ ID NO: 364);
b. an epitope consisting of the sequence LLAVPAAN (SEQ ID NO: 365), and/or an epitope consisting of the sequence VLERSPHRPILQAG (SEQ ID NO: 366) and/or an epitope consisting of the sequence YVTVLKSWISE (SEQ ID NO: 367) and/or or an epitope consisting of the sequence ADVRLR (SEQ ID NO: 368) and/or an epitope consisting of the sequence LCRATNFIGVAEKAFW (SEQ ID NO: 369);
c. an epitope consisting of the sequence GQQEQLVFGSGDAVE (SEQ ID NO: 370) and/or an epitope consisting of the sequence VLVGPQRL (SEQ ID NO: 371); and
d. an epitope consisting of the sequence VLERSPHRPILQAG (SEQ ID NO: 372) and/or an epitope consisting of the sequence HCKVYSDAQP (SEQ ID NO: 373) and/or an epitope consisting of the sequence YVTVLKSWISESVEADVRLR (SEQ ID NO: 374) and/or an epitope consisting of the sequence LCRATNFIGVAEKAF (SEQ ID NO: 375).

39. The protein-drug conjugate of any one of claims 35-38, wherein the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BITE), trispecific antibody, or chemically modified derivatives thereof.

40. The protein-drug conjugate of any one of claims 1-39, wherein said molecular cargo is conjugated to:
(i) a HCVR of the antigen-binding protein,
(ii) a LCVR of the antigen-binding protein,
(iii) a heavy chain of the antigen-binding protein, and/or
(iv) a light chain of the antigen-binding protein.

41. The protein-drug conjugate of any one of claims 1-40, wherein said molecular cargo is conjugated to the antigen-binding protein via a glutamine residue and/or a lysine residue.

42. The protein-drug conjugate of claim 41, wherein the glutamine residue is:
(i) introduced to the N-terminus and/or C-terminus of a heavy chain of the antigen-binding protein,
(ii) introduced to the N-terminus and/or C-terminus of a light chain of the antigen-binding protein,
(iii) naturally present in a CH2 or CH3 domain of the antigen-binding protein,
(iv) introduced to the antigen-binding protein by modifying one or more amino acids, and/or
(v) Q295 or mutated from N297 to Q297 (N297Q).

43. The protein-drug conjugate of claim 41 or 42, wherein the antigen-binding protein comprises a glutamine-containing tag, and the molecular cargo is conjugated to the antigen-binding protein via a glutamine residue of the glutamine-containing tag.

44. The protein-drug conjugate of claim 43, wherein the glutamine-containing tag comprises an amino acid sequence selected from the group consisting of LLQGG (SEQ ID NO: 290), LLQG (SEQ ID NO: 291), LSLSQG (SEQ ID NO: 292), GGGLLQGG (SEQ ID NO: 293), GLLQG (SEQ ID NO: 294), LLQ, GSPLAQSHGG (SEQ ID NO: 295), GLLQGGG (SEQ ID NO: 296), GLLQGG (SEQ ID NO: 297), GLLQ (SEQ ID NO: 298), LLQLLQGA (SEQ ID NO: 299), LLQGA (SEQ ID NO: 300), LLQYQGA (SEQ ID NO: 301), LLQGSG (SEQ ID NO: 302), LLQYQG (SEQ ID NO: 303), LLQLLQG (SEQ ID NO: 304), SLLQG (SEQ ID NO: 305), LLQLQ (SEQ ID NO: 306), LLQLLQ (SEQ ID NO: 307), and LLQGR (SEQ ID NO: 308).

45. The protein-drug conjugate of any one of claims 1-44, wherein the antigen-binding protein and the molecular cargo are conjugated via a linker.

46. The protein-drug conjugate of any one of claims 1-45, wherein the molecular cargo comprises a polynucleotide molecule, a polypeptide molecule, a carrier, a viral particle, a viral capsid protein, or a small molecule.

47. The protein-drug conjugate of claim 46, wherein the molecular cargo comprises a polynucleotide molecule.

48. The protein-drug conjugate of claim 47, wherein the polynucleotide molecule is an interfering nucleic acid molecule, a guide RNA, a ribozyme, an aptamer, a mixmer, a multimer, or an mRNA.

49. The protein-drug conjugate of claim 48, wherein the interfering nucleic acid molecule is an siRNA, an shRNA, a miRNA, an antisense oligonucleotide, or a gapmer.

50. The protein-drug conjugate of claim 48 or 49, wherein the interfering nucleic acid molecule is an siRNA.

51. The protein-drug conjugate of claim 50, wherein the siRNA comprises a sense strand of 21 nucleotides in length.

52. The protein-drug conjugate of claim 50 or 51, wherein the siRNA comprises an antisense strand of 23 nucleotides in length.

53. The protein-drug conjugate of any one of claims 50-52, wherein the siRNA comprises two phosphorothioate linkages at the first and second internucleoside linkages at the 5′ end of the sense strand.

54. The protein-drug conjugate of any one of claims 50-53, wherein the siRNA comprises two phosphorothioate linkages at the first and second internucleoside linkages at the 3′ and/or 5′ ends of the antisense strand.

55. The protein-drug conjugate of claim 48 or 49, wherein the interfering nucleic acid molecule is an antisense oligonucleotide.

56. The protein-drug conjugate of claim 48, wherein the polynucleotide molecule is a guide RNA.

57. The protein-drug conjugate of any one of claims 48-56, wherein the polynucleotide molecule targets a gene or gene product associated with a neurological disease and/or disorder.

58. The protein-drug conjugate of claim 57, wherein the gene is APOE4, GFAP, MECP2, AQP4, or STAT3.

59. The protein-drug conjugate of any one of claims 48-58, wherein the polynucleotide molecule comprises one or more modified nucleotides.

60. The protein-drug conjugate of claim 46, wherein the molecular cargo comprises a polypeptide molecule.

61. The protein-drug conjugate of claim 60, wherein the polypeptide molecule is an enzyme, a neuroprotective molecule, or an antigen-binding protein that binds to a target other than FGFR3.

62. The protein-drug conjugate of claim 60 or 61, wherein the polypeptide molecule is associated with a neurological disease and/or disorder.

63. The protein-drug conjugate of claim 62, wherein the polypeptide molecule is a protective ApoE isoform or variant, ATPase 13A2 (encoded by ATP13A2), sulfatase modifying factor 1 (encoded by SUMF1), fragile X messenger ribonucleoprotein (FMRP) (encoded by FMR1), or glutamate transporter-1 (encoded by GLT1).

64. The protein-drug conjugate of claim 63, wherein the protective ApoE isoform or variant is ApoE2, ApoE Christchurch, or ApoE Jacksonville.

65. The protein-drug conjugate of claim 62, wherein the polypeptide molecule is a neurotrophic factor, an antibody or antibody fragment, an antibody receptor fusion protein, or a suppressor of cytokine signaling.

66. The protein-drug conjugate of claim 65, wherein the neurotrophic factor is ciliary neurotrophic factor (CNTF), brain derived neurotrophic factor (BDNF), nerve growth factor (NGF), glial cell line-derived neurotrophic factor (GDNF), or insulin-like growth factor 1 (IGF).

67. The protein-drug conjugate of claim 65, wherein the antibody receptor fusion protein is anti-amyloid beta Gas6 fusion protein.

68. The protein-drug conjugate of claim 65, wherein the suppressor of cytokine signaling is suppressor of cytokine signaling 3 (Socs3).

69. The protein-drug conjugate of any one of claims 1-68, wherein the molecular cargo is conjugated to the antigen-binding protein at a drug-to-antibody ratio (DAR) of a least 1 to at least 10.

70. The protein-drug conjugate of claim 69, wherein the molecular cargo is conjugated to the antigen-binding protein at a DAR of 1, 2, 3, or 4.

71. The protein-drug conjugate of claim 69 or 70, wherein the molecular cargo is conjugated to the antigen-binding protein at a DAR of 2.

72. The protein-drug conjugate of claim 69 or 70, wherein the molecular cargo is conjugated to the antigen-binding protein at a DAR of 4.

73. The protein-drug conjugate of any one of claims 1-72 for use in treating or preventing a neurological disease or disorder.

74. The protein-drug conjugate of any one of claims 57, 62, and 73, wherein the neurological disease or disorder is a neurodegenerative disease, a neurodevelopmental disease, a physical injury, a neuropsychiatric disease, or a brain cancer.

75. The protein-drug conjugate of claim 74, wherein the neurodegenerative disease is Alzheimer's disease, Parkinson's disease, Huntington's disease, amyotrophic lateral sclerosis (ALS), or prion disease (transmissible spongiform encephalopathy).

76. The protein-drug conjugate of claim 74, wherein the neurodevelopmental disease is Alexander disease, multiple sulfatase deficiency, autism, epilepsy, Rett syndrome, or Fragile X.

77. The protein-drug conjugate of claim 74, wherein the physical injury is traumatic brain injury, spinal cord injury, stroke, or brain edema.

78. The protein-drug conjugate of claim 74, wherein the neuropsychiatric disease or disorder is major depressive disorder, anxiety disorder, or bipolar disorder.

79. The protein-drug conjugate of claim 74, wherein the brain cancer is glioma.

80. The protein-drug conjugate of claim 79, wherein the glioma is an astrocytoma.

81. The protein-drug conjugate of claim 46, wherein the molecular cargo comprises a carrier.

82. The protein-drug conjugate of claim 81, wherein the carrier is a lipid-based carrier.

83. The protein-drug conjugate of claim 82, wherein the lipid-based carrier is a lipid nanoparticle (LNP), a liposome, a lipidoid, or a lipoplex.

84. The protein-drug conjugate of claim 83, wherein the lipid-based carrier is a lipid nanoparticle (LNP).

85. The protein-drug conjugate of claim 84, wherein the LNP further comprises a polynucleotide molecule and/or a polypeptide molecule.

86. The protein-drug conjugate of claim 84 or 85, wherein the LNP comprises one or more components of a gene editing system.

87. The protein-drug conjugate of claim 86, wherein the LNP comprises
(a) a Cas nuclease, or a nucleic acid encoding the Cas nuclease, and/or
(b) a guide RNA, or one or more DNAs encoding the guide RNA.

88. The protein-drug conjugate of claim 87, wherein the Cas nuclease is a Cas9 protein.

89. The protein-drug conjugate of claim 88, wherein the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein.

90. The protein-drug conjugate of any one of claims 87-89, wherein the nucleic acid encoding the Cas nuclease is codon-optimized for expression in a mammalian cell.

91. The protein-drug conjugate of claim 90, wherein the nucleic acid encoding the Cas nuclease is codon-optimized for expression in a human cell.

92. The protein-drug conjugate of any one of claims 87-91, wherein the nucleic acid encoding the Cas nuclease is an mRNA.

93. The protein-drug conjugate of any one of claims 48, 56, and 87-92, wherein the guide RNA is a single guide RNA (sgRNA).

94. The protein-drug conjugate of claim 86, wherein the LNP comprises a zinc finger nuclease (ZFN), or a transcription activator-like effector nuclease (TALEN).

95. The protein-drug conjugate of any one of claims 84-94, wherein the LNP comprises a cationic lipid, a neutral lipid, a helper lipid, a stealth lipid, or any combinations thereof.

96. The protein-drug conjugate of claim 95, wherein the neutral lipid is distearoylphosphatidylcholine (DSPC).

97. The protein-drug conjugate of claim 95, wherein the helper lipid is cholesterol.

98. The protein-drug conjugate of claim 95, wherein the stealth lipid is PEG2k-DMG.

99. A pharmaceutical composition comprising the protein-drug conjugate of any one of claims 1-98 and a pharmaceutically acceptable carrier.

100. A composition or kit comprising the protein-drug conjugate of any one of claims 1-98 or pharmaceutical composition of claim 99 and a further therapeutic agent.

101. A complex comprising the protein-drug conjugate of any one of claims 1-98 bound to fibroblast growth factor receptor 3 (FGFR3).

102. A method for making a protein-drug conjugate of any one of claims 1-98 comprising:
(a) contacting the antigen-binding protein, with the molecular cargo under the conditions favorable for conjugation of the antigen-binding protein to the molecular cargo; and
(b) optionally, isolating the protein-drug conjugate produced in step (a).

103. A method for making a protein-drug conjugate of any one of claims 60-68, wherein the molecular cargo comprises a polypeptide molecule, comprising:
(a) culturing a host cell comprising a polynucleotide encoding the protein-drug conjugate under conditions that allow expression of the protein-drug conjugate; and
(b) optionally, isolating the protein-drug conjugate produced in step (a).

104. A protein-drug conjugate which is produced by or obtainable by the method of claim 102 or 103.

105. A vessel or injection device comprising the protein-drug conjugate of any one of claims 1-98 and 104.

106. A method for administering the protein-drug conjugate of any one of claims 1-98 and 104 to a subject comprising introducing the protein-drug conjugate into the body of the subject.

107. A method for delivering a molecular cargo to a tissue or cell type expressing FGFR3 in the body of a subject comprising administering to the subject the protein-drug conjugate of any one of claims 1-98 and 104 or the pharmaceutical composition of claim 99.

108. The method of claim 107, wherein the tissue is brain, spinal cord, or eye.

109. The method of claim 107, wherein the cell type is astrocyte or astrocyte-derived tumor cell.

110. A method for treating or preventing a neurological disease or disorder in a subject in need thereof comprising administering to the subject an effective amount of the protein-drug conjugate of any one of claims 1-98 and 104.

111. The method of claim 110, wherein the neurological disease or disorder is associated with astrocytes.

112. The method of claim 110 or 111, wherein the neurological disease or disorder is a neurodegenerative disease, neurodevelopmental disease, physical injury, neuropsychiatric disease, or a brain cancer.

113. The method of claim 112, wherein the neurodegenerative disease is Alzheimer's disease, Parkinson's disease, Huntington's disease, amyotrophic lateral sclerosis, or prion disease (transmissible spongiform encephalopathy).

114. The method of claim 112, wherein the neurodevelopmental disease is an Alexander disease, multiple sulfatase deficiency, autism, epilepsy, Rett syndrome, or Fragile X.

115. The method of claim 112, wherein the physical injury is traumatic brain injury, spinal cord injury, stroke, or brain edema.

116. The method of claim 112, wherein the neuropsychiatric disease or disorder is major depressive disorder, anxiety disorder, or bipolar disorder.

117. The method of claim 112, wherein the brain cancer is glioma.

118. The method of claim 117, wherein the glioma is an astrocytoma.

119. The method of any one of claims 110-118, further comprising administering an additional treatment to the subject.

120. The method of any one of claims 106-119, wherein the protein-drug conjugate is administered into the body of the subject via intrathecal, intracisternal, intracerebroventricular, or intraparenchymal administration into the central nervous system.

121. The method of any one of claims 106-119, wherein the protein-drug conjugate is administered into the body of the subject via intravitreal or intraocular administration into the eye.

122. The method of any one of claims 106-119, wherein the protein-drug conjugate is administered into the body of the subject via systemic administration.

123. The method of claim 122, wherein the protein-drug conjugate is administered into the body of the subject via intranasal administration.


SEQUENCE LISTING (396 sequences)

SEQ ID NO: 1 - DNA, 345 bp, synthetic construct
  source 1..345: mol_type = other DNA
caggtgcagctgcaggagtcgggcccaggactggtgaagccttcggagaccctgtccctcacctgcactgtctctggtgactccattaatagttacttctggagctggatccggcagttgccagggaaggaactggagtggattggccatatctattctagtgggagtaccagatacaacccctccctccagagtcgagtcaccatatcaatagacacgtccaagaaccagttctccctgaagctgagttctgtgaccgctgcggacacggccgtatattactgtgcgaggggcgccagcgcagttgactactggggccagggaaccctggtcaccgtctcctca

SEQ ID NO: 2 - protein, 115 aa, synthetic construct
QVQLQESGPGLVKPSETLSLTCTVSGDSINSYFWSWIRQLPGKELEWIGHIYSSGSTRYNPSLQSRVTISIDTSKNQFSLKLSSVTAADTAVYYCARGASAVDYWGQGTLVTVSS

SEQ ID NO: 3 - DNA, 24 bp, synthetic construct
  source 1..24: mol_type = other DNA
ggtgactccattaatagttacttc

SEQ ID NO: 4 - protein, 8 aa, synthetic construct
GDSINSYF

SEQ ID NO: 5 - DNA, 21 bp, synthetic construct
  source 1..21: mol_type = other DNA
atctattctagtgggagtacc

SEQ ID NO: 6 - protein, 7 aa, synthetic construct
IYSSGST

SEQ ID NO: 7 - DNA, 27 bp, synthetic construct
  source 1..27: mol_type = other DNA
gcgaggggcgccagcgcagttgactac

SEQ ID NO: 8 - protein, 9 aa, synthetic construct
ARGASAVDY

SEQ ID NO: 9 - DNA, 324 bp, synthetic construct
  source 1..324: mol_type = other DNA
gaaattgtgttgacgcagtctccaggcaccctgtctttgtctccaggggaaagagccaccctctcctgcaggaccagtcagagtattagcagcggctatttagcctggtaccagcagaaacctggccaggctcccaggctcctcatctatggtgcatccagaagggccactggcatcccagacaggttcagtggcagtgggtctgggacagacttcactctcaccatcagcagactggagcctgaagactttgtagtgtattactgtcaacaatatggtagctcaccatacacttttggccaggggaccaagctggagatcaaa

SEQ ID NO: 10 - protein, 108 aa, synthetic construct
EIVLTQSPGTLSLSPGERATLSCRTSQSISSGYLAWYQQKPGQAPRLLIYGASRRATGIPDRFSGSGSGTDFTLTISRLEPEDFVVYYCQQYGSSPYTFGQGTKLEIK

SEQ ID NO: 11 - DNA, 21 bp, synthetic construct
  source 1..21: mol_type = other DNA
cagagtattagcagcggctat

SEQ ID NO: 12 - protein, 7 aa, synthetic construct
QSISSGY

SEQ ID NO: 13-14 - skipped (2 entries)

SEQ ID NO: 15 - DNA, 27 bp, synthetic construct
  source 1..27: mol_type = other DNA
caacaatatggtagctcaccatacact

SEQ ID NO: 16 - protein, 9 aa, synthetic construct
QQYGSSPYT

SEQ ID NO: 17 - DNA, 1329 bp, synthetic construct
  source 1..1329: mol_type = other DNA
caggtgcagctgcaggagtcgggcccaggactggtgaagccttcggagaccctgtccctcacctgcactgtctctggtgactccattaatagttacttctggagctggatccggcagttgccagggaaggaactggagtggattggccatatctattctagtgggagtaccagatacaacccctccctccagagtcgagtcaccatatcaatagacacgtccaagaaccagttctccctgaagctgagttctgtgaccgctgcggacacggccgtatattactgtgcgaggggcgccagcgcagttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 18 - protein, 442 aa, synthetic construct
QVQLQESGPGLVKPSETLSLTCTVSGDSINSYFWSWIRQLPGKELEWIGHIYSSGSTRYNPSLQSRVTISIDTSKNQFSLKLSSVTAADTAVYYCARGASAVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 648 bp, synthetic construct
  source 1..648: mol_type = other DNA
gaaattgtgttgacgcagtctccaggcaccctgtctttgtctccaggggaaagagccaccctctcctgcaggaccagtcagagtattagcagcggctatttagcctggtaccagcagaaacctggccaggctcccaggctcctcatctatggtgcatccagaagggccactggcatcccagacaggttcagtggcagtgggtctgggacagacttcactctcaccatcagcagactggagcctgaagactttgtagtgtattactgtcaacaatatggtagctcaccatacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 20 - protein, 215 aa, synthetic construct
EIVLTQSPGTLSLSPGERATLSCRTSQSISSGYLAWYQQKPGQAPRLLIYGASRRATGIPDRFSGSGSGTDFTLTISRLEPEDFVVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 378 bp, synthetic construct
  source 1..378: mol_type = other DNA
caggtgcagctggtacagtctggggctgaggtgaagaagcctggggcctcagtgaaggtctcctgcaaggtttccggatacaccctcactgaattatccatgcactgggtgcgacaagctcctggaaaagggcttgagtggatgggaggttttgatcctgaagatggtgaaataatctacgcacagaagttccagggcagagtcaccatgaccgaggacacatctacagacacagcctacatggacctgagcagtctgacatctgaagacacggccgtgtattactgtgcaacggagaagcagcaactggtacgaaaatactacttctactacggtttggccgtctggggccaagggaccacggtcaccgtctcctca

SEQ ID NO: 22 - protein, 126 aa, synthetic construct
QVQLVQSGAEVKKPGASVKVSCKVSGYTLTELSMHWVRQAPGKGLEWMGGFDPEDGEIIYAQKFQGRVTMTEDTSTDTAYMDLSSLTSEDTAVYYCATEKQQLVRKYYFYYGLAVWGQGTTVTVSS

SEQ ID NO: 23 - DNA, 24 bp, synthetic construct
  source 1..24: mol_type = other DNA
ggatacaccctcactgaattatcc

SEQ ID NO: 24 - protein, 8 aa, synthetic construct
GYTLTELS

SEQ ID NO: 25 - DNA, 24 bp, synthetic construct
  source 1..24: mol_type = other DNA
tttgatcctgaagatggtgaaata

SEQ ID NO: 26 - protein, 8 aa, synthetic construct
FDPEDGEI

SEQ ID NO: 27 - DNA, 57 bp, synthetic construct
  source 1..57: mol_type = other DNA
gcaacggagaagcagcaactggtacgaaaatactacttctactacggtttggccgtc

SEQ ID NO: 28 - protein, 19 aa, synthetic construct
ATEKQQLVRKYYFYYGLAV

SEQ ID NO: 29 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagttatttaaattggtatcagcagaaaccagggaaagtccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtcccccattcactttcggccctgggaccaaagtggatatcaaa

SEQ ID NO: 30 - protein, 107 aa, synthetic construct
DIQMTQSPSSLSASVGDRVTITCRASQSISSYLNWYQQKPGKVPKLLIYAASSLQSGVPSRFSGSGSGTDFTLTISSLQPEDFATYYCQQSYSPPFTFGPGTKVDIK

SEQ ID NO: 31 - DNA, 18 bp, synthetic construct
  source 1..18: mol_type = other DNA
cagagcattagcagttat

SEQ ID NO: 32 - protein, 6 aa, synthetic construct
QSISSY

SEQ ID NO: 33-34 - skipped (2 entries)

SEQ ID NO: 35 - DNA, 27 bp, synthetic construct
  source 1..27: mol_type = other DNA
caacagagttacagtcccccattcact

SEQ ID NO: 36 - protein, 9 aa, synthetic construct
QQSYSPPFT

SEQ ID NO: 37 - DNA, 1362 bp, synthetic construct
  source 1..1362: mol_type = other DNA
caggtgcagctggtacagtctggggctgaggtgaagaagcctggggcctcagtgaaggtctcctgcaaggtttccggatacaccctcactgaattatccatgcactgggtgcgacaagctcctggaaaagggcttgagtggatgggaggttttgatcctgaagatggtgaaataatctacgcacagaagttccagggcagagtcaccatgaccgaggacacatctacagacacagcctacatggacctgagcagtctgacatctgaagacacggccgtgtattactgtgcaacggagaagcagcaactggtacgaaaatactacttctactacggtttggccgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 38 - protein, 453 aa, synthetic construct
QVQLVQSGAEVKKPGASVKVSCKVSGYTLTELSMHWVRQAPGKGLEWMGGFDPEDGEIIYAQKFQGRVTMTEDTSTDTAYMDLSSLTSEDTAVYYCATEKQQLVRKYYFYYGLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 645 bp, synthetic construct
  source 1..645: mol_type = other DNA
gacatccagatgacccagtctccatcctccctgtctgcatctgtaggagacagagtcaccatcacttgccgggcaagtcagagcattagcagttatttaaattggtatcagcagaaaccagggaaagtccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtcccccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 40 - protein, 214 aa, synthetic construct
DIQMTQSPSSLSASVGDRVTITCRASQSISSYLNWYQQKPGKVPKLLIYAASSLQSGVPSRFSGSGSGTDFTLTISSLQPEDFATYYCQQSYSPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 369 bp, synthetic construct
  source 1..369: mol_type = other DNA
caggtacagctgcagcagtcaggtccaggactggtgaagccctcgcagaccctctcactcacctgtgccatctccggggacagtgtctctaggaacagtgctgcttggaactggatcaggcagtccccatcgagtggccttgagtggctgggaaggacatactacaggtccaagtggttttatgattatgcaatatctgtgaaaagtcgaataaccgtcaacccagacacatccaagaaccaattctcccttcacctgaactctgtgactcccgaagacacggctgtctattactgtgcgagaggctacggtggctacgaggactactttgactactggggccagggaaccctggtcaccgtctcctca

SEQ ID NO: 42 - protein, 123 aa, synthetic construct
QVQLQQSGPGLVKPSQTLSLTCAISGDSVSRNSAAWNWIRQSPSSGLEWLGRTYYRSKWFYDYAISVKSRITVNPDTSKNQFSLHLNSVTPEDTAVYYCARGYGGYEDYFDYWGQGTLVTVSS

SEQ ID NO: 43 - DNA, 30 bp, synthetic construct
  source 1..30: mol_type = other DNA
ggggacagtgtctctaggaacagtgctgct

SEQ ID NO: 44 - protein, 10 aa, synthetic construct
GDSVSRNSAA

SEQ ID NO: 45 - DNA, 27 bp, synthetic construct
  source 1..27: mol_type = other DNA
acatactacaggtccaagtggttttat

SEQ ID NO: 46 - protein, 9 aa, synthetic construct
TYYRSKWFY

SEQ ID NO: 47 - DNA, 39 bp, synthetic construct
  source 1..39: mol_type = other DNA
gcgagaggctacggtggctacgaggactactttgactac

SEQ ID NO: 48 - protein, 13 aa, synthetic construct
ARGYGGYEDYFDY

SEQ ID NO: 49 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaagtggggtcccatcaaggttcagcggcagtggatctgggacagatttcactctcaccatcagcagcctgcagcctgaagattttgcaacttacttttgtcaacagggtagcagtttcccgtacacttttggccaggggaccaagctggagatcaaa

SEQ ID NO: 50 - protein, 107 aa, synthetic construct
DIQMTQSPSSVSASVGDRVTITCRASQGISSWLAWYQQKPGKAPKLLIYGASSLQSGVPSRFSGSGSGTDFTLTISSLQPEDFATYFCQQGSSFPYTFGQGTKLEIK

SEQ ID NO: 51 - DNA, 18 bp, synthetic construct
  source 1..18: mol_type = other DNA
cagggtattagcagctgg

SEQ ID NO: 52 - protein, 6 aa, synthetic construct
QGISSW

SEQ ID NO: 53-54 - skipped (2 entries)

SEQ ID NO: 55 - DNA, 27 bp, synthetic construct
  source 1..27: mol_type = other DNA
caacagggtagcagtttcccgtacact

SEQ ID NO: 56 - protein, 9 aa, synthetic construct
QQGSSFPYT

SEQ ID NO: 57 - DNA, 1353 bp, synthetic construct
  source 1..1353: mol_type = other DNA
caggtacagctgcagcagtcaggtccaggactggtgaagccctcgcagaccctctcactcacctgtgccatctccggggacagtgtctctaggaacagtgctgcttggaactggatcaggcagtccccatcgagtggccttgagtggctgggaaggacatactacaggtccaagtggttttatgattatgcaatatctgtgaaaagtcgaataaccgtcaacccagacacatccaagaaccaattctcccttcacctgaactctgtgactcccgaagacacggctgtctattactgtgcgagaggctacggtggctacgaggactactttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 58 - protein, 450 aa, synthetic construct
QVQLQQSGPGLVKPSQTLSLTCAISGDSVSRNSAAWNWIRQSPSSGLEWLGRTYYRSKWFYDYAISVKSRITVNPDTSKNQFSLHLNSVTPEDTAVYYCARGYGGYEDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - DNA, 645 bp, synthetic construct
  source 1..645: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatggtgcatccagtttgcaaagtggggtcccatcaaggttcagcggcagtggatctgggacagatttcactctcaccatcagcagcctgcagcctgaagattttgcaacttacttttgtcaacagggtagcagtttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 60 - protein, 214 aa, synthetic construct
DIQMTQSPSSVSASVGDRVTITCRASQGISSWLAWYQQKPGKAPKLLIYGASSLQSGVPSRFSGSGSGTDFTLTISSLQPEDFATYFCQQGSSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - DNA, 357 bp, synthetic construct
  source 1..357: mol_type = other DNA
gaggtgcagctggtggagtctgggggaggcttggtacagcctggagggtccctgagactctcctgtatagtctctggattcatcttcagtagttatgaaatgagctggctccgccaggctccagggaagggcctggagtggatttcatatattagtagtagtggtcgtgtcatatactatgcagactctgtgaagggccgattcaccatctccagagacagcgccaagaattcactgtatctggaaatgaatagtctgagagccgaagacacggctatatattattgtacgagaaagtgggatagtagtggcccatttgacttctggggccagggaaccctggtcaccgtctcctca

SEQ ID NO: 62 - protein, 119 aa, synthetic construct
EVQLVESGGGLVQPGGSLRLSCIVSGFIFSSYEMSWLRQAPGKGLEWISYISSSGRVIYYADSVKGRFTISRDSAKNSLYLEMNSLRAEDTAIYYCTRKWDSSGPFDFWGQGTLVTVSS

SEQ ID NO: 63 - DNA, 24 bp, synthetic construct
  source 1..24: mol_type = other DNA
ggattcatcttcagtagttatgaa

SEQ ID NO: 64 - protein, 8 aa, synthetic construct
GFIFSSYE

SEQ ID NO: 65 - DNA, 24 bp, synthetic construct
  source 1..24: mol_type = other DNA
attagtagtagtggtcgtgtcata

SEQ ID NO: 66 - protein, 8 aa, synthetic construct
ISSSGRVI

SEQ ID NO: 67 - DNA, 36 bp, synthetic construct
  source 1..36: mol_type = other DNA
acgagaaagtgggatagtagtggcccatttgacttc

SEQ ID NO: 68 - protein, 12 aa, synthetic construct
TRKWDSSGPFDF

SEQ ID NO: 69 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 70 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 71 - DNA, 18 bp, synthetic construct
  source 1..18: mol_type = other DNA
cagagcattagcagctat

SEQ ID NO: 72 - protein, 6 aa, synthetic construct
QSISSY

SEQ ID NO: 73-74 - skipped (2 entries)

SEQ ID NO: 75 - DNA, 30 bp, synthetic construct
  source 1..30: mol_type = other DNA
caacagagttacagtacccctccgatcacc

SEQ ID NO: 76 - protein, 10 aa, synthetic construct
QQSYSTPPIT

SEQ ID NO: 77 - DNA, 1341 bp, synthetic construct
  source 1..1341: mol_type = other DNA
gaggtgcagctggtggagtctgggggaggcttggtacagcctggagggtccctgagactctcctgtatagtctctggattcatcttcagtagttatgaaatgagctggctccgccaggctccagggaagggcctggagtggatttcatatattagtagtagtggtcgtgtcatatactatgcagactctgtgaagggccgattcaccatctccagagacagcgccaagaattcactgtatctggaaatgaatagtctgagagccgaagacacggctatatattattgtacgagaaagtgggatagtagtggcccatttgacttctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 78 - protein, 446 aa, synthetic construct
EVQLVESGGGLVQPGGSLRLSCIVSGFIFSSYEMSWLRQAPGKGLEWISYISSSGRVIYYADSVKGRFTISRDSAKNSLYLEMNSLRAEDTAIYYCTRKWDSSGPFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - DNA, 648 bp, synthetic construct
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 80 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 375 bp, synthetic construct
  source 1..375: mol_type = other DNA
caggtgcagctggtggagtctgggggaggcttggtcaagcctggagggtccctaagactctcctgtgcagcctctggattcaccttcaatgactacccaatgagctggatccgccaggctccagggaagggactggagtgggtttcatacattactagcagtagtggtagtaccatatactacgcagactctgtgaagggccgattcaccatctccagggacaacgccaagaactcactgtatctgcaaatgaacagcctgagagccgaggacacggccgtgtattactgtgcgagagaggttgtagtggctacgattgggggctactacggtatggacgtctggggccaagggaccacggtcaccgtctcctca

SEQ ID NO: 82 - protein, 125 aa, synthetic construct
QVQLVESGGGLVKPGGSLRLSCAASGFTFNDYPMSWIRQAPGKGLEWVSYITSSSGSTIYYADSVKGRFTISRDNAKNSLYLQMNSLRAEDTAVYYCAREVVVATIGGYYGMDVWGQGTTVTVSS

SEQ ID NO: 83 - DNA, 24 bp, synthetic construct
  source 1..24: mol_type = other DNA
ggattcaccttcaatgactaccca

SEQ ID NO: 84 - protein, 8 aa, synthetic construct
GFTFNDYP

SEQ ID NO: 85 - DNA, 27 bp, synthetic construct
  source 1..27: mol_type = other DNA
attactagcagtagtggtagtaccata

SEQ ID NO: 86 - protein, 9 aa, synthetic construct
ITSSSGSTI

SEQ ID NO: 87 - DNA, 51 bp, synthetic construct
  source 1..51: mol_type = other DNA
gcgagagaggttgtagtggctacgattgggggctactacggtatggacgtc

SEQ ID NO: 88 - protein, 17 aa, synthetic construct
AREVVVATIGGYYGMDV

SEQ ID NO: 89 - DNA, 324 bp, synthetic construct; identical to SEQ ID NO: 69
  source 1..324: mol_type = other DNA

SEQ ID NO: 90 - protein, 108 aa, synthetic construct; identical to SEQ ID NO: 70

SEQ ID NO: 91 - DNA, 18 bp, synthetic construct
  source 1..18: mol_type = other DNA
cagagcattagcagctat

SEQ ID NO: 92 - protein, 6 aa, synthetic construct
QSISSY

SEQ ID NO: 93-94 - skipped (2 entries)

SEQ ID NO: 95 - DNA, 30 bp, synthetic construct; identical to SEQ ID NO: 75
  source 1..30: mol_type = other DNA

SEQ ID NO: 96 - protein, 10 aa, synthetic construct
QQSYSTPPIT

SEQ ID NO: 97 - DNA, 1359 bp, synthetic construct
  source 1..1359: mol_type = other DNA
caggtgcagctggtggagtctgggggaggcttggtcaagcctggagggtccctaagactctcctgtgcagcctctggattcaccttcaatgactacccaatgagctggatccgccaggctccagggaagggactggagtgggtttcatacattactagcagtagtggtagtaccatatactacgcagactctgtgaagggccgattcaccatctccagggacaacgccaagaactcactgtatctgcaaatgaacagcctgagagccgaggacacggccgtgtattactgtgcgagagaggttgtagtggctacgattgggggctactacggtatggacgtctggggccaagggaccacggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 98 - protein, 452 aa, synthetic construct
QVQLVESGGGLVKPGGSLRLSCAASGFTFNDYPMSWIRQAPGKGLEWVSYITSSSGSTIYYADSVKGRFTISRDNAKNSLYLQMNSLRAEDTAVYYCAREVVVATIGGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - DNA, 648 bp, synthetic construct; identical to SEQ ID NO: 79
  source 1..648: mol_type = other DNA

SEQ ID NO: 100 - protein, 215 aa, synthetic construct; identical to SEQ ID NO: 80

SEQ ID NO: 101 - DNA, 378 bp, synthetic construct
  source 1..378: mol_type = other DNA
gaggtgcagctggtggagtctgggggagacttggtacagcctggagggtccctgagactctcctgtgcagcctctggattcaccttcagtagttatgaaatgaactgggtccgccaggctccagggaaggggctggagtgggtttcatacattagtaatagtggttctaccatatactacgcagactctgtgaagggccgattcaccatctccagagacaacgccgagacctcactgtatctgcaaatgaacagcctgagagccgaggacacggctgtttattactgtgcgagagagggatggggcgcatattgtgctggtgattgctattctggttttgatatttggggccaagggacaatggtcaccgtctcttca

SEQ ID NO: 102 - protein, 126 aa, synthetic construct
EVQLVESGGDLVQPGGSLRLSCAASGFTFSSYEMNWVRQAPGKGLEWVSYISNSGSTIYYADSVKGRFTISRDNAETSLYLQMNSLRAEDTAVYYCAREGWGAYCAGDCYSGFDIWGQGTMVTVSS

SEQ ID NO: 103 - DNA, 24 bp, synthetic construct
  source 1..24: mol_type = other DNA
ggattcaccttcagtagttatgaa

SEQ ID NO: 104 - protein, 8 aa, synthetic construct
GFTFSSYE

SEQ ID NO: 105 - DNA, 24 bp, synthetic construct
  source 1..24: mol_type = other DNA
attagtaatagtggttctaccata

SEQ ID NO: 106 - protein, 8 aa, synthetic construct
ISNSGSTI

SEQ ID NO: 107 - DNA, 57 bp, synthetic construct
  source 1..57: mol_type = other DNA
gcgagagagggatggggcgcatattgtgctggtgattgctattctggttttgatatt

SEQ ID NO: 108 - protein, 19 aa, synthetic construct
AREGWGAYCAGDCYSGFDI

SEQ ID NO: 109 - DNA, 324 bp, synthetic construct; identical to SEQ ID NO: 69
  source 1..324: mol_type = other DNA

SEQ ID NO: 110 - protein, 108 aa, synthetic construct; identical to SEQ ID NO: 70

SEQ ID NO: 111 - DNA, 18 bp, synthetic construct
  source 1..18: mol_type = other DNA
cagagcattagcagctat

SEQ ID NO: 112 - protein, 6 aa, synthetic construct
QSISSY

SEQ ID NO: 113-114 - skipped (2 entries)

SEQ ID NO: 115 - DNA, 30 bp, synthetic construct; identical to SEQ ID NO: 75
  source 1..30: mol_type = other DNA

SEQ ID NO: 116 - protein, 10 aa, synthetic construct
QQSYSTPPIT

SEQ ID NO: 117 - DNA, 1362 bp, synthetic construct
  source 1..1362: mol_type = other DNA
gaggtgcagctggtggagtctgggggagacttggtacagcctggagggtccctgagactctcctgtgcagcctctggattcaccttcagtagttatgaaatgaactgggtccgccaggctccagggaaggggctggagtgggtttcatacattagtaatagtggttctaccatatactacgcagactctgtgaagggccgattcaccatctccagagacaacgccgagacctcactgtatctgcaaatgaacagcctgagagccgaggacacggctgtttattactgtgcgagagagggatggggcgcatattgtgctggtgattgctattctggttttgatatttggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 118 - protein, 453 aa, synthetic construct
EVQLVESGGDLVQPGGSLRLSCAASGFTFSSYEMNWVRQAPGKGLEWVSYISNSGSTIYYADSVKGRFTISRDNAETSLYLQMNSLRAEDTAVYYCAREGWGAYCAGDCYSGFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 119 - DNA, 648 bp, synthetic construct; identical to SEQ ID NO: 79
  source 1..648: mol_type = other DNA

SEQ ID NO: 120 - protein, 215 aa, synthetic construct; identical to SEQ ID NO: 80

SEQ ID NO: 121 - DNA, 357 bp, synthetic construct
  source 1..357: mol_type = other DNA
gaggtgcagctggtggagtctggaggaggcttggtccagcctggggggtccctgagactctcctgtgcagcctctgggttcaccgtcagtagcaactacatgagctgggtccgccaggctccagggaaggggctggagtgggtctcaattatttatagcggtggtcacacatactactcagactccgtgaagggccgattcaccatctccagacacaattccaagaacactctgtatcttcaaatgaacagcctgagaggtggggacacggccgtgtattactgtgcgagagggtataccagtggctggtacggatttgacttctggggccagggaaccctggtcaccgtctcctca

SEQ ID NO: 122 - protein, 119 aa, synthetic construct
EVQLVESGGGLVQPGGSLRLSCAASGFTVSSNYMSWVRQAPGKGLEWVSIIYSGGHTYYSDSVKGRFTISRHNSKNTLYLQMNSLRGGDTAVYYCARGYTSGWYGFDFWGQGTLVTVSS

SEQ ID NO: 123 - DNA, 24 bp, synthetic construct
  source 1..24: mol_type = other DNA
gggttcaccgtcagtagcaactac

SEQ ID NO: 124 - protein, 8 aa, synthetic construct
GFTVSSNY

SEQ ID NO: 125 - DNA, 21 bp, synthetic construct
  source 1..21: mol_type = other DNA
atttatagcggtggtcacaca

SEQ ID NO: 126 - protein, 7 aa, synthetic construct
IYSGGHT

SEQ ID NO: 127 - DNA, 39 bp, synthetic construct
  source 1..39: mol_type = other DNA
gcgagagggtataccagtggctggtacggatttgacttc

SEQ ID NO: 128 - protein, 13 aa, synthetic construct
ARGYTSGWYGFDF

SEQ ID NO: 129 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcacctggttagcctggtatcagcagaaaccagggaaagcccctaagctcctgatctatgctgcatccagtttgcaaagtggggtcccgtcaagattcagcggcactggatctgggacagatttcactctcaccatcagcagcctgcagcctgaagattttgcaacttattattgtcagcagactaacagtttcccgtggacgttcggccaagggaccaaggtggaaatcaaa

SEQ ID NO: 130 - protein, 107 aa, synthetic construct
DIQMTQSPSSVSASVGDRVTITCRASQGISTWLAWYQQKPGKAPKLLIYAASSLQSGVPSRFSGTGSGTDFTLTISSLQPEDFATYYCQQTNSFPWTFGQGTKVEIK

SEQ ID NO: 131 - DNA, 18 bp, synthetic construct
  source 1..18: mol_type = other DNA
cagggtattagcacctgg

SEQ ID NO: 132 - protein, 6 aa, synthetic construct
QGISTW

SEQ ID NO: 133 - DNA, 27 bp, synthetic construct
  source 1..27: mol_type = other DNA
cagcagactaacagtttcccgtggacg

SEQ ID NO: 134 - protein, 9 aa, synthetic construct
QQTNSFPWT

SEQ ID NO: 135 - DNA, 1341 bp, synthetic construct
  source 1..1341: mol_type = other DNA
gaggtgcagctggtggagtctggaggaggcttggtccagcctggggggtccctgagactctcctgtgcagcctctgggttcaccgtcagtagcaactacatgagctgggtccgccaggctccagggaaggggctggagtgggtctcaattatttatagcggtggtcacacatactactcagactccgtgaagggccgattcaccatctccagacacaattccaagaacactctgtatcttcaaatgaacagcctgagaggtggggacacggccgtgtattactgtgcgagagggtataccagtggctggtacggatttgacttctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 136 - protein, 446 aa, synthetic construct
EVQLVESGGGLVQPGGSLRLSCAASGFTVSSNYMSWVRQAPGKGLEWVSIIYSGGHTYYSDSVKGRFTISRHNSKNTLYLQMNSLRGGDTAVYYCARGYTSGWYGFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - DNA, 645 bp, synthetic construct
  source 1..645: mol_type = other DNA
gacatccagatgacccagtctccatcttccgtgtctgcatctgtaggagacagagtcaccatcacttgtcgggcgagtcagggtattagcacctggttagcctggtatcagcagaaaccagggaaagcccctaagctcctgatctatgctgcatccagtttgcaaagtggggtcccgtcaagattcagcggcactggatctgggacagatttcactctcaccatcagcagcctgcagcctgaagattttgcaacttattattgtcagcagactaacagtttcccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 138 - protein, 214 aa, synthetic construct
DIQMTQSPSSVSASVGDRVTITCRASQGISTWLAWYQQKPGKAPKLLIYAASSLQSGVPSRFSGTGSGTDFTLTISSLQPEDFATYYCQQTNSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - DNA, 357 bp, synthetic construct
  source 1..357: mol_type = other DNA
gaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatggggatcatctatcctggtgactctgataccagatacagcccgtccttccaaggccaggtcaccatgtcagccgacaagtccatcaggatcgcctacctgcagtggagcagcctgaaggcctcggacaccgccatgtattattgtgcgagacttgattatagcggcagctggtttgactactggggccagggaaccctggtcaccgtctcctca

SEQ ID NO: 140 - protein, 119 aa, synthetic construct
EVQLVQSGAEVKKPGESLKISCKGSGYSFTSYWIAWVRQMPGKGLEWMGIIYPGDSDTRYSPSFQGQVTMSADKSIRIAYLQWSSLKASDTAMYYCARLDYSGSWFDYWGQGTLVTVSS

SEQ ID NO: 141 - DNA, 24 bp, synthetic construct
  source 1..24: mol_type = other DNA
ggatacagctttaccagctactgg

SEQ ID NO: 142 - protein, 8 aa, synthetic construct
GYSFTSYW

SEQ ID NO: 143 - DNA, 24 bp, synthetic construct
  source 1..24: mol_type = other DNA
atctatcctggtgactctgatacc

SEQ ID NO: 144 - protein, 8 aa, synthetic construct
IYPGDSDT

SEQ ID NO: 145 - DNA, 36 bp, synthetic construct
  source 1..36: mol_type = other DNA
gcgagacttgattatagcggcagctggtttgactac

SEQ ID NO: 146 - protein, 12 aa, synthetic construct
ARLDYSGSWFDY

SEQ ID NO: 147 - DNA, 324 bp, synthetic construct
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 148 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 149 - DNA, 21 bp, synthetic construct
  source 1..21: mol_type = other DNA
cagtcagtctctagctcttat

SEQ ID NO: 150 - protein, 7 aa, synthetic construct
QSVSSSY

SEQ ID NO: 151 - skipped

SEQ ID NO: 152 - DNA, 27 bp, synthetic construct
  source 1..27: mol_type = other DNA
caacagtacggaagcagcccgtggacg

SEQ ID NO: 153 - protein, 9 aa, synthetic construct
QQYGSSPWT

SEQ ID NO: 154 - DNA, 1341 bp, synthetic construct
  source 1..1341: mol_type = other DNA
gaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatggggatcatctatcctggtgactctgataccagatacagcccgtccttccaaggccaggtcaccatgtcagccgacaagtccatcaggatcgcctacctgcagtggagcagcctgaaggcctcggacaccgccatgtattattgtgcgagacttgattatagcggcagctggtttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 155 - protein, 446 aa, synthetic construct
EVQLVQSGAEVKKPGESLKISCKGSGYSFTSYWIAWVRQMPGKGLEWMGIIYPGDSDTRYSPSFQGQVTMSADKSIRIAYLQWSSLKASDTAMYYCARLDYSGSWFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - DNA, 648 bp, synthetic construct
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 157 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - DNA, 366 bp, synthetic construct
  source 1..366: mol_type = other DNA
caggtccagctggtgcagtctggggctgaggtgaagaagcctggggcctcagtgaaggtctcctgcaaggcttctggatacaccttcaacagttatgatgtcaactgggtgcgacaggccactggacaagggcttgagtggatgggatggatgaaccctcacagtggtaacacaggctacgcacagaagttccagggcagagtcactatgaccagggacacctccacaagcacaagctatatggagttgagcagcctgacatctgaggacacggccgtatattactgtgcgagaggccctttttctctactttcagctgaattcttccagcactggggccagggcaccctggtcaccgtctcctca

SEQ ID NO: 159 - protein, 122 aa, synthetic construct
QVQLVQSGAEVKKPGASVKVSCKASGYTFNSYDVNWVRQATGQGLEWMGWMNPHSGNTGYAQKFQGRVTMTRDTSTSTSYMELSSLTSEDTAVYYCARGPFSLLSAEFFQHWGQGTLVTVSS

SEQ ID NO: 160 - DNA, 24 bp, synthetic construct
  source 1..24: mol_type = other DNA
ggatacaccttcaacagttatgat

SEQ ID NO: 161 - protein, 8 aa, synthetic construct
GYTFNSYD

SEQ ID NO: 162 - DNA, 24 bp, synthetic construct
  source 1..24: mol_type = other DNA
atgaaccctcacagtggtaacaca

SEQ ID NO: 163 - protein, 8 aa, synthetic construct
MNPHSGNT

SEQ ID NO: 164 - DNA, 45 bp, synthetic construct
  source 1..45: mol_type = other DNA
gcgagaggccctttttctctactttcagctgaattcttccagcac

SEQ ID NO: 165 - protein, 15 aa, synthetic construct
ARGPFSLLSAEFFQH

SEQ ID NO: 166 - DNA, 1350 bp, synthetic construct
  source 1..1350: mol_type = other DNA
caggtccagctggtgcagtctggggctgaggtgaagaagcctggggcctcagtgaaggtctcctgcaaggcttctggatacaccttcaacagttatgatgtcaactgggtgcgacaggccactggacaagggcttgagtggatgggatggatgaaccctcacagtggtaacacaggctacgcacagaagttccagggcagagtcactatgaccagggacacctccacaagcacaagctatatggagttgagcagcctgacatctgaggacacggccgtatattactgtgcgagaggccctttttctctactttcagctgaattcttccagcactggggccagggcaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 167 - protein, 449 aa, synthetic construct
QVQLVQSGAEVKKPGASVKVSCKASGYTFNSYDVNWVRQATGQGLEWMGWMNPHSGNTGYAQKFQGRVTMTRDTSTSTSYMELSSLTSEDTAVYYCARGPFSLLSAEFFQHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - DNA, 357 bp, synthetic construct
  source 1..357: mol_type = other DNA
caggtccagctggtacagtctggggctgaggtgaagaagcctgggtcctcggtgaaggtctcctgcaaggcttctggagacaccttcagtaactatgttatcggctgggtgcgacaggcccctggacaagggcttgagtggatgggagggatcatccctatctttggtacaacaaactacgcacagcagttccagggcagagtcacgattaccacggacgaatccacgagcacggcctacatggagctgagcagcctgagatctgaggacacggccgtgtattactgtgcgagagatgggaactacggtgactactttgactactggggccagggaaccctggtcaccgtctcctca

SEQ ID NO: 169 - protein, 119 aa, synthetic construct
QVQLVQSGAEVKKPGSSVKVSCKASGDTFSNYVIGWVRQAPGQGLEWMGGIIPIFGTTNYAQQFQGRVTITTDESTSTAYMELSSLRSEDTAVYYCARDGNYGDYFDYWGQGTLVTVSS

SEQ ID NO: 170 - DNA, 24 bp, synthetic construct
  source 1..24: mol_type = other DNA
ggagacaccttcagtaactatgtt

SEQ ID NO: 171 - protein, 8 aa, synthetic construct
GDTFSNYV

SEQ ID NO: 172 - DNA, 24 bp, synthetic construct
  source 1..24: mol_type = other DNA
atcatccctatctttggtacaaca

SEQ ID NO: 173 - protein, 8 aa, synthetic construct
IIPIFGTT

SEQ ID NO: 174 - DNA, 36 bp, synthetic construct
  source 1..36: mol_type = other DNA
gcgagagatgggaactacggtgactactttgactac

SEQ ID NO: 175 - protein, 12 aa, synthetic construct
ARDGNYGDYFDY

SEQ ID NO: 176 - DNA, 1341 bp, synthetic construct
  source 1..1341: mol_type = other DNA
caggtccagctggtacagtctggggctgaggtgaagaagcctgggtcctcggtgaaggtctcctgcaaggcttctggagacaccttcagtaactatgttatcggctgggtgcgacaggcccctggacaagggcttgagtggatgggagggatcatccctatctttggtacaacaaactacgcacagcagttccagggcagagtcacgattaccacggacgaatccacgagcacggcctacatggagctgagcagcctgagatctgaggacacggccgtgtattactgtgcgagagatgggaactacggtgactactttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 177 - protein, 446 aa, synthetic construct
QVQLVQSGAEVKKPGSSVKVSCKASGDTFSNYVIGWVRQAPGQGLEWMGGIIPIFGTTNYAQQFQGRVTITTDESTSTAYMELSSLRSEDTAVYYCARDGNYGDYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 178 - DNA, 351 bp, synthetic construct
  source 1..351: mol_type = other DNA
caggttcagctggtgcagtctggagctgaggtgaagaagcctggggcctcagtgaaggtctcctgcaagacttctggttacagattcgtcaactatggtttcagctgggtgcgccaggcccctggacaaggccttgaatggatgggatggatcagcccttataatggtaacacaaactatatacagaatctccaggacagaatcaccatgaccacagacacctctacgaacacagcctacatggaactgacgaacctgagatctgacgacacggccgtatattactgtgcgaccttaactggggttcactttgactactggggccagggaaccctggtcaccgtctcctca

SEQ ID NO: 179 - protein, 117 aa, synthetic construct
QVQLVQSGAEVKKPGASVKVSCKTSGYRFVNYGFSWVRQAPGQGLEWMGWISPYNGNTNYIQNLQDRITMTTDTSTNTAYMELTNLRSDDTAVYYCATLTGVHFDYWGQGTLVTVSS

SEQ ID NO: 180 - DNA, 24 bp, synthetic construct
  source 1..24: mol_type = other DNA
ggttacagattcgtcaactatggt

SEQ ID NO: 181 - protein, 8 aa, synthetic construct
GYRFVNYG

SEQ ID NO: 182 - DNA, 24 bp, synthetic construct
  source 1..24: mol_type = other DNA
atcagcccttataatggtaacaca

SEQ ID NO: 183 - protein, 8 aa, synthetic construct
ISPYNGNT

SEQ ID NO: 184 - DNA, 30 bp, synthetic construct
  source 1..30: mol_type = other DNA
gcgaccttaactggggttcactttgactac

SEQ ID NO: 185 - protein, 10 aa, synthetic construct
ATLTGVHFDY

SEQ ID NO: 186 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaatctcctgatctttgaaacatctcgtttacaaagtggggtcccttcgaggttcagcggcagtggttctggcacagatttcactctcaccatcaacagccttcagcctgaagattttgcaacttattactgtctacaagattacaattacccgtggacgttcggccaagggaccaaggtggaaatcaaa

SEQ ID NO: 187 - protein, 107 aa, synthetic construct
AIQMTQSPSSLSASVGDRVTITCRASQGIRNDLGWYQQKPGKAPNLLIFETSRLQSGVPSRFSGSGSGTDFTLTINSLQPEDFATYYCLQDYNYPWTFGQGTKVEIK

SEQ ID NO: 188 - DNA, 18 bp, synthetic construct
  source 1..18: mol_type = other DNA
cagggcattagaaatgat

SEQ ID NO: 189 - protein, 6 aa, synthetic construct
QGIRND

SEQ ID NO: 190-191 - skipped (2 entries)

SEQ ID NO: 192 - DNA, 27 bp, synthetic construct
  source 1..27: mol_type = other DNA
ctacaagattacaattacccgtggacg

SEQ ID NO: 193 - protein, 9 aa, synthetic construct
LQDYNYPWT

SEQ ID NO: 194 - DNA, 1335 bp, synthetic construct
  source 1..1335: mol_type = other DNA
caggttcagctggtgcagtctggagctgaggtgaagaagcctggggcctcagtgaaggtctcctgcaagacttctggttacagattcgtcaactatggtttcagctgggtgcgccaggcccctggacaaggccttgaatggatgggatggatcagcccttataatggtaacacaaactatatacagaatctccaggacagaatcaccatgaccacagacacctctacgaacacagcctacatggaactgacgaacctgagatctgacgacacggccgtatattactgtgcgaccttaactggggttcactttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 195 - protein, 444 aa, synthetic construct
QVQLVQSGAEVKKPGASVKVSCKTSGYRFVNYGFSWVRQAPGQGLEWMGWISPYNGNTNYIQNLQDRITMTTDTSTNTAYMELTNLRSDDTAVYYCATLTGVHFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - DNA, 645 bp, synthetic construct
  source 1..645: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaatctcctgatctttgaaacatctcgtttacaaagtggggtcccttcgaggttcagcggcagtggttctggcacagatttcactctcaccatcaacagccttcagcctgaagattttgcaacttattactgtctacaagattacaattacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 197 - protein, 214 aa, synthetic construct
AIQMTQSPSSLSASVGDRVTITCRASQGIRNDLGWYQQKPGKAPNLLIFETSRLQSGVPSRFSGSGSGTDFTLTINSLQPEDFATYYCLQDYNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - DNA, 351 bp, synthetic construct
  source 1..351: mol_type = other DNA
gaggtgcagctggtggagtctgggggaggcttggcccagcctggggggtccctgaaactctcttgtgaagcctctggattcacgttcagtgattctgcaatgcactgggtccgccaggcttccggaaaagggctggagtgggttggtcgtattagaagcaaagctaatagttacgcgacaggatatgctgcgtcggtgaaaggcaggttcaccatctccagagatgattcaaagaacatggcgtttctggaaatgaacagcctgaaaaccgaagacacggccgtatattactgtctccgacaaacttacggtgacccctggggccagggaaccctggtcaccgtctcctca

SEQ ID NO: 199 - protein, 117 aa, synthetic construct
EVQLVESGGGLAQPGGSLKLSCEASGFTFSDSAMHWVRQASGKGLEWVGRIRSKANSYATGYAASVKGRFTISRDDSKNMAFLEMNSLKTEDTAVYYCLRQTYGDPWGQGTLVTVSS

SEQ ID NO: 200 - DNA, 24 bp, synthetic construct
  source 1..24: mol_type = other DNA
ggattcacgttcagtgattctgca

SEQ ID NO: 201 - protein, 8 aa, synthetic construct
GFTFSDSA

SEQ ID NO: 202 - DNA, 30 bp, synthetic construct
  source 1..30: mol_type = other DNA
attagaagcaaagctaatagttacgcgaca

SEQ ID NO: 203 - protein, 10 aa, synthetic construct
IRSKANSYAT

SEQ ID NO: 204 - DNA, 24 bp, synthetic construct
  source 1..24: mol_type = other DNA
ctccgacaaacttacggtgacccc

SEQ ID NO: 205 - protein, 8 aa, synthetic construct
LRQTYGDP

SEQ ID NO: 206 - DNA, 336 bp, synthetic construct
  source 1..336: mol_type = other DNA
gatgttgtgatgactcagtctccactctccctgtccgtcacccttggacagccggcctccatctcctgcaggtctagtctaagcctcgtatacagtgatggaaacaactacttgaattggtttcagcagaggccaggccaatctccaaggcgcctactttataaagtttttaaccgggactctggggtcccagacagattcagcggcagtgggtcaggcactgatttcacactgaaaatcagcagggtggaggctgaggatgttggggtttattactgcatgcaaggaacacactggccgtggacgttcggccaagggaccaaggtggaaatcaaa

SEQ ID NO: 207 - protein, 112 aa, synthetic construct
DVVMTQSPLSLSVTLGQPASISCRSSLSLVYSDGNNYLNWFQQRPGQSPRRLLYKVFNRDSGVPDRFSGSGSGTDFTLKISRVEAEDVGVYYCMQGTHWPWTFGQGTKVEIK

SEQ ID NO: 208 - DNA, 33 bp, synthetic construct
  source 1..33: mol_type = other DNA
ctaagcctcgtatacagtgatggaaacaactac

SEQ ID NO: 209 - protein, 11 aa, synthetic construct
LSLVYSDGNNY

SEQ ID NO: 210-211 - skipped (2 entries)

SEQ ID NO: 212 - DNA, 27 bp, synthetic construct
  source 1..27: mol_type = other DNA
atgcaaggaacacactggccgtggacg

SEQ ID NO: 213 - protein, 9 aa, synthetic construct
MQGTHWPWT

SEQ ID NO: 214 - DNA, 1335 bp, synthetic construct
  source 1..1335: mol_type = other DNA
gaggtgcagctggtggagtctgggggaggcttggcccagcctggggggtccctgaaactctcttgtgaagcctctggattcacgttcagtgattctgcaatgcactgggtccgccaggcttccggaaaagggctggagtgggttggtcgtattagaagcaaagctaatagttacgcgacaggatatgctgcgtcggtgaaaggcaggttcaccatctccagagatgattcaaagaacatggcgtttctggaaatgaacagcctgaaaaccgaagacacggccgtatattactgtctccgacaaacttacggtgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 215 - protein, 444 aa, synthetic construct
EVQLVESGGGLAQPGGSLKLSCEASGFTFSDSAMHWVRQASGKGLEWVGRIRSKANSYATGYAASVKGRFTISRDDSKNMAFLEMNSLKTEDTAVYYCLRQTYGDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 216 - DNA, 660 bp, synthetic construct
  source 1..660: mol_type = other DNA
gatgttgtgatgactcagtctccactctccctgtccgtcacccttggacagccggcctccatctcctgcaggtctagtctaagcctcgtatacagtgatggaaacaactacttgaattggtttcagcagaggccaggccaatctccaaggcgcctactttataaagtttttaaccgggactctggggtcccagacagattcagcggcagtgggtcaggcactgatttcacactgaaaatcagcagggtggaggctgaggatgttggggtttattactgcatgcaaggaacacactggccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 217 - protein, 219 aa, synthetic construct
DVVMTQSPLSLSVTLGQPASISCRSSLSLVYSDGNNYLNWFQQRPGQSPRRLLYKVFNRDSGVPDRFSGSGSGTDFTLKISRVEAEDVGVYYCMQGTHWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - DNA, 366 bp, synthetic construct
  source 1..366: mol_type = other DNA
caggttcagctggtgcagtctggagttgaggtgaagaagcctggggcctcagtgaaggtctcctgcaaggcttctggttacacctttaagttttatggtatcagttgggtgcgacaggcccctggacaagggcttgagtggatgggctggatcagtgtttacaatggtaaaacaaagtatgcacagaagctccagggcagagtcaccatgacaacagacacatccacgagcacagcctacatggagctgaggagcctgagatctgacgacacggccgtgtatttttgtgcgagagatggggactatgagagtagtggttatccgtttgactactggggccagggaaccctggtcaccgtctcctca

SEQ ID NO: 219 - protein, 122 aa, synthetic construct
QVQLVQSGVEVKKPGASVKVSCKASGYTFKFYGISWVRQAPGQGLEWMGWISVYNGKTKYAQKLQGRVTMTTDTSTSTAYMELRSLRSDDTAVYFCARDGDYESSGYPFDYWGQGTLVTVSS

SEQ ID NO: 220 - DNA, 24 bp, synthetic construct
  source 1..24: mol_type = other DNA
ggttacacctttaagttttatggt

SEQ ID NO: 221 - protein, 8 aa, synthetic construct
GYTFKFYG

SEQ ID NO: 222 - DNA, 24 bp, synthetic construct
  source 1..24: mol_type = other DNA
atcagtgtttacaatggtaaaaca

SEQ ID NO: 223 - protein, 8 aa, synthetic construct
ISVYNGKT

SEQ ID NO: 224 - DNA, 45 bp, synthetic construct
  source 1..45: mol_type = other DNA
gcgagagatggggactatgagagtagtggttatccgtttgactac

SEQ ID NO: 225 - protein, 15 aa, synthetic construct
ARDGDYESSGYPFDY

SEQ ID NO: 226 - DNA, 324 bp, synthetic construct; identical to SEQ ID NO: 69
  source 1..324: mol_type = other DNA

SEQ ID NO: 227 - protein, 108 aa, synthetic construct; identical to SEQ ID NO: 70

SEQ ID NO: 228 - DNA, 1350 bp, synthetic construct
  source 1..1350: mol_type = other DNA
caggttcagctggtgcagtctggagttgaggtgaagaagcctggggcctcagtgaaggtctcctgcaaggcttctggttacacctttaagttttatggtatcagttgggtgcgacaggcccctggacaagggcttgagtggatgggctggatcagtgtttacaatggtaaaacaaagtatgcacagaagctccagggcagagtcaccatgacaacagacacatccacgagcacagcctacatggagctgaggagcctgagatctgacgacacggccgtgtatttttgtgcgagagatggggactatgagagtagtggttatccgtttgactactggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 229 - protein, 449 aa, synthetic construct
QVQLVQSGVEVKKPGASVKVSCKASGYTFKFYGISWVRQAPGQGLEWMGWISVYNGKTKYAQKLQGRVTMTTDTSTSTAYMELRSLRSDDTAVYFCARDGDYESSGYPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 230 - DNA, 648 bp, synthetic construct; identical to SEQ ID NO: 79
  source 1..648: mol_type = other DNA

SEQ ID NO: 231 - protein, 215 aa, synthetic construct; identical to SEQ ID NO: 80

SEQ ID NO: 232 - protein, 353 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAG

SEQ ID NO: 233 - protein, 355 aa, Homo sapiens
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAG

SEQ ID NO: 234 - protein, 12 aa, synthetic construct
EQKLISEEDLGG

SEQ ID NO: 235 - protein, 6 aa, synthetic construct
HHHHHH

SEQ ID NO: 236 - protein, 28 aa, synthetic construct
EQKLISEEDLGGEQKLISEEDLHHHHHH

SEQ ID NO: 237 - protein, 233 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 238 - protein, 383 aa, synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGEQKLISEEDLGGEQKLISEEDLHHHHHH

SEQ ID NO: 239 - protein, 588 aa, synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 240 - protein, 379 aa, synthetic construct
EPPGPEQRVVRRAAEVPGPEPSQQEQVAFGSGDTVELSCHPPGGAPTGPTVWAKDGTGLVASHRILVGPQRLQVLNASHEDAGVYSCQHRLTRRVLCHFSVRVTDAPSSGDDEDGEDVAEDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAILGSDVEFHCKVYSDAQPHIQWLKHVEVNGSKVGPDGTPYVTVLKSWISENVEADARLRLANVSERDGGEYLCRATNFIGVAEKAFWLRVHGPQAAEEELMETDEAGSVYAGEQKLISEEDLGGEQKLISEEDLHHHHHH

SEQ ID NO: 241 - protein, 381 aa, synthetic construct
ESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEQKLISEEDLGGEQKLISEEDLHHHHHH

SEQ ID NO: 242 - protein, 383 aa, synthetic construct
ESLGTEQRVVGRVAEVSGPEPSQQEQLVFGSGDAVELSCPPPGGGPMGPTVWVKDGAGLVPSERVLVGPQRLQVLNASHEDSGAYSCRQRLTQLVLCHFSVRVTDAPSSGDDEDGEDEAEDTGVDTGAPYWTRPERMDKKLLAVPAANTVRFRCPAAGNPTPSISWLKNGKEFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGEQKLISEEDLGGEQKLISEEDLHHHHHH

SEQ ID NO: 243 - protein, 457 aa, synthetic construct
EVQLVESGGGLVQPGGSLRLSCAASGFTFTSTGISWVRQAPGKGLEWVGRIYPTSGSTNYADSVKGRFTISADTSKNTAYLQMNSLRAEDTAVYYCARTYGIYDLYVDYTEYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, synthetic construct
DIQMTQSPSSLSASVGDRVTITCRASQDVDTSLAWYKQKPGKAPKLLIYSASFLYSGVPSRFSGSGSGTDFTLTISSLQPEDFATYYCQQSTGHPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 29 aa, Mus musculus
MHRPRRRGTRPPPLALLAALLLAARGADA

SEQ ID NO: 246 - protein, 15 aa, synthetic construct
GGGGSGGGGSGGGGS

SEQ ID NO: 247 - protein, 6 aa, synthetic construct
LLQGSG

SEQ ID NO: 248 - RNA, 16 nt, synthetic construct
  source 1..16: mol_type = other RNA
gttttagagctatgct

SEQ ID NO: 249 - RNA, 22 nt, synthetic construct
  source 1..22: mol_type = other RNA
gttttagagctatgctgttttg

SEQ ID NO: 250 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 251 - RNA, 72 nt, synthetic construct
  source 1..72: mol_type = other RNA
aaacagcatagcaagttaaaataaggctagtccgttatcaacttgaaaaagtggcaccgagtcggtgctttt

SEQ ID NO: 252 - RNA, 82 nt, synthetic construct
  source 1..82: mol_type = other RNA
gttggaaccattcaaaacagcatagcaagttaaaataaggctagtccgttatcaacttgaaaaagtggcaccgagtcggtgc

SEQ ID NO: 253 - RNA, 77 nt, synthetic construct
  source 1..77: mol_type = other RNA
gttttagagctagaaatagcaagttaaaataaggctagtccgttatcaacttgaaaaagtggcaccgagtcggtgct

SEQ ID NO: 254 - RNA, 82 nt, synthetic construct; identical to SEQ ID NO: 252
  source 1..82: mol_type = other RNA

SEQ ID NO: 255 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 256 - RNA, 86 nt, synthetic construct
  source 1..86: mol_type = other RNA
gtttaagagctatgctggaaacagcatagcaagtttaaataaggctagtccgttatcaacttgaaaaagtggcaccgagtcggtgc

SEQ ID NO: 257 - RNA, 83 nt, synthetic construct
  source 1..83: mol_type = other RNA
gttttagagctagaaatagcaagttaaaataaggctagtccgttatcaacttgaaaaagtggcaccgagtcggtgcttttttt

SEQ ID NO: 258 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 259 - RNA, 92 nt, synthetic construct
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 260 - RNA, 68 nt, synthetic construct
  source 1..68: mol_type = other RNA
gttttagagctagaaatagcaagttaaaataaggctagtccgttatcaacttggcaccgagtcggtgc

SEQ ID NO: 261 - RNA, 100 nt, synthetic construct
  source 1..100: mol_type = other RNA
  misc_difference 1: note = 2'-O-methyl modified adenosine, cytidine, uridine, guanosine, unknown or other nucleotide with phosphorothioate linkage
  misc_difference 2: note = 2'-O-methyl modified adenosine, cytidine, uridine, guanosine, unknown or other nucleotide with phosphorothioate linkage
  misc_difference 3: note = 2'-O-methyl modified adenosine, cytidine, uridine, guanosine, unknown or other nucleotide with phosphorothioate linkage
  misc_difference 4: note = a, c, u, g, unknown or other
  misc_difference 5: note = a, c, u, g, unknown or other
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
  misc_difference 10: note = a, c, u, g, unknown or other
  misc_difference 11: note = a, c, u, g, unknown or other
  misc_difference 12: note = a, c, u, g, unknown or other
  misc_difference 13: note = a, c, u, g, unknown or other
  misc_difference 14: note = a, c, u, g, unknown or other
  misc_difference 15: note = a, c, u, g, unknown or other
  misc_difference 16: note = a, c, u, g, unknown or other
  misc_difference 17: note = a, c, u, g, unknown or other
  misc_difference 18: note = a, c, u, g, unknown or other
  misc_difference 19: note = a, c, u, g, unknown or other
  misc_difference 20: note = a, c, u, g, unknown or other
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide
  modified_base 69: mod_base = OTHER; note = 2'-O-methyl modified nucleotide
  modified_base 70: mod_base = OTHER; note = 2'-O-methyl modified nucleotide
  modified_base 71: mod_base = OTHER; note = 2'-O-methyl modified nucleotide
  modified_base 72: mod_base = OTHER; note = 2'-O-methyl modified nucleotide
  modified_base 73: mod_base = OTHER; note = 2'-O-methyl modified nucleotide
  modified_base 74: mod_base = OTHER; note = 2'-O-methyl modified nucleotide
  modified_base 75: mod_base = OTHER; note = 2'-O-methyl modified nucleotide
  modified_base 76: mod_base = OTHER; note = 2'-O-methyl modified nucleotide
  modified_base 77: mod_base = OTHER; note = 2'-O-methyl modified nucleotide
  modified_base 78: mod_base = OTHER; note = 2'-O-methyl modified nucleotide
  modified_base 79: mod_base = OTHER; note = 2'-O-methyl modified nucleotide
  modified_base 80: mod_base = OTHER; note = 2'-O-methyl modified nucleotide
  modified_base 81: mod_base = OTHER; note = 2'-O-methyl modified nucleotide
  modified_base 82: mod_base = OTHER; note = 2'-O-methyl modified nucleotide
  modified_base 83: mod_base = OTHER; note = 2'-O-methyl modified nucleotide
  modified_base 84: mod_base = OTHER; note = 2'-O-methyl modified nucleotide
  modified_base 85: mod_base = OTHER; note = 2'-O-methyl modified nucleotide
  modified_base 86: mod_base = OTHER; note = 2'-O-methyl modified nucleotide
  modified_base 87: mod_base = OTHER; note = 2'-O-methyl modified nucleotide
  modified_base 88: mod_base = OTHER; note = 2'-O-methyl modified nucleotide
  modified_base 89: mod_base = OTHER; note = 2'-O-methyl modified nucleotide
  modified_base 90: mod_base = OTHER; note = 2'-O-methyl modified nucleotide
  modified_base 91: mod_base = OTHER; note = 2'-O-methyl modified nucleotide
  modified_base 92: mod_base = OTHER; note = 2'-O-methyl modified nucleotide
  modified_base 93: mod_base = OTHER; note = 2'-O-methyl modified nucleotide
  modified_base 94: mod_base = OTHER; note = 2'-O-methyl modified nucleotide
  modified_base 95: mod_base = OTHER; note = 2'-O-methyl modified nucleotide
  modified_base 96: mod_base = OTHER; note = 2'-O-methyl modified nucleotide
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide
nnnnnnnnnnnnnnnnnnnngttttagagctagaaatagcaagttaaaataaggctagtccgttatcaacttgaaaaagtggcaccgagtcggtgctttt

SEQ ID NO: 262-265 - skipped (4 entries)

SEQ ID NO: 266 - protein, 12 aa, synthetic construct
GSGEGEGSEGSG

SEQ ID NO: 267 - protein, 12 aa, synthetic construct
GGSEGEGSEGGS

SEQ ID NO: 268 - protein, 5 aa, synthetic construct
GGGGS

SEQ ID NO: 269 - protein, 4 aa, synthetic construct
GGGS

SEQ ID NO: 270 - protein, 7 aa, synthetic construct
PKKKRKV

SEQ ID NO: 271 - protein, 7 aa, synthetic construct
PKKKRRV

SEQ ID NO: 272 - protein, 16 aa, synthetic construct
KRPAATKKAGQAKKKK

SEQ ID NO: 273 - protein, 13 aa, synthetic construct
AHIVMVDAYKPTK

SEQ ID NO: 274 - protein, 113 aa, synthetic construct
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 275 - protein, 104 aa, synthetic construct
HHHHHHDYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 276 - protein, 10 aa, synthetic construct
ATHIKFSKRD

SEQ ID NO: 277 - protein, 14 aa, synthetic construct
VPTIVMVDAYKRYK

SEQ ID NO: 278 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 279 - protein, 12 aa, synthetic construct
KLGDIEFIKVNK

SEQ ID NO: 280 - protein, 133 aa, synthetic construct
MGSSHHHHHHSSGLVPRGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 281 - protein, 16 aa, synthetic construct
TDKDMTITFTNKKDAE

SEQ ID NO: 282 - protein, 10 aa, synthetic construct
EQKLISEEDL

SEQ ID NO: 283 - protein, 8 aa, synthetic construct
IGTRYLTR

SEQ ID NO: 284 - protein, 4 aa, synthetic construct
GLSG

SEQ ID NO: 285 - protein, 6 aa, synthetic construct
GLSGSG

SEQ ID NO: 286 - protein, 8 aa, synthetic construct
GLSGLSGS

SEQ ID NO: 287 - protein, 10 aa, synthetic construct
GLSGLSGLSG

SEQ ID NO: 288 - protein, 10 aa, synthetic construct
GLSGGSGLSG

SEQ ID NO: 289 - skipped

SEQ ID NO: 290 - protein, 5 aa, synthetic construct
LLQGG

SEQ ID NO: 291 - protein, 4 aa, synthetic construct
LLQG

SEQ ID NO: 292 - protein, 6 aa, synthetic construct
LSLSQG

SEQ ID NO: 293 - protein, 8 aa, synthetic construct
GGGLLQGG

SEQ ID NO: 294 - protein, 5 aa, synthetic construct
GLLQG

SEQ ID NO: 295 - protein, 10 aa, synthetic construct
GSPLAQSHGG

SEQ ID NO: 296 - protein, 7 aa, synthetic construct
GLLQGGG

SEQ ID NO: 297 - protein, 6 aa, synthetic construct
GLLQGG

SEQ ID NO: 298 - protein, 4 aa, synthetic construct
GLLQ

SEQ ID NO: 299 - protein, 8 aa, synthetic construct
LLQLLQGA

SEQ ID NO: 300 - protein, 5 aa, synthetic construct
LLQGA

SEQ ID NO: 301 - protein, 7 aa, synthetic construct
LLQYQGA

SEQ ID NO: 302 - protein, 6 aa, synthetic construct
LLQGSG

SEQ ID NO: 303 - protein, 6 aa, synthetic construct
LLQYQG

SEQ ID NO: 304 - protein, 7 aa, synthetic construct
LLQLLQG

SEQ ID NO: 305 - protein, 5 aa, synthetic construct
SLLQG

SEQ ID NO: 306 - protein, 5 aa, synthetic construct
LLQLQ

SEQ ID NO: 307 - protein, 6 aa, synthetic construct
LLQLLQ

SEQ ID NO: 308 - protein, 5 aa, synthetic construct
LLQGR

SEQ ID NO: 309 - DNA, 21 bp, synthetic construct
  source 1..21: mol_type = other DNA
ttgcacaacgtcacctttgag

SEQ ID NO: 310 - DNA, 20 bp, synthetic construct
  source 1..20: mol_type = other DNA
cagccacgcagagtgatgag

SEQ ID NO: 311 - DNA, 24 bp, synthetic construct
  source 1..24: mol_type = other DNA
agtacacctgcctggcgggcaatt

SEQ ID NO: 312 - DNA, 23 bp, synthetic construct
  source 1..23: mol_type = other DNA
caagtcctggatcagtgagagtg

SEQ ID NO: 313 - DNA, 18 bp, synthetic construct
  source 1..18: mol_type = other DNA
tcccgctccgacacattg

SEQ ID NO: 314 - DNA, 18 bp, synthetic construct
  source 1..18: mol_type = other DNA
aggccgacgtgcgcctcc

SEQ ID NO: 315 - DNA, 22 bp, synthetic construct
  source 1..22: mol_type = other DNA
caccgacaaggagctagaggtt

SEQ ID NO: 316 - DNA, 20 bp, synthetic construct
  source 1..20: mol_type = other DNA
accagccacgcagagtgatg

SEQ ID NO: 317 - DNA, 25 bp, synthetic construct
  source 1..25: mol_type = other DNA
tgtccttgcacaatgtcacctttga

SEQ ID NO: 318 - DNA, 22 bp, synthetic construct
  source 1..22: mol_type = other DNA
cctggatcagtgagaatgtgga

SEQ ID NO: 319 - DNA, 20 bp, synthetic construct
  source 1..20: mol_type = other DNA
ggtggctcgacagaggtact

SEQ ID NO: 320 - DNA, 20 bp, synthetic construct
  source 1..20: mol_type = other DNA
acgcctccgcctggccaatg

SEQ ID NO: 321 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 322 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 323 - protein, 735 aa, Adeno-associated virus [sequence omitted]

SEQ ID NO: 324 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacgcgcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 325 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 326 - DNA, 2244 bp, synthetic construct
  source 1..2244: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacgcccacatcgtgatggtggacgcctacaagccgacgaagcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 327 - protein, 747 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNAHIVMVDAYKPTKQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 328 - DNA, 2253 bp, synthetic construct
  source 1..2253: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacgcgagcgcccacatcgtgatggtggacgcctacaagccgacgaaggcgcaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 329 - protein, 750 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNASAHIVMVDAYKPTKAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 330 - DNA, 2256 bp, synthetic construct
  source 1..2256: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcagcgcccacatcgtgatggtggacgcctacaagccgacgaagggcagccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 331 - protein, 751 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGSAHIVMVDAYKPTKGSQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 332 - DNA, 2268 bp, synthetic construct
  source 1..2268: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcgcccacatcgtgatggtggacgcctacaagccgacgaagggcctgagcggccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 333 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGAHIVMVDAYKPTKGLSGQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 334 - DNA, 2280 bp, synthetic construct
  source 1..2280: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcagcggcgcccacatcgtgatggtggacgcctacaagccgacgaagggcctgagcggcagcggccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 335 - protein, 759 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGSGAHIVMVDAYKPTKGLSGSGQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 336 - DNA, 2292 bp, synthetic construct
  source 1..2292: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcctgagcggcagcgcccacatcgtgatggtggacgcctacaagccgacgaagggcctgagcggcctgagcggcagccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 337 - protein, 763 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGLSGSAHIVMVDAYKPTKGLSGLSGSQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 338 - DNA, 2304 bp, synthetic construct
  source 1..2304: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggcgggcaacggcctgagcggcctgagcggcctgagcggcgcccacatcgtgatggtggacgcctacaagccgacgaagggcctgagcggcctgagcggcctgagcggccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 339 - protein, 767 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQAGNGLSGLSGLSGAHIVMVDAYKPTKGLSGLSGLSGQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 340 - DNA, 2247 bp, synthetic construct
  source 1..2247: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggagcccacatcgtgatggtggacgcctacaagccgacgaagaccaccacgcagtcaaggcttcagttttctcaggccggagcgagtgacattcgggaccagtctaggaactggcttcctggaccctgttacgcccagcaggccgtatcaaagacatctgcggataacaacaacagtgaatactcgtggactggagctaccaagtaccacctcaatggcagagactctctggtgaatccgggcccggccatggcaagccacaaggacgatgaagaagccttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggccggcaacgcccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 341 - protein, 748 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGAHIVMVDAYKPTKTTTQSRLQFSQAGASDIRDQSRNWLPGPCYAQQAVSKTSADNNNSEYSWTGATKYHLNGRDSLVNPGPAMASHKDDEEAFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 342 - DNA, 2307 bp, synthetic construct
  source 1..2307: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggcctgagcggcctgagcggcctgagcggcgcccacatcgtgatggtggacgcctacaagccgacgaagggcctgagcggcctgagcggcctgagcggcaccaccacgcagtcaaggcttcagttttctcaggccggagcgagtgacattcgggaccagtctaggaactggcttcctggaccctgttacgcccagcaggccgtatcaaagacatctgcggataacaacaacagtgaatactcgtggactggagctaccaagtaccacctcaatggcagagactctctggtgaatccgggcccggccatggcaagccacaaggacgatgaagaagccttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggccggcaacgcccaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 343 - protein, 768 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGLSGLSGLSGAHIVMVDAYKPTKGLSGLSGLSGTTTQSRLQFSQAGASDIRDQSRNWLPGPCYAQQAVSKTSADNNNSEYSWTGATKYHLNGRDSLVNPGPAMASHKDDEEAFFPQSGVLIFGKQGSEKTNVDIEKVMITDEEEIRTTNPVATEQYGSVSTNLQAGNAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 344 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 345 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 346 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 347 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 348 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacgccgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 349 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDAAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 350 - DNA, 2250 bp, synthetic construct
  source 1..2250: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcagcccacatcgtgatggtggacgcctacaagccgacgaagcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 351 - protein, 749 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAAHIVMVDAYKPTKQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 352 - DNA, 2310 bp, synthetic construct
  source 1..2310: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcaggcctgagcagcggcagcggcctgagcggcgcccacatcgtgatggtggacgcctacaagccgacgaagggcctgagcggcggcagcggcctgagcggccaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 353 - protein, 769 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAGLSSGSGLSGAHIVMVDAYKPTKGLSGGSGLSGQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 354 - DNA, 2310 bp, synthetic construct
  source 1..2310: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggtggcctgagcagcggcagcggcctgagcggcgcccacatcgtgatggtggacgcctacaagccgacgaagggcctgagcggcggcagcggcctgagcggctctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgctgctcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 355 - protein, 769 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGGLSSGSGLSGAHIVMVDAYKPTKGLSGGSGLSGSGQNQQTLKFSVAGPSNMAVQGRNYIPGPSYRQQRVSTTVTQNNNSEFAAPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 356 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 357 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 358 - DNA, 2211 bp, synthetic construct
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 359 - protein, 735 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 360 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 361 - DNA, 2238 bp, synthetic construct
  source 1..2238: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacgaacaaaaactcatctcagaagaggatctgagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 362 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNEQKLISEEDLRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 363 - protein, 27 aa, Homo sapiens
SCPPPGGGPMGPTVWVKDGTGLVPSER

SEQ ID NO: 364 - protein, 12 aa, Homo sapiens
YSCRQRLTQRVL

SEQ ID NO: 365 - protein, 8 aa, Homo sapiens
LLAVPAAN

SEQ ID NO: 366 - protein, 14 aa, Homo sapiens
VLERSPHRPILQAG

SEQ ID NO: 367 - protein, 11 aa, Homo sapiens
YVTVLKSWISE

SEQ ID NO: 368 - protein, 6 aa, Homo sapiens
ADVRLR

SEQ ID NO: 369 - protein, 16 aa, Homo sapiens
LCRATNFIGVAEKAFW

SEQ ID NO: 370 - protein, 15 aa, Homo sapiens
GQQEQLVFGSGDAVE

SEQ ID NO: 371 - protein, 8 aa, Homo sapiens
VLVGPQRL

SEQ ID NO: 372 - protein, 14 aa, Homo sapiens
VLERSPHRPILQAG

SEQ ID NO: 373 - protein, 10 aa, Homo sapiens
HCKVYSDAQP

SEQ ID NO: 374 - protein, 20 aa, Homo sapiens
YVTVLKSWISESVEADVRLR

SEQ ID NO: 375 - protein, 15 aa, Homo sapiens
LCRATNFIGVAEKAF

SEQ ID NO: 376 - protein, 4 aa, Homo sapiens
ADVR

SEQ ID NO: 377 - protein, 6 aa, Homo sapiens
IGVAEK

SEQ ID NO: 378 - protein, 7 aa, Homo sapiens
GPTVWVK

SEQ ID NO: 379 - protein, 5 aa, Homo sapiens
HCKVY

SEQ ID NO: 380 - protein, 6 aa, Homo sapiens
KSWISE

SEQ ID NO: 381 - RNA, 22 nt, synthetic construct
  source 1..22: mol_type = other RNA
gcaagctgaccctgaagttcat

SEQ ID NO: 382 - RNA, 22 nt, synthetic construct
  source 1..22: mol_type = other RNA
gaacttcagggtcagcttgccg

SEQ ID NO: 383 - RNA, 21 nt, synthetic construct
  source 1..21: mol_type = other RNA
cattctatcctctagaggatg

SEQ ID NO: 384 - RNA, 19 nt, synthetic construct
  source 1..19: mol_type = other RNA
atcctctagaggatagaat

SEQ ID NO: 385 - RNA, 22 nt, synthetic construct
  source 1..22: mol_type = other RNA
  modified_base 1: mod_base = OTHER; note = 6-aminohexyl hydrogen phosphonate modified nucleotide
gcaagctgaccctgaagttctt

SEQ ID NO: 386 - RNA, 22 nt, synthetic construct
  source 1..22: mol_type = other RNA
gaacttcagggtcagcttgctt

SEQ ID NO: 387 - RNA, 21 nt, synthetic construct
  source 1..21: mol_type = other RNA
  modified_base 1: mod_base = OTHER; note = 6-aminohexyl hydrogen phosphonate modified nucleotide
caagctgaccctgaagttctt

SEQ ID NO: 388 - RNA, 21 nt, synthetic construct
  source 1..21: mol_type = other RNA
  modified_base 1: mod_base = OTHER; note = Phosphorylated nucleotide
gaacttcagggtcagcttgtt

SEQ ID NO: 389 - RNA, 21 nt, synthetic construct
  source 1..21: mol_type = other RNA
  modified_base 1: mod_base = OTHER; note = 6-aminohexyl hydrogen phosphonate modified nucleotide
gcaagctgaccctgaagtttt

SEQ ID NO: 390 - RNA, 21 nt, synthetic construct
  source 1..21: mol_type = other RNA
  modified_base 1: mod_base = OTHER; note = Phosphorylated nucleotide
aacttcagggtcagcttgctt

SEQ ID NO: 391 - RNA, 100 nt, synthetic construct
  source 1..100: mol_type = other RNA
  misc_difference 1..100: note = This sequence may encompass 95-100 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 392 - protein, 8 aa, synthetic construct
HHHHHHHH

SEQ ID NO: 393 - protein, 10 aa, synthetic construct
HHHHHHHHHH

SEQ ID NO: 394 - protein, 4 aa, synthetic construct
GGFG

SEQ ID NO: 395 - protein, 4 aa, synthetic construct
ALAL

SEQ ID NO: 396 - protein, 4 aa, synthetic construct
GFLG